(19) United States
(12) Patent Application Publication
(10) Pub. No.: US 2025/0366365 A1
(43) Pub. Date: Nov. 27, 2025

(54) ORGANIC COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., Xi'an, Shaanxi (CN)

(72) Inventors: Lingang LI, Xi'an, Shaanxi (CN); Youngkook KIM, Xi'an, Shaanxi (CN); Heming ZHANG, Xi'an, Shaanxi (CN)

(21) Appl. No.: 18/866,972

(22) PCT Filed: Jun. 9, 2023
(86) PCT No.: PCT/CN2023/099554

§ 371 Date: Nov. 18, 2024

(30) Foreign Application Priority Data
Sep. 15, 2022 (CN) 202211121384.3

(51) Int. Cl. H10K 85/60; C07B 59/00; C07D 405/12; C07D 405/14; C07D 409/12; C07D 409/14; H10K 50/15

(52) U.S. Cl. CPC H10K 85/636; C07B 59/002; C07D 405/12; C07D 405/14; C07D 409/12; C07D 409/14; H10K 85/633; H10K 50/156; H10K 85/615; H10K 85/626; H10K 85/6572; H10K 85/6574; H10K 85/6576

(57) ABSTRACT

The present invention belongs to the field of organic light-emitting materials, and particularly relates to an organic compound, an organic electroluminescent device using the organic compound, and an electronic apparatus. The organic compound has a structure represented by formula (1). The organic compound is used in the organic electroluminescent device, such that the performance of the device can be improved.

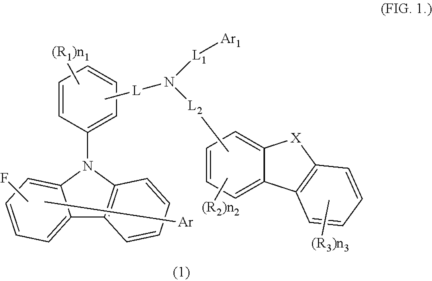

### CROSS-REFERENCE TO RELATED DISCLOSURES

The present disclosure claims the priority of Chinese patent disclosure No. 202211121384.3 filed on Sep. 15, 2022, which is incorporated herein by reference in its entirety as a part of the disclosure.

### FIELD OF THE INVENTION

The present disclosure belongs to the field of organic light-emitting materials, and particularly provides an organic compound, an organic electroluminescent device using the same, and an electronic apparatus.

### BACKGROUND OF THE INVENTION

Organic electroluminescent devices (OLED), also known as organic light-emitting diodes, refer to the phenomenon that organic light-emitting materials emit light when excited by an electric current under the influence of an electric field. It is a process of converting electrical energy into light energy. Compared to inorganic light-emitting materials, organic electroluminescent diodes (OLED) have the advantages such as active light emission, a wide range of light range, low driving voltage, high brightness, high efficiency, low energy consumption and simple fabrication process. It is because of these advantages, organic light-emitting materials and devices have become one of the most popular scientific research topics in the scientific community and industry.

Organic electroluminescent devices generally comprise an anode, a hole transport layer, an electroluminescent layer as an energy conversion layer, an electron transport layer, and a cathode sequentially stacked. When a voltage is applied to the anode and cathode, an electric field is generated between the two electrodes. Under the influence of the electric field, electrons on the cathode side move towards the electroluminescent layer, and holes on the anode side also move towards the luminescent layer. Electrons and holes combine in the electroluminescent layer to form excitons, which are in an excited state and release energy outward, thereby causing the electroluminescent layer to emit light externally.

Currently, OLED display technology has been applied in smart phones, tablet PCs and other fields, and will be further expanded to large-size applications such as TVs, but compared with the actual product application requirements, the luminous efficiency, service life and other performance of OLED devices need to be further improved. Research on the improvement of the performance of OLED light-emitting devices include: reducing the operating voltage of the device, improving the luminous efficiency of the device, improving the service life of the device and so on. In order to realize the continuous improvement of OLED device performance, it not only needs innovation in the OLED device structure and production process, but also requires continued research and innovation in optoelectronic functional materials to create higher-performance OLED functional materials.

### SUMMARY OF THE INVENTION

In view of the above problems existing in the prior art, the objective of the present disclosure is to provide an organic compound, an organic electroluminescent device using the same, and an electronic apparatus. The organic compound, when used in the organic electroluminescent device, can improve the performance of the device.

In order to achieve the above objective, according to a first aspect of the present disclosure, there is provided an organic compound having a structure as shown in Formula 1:

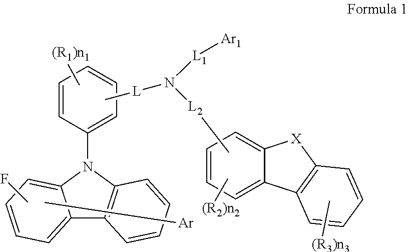

wherein X is selected from O and S;
Ar is selected from a substituted or unsubstituted aryl having 6 to 15 carbon atoms, and substituent(s) in Ar are each independently selected from a deuterium, a cyano, and an alkyl having 1 to 4 carbon atoms;
L, L₁, and L₂ are the same or different, and are each independently selected from a single bond, a substituted or unsubstituted arylene having 6 to 30 carbon atoms, and a substituted or unsubstituted heteroarylene having 5 to 30 carbon atoms;
Ar₁ is selected from a substituted or unsubstituted aryl having 6 to 40 carbon atoms, and a substituted or unsubstituted heteroaryl having 5 to 40 carbon atoms;
substituent(s) in L, L₁, L₂, and Ar₁, as well as R₁, R₂, and R₃, are the same or different, and are each independently selected from a deuterium, a cyano, an aryl having 6 to 18 carbon atoms, a heteroaryl having 5 to 18 carbon atoms, a trialkylsilyl having 3 to 12 carbon atoms, an alkyl having 1 to 10 carbon atoms, a deuteroalkyl having 1 to 10 carbon atoms, a cycloalkyl having 3 to 10 carbon atoms, and a triphenylsilyl; optionally, any two adjacent substituents in Ar₁ form a 3- to 15-membered saturated or unsaturated ring;
n₁ represents the number of R₁, and is selected from 0, 1, 2, 3, and 4; when n₁ is greater than 1, each R₁ is the same or different;
n₂ represents the number of R₂, and is selected from 0, 1, 2, and 3; when n₂ is greater than 1, each R₂ is the same or different;
n₃ represents the number of R₃, and is selected from 0, 1, 2, 3, and 4; when n₃ is greater than 1, each R₃ is the same or different.

According to a second aspect of the present disclosure, there is provided an organic electroluminescent device comprising the organic compound described in the first aspect of the present disclosure.

According to a third aspect of the present disclosure, there is provided an electronic apparatus, comprising the organic electroluminescent device described in the second aspect of the present disclosure.

The organic compound of the present disclosure has a triarylamine structure with N-phenylcarbazolyl and dibenzofuranyl(dibenzothienyl), in which a fluorine (F) and an aromatic substituent are simultaneously introduced on the benzene ring of carbazole, making the organic compound of the present disclosure both effective in preventing electron migration and promoting hole transport, and having high hole transport efficiency. In addition, the compound of the present disclosure has appropriate torque in space, thereby improving the thermal stability of the compound. Application of the compound of the present disclosure to organic electroluminescent devices can improve the lifetime and luminous efficiency of the devices.

The other features and advantages of the present disclosure will be described in detail in the following detailed description of the embodiments.

### BRIEF DESCRIPTION OF THE DRAWINGS

FIG. 1 is a schematic structural diagram of an organic electroluminescent device according to an embodiment of the present disclosure.

FIG. 2 is a schematic diagram of an electronic apparatus according to an embodiment of the present disclosure.

### LIST OF REFERENCE SIGNS

100. Anode; 200. Cathode; 300. Functional layer; 310. Hole injection layer; 320, Hole transport layer; 321. First hole transport layer; 322. Second hole transport layer; 330. Organic light emitting layer; 340. Electron transport layer; 350. Electron injection layer; 400. Electronic apparatus.

### DETAILED DESCRIPTION OF THE EMBODIMENTS

Specific embodiments of the present disclosure are described in detail below in conjunction with the accompanying drawings. It should be understood that the specific embodiments described herein are only used for the purpose of illustration and explanation of the present disclosure and are not intended to limit the present disclosure.

In a first aspect, the present disclosure provides an organic compound having a structure shown in Formula 1:

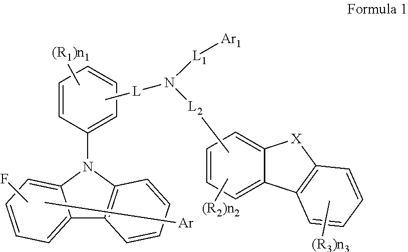

Ar is selected from a substituted or unsubstituted aryl having 6 to 15 carbon atoms, and substituent(s) in Ar are each independently selected from a deuterium, a cyano, and an alkyl having 1 to 4 carbon atoms;
L, L₁, and L₂ are the same or different, and are each independently selected from a single bond, a substituted or unsubstituted arylene having 6 to 30 carbon atoms, and a substituted or unsubstituted heteroarylene having 5 to 30 carbon atoms; Ar₁ is selected from a substituted or unsubstituted aryl having 6 to 40 carbon atoms, and a substituted or unsubstituted heteroaryl having 5 to 40 carbon atoms;
substituent(s) in L, L₁, L₂, and Ar₁, as well as R₁, R₂, and R₃, are the same or different, and are each independently selected from a deuterium, a cyano, an aryl having 6 to 18 carbon atoms, a heteroaryl having 5 to 18 carbon atoms, a trialkylsilyl having 3 to 12 carbon atoms, an alkyl having 1 to 10 carbon atoms, a deuteroalkyl having 1 to 10 carbon atoms, a cycloalkyl having 3 to 10 carbon atoms, and a triphenylsilyl; optionally, any two adjacent substituents in Ar₁ form a 3- to 15-membered saturated or unsaturated ring;
n₁ represents the number of R₁, and is selected from 0, 1, 2, 3, and 4; when n₁ is greater than 1, each R₁ is the same or different;
n₂ represents the number of R₂, and is selected from 0, 1, 2, and 3; when n₂ is greater than 1, each R₂ is the same or different;
n₃ represents the number of R₃, and is selected from 0, 1, 2, 3, and 4; when n₃ is greater than 1, each R₃ is the same or different.

In the present disclosure, the descriptive expression “be . . . each independently” may be used interchangeably with the descriptive expressions “be . . . respectively independently” and “be . . . independently selected from”, and all these expressions should be interpreted in a broad sense. They can not only mean that, in different groups, specific options expressed by the same symbols are mutul non-influential, but also mean that in the same group, specific options expressed by the same symbols mutul are non-influential. For example,

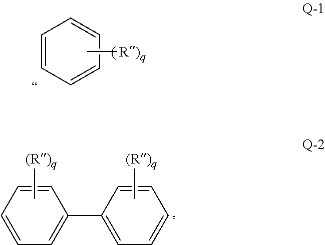

in which each q is independently 0, 1, 2, or 3, and each R″ is independently selected from a hydrogen, a deuterium, a fluorine, and a chlorine” means that the benzene ring represented by Formula Q-1 has q substituents R″, and each R″ may be the same or different, with mutual non-influence between the options for each R″; and that each of benzene rings of the biphenyl represented by Formula Q-2 has q substituents R″, and the number q of R″ on each of the two benzene rings may be the same or different and each R″ may be the same or different, with mutual non-influence between the options for each R″.

In the present disclosure, the terms “optional” and “optionally” mean that the event or circumstance described later can but not necessarily occur, and the description includes the instances where the event or circumstance does or does not occur. For example, “optionally, any two adjacent substituents form a ring” means that these two substituents may form a ring but not necessarily, including instances both where two adjacent substituents form a ring and where two adjacent substituents do not form a ring.

In the present disclosure, the term “substituted or unsubstituted” means that the functional group defined by the term may or may not have a substituent (hereinafter referred to as Rc for ease of description). For example, “a substituted or unsubstituted aryl” means an aryl having a substituent Rc or an unsubstituted aryl. Among them, the above substituent, i.e., Rc, may be, for example, a deuterium, a cyano, a heteroaryl, an aryl, an alkyl, a cycloalkyl, a deuteroalkyl, a trialkylsilyl, a triphenylsilyl, etc. A “substituted” functional group may be substituted by one or more of the above Rc substituent(s); when the same atom is connected with two substituents Rc, these two substituents may be existing independently, or may be interconnected to form a spiro ring with the atom to which they are connected; when a functional group has two adjacent substituents Rc, these two adjacent substituents may be existing independently, or may be interconnected to form a fused ring with the functional group to which they are connected.

The “ring” in the present disclosure includes a saturated ring and an unsaturated ring; the saturated ring includes a cycloalkyl and a heterocycloalkyl, while the unsaturated ring includes a cycloalkenyl, a heterocycloalkenyl, an aryl, and a heteroaryl. In the present disclosure, a ring system that is formed of n atoms is n-membered ring. For example, a phenyl is a 6-membered aryl; a fluorene ring belongs to a 13-membered ring; a cyclohexane belongs to a 6-membered ring, and an adamantane belongs to a 10-membered ring.

In the present disclosure, “any two adjacent substituents form a 3- to 15-membered saturated or unsaturated ring” and “any two adjacent substituents form a 5- to 15-membered saturated or unsaturated ring” indicate that the formed ring is a saturated or unsaturated ring, in which, the saturated ring is for example, a cyclopentane

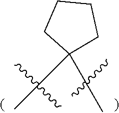 [SMILES: *C1(*)CCCC1]

and a cyclohexane

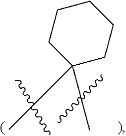 [SMILES: *C1(*)CCCCC1]

while the unsaturated ring includes benzene ring, naphthalene ring, or fluorene ring

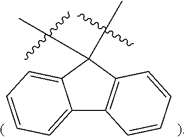 [SMILES: *C1(*)c2ccccc2-c2ccccc21]

In the present disclosure, the number of carbon atoms of a substituted or unsubstituted functional group refers to the total number of carbon atoms. For example, if L₁ is selected from a substituted arylene having 12 carbon atoms, the total number of carbon atoms in the arylene and its substituents is 12. For example, Ar₁ is

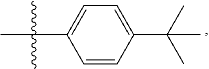 [SMILES: *c1ccc(C(C)(C)C)cc1]

then the number of carbon atoms thereof is 10; L₁ is

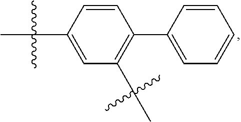 [SMILES: *c1ccc(-c2ccccc2)c(*)c1]

the number of carbon atoms thereof is 12.

In the present disclosure, an aryl refers to an optional functional group or a substituent derived from an aromatic carbon ring. The aryl can be a monocyclic aryl (e.g. a phenyl) or a polycyclic aryl. In other words, the aryl may be a monocyclic aryl, a fused aryl, two or more monocyclic aryls linked by carbon-carbon bond conjugation, a monocyclic aryl and a fused aryl linked by carbon-carbon bond conjugation, or two or more fused aryls linked by carbon-carbon bond conjugation. That is, unless otherwise specified, two or more aromatic groups linked by carbon-carbon bond conjugation can also be considered as an aryl in the present disclosure. Among them, a fused aryl may include, for example, a fused bicyclic aryl (e.g., naphthyl), a fused tricyclic aryl (e.g., phenanthryl, fluorenyl, anthryl), etc. The aryl does not contain heteroatoms such as B, N, O, S, P, Se, and Si. Examples of an aryl include, but are not limited to, a phenyl, a naphthyl, a fluorenyl, an anthryl, a phenanthryl, a biphenyl, a terphenyl, a benzo[9,10] phenanthryl, a pyrenyl, a benzofluoranthryl, and a chrysenyl, etc.

In the present disclosure, a substituted aryl may be an aryl in which one or more hydrogen atom(s) are replaced by a group such as a deuterium, a cyano, an aryl, a heteroaryl, a trialkylsilyl, an alkyl, a deuteroalkyl, a cycloalkyl, and a triphenylsilyl. It should be understood that the number of carbon atoms in the substituted aryl refers to the total number of carbon atoms in the aryl and the substituents thereon. For example, a substituted aryl having 18 carbon atoms refers to a total of 18 carbon atoms in the aryl and its substituents. In addition, in the present disclosure, the fluorenyl may be substituted. When there are two substituents, the two substituents may combine with each other to form a spiro structure. Specific examples of a substituted fluorenyl include, but are not limited to:

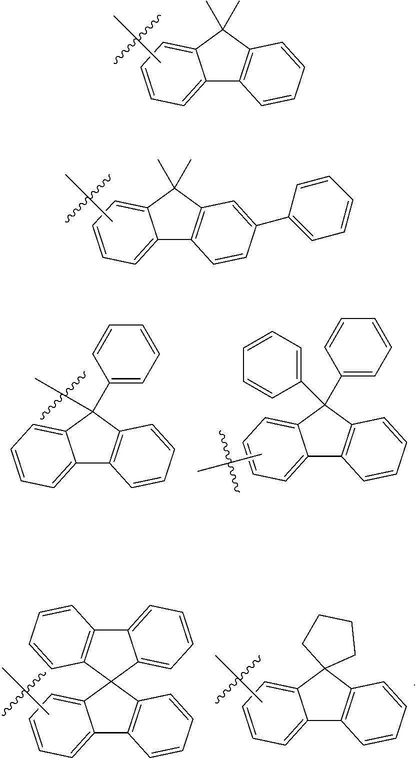

In the present disclosure, “an arylene” involved refers to a divalent group formed by further removing one hydrogen atom from an aryl.

In the present disclosure, the number of carbon atoms of a substituted or unsubstituted aryl may be 6 to 40. For example, the number of carbon atoms of the substituted or unsubstituted aryl may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40.

In the present disclosure, a heteroaryl refers to a monovalent aromatic ring or its derivative containing 1, 2, 3, 4, 5 or more heteroatoms in the ring, and the heteroatoms may be one or more of B, O, N, P, Si, Se, and S. A heteroaryl may be a monocyclic heteroaryl or a polycyclic heteroaryl. In other words, a heteroaryl may be a single aromatic ring system, or a plurality of aromatic ring systems linked by carbon-carbon bond conjugation, with any one of the aromatic ring systems being an aromatic monocyclic ring or a fused aromatic ring. For example, a heteroaryl may include, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, and N-phenylcarbazolyl, N-pyridylcarbazolyl, and N-methylcarbazolyl, etc, but not limited to thereto. In the present disclosure, “a heteroarylene” involved refers to a divalent group formed by further removing one hydrogen atom from a heteroaryl.

In the present disclosure, a substituted heteroaryl may be a heteroaryl in which one or more hydrogen atom(s) are replaced by a group such as a deuterium, a cyano, an aryl, a heteroaryl, a trialkylsilyl, an alkyl, a deuteroalkyl, a cycloalkyl, and a triphenylsilyl, etc. It should be understood that, the number of carbon atoms in the substituted heteroaryl refers to the total number of carbon atoms in the heteroaryl and the substituents thereon.

In the present disclosure, the number of carbon atoms of a substituted or unsubstituted heteroaryl may be 5 to 40. For example, the number of carbon atoms of the substituted or unsubstituted heteroaryl may be 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, and 40.

In the present disclosure, a non-positional linkage bond refers to a single bond

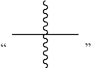

extending from a ring system, which represents that one end of the linkage bond can be linked to any position in the ring system through which the bond passes, and the other end is linked to the rest of the compound molecule.

For example, as shown in Formula (f) below, the naphthyl represented by Formula (f) is linked to other positions of the molecule through two non-positional bonds passing through the two rings, which indicates any of possible linkages shown in Formulae (f-1) to (f-10):

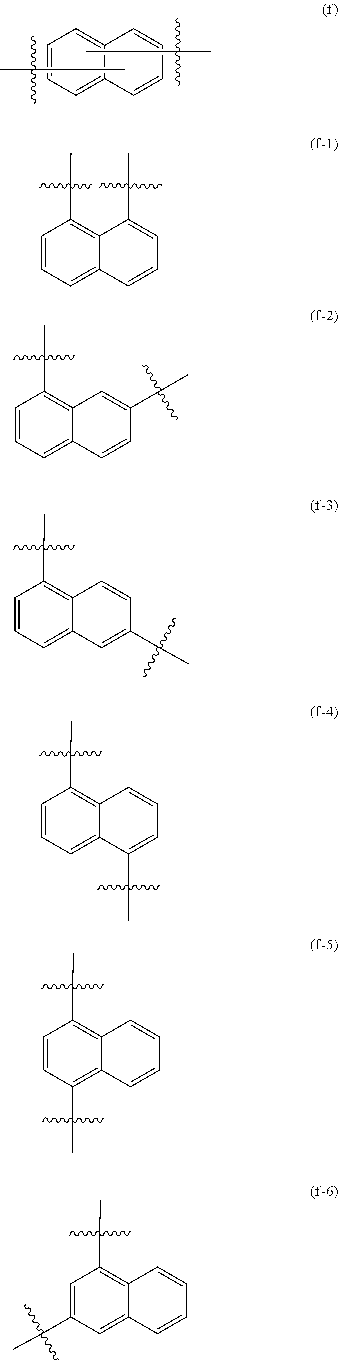

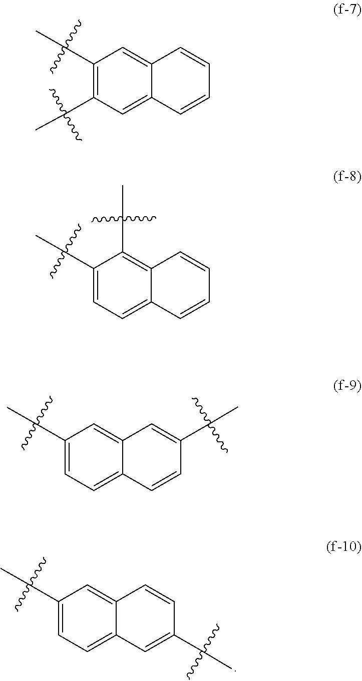 [SMILES: *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl represented by Formula (X′) is linked to other positions of the molecule via a non-positional linkage bond extending from the center of a side benzene ring, which indicates any of possible linkages shown in Formulae (X′-1) to (X′-4):

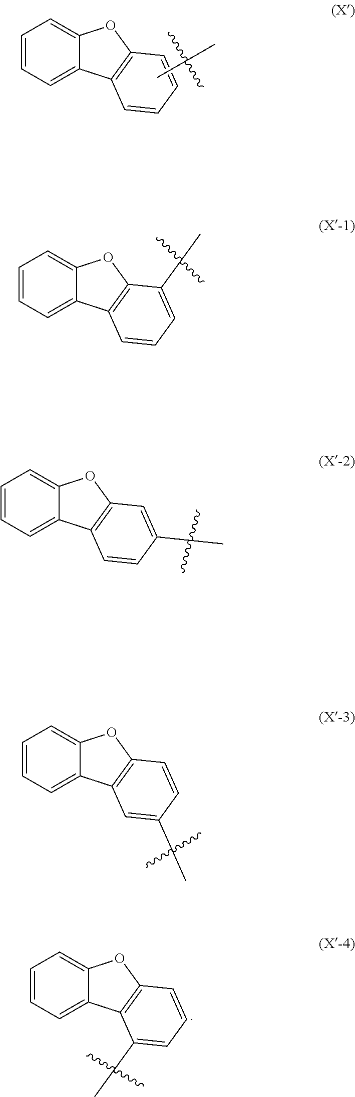

The non-positional substituent in the present disclosure refers to a substituent linked by a single bond extending from the center of the ring system, indicating that the substituent can be linked to any possible position in the ring system. For example, as shown in Formula (Y) below, the substituent R′ represented by Formula (Y) is linked to a quinoline ring via a non-positional linkage bond, which indicates any of possible linkages shown in Formulae (Y-1) to (Y-7):

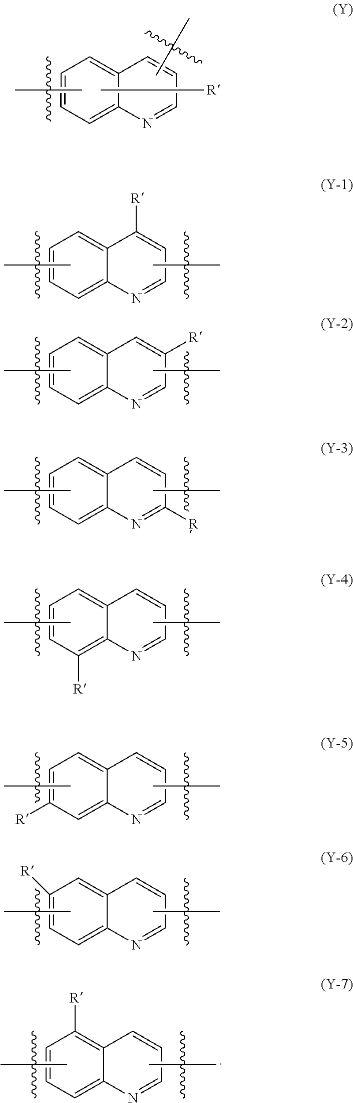

In the present disclosure, the number of carbon atoms in an alkyl may be 1 to 10, and specifically 1, 2, 3, 4, 5, 6, 7, 8, 9, and 10. The alkyl may include a straight-chain alkyl group and a branched alkyl. Specific examples of an alkyl include, but are not limited to, a methyl, an ethyl, a n-propyl, an isopropyl, a n-butyl, an isobutyl, a tert-butyl, a n-pentyl, an isopentyl, a cyclopentyl, a n-hexyl, a heptyl, a n-octyl, a 2-ethylhexyl, a nonyl, a decyl, a 3,7-dimethyloctyl, etc.

In the present disclosure, the number of carbon atoms in an aryl as a substituent may be 6 to 18, and specifically the number of carbon atoms is for example 6, 10, 12, 13, 14, and 15, etc. Specific examples of an aryl include but are not limited to a phenyl, a naphthyl, a biphenyl, a phenanthryl, an anthryl, etc.

In the present disclosure, the number of the carbon atoms of a heteroaryl as a substituent may be 5 to 18, and specifically the number of carbon atoms is for example 5, 8, 9, 10, 12, 13, 14, and 15, etc. The specific examples of the heteroaryl include, but are not limited to a pyridyl, a quinolyl, an isoquinolinyl, a dibenzofuranyl, a dibenzothienyl, and a carbazolyl, etc.

In the present disclosure, the number of carbon atoms in a trialkylsilyl as a substituent may be 3 to 12, such as 3, 6, 7, 8, and 9, etc. Specific examples thereof include but are not limited to a trimethylsilyl, an ethyldimethylsilyl, and a triethylsilyl, etc.

In the present disclosure, the number of carbon atoms in a cycloalkyl as a substituent may be 3 to 10, such as 5, 6, 8, and 10. Specific examples include, but are not limited to, a cyclopentyl, a cyclohexyl, and an adamantyl, etc.

In the present disclosure, the number of carbon atoms of a deuteroalkyl may be 1 to 10. For example, a deuteroalkyl may be a deuteroalkyl having 1 to 4 carbon atoms. Specific examples of a deuteroalkyl include, but are not limited to a trideuteromethyl.

In the present disclosure, the structure of the organic compound may be represented by Formula 1-1 or Formula 1-2:

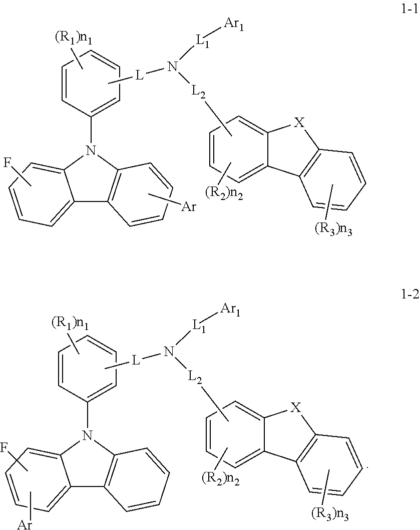

Optionally, Ar is selected from a substituted or unsubstituted phenyl, a substituted or unsubstituted naphthyl, and a substituted or unsubstituted biphenyl, and substituent(s) in Ar are each independently selected from a deuterium, a cyano, a methyl, an ethyl, an isopropyl, and a tert-butyl.

In some embodiments, Ar is selected from the group consisting of the following groups:

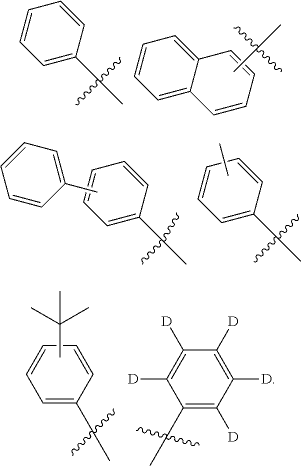

Optionally, Ar is selected from the group consisting of the following groups:

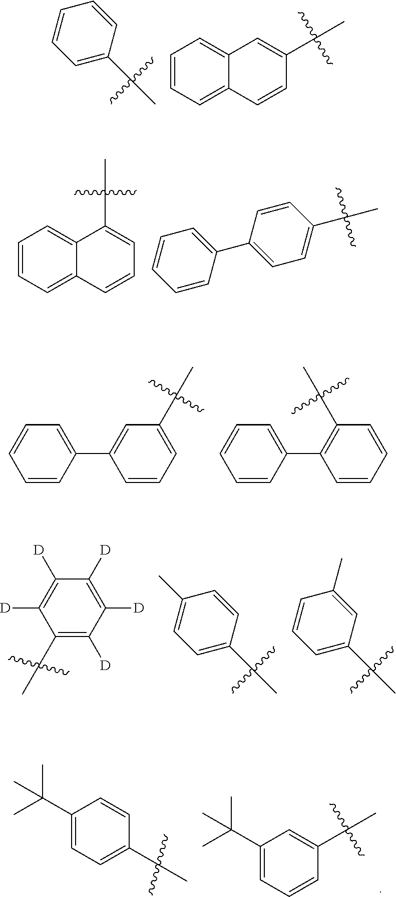

In the present disclosure, L, L₁ and L₂ are each independently selected from a single bond, a substituted or unsubstituted arylene having 6, 7, 8, 9, 10, 11, 12, 13, 14, 15, 16, 17, 18, 19, 20, 21, 22, 23, 24, 25, 26, 27, 28, 29, and 30 carbon atoms, and a substituted or unsubstituted heteroarylene having 5, 6, 7, 8, 9, 10, 11, 12, 13, 14, 15, 16, 17, 18, 19, 20, 21, 22, 23, 24, 25, 26, 27, 28, 29, and 30 carbon atoms.

In some embodiments, L, L₁, and L₂ are the same or different, and are each independently selected from a single bond, a substituted or unsubstituted arylene having 6 to 18 carbon atoms, and a substituted or unsubstituted heteroarylene having 12 to 18 carbon atoms.

Optionally, L and L₂ are each independently selected from a single bond, and a substituted or unsubstituted arylene having 6 to 15 carbon atoms.

Optionally, L₁ is selected from a single bond, a substituted or unsubstituted arylene having 6 to 15 carbon atoms, and a substituted or unsubstituted heteroarylene having 12 to 18 carbon atoms.

In some embodiments, L, L₁ and L₂ are the same or different, and are each independently selected from a single bond, a substituted or unsubstituted phenylene, a substituted or unsubstituted naphthylene, a substituted or unsubstituted biphenylene, a substituted or unsubstituted fluorenylene, a substituted or unsubstituted phenanthrylene, a substituted or unsubstituted dibenzofuranylene, a substituted or unsubstituted dibenzothienylene, and a substituted or unsubstituted carbazolylene.

In some embodiments, substituent(s) in L, L₁ and L₂ are each independently selected from a deuterium, a cyano, an aryl having 6 to 12 carbon atoms, a heteroaryl having 5 to 12 carbon atoms, a trialkylsilyl having 3 to 7 carbon atoms, and an alkyl having 1 to 4 carbon atoms.

Optionally, substituent(s) in L, L₁ and L₂ are each independently selected from a deuterium, a cyano, a methyl, an ethyl, an isopropyl, a tert-butyl, a trimethylsilyl, a phenyl, a naphthyl, a biphenyl, a dibenzofuranyl, a dibenzothienyl, and a carbazolyl.

In some embodiments, L, L₁ and L₂ are the same or different, and each independently selected from a single bond, a substituted or unsubstituted group Z, wherein the unsubstituted group Z is selected from the group consisting of the following groups:

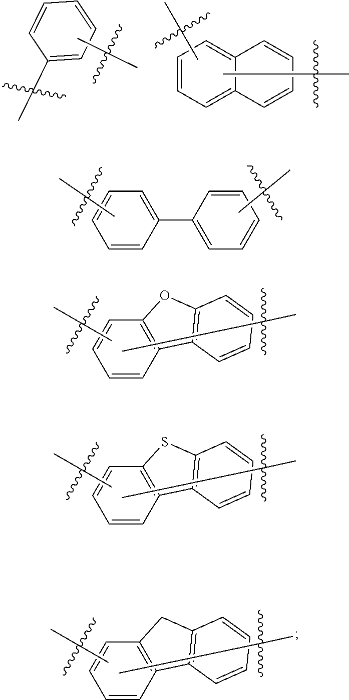

substituent(s) in the substituent group Z are each independently selected from a deuterium, a cyano, a methyl, an ethyl, an isopropyl, a tert-butyl, a trimethylsilyl, a phenyl, and a naphthyl, when the number of the substituents is greater than 1, each substituent is the same or different.

In some embodiments, L, L₁ and L₂ are each independently selected from a single bond and the group consisting of the following groups:

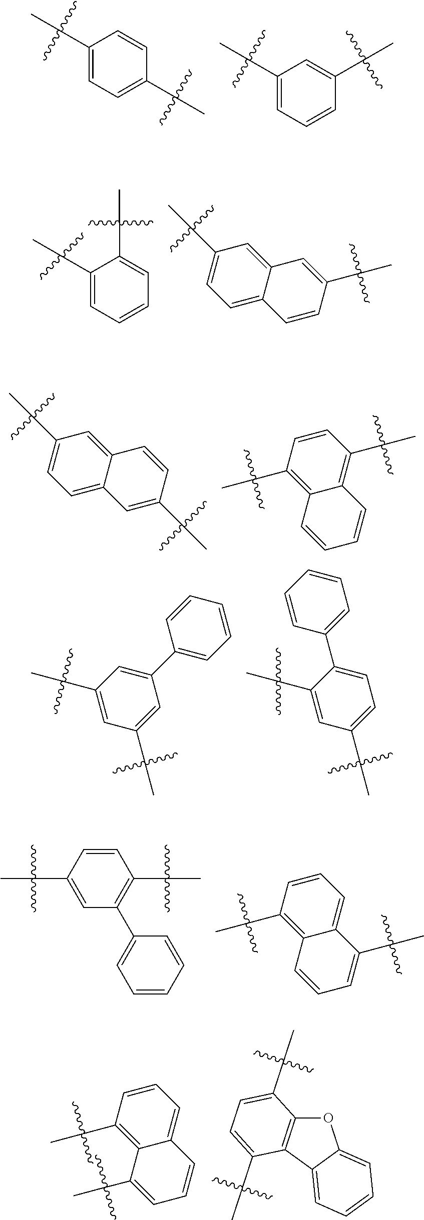

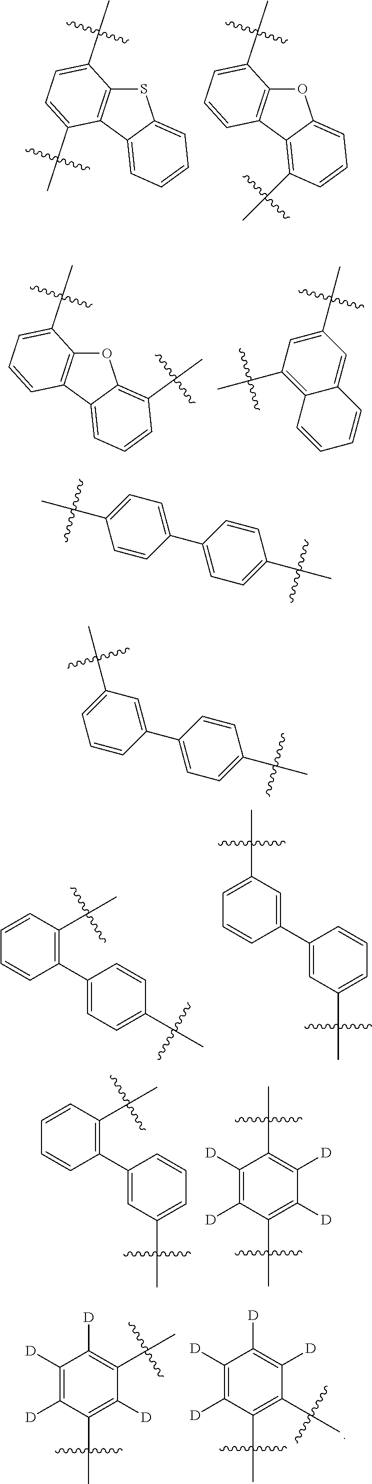

In some more specific embodiments, L and L₂ are each independently selected from a single bond and the group consisting of the following groups:

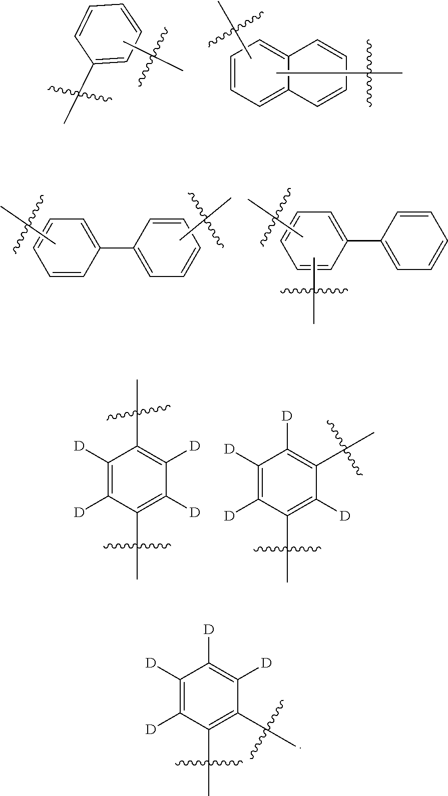

In some more specific embodiments, L₁ is selected from a single bond, and the group consisting of the following groups:

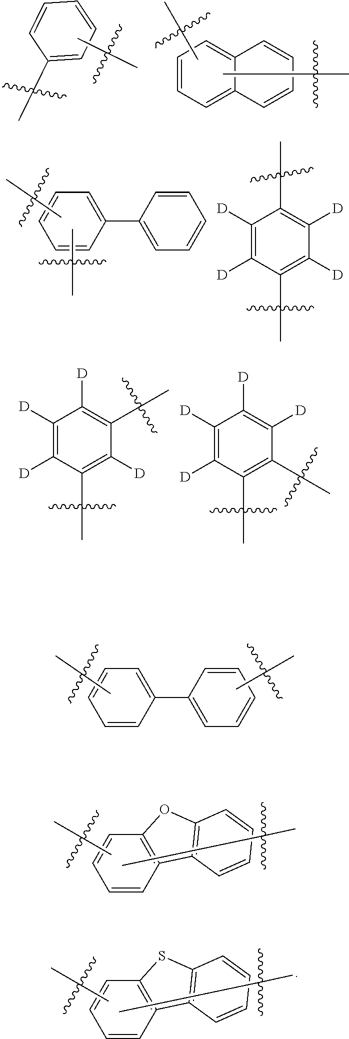

Optionally, Ar₁ is selected from a substituted or unsubstituted aryl having 6, 7, 8, 9, 10, 11, 12, 13, 14, 15, 16, 17, 18, 19, 20, 21, 22, 23, 24, 25, 26, 27, 28, 29, and 30 carbon atoms, and a substituted or unsubstituted heteroaryl having 5, 6, 7, 8, 9, 10, 11, 12, 13, 14, 15, 16, 17, 18, 19, 20, 21, 22, 23, 24, 25, 26, 27, 28, 29, and 30 carbon atoms.

In some embodiments, Ar₁ is selected from a substituted or unsubstituted aryl having 6 to 25 carbon atoms, and a substituted or unsubstituted heteroaryl having 12 to 25 carbon atoms.

In some embodiments, Ar₁ is selected from a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted anthryl, a substituted or unsubstituted phenanthryl, a substituted or unsubstituted triphenylene, a substituted or unsubstituted dibenzofuranyl, a substituted or unsubstituted dibenzothienyl, and a substituted or unsubstituted carbazolyl.

In some embodiments, substituent(s) in Ar₁ are each independently selected from a deuterium, a cyano, an aryl having 6 to 12 carbon atoms, a heteroaryl having 5 to 12 carbon atoms, a trialkylsilyl having 3 to 7 carbon atoms, an alkyl having 1 to 4 carbon atoms, a deuteroalkyl having 1 to 4 carbon atoms, and a cycloalkyl having 5 to 10 carbon atoms; optionally, any two adjacent substituents in Ar₁ form a saturated or unsaturated 5- to 15-membered ring.

Optionally, substituent(s) in Ar₁ are each independently selected from a deuterium, a cyano, a methyl, an ethyl, an isopropyl, a tert-butyl, a cyclopentyl, a cyclohexyl, a trimethylsilyl, a trideuteromethyl, a phenyl, a naphthyl, a biphenyl, a pyridyl, a dibenzofuranyl, a dibenzothienyl, and a carbazolyl; optionally, any two adjacent substituents form a benzene ring, a cyclopentane, a cyclohexane, and a fluorene ring.

In some embodiments, Ar₁ is selected from a substituted or unsubstituted group W, wherein, the unsubstituted group W is selected from the group consisting of the following groups:

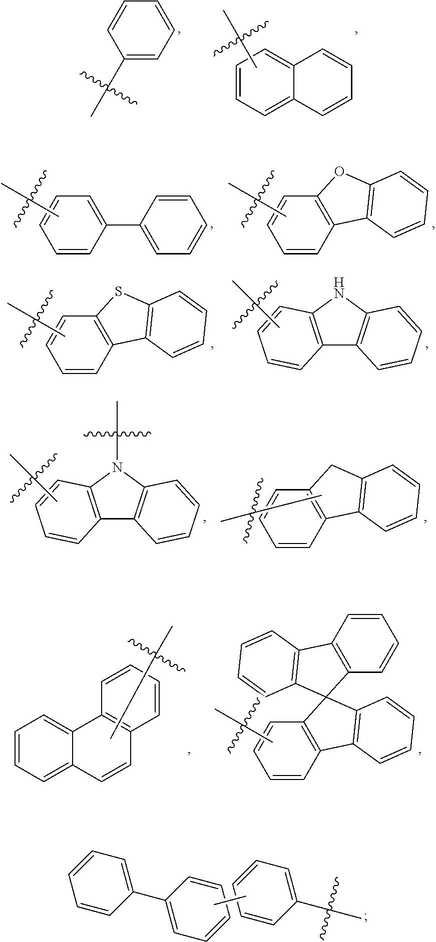

substituent(s) in the substituted group W are each independently selected from a deuterium, a cyano, a methyl, an ethyl, an isopropyl, a tert-butyl, a trideuteromethyl, a trimethylsilyl, a phenyl, and a naphthyl. When the number of substituents is greater than 1, each substituent is the same or different.

Optionally, Ar₁ is selected from the group consisting of the following groups:

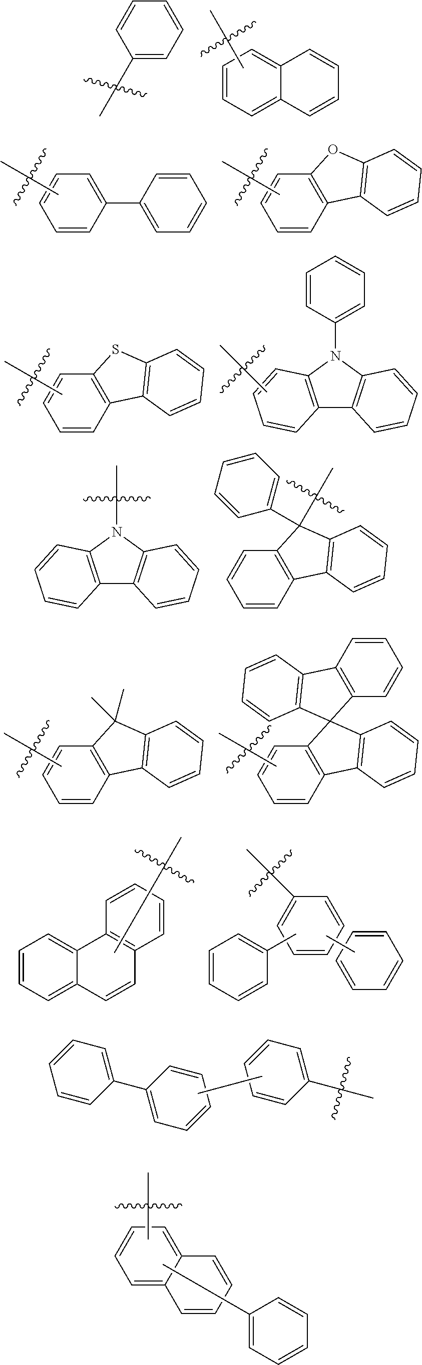

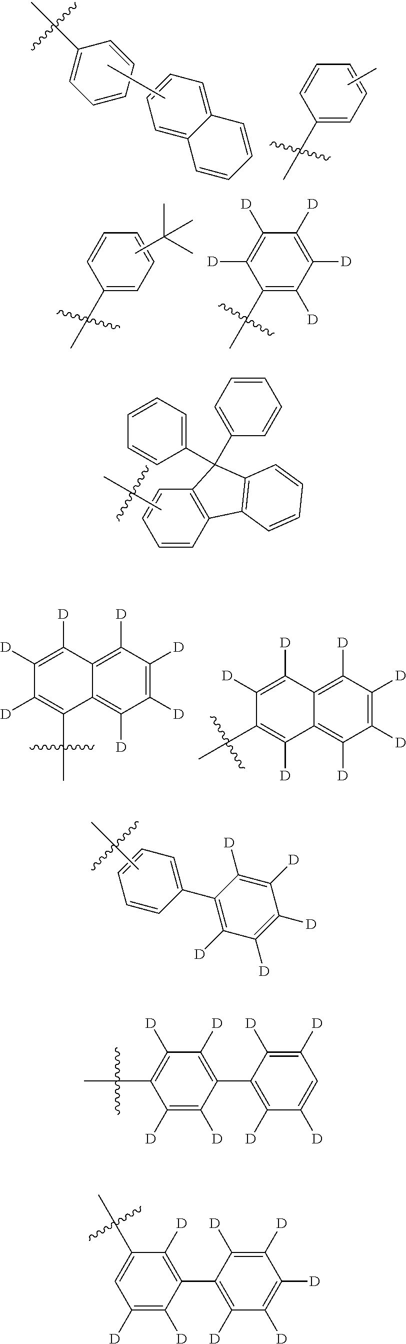

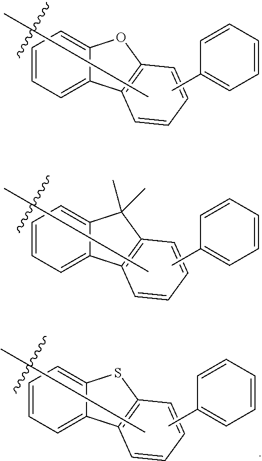

Further optionally, Ar₁ is selected from the group consisting of the following groups:

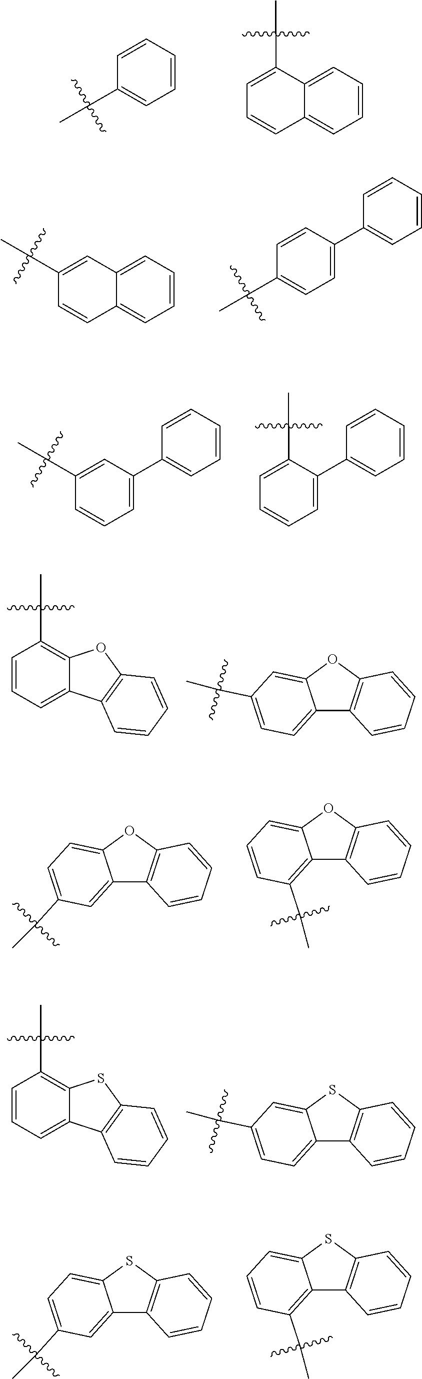

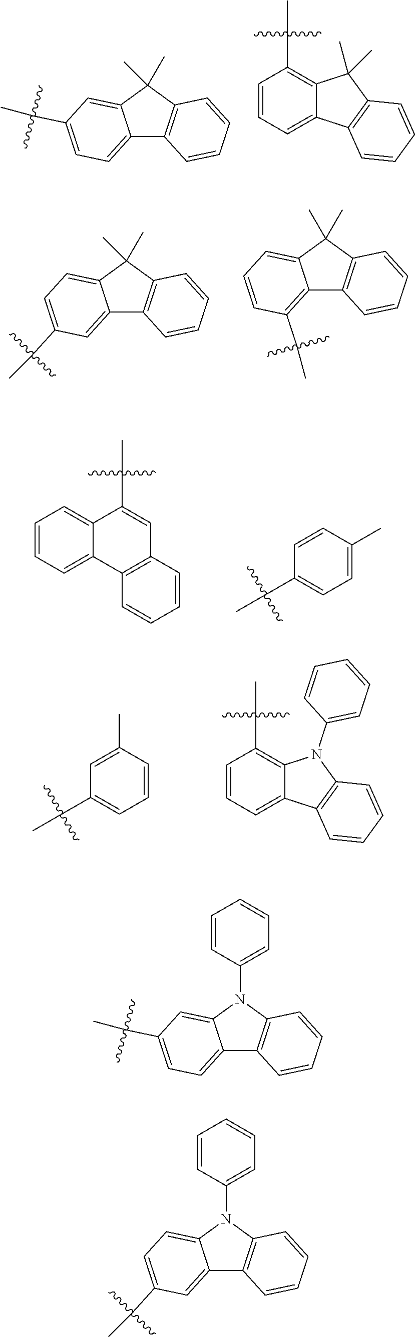

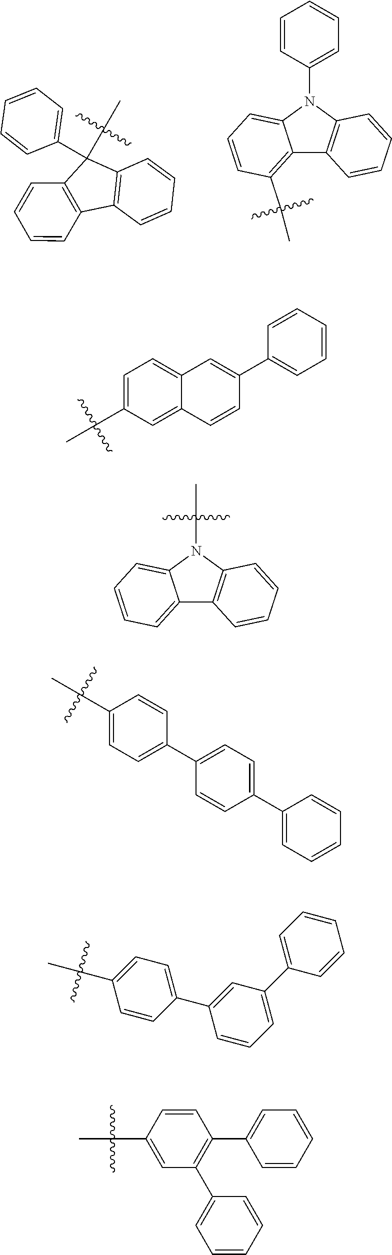

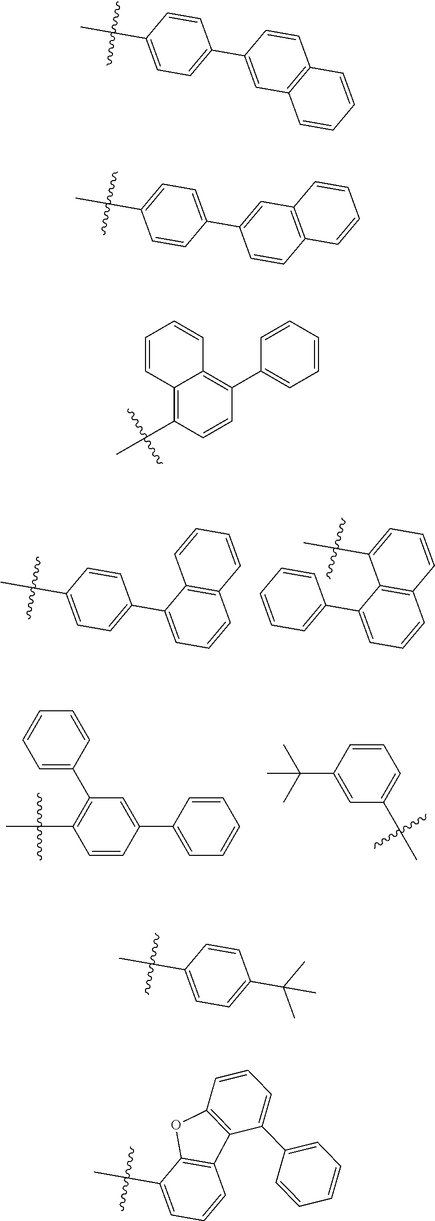

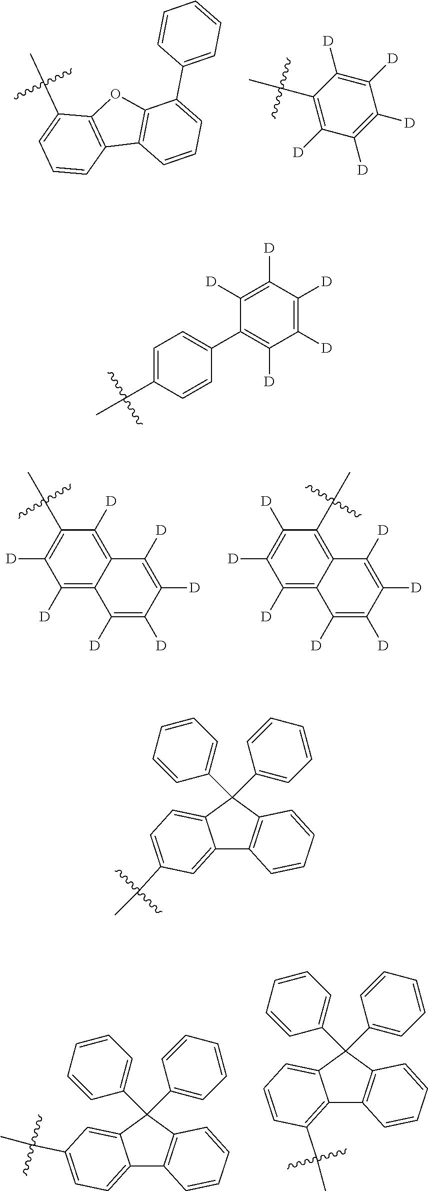

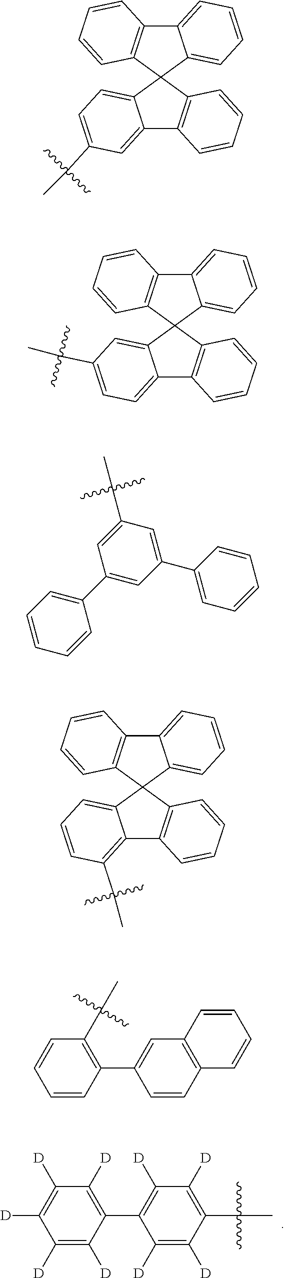 [SMILES: *c1ccc2c(c1)-c1ccccc1C21c2ccccc2-c2ccccc21 ; *c1ccc2c(c1)C1(c3ccccc3-c3ccccc31)c1ccccc1-2 ; *c1cc(-c2ccccc2)cc(-c2ccccc2)c1 ; *c1cccc2c1-c1ccccc1C21c2ccccc2-c2ccccc21 ; *c1ccccc1-c1ccc2ccccc2c1 ; *c1c([2H])c([2H])c(-c2c([2H])c([2H])c([2H])c([2H])c2[2H])c([2H])c1[2H]]

In some embodiments,

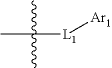

is selected from the group consisting of the following groups:

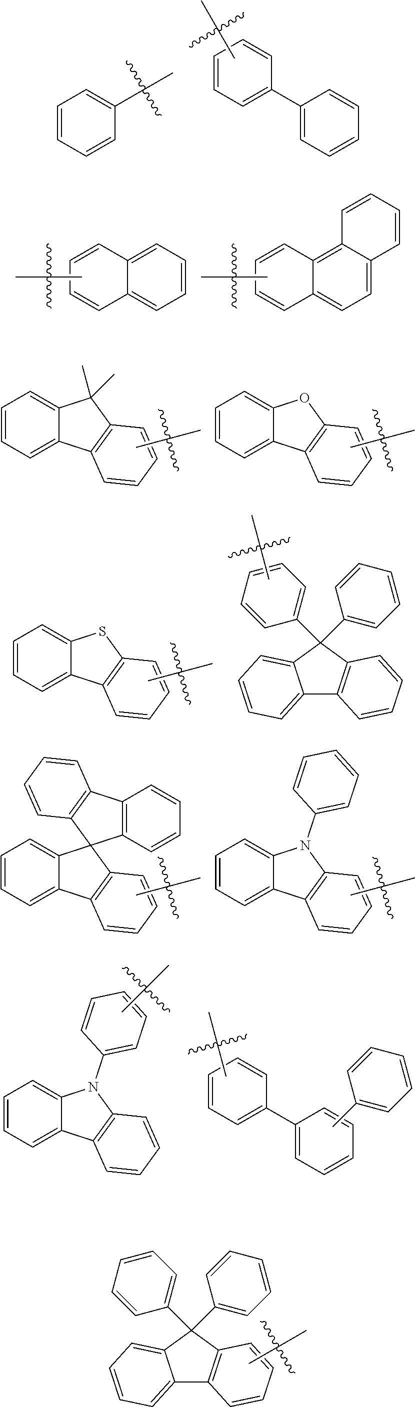

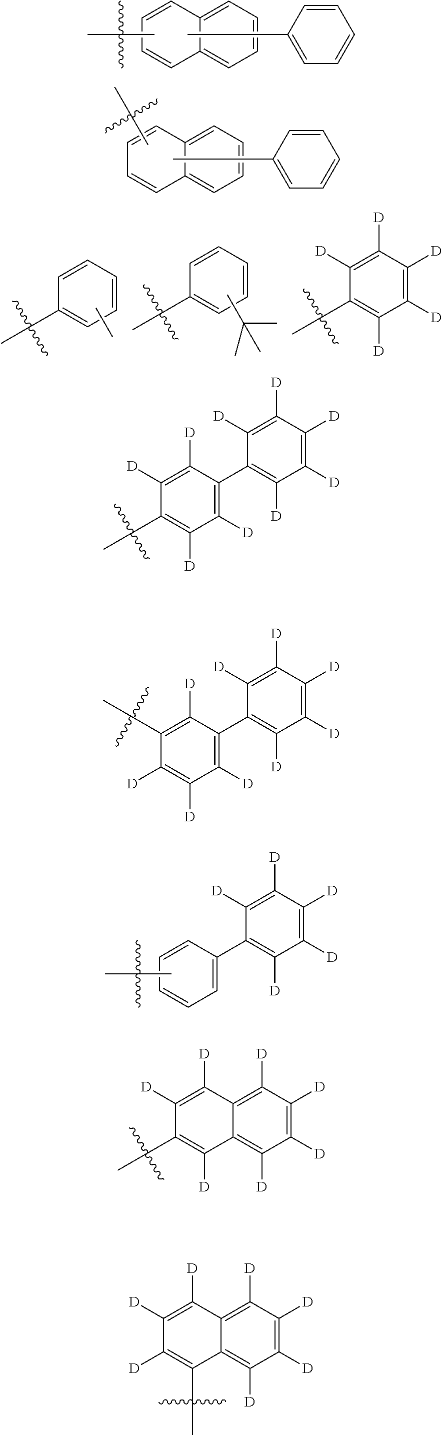

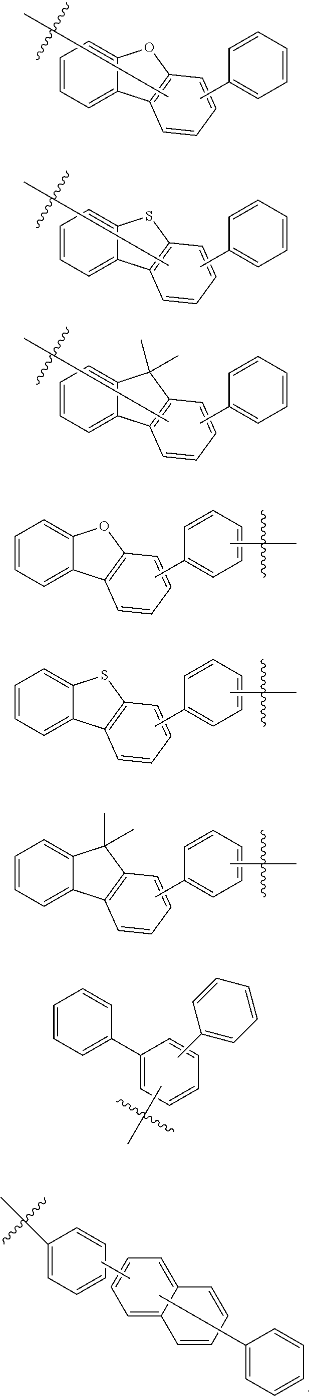

In some embodiments, R₁, R₂ and R₃ are the same or different, and are each independently selected from a deuterium, a cyano, an aryl having 6 to 12 carbon atoms, a trialkylsilyl having 3 to 7 carbon atoms, an alkyl having 1 to 4 carbon atoms, and a deuteroalkyl having 1 to 4 carbon atoms.

Optionally, R₁, R₂ and R₃ are the same or different and are each independently selected from a deuterium, a cyano, a methyl, an ethyl, an isopropyl, a tert-butyl, a trideuteromethyl, a phenyl, and a naphthyl.

Optionally, the organic compound are selected from the group consisting of the following compounds:

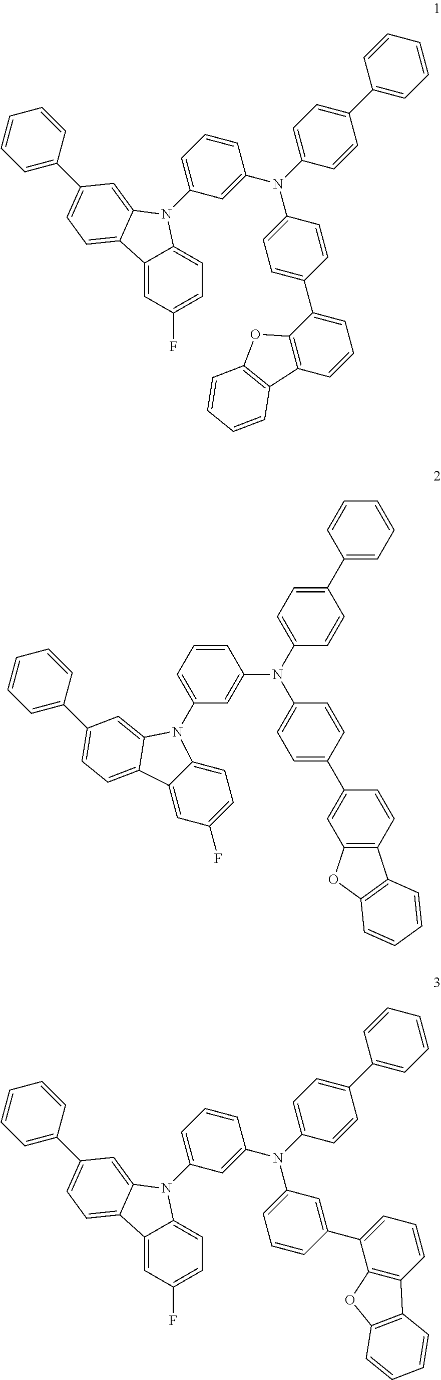 [SMILES: Fc1ccc2c(c1)c1ccc(-c3ccccc3)cc1n2-c1cccc(N(c2ccc(-c3ccccc3)cc2)c2ccc(-c3cccc4c3oc3ccccc34)cc2)c1 ; Fc1ccc2c(c1)c1ccc(-c3ccccc3)cc1n2-c1cccc(N(c2ccc(-c3ccccc3)cc2)c2ccc(-c3ccc4c(c3)oc3ccccc34)cc2)c1 ; Fc1ccc2c(c1)c1ccc(-c3ccccc3)cc1n2-c1cccc(N(c2ccc(-c3ccccc3)cc2)c2cccc(-c3cccc4c3oc3ccccc34)c2)c1]

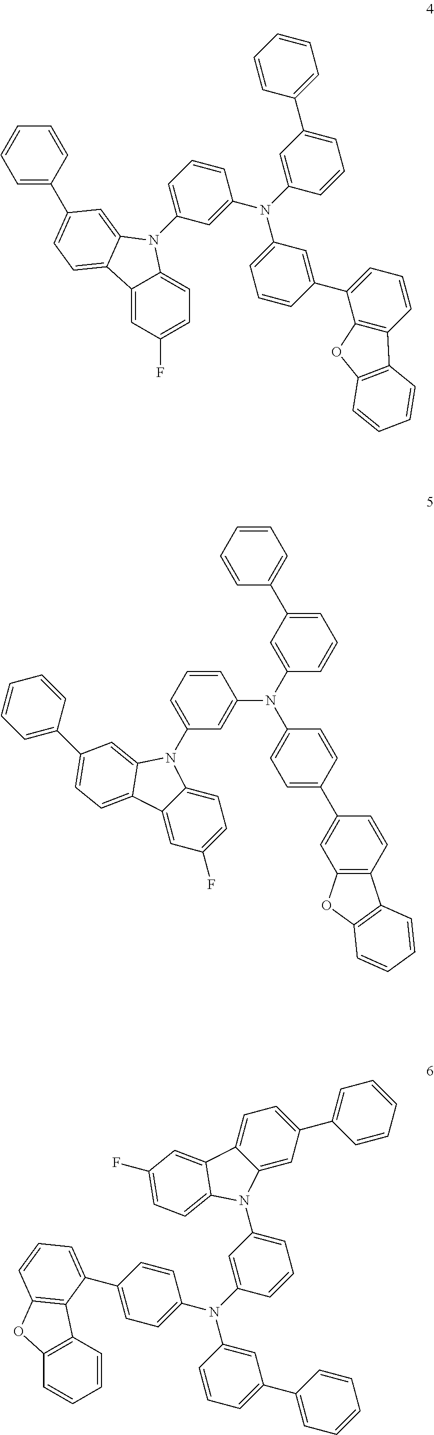 [SMILES: Fc1ccc2c(c1)c1ccc(-c3ccccc3)cc1n2-c1cccc(N(c2cccc(-c3ccccc3)c2)c2cccc(-c3cccc4c3oc3ccccc34)c2)c1 ; Fc1ccc2c(c1)c1ccc(-c3ccccc3)cc1n2-c1cccc(N(c2ccc(-c3ccc4c(c3)oc3ccccc34)cc2)c2cccc(-c3ccccc3)c2)c1 ; Fc1ccc2c(c1)c1ccc(-c3ccccc3)cc1n2-c1cccc(N(c2ccc(-c3cccc4oc5ccccc5c34)cc2)c2cccc(-c3ccccc3)c2)c1]

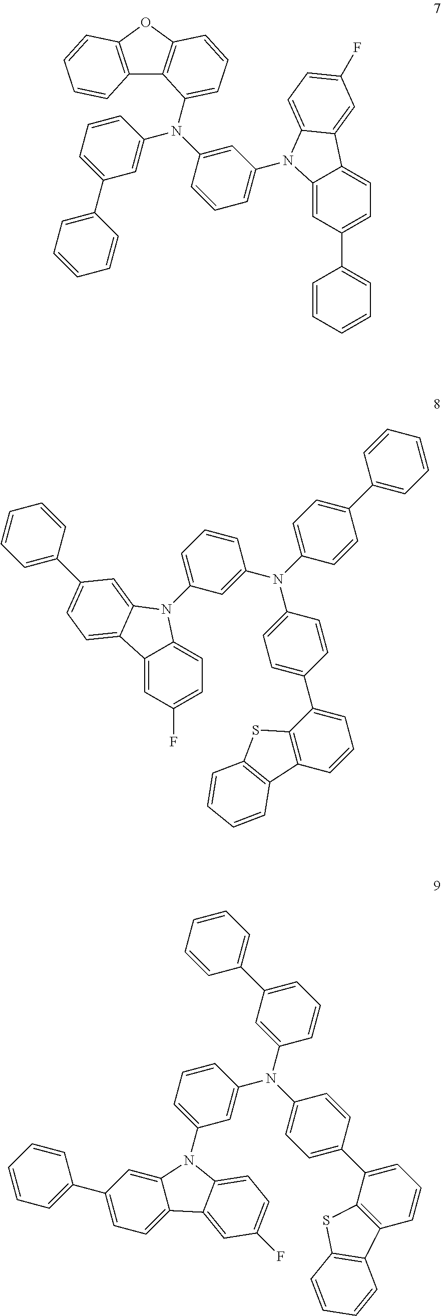 [SMILES: Fc1ccc2c(c1)c1ccc(-c3ccccc3)cc1n2-c1cccc(N(c2cccc(-c3ccccc3)c2)c2cccc3oc4ccccc4c23)c1 ; Fc1ccc2c(c1)c1ccc(-c3ccccc3)cc1n2-c1cccc(N(c2ccc(-c3ccccc3)cc2)c2ccc(-c3cccc4c3sc3ccccc34)cc2)c1 ; Fc1ccc2c(c1)c1ccc(-c3ccccc3)cc1n2-c1cccc(N(c2ccc(-c3cccc4c3sc3ccccc34)cc2)c2cccc(-c3ccccc3)c2)c1]

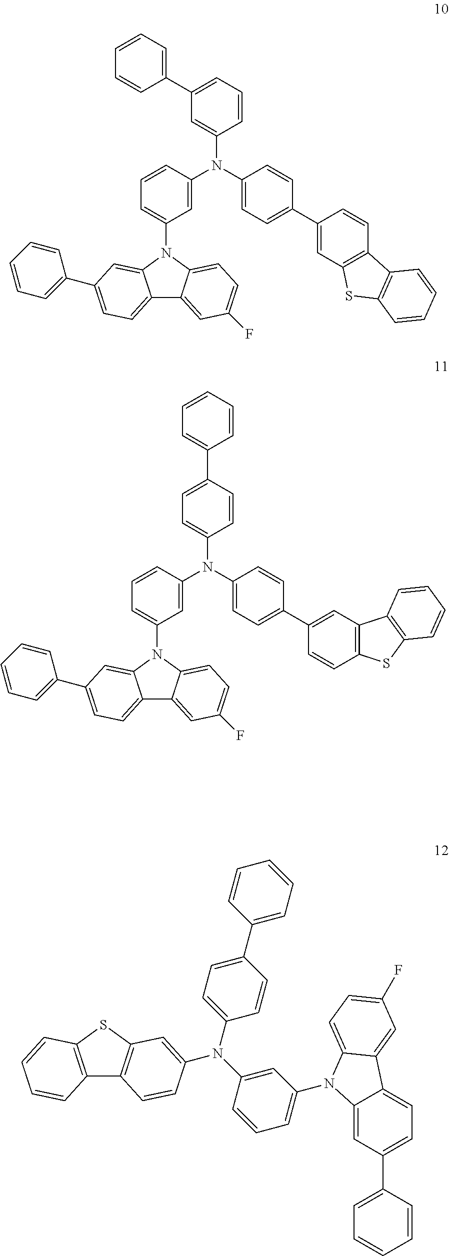 [SMILES: Fc1ccc2c(c1)c1ccc(-c3ccccc3)cc1n2-c1cccc(N(c2ccc(-c3ccc4c(c3)sc3ccccc34)cc2)c2cccc(-c3ccccc3)c2)c1 ; Fc1ccc2c(c1)c1ccc(-c3ccccc3)cc1n2-c1cccc(N(c2ccc(-c3ccccc3)cc2)c2ccc(-c3ccc4sc5ccccc5c4c3)cc2)c1 ; Fc1ccc2c(c1)c1ccc(-c3ccccc3)cc1n2-c1cccc(N(c2ccc(-c3ccccc3)cc2)c2ccc3c(c2)sc2ccccc23)c1]

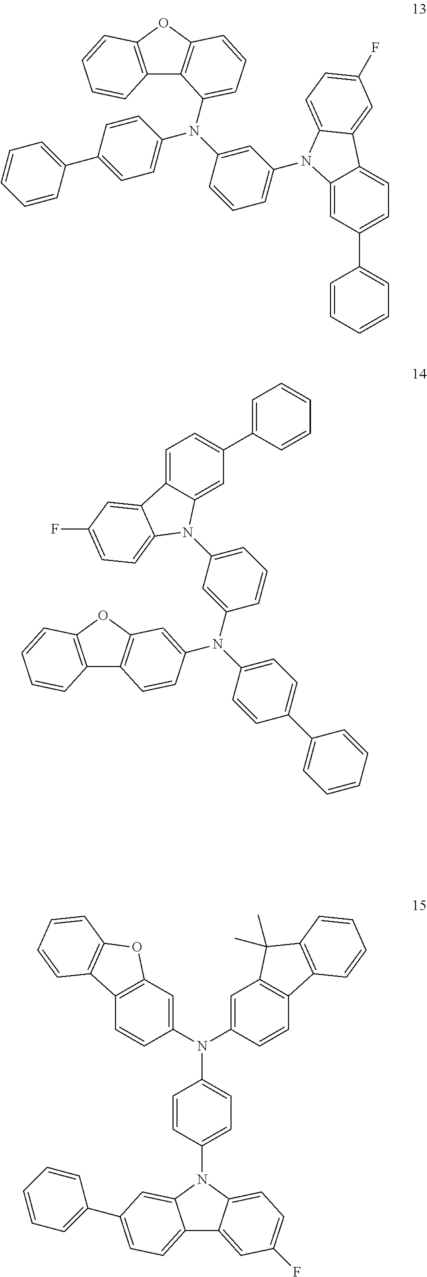 [SMILES: Fc1ccc2c(c1)c1ccc(-c3ccccc3)cc1n2-c1cccc(N(c2ccc(-c3ccccc3)cc2)c2cccc3oc4ccccc4c23)c1 ; Fc1ccc2c(c1)c1ccc(-c3ccccc3)cc1n2-c1cccc(N(c2ccc(-c3ccccc3)cc2)c2ccc3c(c2)oc2ccccc23)c1 ; CC1(C)c2ccccc2-c2ccc(N(c3ccc(-n4c5ccc(F)cc5c5ccc(-c6ccccc6)cc54)cc3)c3ccc4c(c3)oc3ccccc34)cc21]

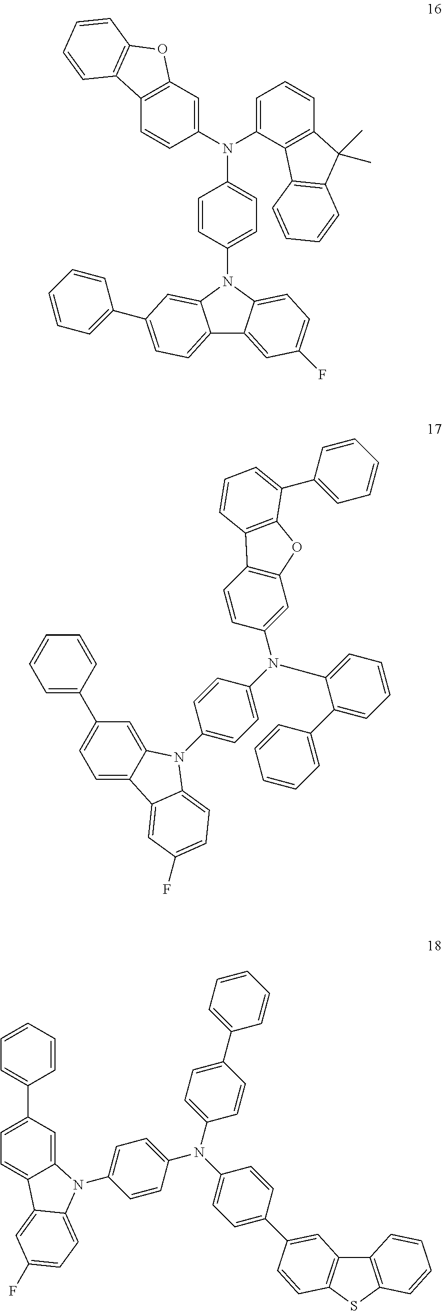 [SMILES: CC1(C)c2ccccc2-c2c(N(c3ccc(-n4c5ccc(F)cc5c5ccc(-c6ccccc6)cc54)cc3)c3ccc4c(c3)oc3ccccc34)cccc21 ; Fc1ccc2c(c1)c1ccc(-c3ccccc3)cc1n2-c1ccc(N(c2ccc3c(c2)oc2c(-c4ccccc4)cccc23)c2ccccc2-c2ccccc2)cc1 ; Fc1ccc2c(c1)c1ccc(-c3ccccc3)cc1n2-c1ccc(N(c2ccc(-c3ccccc3)cc2)c2ccc(-c3ccc4sc5ccccc5c4c3)cc2)cc1]

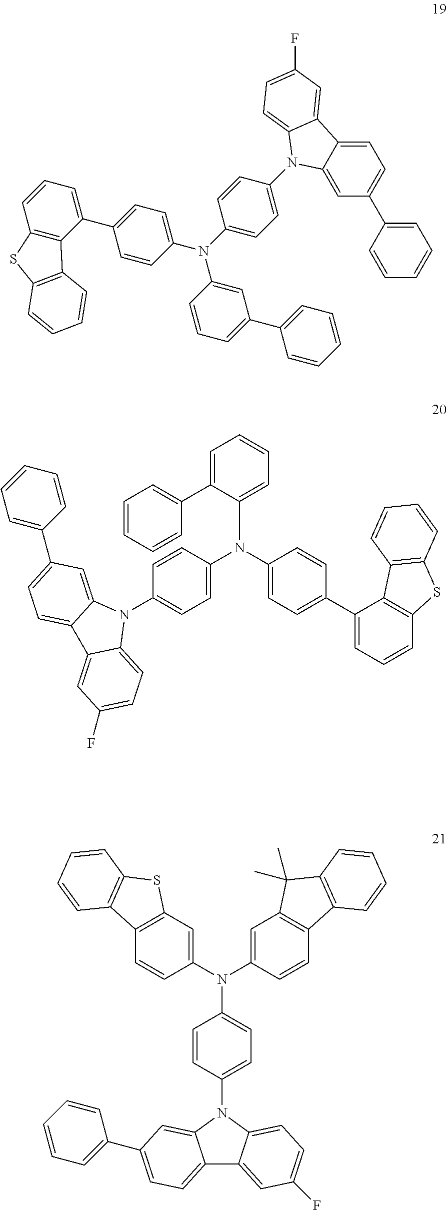 [SMILES: Fc1ccc2c(c1)c1ccc(-c3ccccc3)cc1n2-c1ccc(N(c2ccc(-c3cccc4sc5ccccc5c34)cc2)c2cccc(-c3ccccc3)c2)cc1 ; Fc1ccc2c(c1)c1ccc(-c3ccccc3)cc1n2-c1ccc(N(c2ccc(-c3cccc4sc5ccccc5c34)cc2)c2ccccc2-c2ccccc2)cc1 ; CC1(C)c2ccccc2-c2ccc(N(c3ccc(-n4c5ccc(F)cc5c5ccc(-c6ccccc6)cc54)cc3)c3ccc4c(c3)sc3ccccc34)cc21]

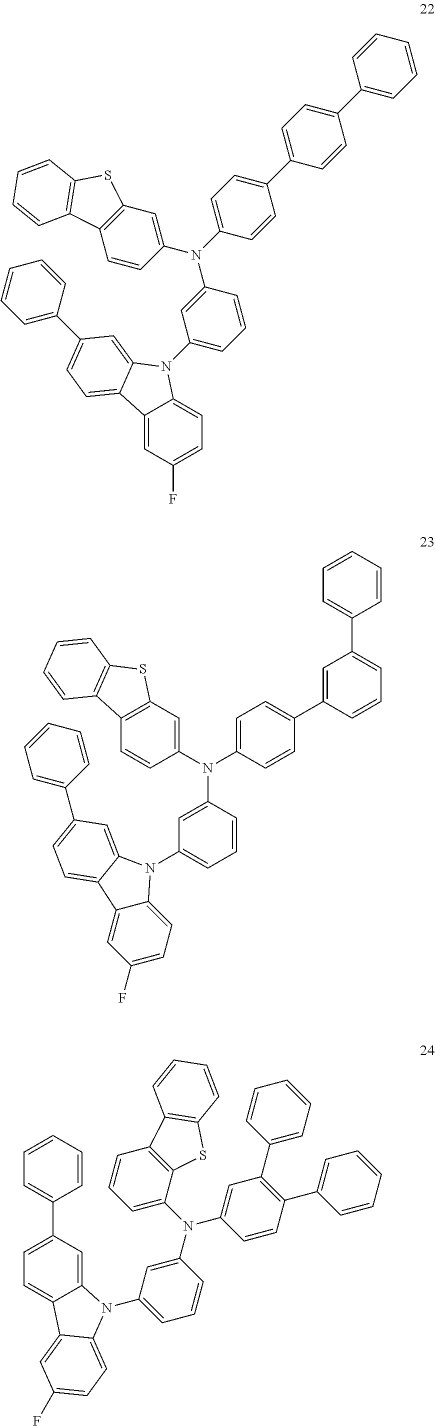 [SMILES: Fc1ccc2c(c1)c1ccc(-c3ccccc3)cc1n2-c1cccc(N(c2ccc(-c3ccc(-c4ccccc4)cc3)cc2)c2ccc3c(c2)sc2ccccc23)c1 ; Fc1ccc2c(c1)c1ccc(-c3ccccc3)cc1n2-c1cccc(N(c2ccc(-c3cccc(-c4ccccc4)c3)cc2)c2ccc3c(c2)sc2ccccc23)c1 ; Fc1ccc2c(c1)c1ccc(-c3ccccc3)cc1n2-c1cccc(N(c2ccc(-c3ccccc3)c(-c3ccccc3)c2)c2cccc3c2sc2ccccc23)c1]

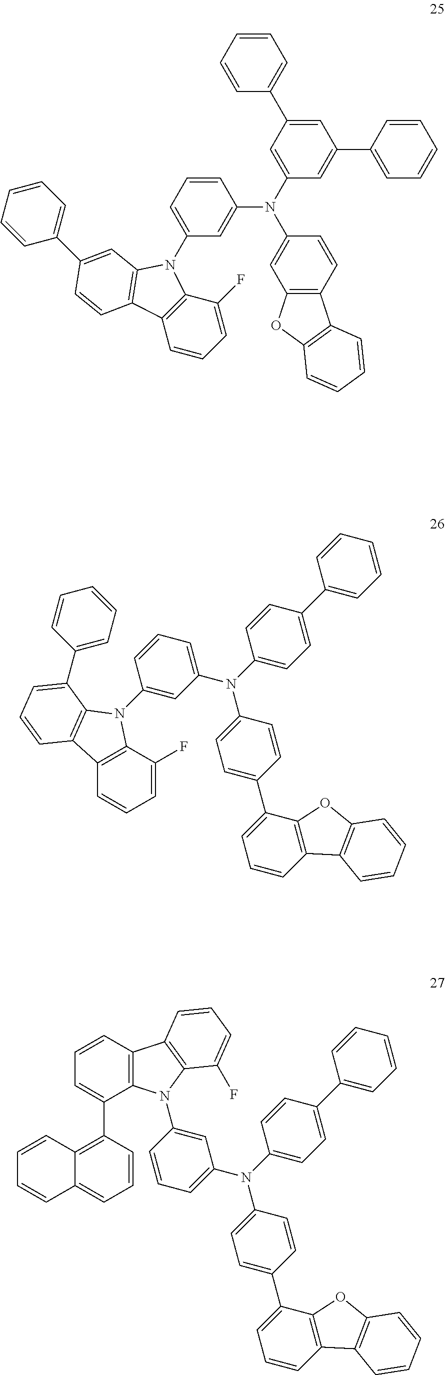 [SMILES: Fc1cccc2c3ccc(-c4ccccc4)cc3n(-c3cccc(N(c4cc(-c5ccccc5)cc(-c5ccccc5)c4)c4ccc5c(c4)oc4ccccc45)c3)c12 ; Fc1cccc2c3cccc(-c4ccccc4)c3n(-c3cccc(N(c4ccc(-c5ccccc5)cc4)c4ccc(-c5cccc6c5oc5ccccc56)cc4)c3)c12 ; Fc1cccc2c3cccc(-c4cccc5ccccc45)c3n(-c3cccc(N(c4ccc(-c5ccccc5)cc4)c4ccc(-c5cccc6c5oc5ccccc56)cc4)c3)c12]

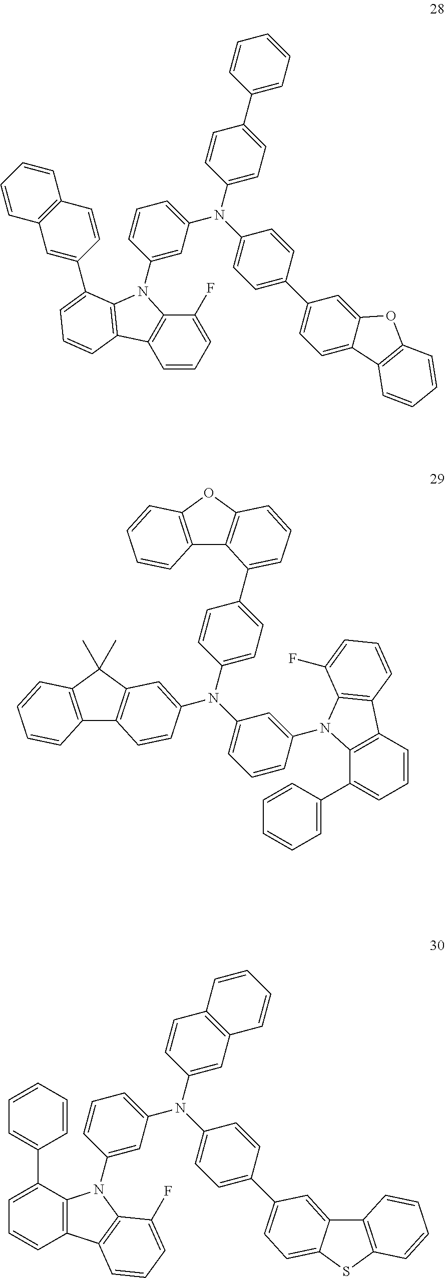 [SMILES: Fc1cccc2c3cccc(-c4ccc5ccccc5c4)c3n(-c3cccc(N(c4ccc(-c5ccccc5)cc4)c4ccc(-c5ccc6c(c5)oc5ccccc56)cc4)c3)c12 ; CC1(C)c2ccccc2-c2ccc(N(c3ccc(-c4cccc5oc6ccccc6c45)cc3)c3cccc(-n4c5c(F)cccc5c5cccc(-c6ccccc6)c54)c3)cc21 ; Fc1cccc2c3cccc(-c4ccccc4)c3n(-c3cccc(N(c4ccc(-c5ccc6sc7ccccc7c6c5)cc4)c4ccc5ccccc5c4)c3)c12]

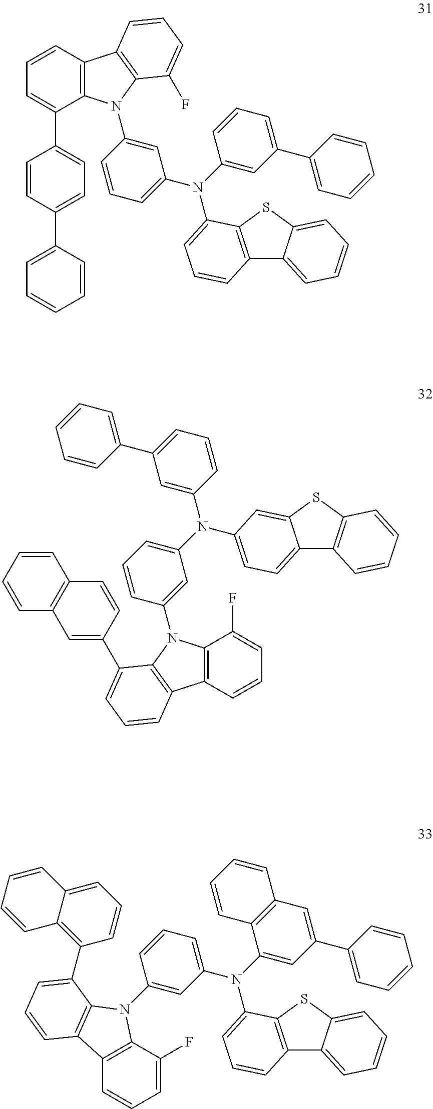 [SMILES: Fc1cccc2c3cccc(-c4ccc(-c5ccccc5)cc4)c3n(-c3cccc(N(c4cccc(-c5ccccc5)c4)c4cccc5c4sc4ccccc45)c3)c12 ; Fc1cccc2c3cccc(-c4ccc5ccccc5c4)c3n(-c3cccc(N(c4cccc(-c5ccccc5)c4)c4ccc5c(c4)sc4ccccc45)c3)c12 ; Fc1cccc2c3cccc(-c4cccc5ccccc45)c3n(-c3cccc(N(c4cc(-c5ccccc5)cc5ccccc45)c4cccc5c4sc4ccccc45)c3)c12]

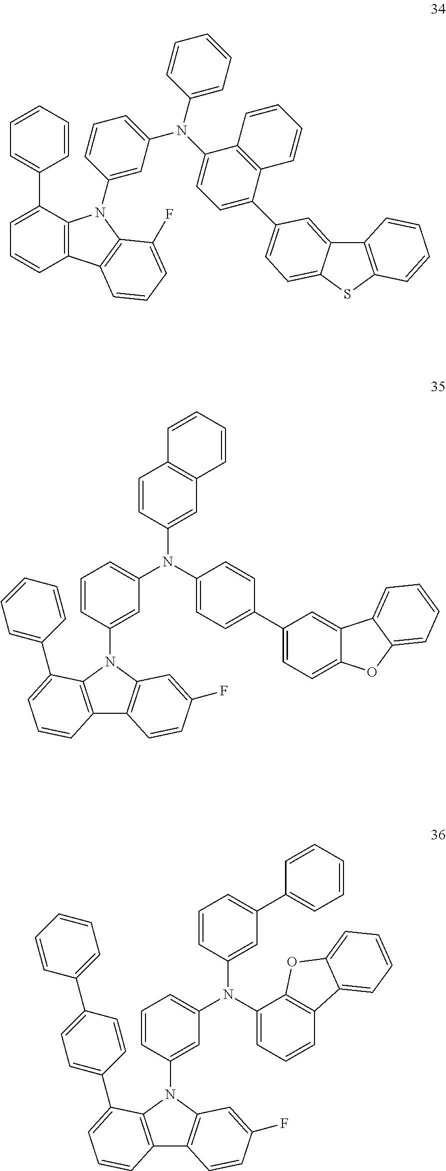 [SMILES: Fc1cccc2c3cccc(-c4ccccc4)c3n(-c3cccc(N(c4ccccc4)c4ccc(-c5ccc6sc7ccccc7c6c5)c5ccccc45)c3)c12 ; Fc1ccc2c3cccc(-c4ccccc4)c3n(-c3cccc(N(c4ccc(-c5ccc6oc7ccccc7c6c5)cc4)c4ccc5ccccc5c4)c3)c2c1 ; Fc1ccc2c3cccc(-c4ccc(-c5ccccc5)cc4)c3n(-c3cccc(N(c4cccc(-c5ccccc5)c4)c4cccc5c4oc4ccccc45)c3)c2c1]

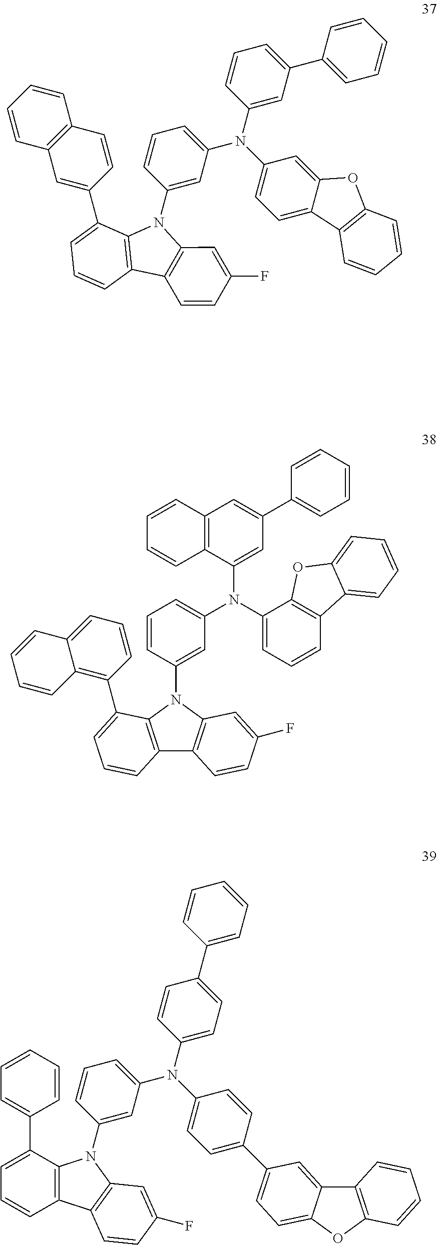 [SMILES: Fc1ccc2c3cccc(-c4ccc5ccccc5c4)c3n(-c3cccc(N(c4cccc(-c5ccccc5)c4)c4ccc5c(c4)oc4ccccc45)c3)c2c1 ; Fc1ccc2c3cccc(-c4cccc5ccccc45)c3n(-c3cccc(N(c4cc(-c5ccccc5)cc5ccccc45)c4cccc5c4oc4ccccc45)c3)c2c1 ; Fc1ccc2c3cccc(-c4ccccc4)c3n(-c3cccc(N(c4ccc(-c5ccccc5)cc4)c4ccc(-c5ccc6oc7ccccc7c6c5)cc4)c3)c2c1]

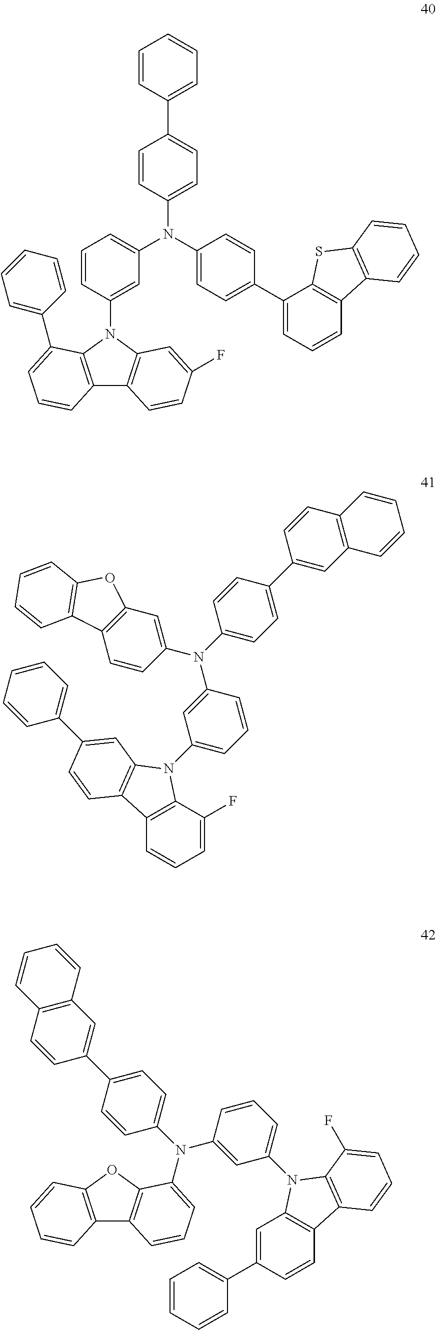 [SMILES: Fc1ccc2c3cccc(-c4ccccc4)c3n(-c3cccc(N(c4ccc(-c5ccccc5)cc4)c4ccc(-c5cccc6c5sc5ccccc56)cc4)c3)c2c1 ; Fc1cccc2c3ccc(-c4ccccc4)cc3n(-c3cccc(N(c4ccc(-c5ccc6ccccc6c5)cc4)c4ccc5c(c4)oc4ccccc45)c3)c12 ; Fc1cccc2c3ccc(-c4ccccc4)cc3n(-c3cccc(N(c4ccc(-c5ccc6ccccc6c5)cc4)c4cccc5c4oc4ccccc45)c3)c12]

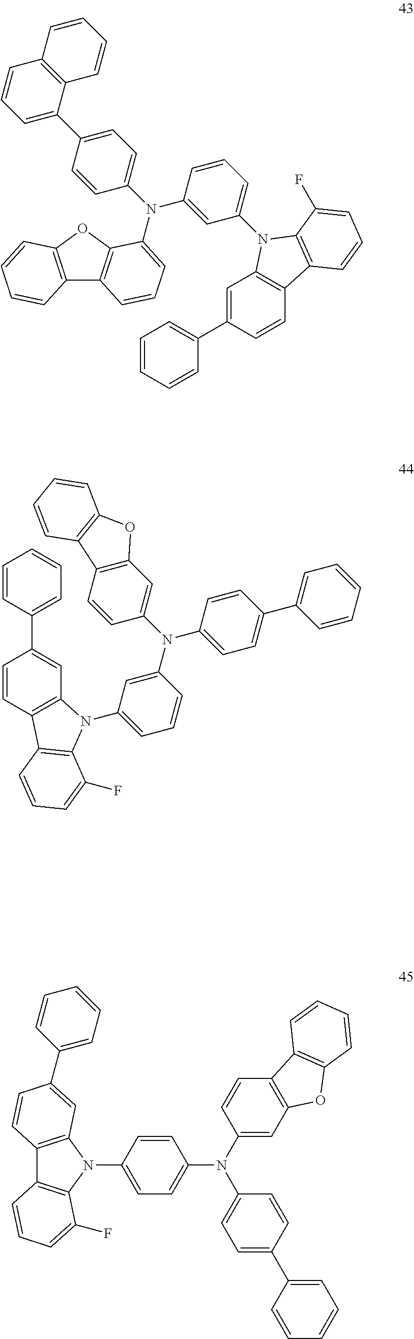 [SMILES: Fc1cccc2c3ccc(-c4ccccc4)cc3n(-c3cccc(N(c4ccc(-c5cccc6ccccc56)cc4)c4cccc5c4oc4ccccc45)c3)c12 ; Fc1cccc2c3ccc(-c4ccccc4)cc3n(-c3cccc(N(c4ccc(-c5ccccc5)cc4)c4ccc5c(c4)oc4ccccc45)c3)c12 ; Fc1cccc2c3ccc(-c4ccccc4)cc3n(-c3ccc(N(c4ccc(-c5ccccc5)cc4)c4ccc5c(c4)oc4ccccc45)cc3)c12]

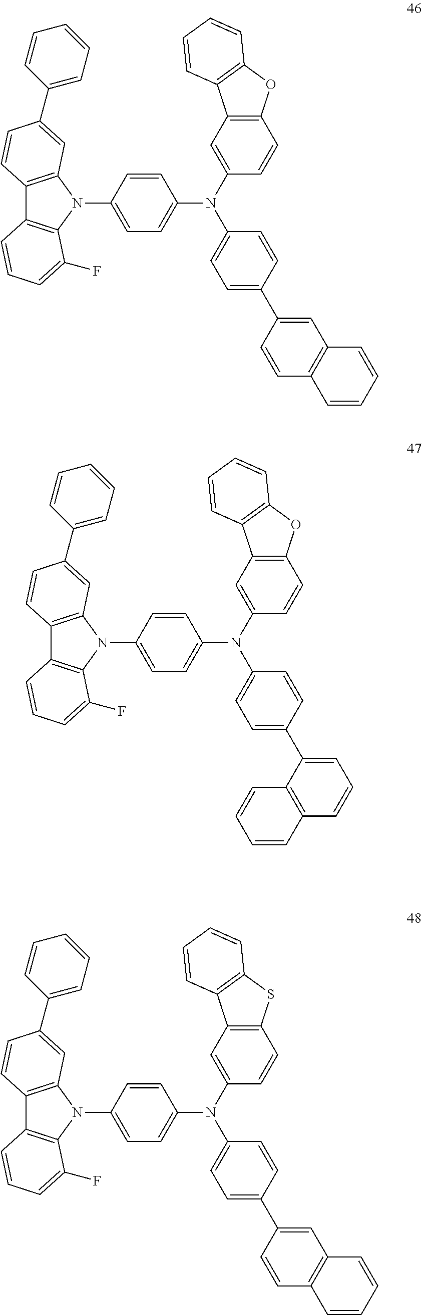 [SMILES: Fc1cccc2c3ccc(-c4ccccc4)cc3n(-c3ccc(N(c4ccc(-c5ccc6ccccc6c5)cc4)c4ccc5oc6ccccc6c5c4)cc3)c12 ; Fc1cccc2c3ccc(-c4ccccc4)cc3n(-c3ccc(N(c4ccc(-c5cccc6ccccc56)cc4)c4ccc5oc6ccccc6c5c4)cc3)c12 ; Fc1cccc2c3ccc(-c4ccccc4)cc3n(-c3ccc(N(c4ccc(-c5ccc6ccccc6c5)cc4)c4ccc5sc6ccccc6c5c4)cc3)c12]

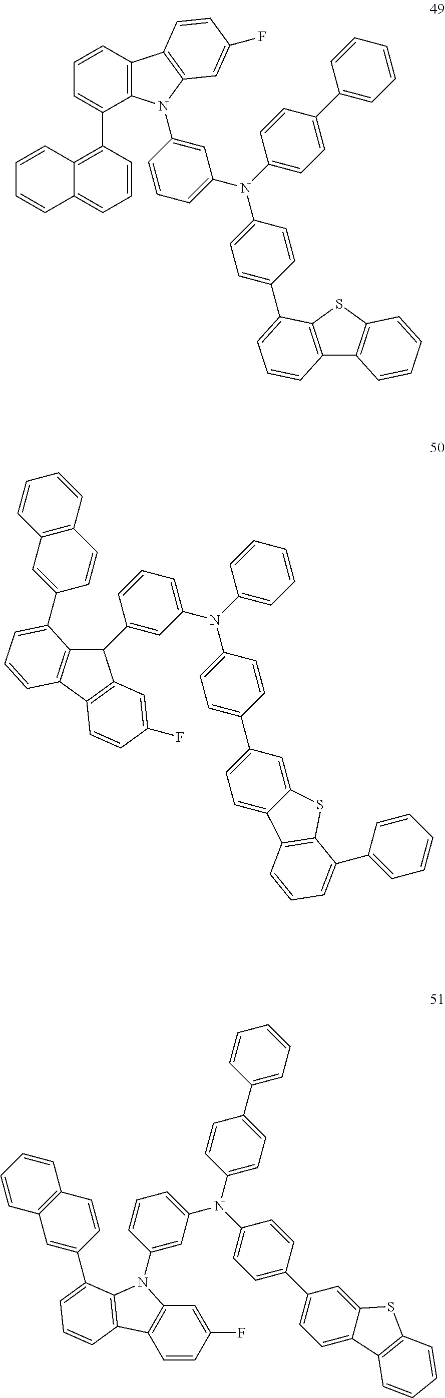 [SMILES: Fc1ccc2c3cccc(-c4cccc5ccccc45)c3n(-c3cccc(N(c4ccc(-c5ccccc5)cc4)c4ccc(-c5cccc6c5sc5ccccc56)cc4)c3)c2c1 ; Fc1ccc2c(c1)C(c1cccc(N(c3ccccc3)c3ccc(-c4ccc5c(c4)sc4c(-c6ccccc6)cccc45)cc3)c1)c1c(-c3ccc4ccccc4c3)cccc1-2 ; Fc1ccc2c3cccc(-c4ccc5ccccc5c4)c3n(-c3cccc(N(c4ccc(-c5ccccc5)cc4)c4ccc(-c5ccc6c(c5)sc5ccccc56)cc4)c3)c2c1]

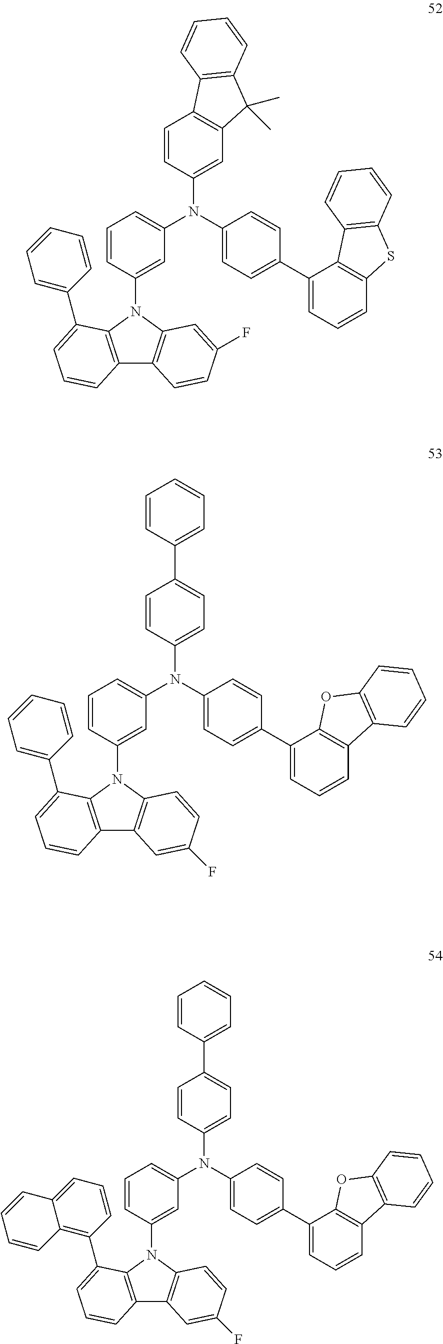 [SMILES: CC1(C)c2ccccc2-c2ccc(N(c3ccc(-c4cccc5sc6ccccc6c45)cc3)c3cccc(-n4c5cc(F)ccc5c5cccc(-c6ccccc6)c54)c3)cc21 ; Fc1ccc2c(c1)c1cccc(-c3ccccc3)c1n2-c1cccc(N(c2ccc(-c3ccccc3)cc2)c2ccc(-c3cccc4c3oc3ccccc34)cc2)c1 ; Fc1ccc2c(c1)c1cccc(-c3cccc4ccccc34)c1n2-c1cccc(N(c2ccc(-c3ccccc3)cc2)c2ccc(-c3cccc4c3oc3ccccc34)cc2)c1]

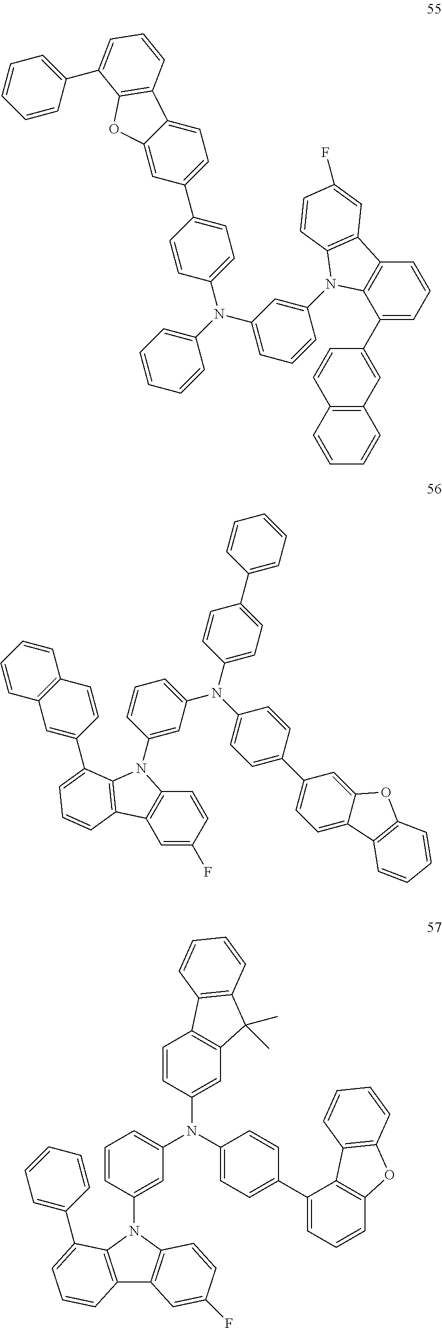 [SMILES: Fc1ccc2c(c1)c1cccc(-c3ccc4ccccc4c3)c1n2-c1cccc(N(c2ccccc2)c2ccc(-c3ccc4c(c3)oc3c(-c5ccccc5)cccc34)cc2)c1 ; Fc1ccc2c(c1)c1cccc(-c3ccc4ccccc4c3)c1n2-c1cccc(N(c2ccc(-c3ccccc3)cc2)c2ccc(-c3ccc4c(c3)oc3ccccc34)cc2)c1 ; CC1(C)c2ccccc2-c2ccc(N(c3ccc(-c4cccc5oc6ccccc6c45)cc3)c3cccc(-n4c5ccc(F)cc5c5cccc(-c6ccccc6)c54)c3)cc21]

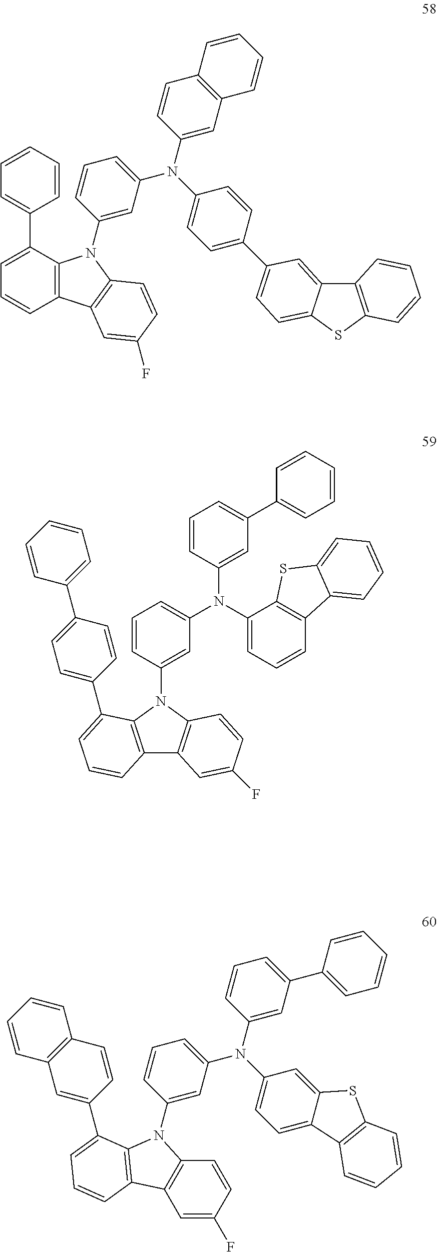 [SMILES: Fc1ccc2c(c1)c1cccc(-c3ccccc3)c1n2-c1cccc(N(c2ccc(-c3ccc4sc5ccccc5c4c3)cc2)c2ccc3ccccc3c2)c1 ; Fc1ccc2c(c1)c1cccc(-c3ccc(-c4ccccc4)cc3)c1n2-c1cccc(N(c2cccc(-c3ccccc3)c2)c2cccc3c2sc2ccccc23)c1 ; Fc1ccc2c(c1)c1cccc(-c3ccc4ccccc4c3)c1n2-c1cccc(N(c2cccc(-c3ccccc3)c2)c2ccc3c(c2)sc2ccccc23)c1]

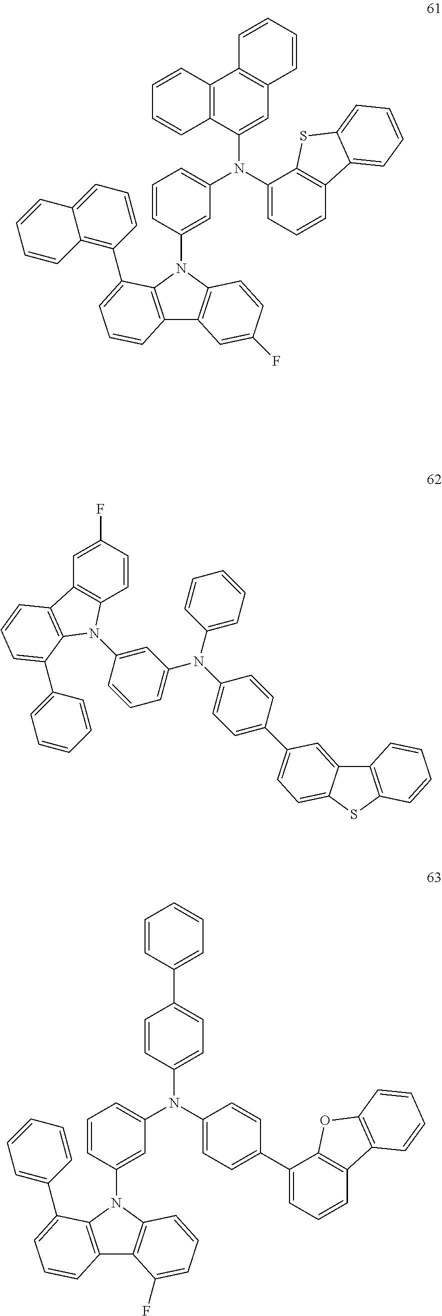 [SMILES: Fc1ccc2c(c1)c1cccc(-c3cccc4ccccc34)c1n2-c1cccc(N(c2cc3ccccc3c3ccccc23)c2cccc3c2sc2ccccc23)c1 ; Fc1ccc2c(c1)c1cccc(-c3ccccc3)c1n2-c1cccc(N(c2ccccc2)c2ccc(-c3ccc4sc5ccccc5c4c3)cc2)c1 ; Fc1cccc2c1c1cccc(-c3ccccc3)c1n2-c1cccc(N(c2ccc(-c3ccccc3)cc2)c2ccc(-c3cccc4c3oc3ccccc34)cc2)c1]

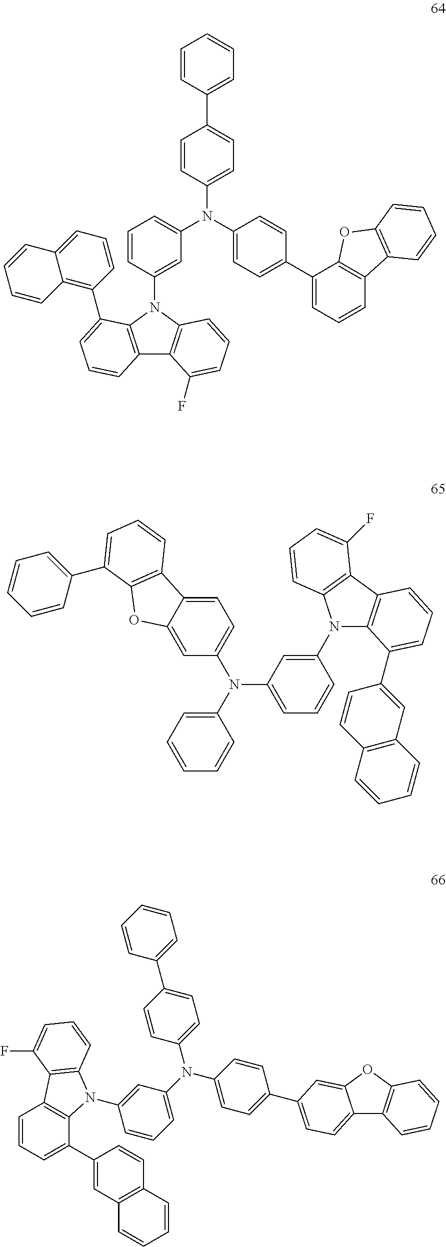 [SMILES: Fc1cccc2c1c1cccc(-c3cccc4ccccc34)c1n2-c1cccc(N(c2ccc(-c3ccccc3)cc2)c2ccc(-c3cccc4c3oc3ccccc34)cc2)c1 ; Fc1cccc2c1c1cccc(-c3ccc4ccccc4c3)c1n2-c1cccc(N(c2ccccc2)c2ccc3c(c2)oc2c(-c4ccccc4)cccc23)c1 ; Fc1cccc2c1c1cccc(-c3ccc4ccccc4c3)c1n2-c1cccc(N(c2ccc(-c3ccccc3)cc2)c2ccc(-c3ccc4c(c3)oc3ccccc34)cc2)c1]

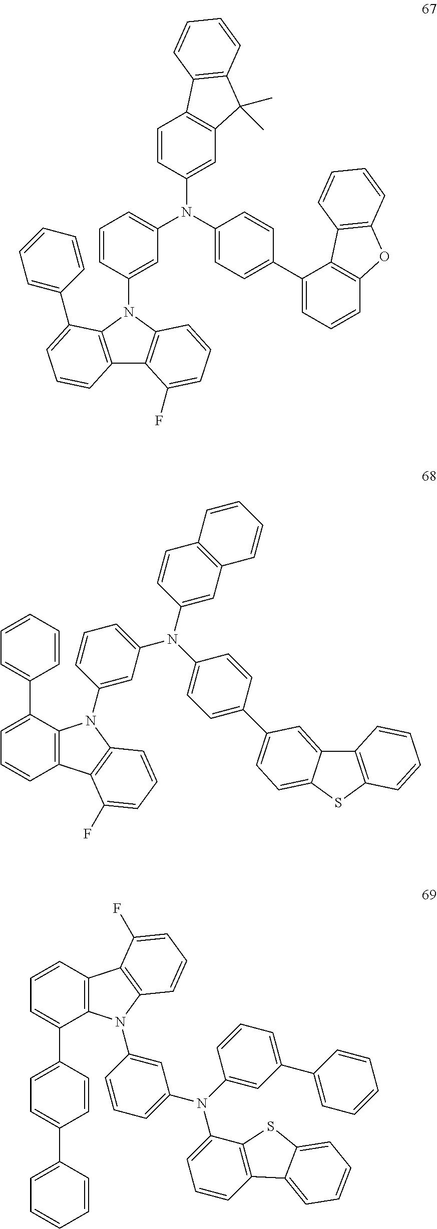 [SMILES: CC1(C)c2ccccc2-c2ccc(N(c3ccc(-c4cccc5oc6ccccc6c45)cc3)c3cccc(-n4c5cccc(F)c5c5cccc(-c6ccccc6)c54)c3)cc21 ; Fc1cccc2c1c1cccc(-c3ccccc3)c1n2-c1cccc(N(c2ccc(-c3ccc4sc5ccccc5c4c3)cc2)c2ccc3ccccc3c2)c1 ; Fc1cccc2c1c1cccc(-c3ccc(-c4ccccc4)cc3)c1n2-c1cccc(N(c2cccc(-c3ccccc3)c2)c2cccc3c2sc2ccccc23)c1]

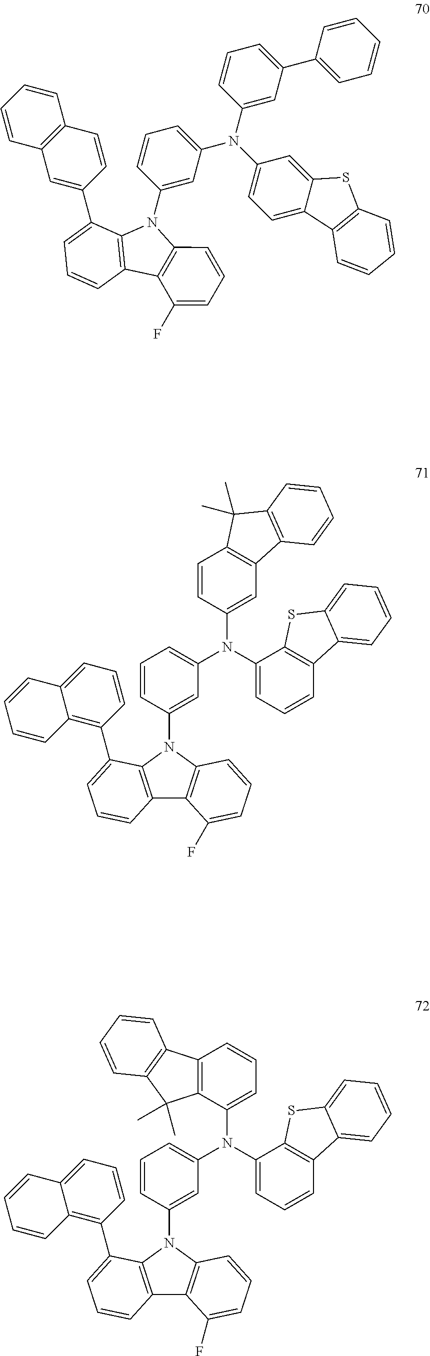 [SMILES: Fc1cccc2c1c1cccc(-c3ccc4ccccc4c3)c1n2-c1cccc(N(c2cccc(-c3ccccc3)c2)c2ccc3c(c2)sc2ccccc23)c1 ; CC1(C)c2ccccc2-c2cc(N(c3cccc(-n4c5cccc(F)c5c5cccc(-c6cccc7ccccc67)c54)c3)c3cccc4c3sc3ccccc34)ccc21 ; CC1(C)c2ccccc2-c2cccc(N(c3cccc(-n4c5cccc(F)c5c5cccc(-c6cccc7ccccc67)c54)c3)c3cccc4c3sc3ccccc34)c21]

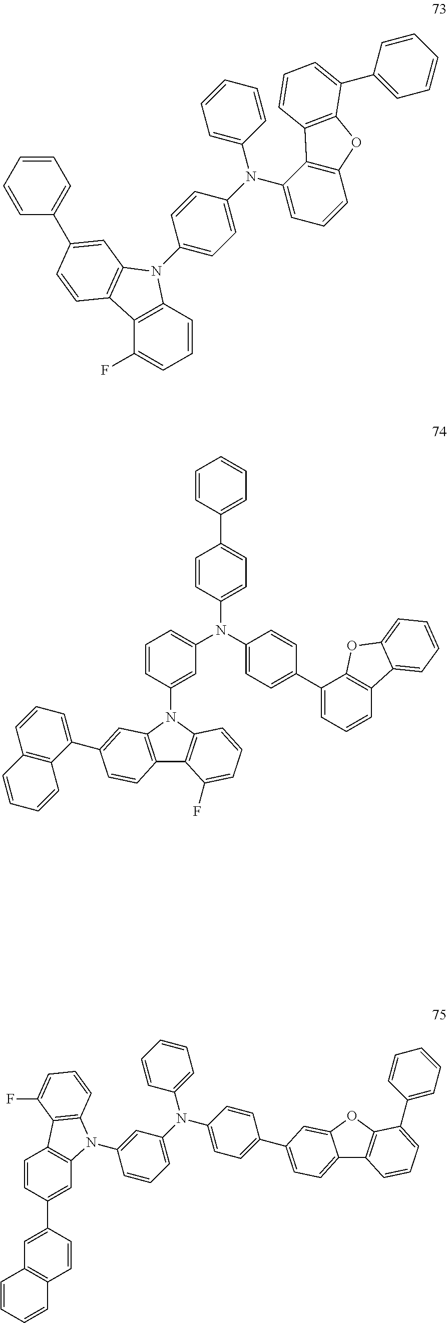 [SMILES: Fc1cccc2c1c1ccc(-c3ccccc3)cc1n2-c1ccc(N(c2ccccc2)c2cccc3oc4c(-c5ccccc5)cccc4c23)cc1 ; Fc1cccc2c1c1ccc(-c3cccc4ccccc34)cc1n2-c1cccc(N(c2ccc(-c3ccccc3)cc2)c2ccc(-c3cccc4c3oc3ccccc34)cc2)c1 ; Fc1cccc2c1c1ccc(-c3ccc4ccccc4c3)cc1n2-c1cccc(N(c2ccccc2)c2ccc(-c3ccc4c(c3)oc3c(-c5ccccc5)cccc34)cc2)c1]

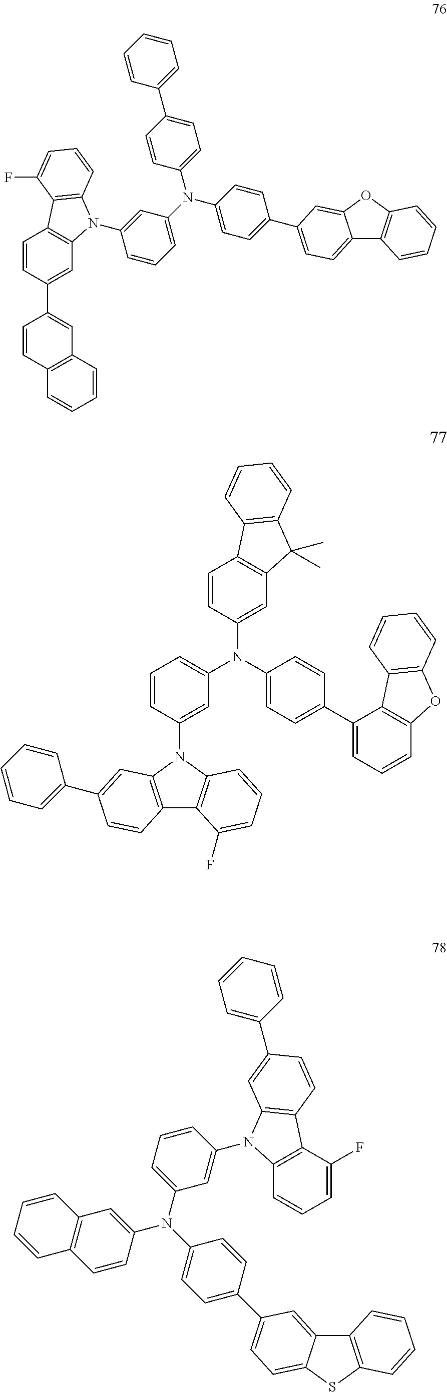 [SMILES: Fc1cccc2c1c1ccc(-c3ccc4ccccc4c3)cc1n2-c1cccc(N(c2ccc(-c3ccccc3)cc2)c2ccc(-c3ccc4c(c3)oc3ccccc34)cc2)c1 ; CC1(C)c2ccccc2-c2ccc(N(c3ccc(-c4cccc5oc6ccccc6c45)cc3)c3cccc(-n4c5cc(-c6ccccc6)ccc5c5c(F)cccc54)c3)cc21 ; Fc1cccc2c1c1ccc(-c3ccccc3)cc1n2-c1cccc(N(c2ccc(-c3ccc4sc5ccccc5c4c3)cc2)c2ccc3ccccc3c2)c1]

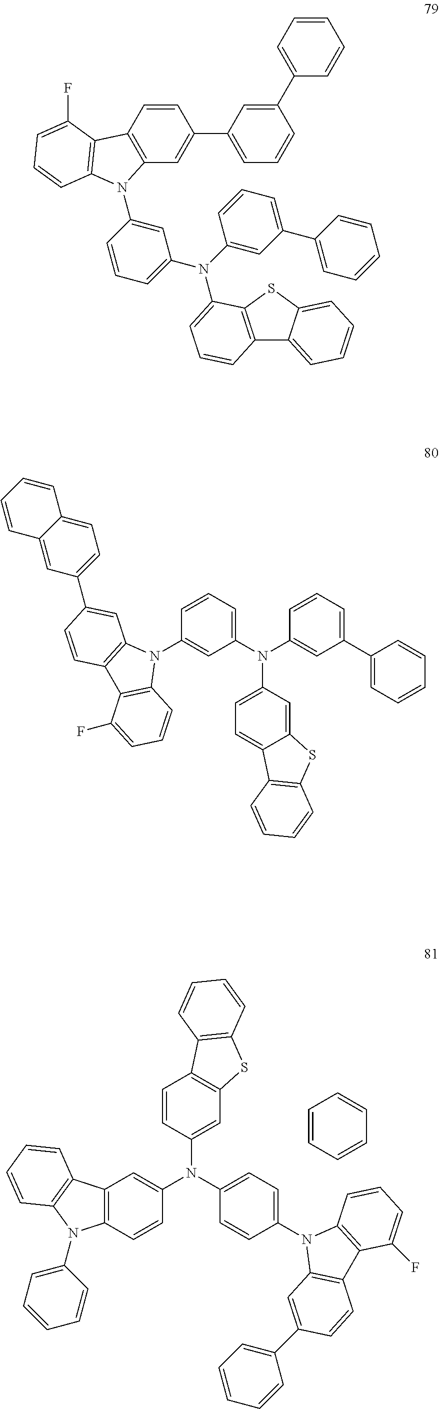

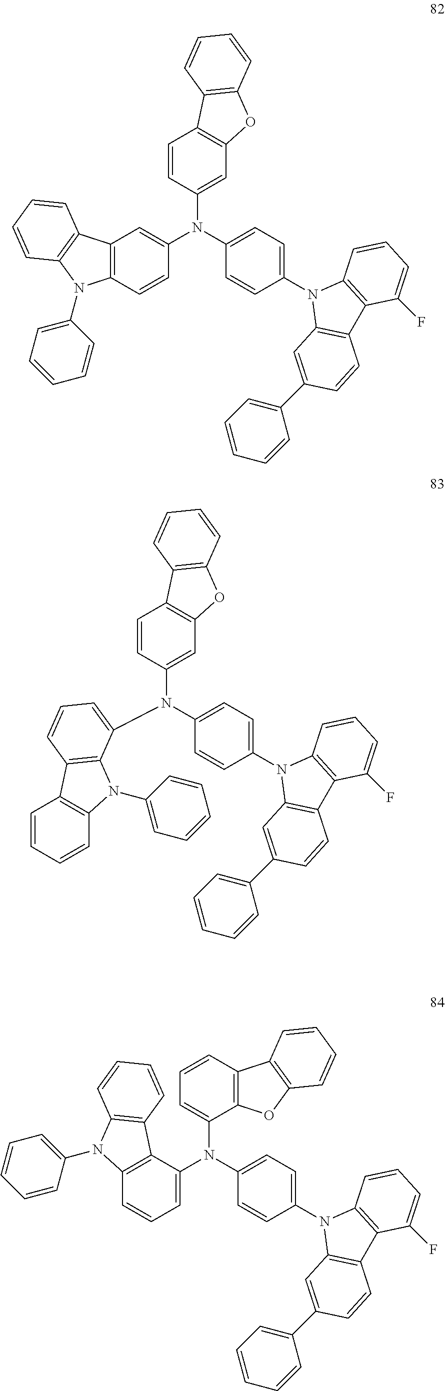 [SMILES: Fc1cccc2c1c1ccc(-c3ccccc3)cc1n2-c1ccc(N(c2ccc3c(c2)oc2ccccc23)c2ccc3c(c2)c2ccccc2n3-c2ccccc2)cc1 ; Fc1cccc2c1c1ccc(-c3ccccc3)cc1n2-c1ccc(N(c2ccc3c(c2)oc2ccccc23)c2cccc3c4ccccc4n(-c4ccccc4)c23)cc1 ; Fc1cccc2c1c1ccc(-c3ccccc3)cc1n2-c1ccc(N(c2cccc3c2oc2ccccc23)c2cccc3c2c2ccccc2n3-c2ccccc2)cc1]

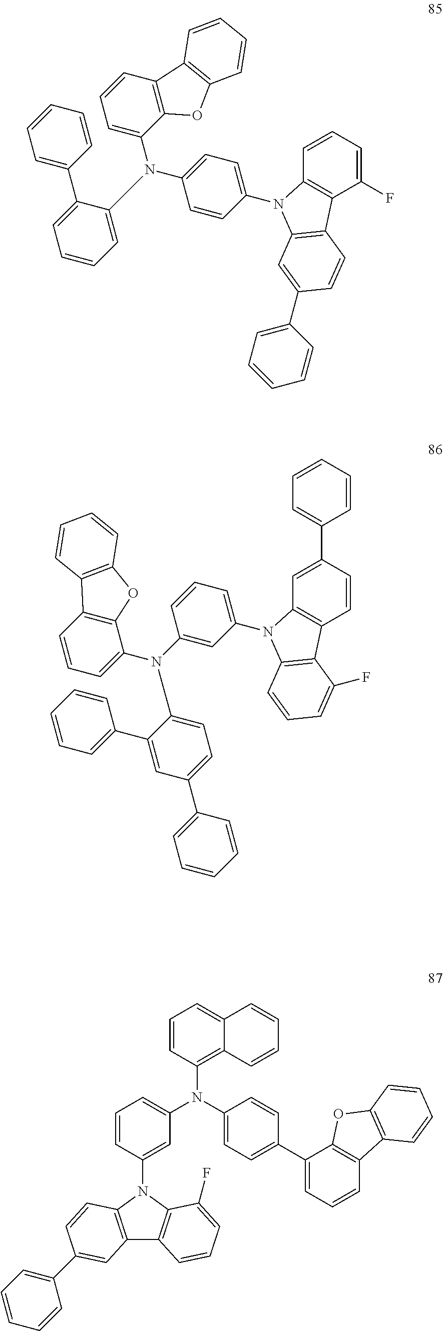 [SMILES: Fc1cccc2c1c1ccc(-c3ccccc3)cc1n2-c1ccc(N(c2ccccc2-c2ccccc2)c2cccc3c2oc2ccccc23)cc1 ; Fc1cccc2c1c1ccc(-c3ccccc3)cc1n2-c1cccc(N(c2ccc(-c3ccccc3)cc2-c2ccccc2)c2cccc3c2oc2ccccc23)c1 ; Fc1cccc2c3cc(-c4ccccc4)ccc3n(-c3cccc(N(c4ccc(-c5cccc6c5oc5ccccc56)cc4)c4cccc5ccccc45)c3)c12]

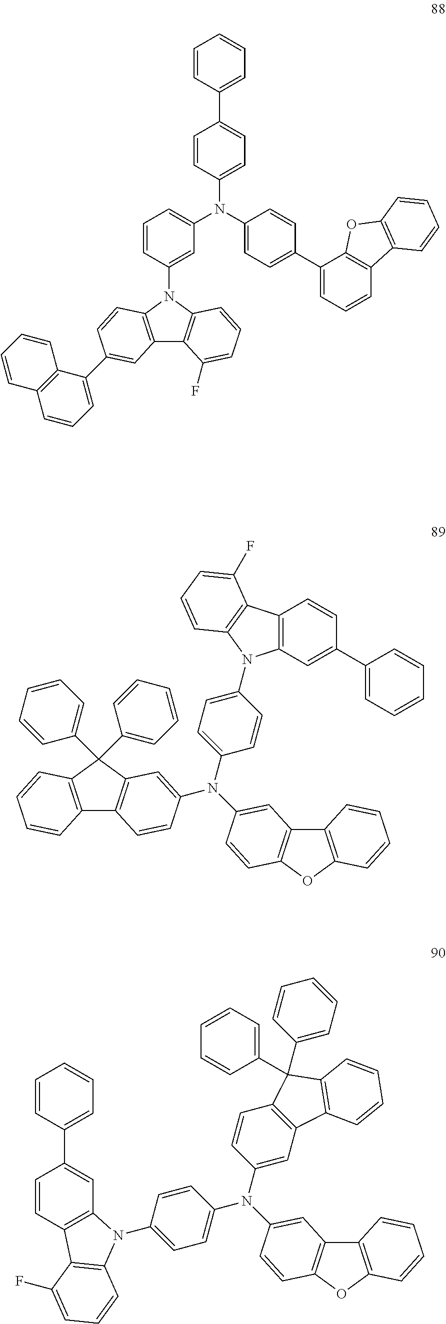 [SMILES: Fc1cccc2c1c1cc(-c3cccc4ccccc34)ccc1n2-c1cccc(N(c2ccc(-c3ccccc3)cc2)c2ccc(-c3cccc4c3oc3ccccc34)cc2)c1 ; Fc1cccc2c1c1ccc(-c3ccccc3)cc1n2-c1ccc(N(c2ccc3c(c2)C(c2ccccc2)(c2ccccc2)c2ccccc2-3)c2ccc3oc4ccccc4c3c2)cc1 ; Fc1cccc2c1c1ccc(-c3ccccc3)cc1n2-c1ccc(N(c2ccc3c(c2)-c2ccccc2C3(c2ccccc2)c2ccccc2)c2ccc3oc4ccccc4c3c2)cc1]

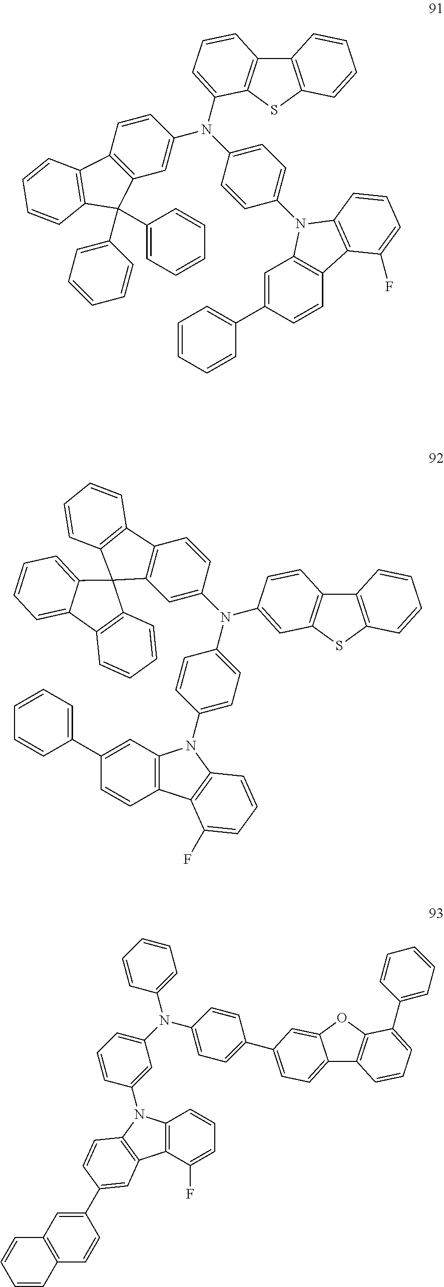 [SMILES: Fc1cccc2c1c1ccc(-c3ccccc3)cc1n2-c1ccc(N(c2ccc3c(c2)C(c2ccccc2)(c2ccccc2)c2ccccc2-3)c2cccc3c2sc2ccccc23)cc1 ; Fc1cccc2c1c1ccc(-c3ccccc3)cc1n2-c1ccc(N(c2ccc3c(c2)C2(c4ccccc4-c4ccccc42)c2ccccc2-3)c2ccc3c(c2)sc2ccccc23)cc1 ; Fc1cccc2c1c1cc(-c3ccc4ccccc4c3)ccc1n2-c1cccc(N(c2ccccc2)c2ccc(-c3ccc4c(c3)oc3c(-c5ccccc5)cccc34)cc2)c1]

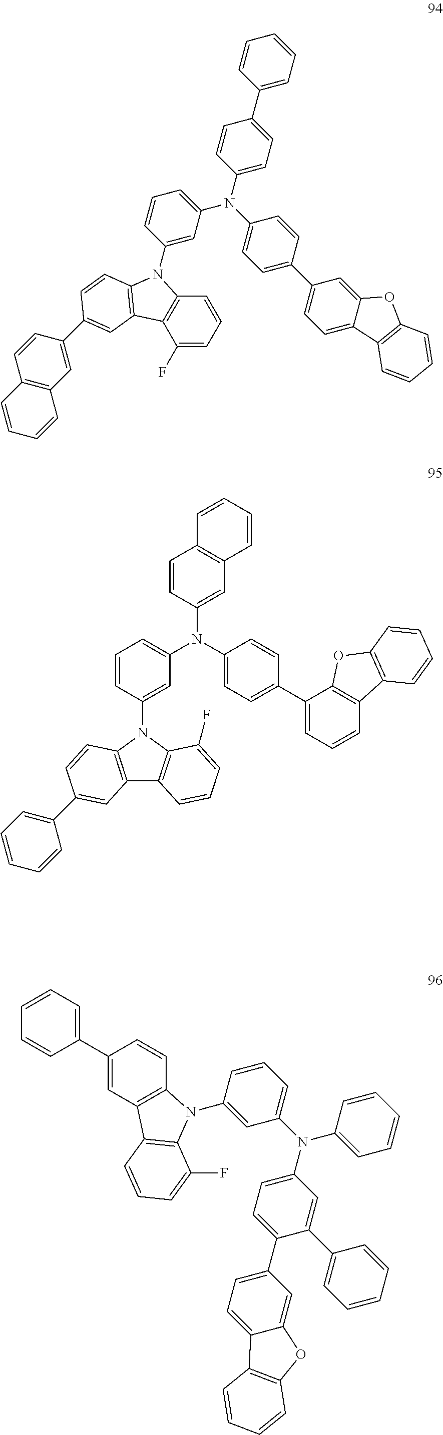 [SMILES: Fc1cccc2c1c1cc(-c3ccc4ccccc4c3)ccc1n2-c1cccc(N(c2ccc(-c3ccccc3)cc2)c2ccc(-c3ccc4c(c3)oc3ccccc34)cc2)c1 ; Fc1cccc2c3cc(-c4ccccc4)ccc3n(-c3cccc(N(c4ccc(-c5cccc6c5oc5ccccc56)cc4)c4ccc5ccccc5c4)c3)c12 ; Fc1cccc2c3cc(-c4ccccc4)ccc3n(-c3cccc(N(c4ccccc4)c4ccc(-c5ccc6c(c5)oc5ccccc56)c(-c5ccccc5)c4)c3)c12]

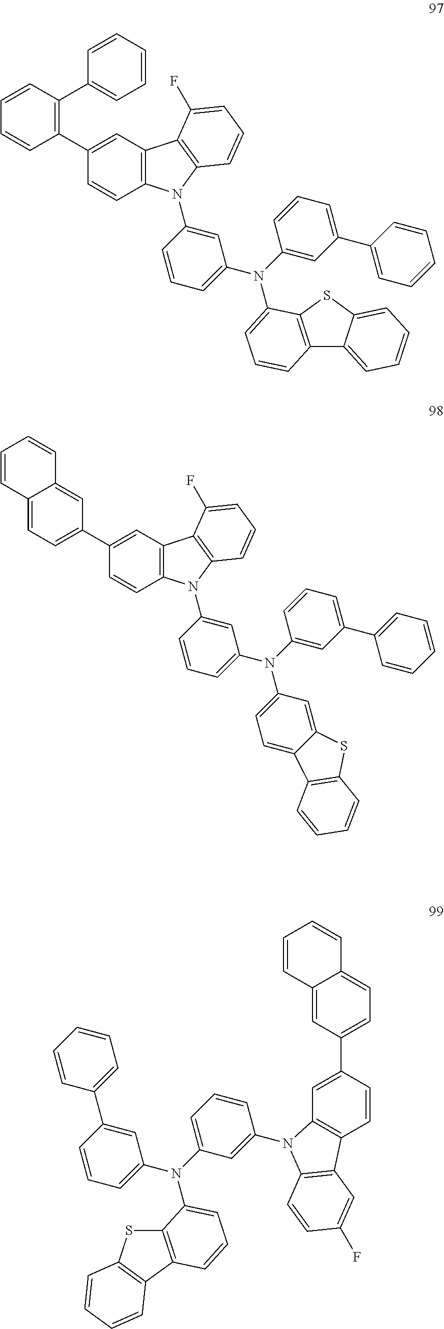 [SMILES: Fc1cccc2c1c1cc(-c3ccccc3-c3ccccc3)ccc1n2-c1cccc(N(c2cccc(-c3ccccc3)c2)c2cccc3c2sc2ccccc23)c1 ; Fc1cccc2c1c1cc(-c3ccc4ccccc4c3)ccc1n2-c1cccc(N(c2cccc(-c3ccccc3)c2)c2ccc3c(c2)sc2ccccc23)c1 ; Fc1ccc2c(c1)c1ccc(-c3ccc4ccccc4c3)cc1n2-c1cccc(N(c2cccc(-c3ccccc3)c2)c2cccc3c2sc2ccccc23)c1]

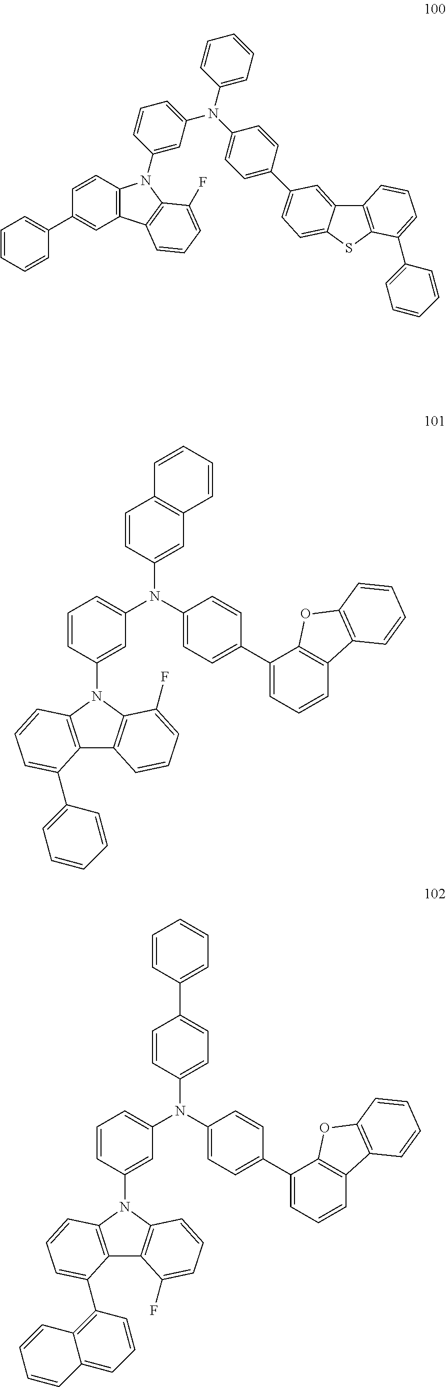 [SMILES: Fc1cccc2c3cc(-c4ccccc4)ccc3n(-c3cccc(N(c4ccccc4)c4ccc(-c5ccc6sc7c(-c8ccccc8)cccc7c6c5)cc4)c3)c12 ; Fc1cccc2c3c(-c4ccccc4)cccc3n(-c3cccc(N(c4ccc(-c5cccc6c5oc5ccccc56)cc4)c4ccc5ccccc5c4)c3)c12 ; Fc1cccc2c1c1c(-c3cccc4ccccc34)cccc1n2-c1cccc(N(c2ccc(-c3ccccc3)cc2)c2ccc(-c3cccc4c3oc3ccccc34)cc2)c1]

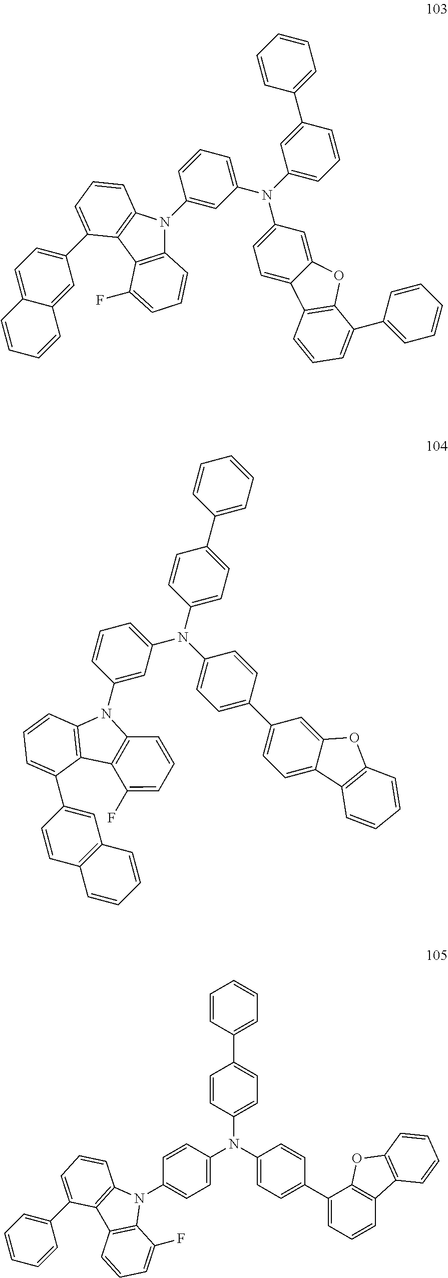 [SMILES: Fc1cccc2c1c1c(-c3ccc4ccccc4c3)cccc1n2-c1cccc(N(c2cccc(-c3ccccc3)c2)c2ccc3c(c2)oc2c(-c4ccccc4)cccc23)c1 ; Fc1cccc2c1c1c(-c3ccc4ccccc4c3)cccc1n2-c1cccc(N(c2ccc(-c3ccccc3)cc2)c2ccc(-c3ccc4c(c3)oc3ccccc34)cc2)c1 ; Fc1cccc2c3c(-c4ccccc4)cccc3n(-c3ccc(N(c4ccc(-c5ccccc5)cc4)c4ccc(-c5cccc6c5oc5ccccc56)cc4)cc3)c12]

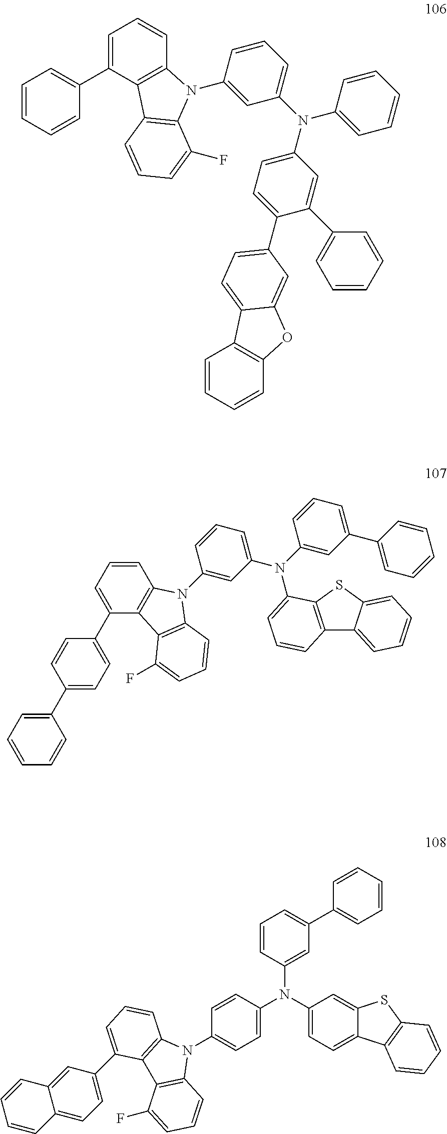 [SMILES: Fc1cccc2c3c(-c4ccccc4)cccc3n(-c3cccc(N(c4ccccc4)c4ccc(-c5ccc6c(c5)oc5ccccc56)c(-c5ccccc5)c4)c3)c12 ; Fc1cccc2c1c1c(-c3ccc(-c4ccccc4)cc3)cccc1n2-c1cccc(N(c2cccc(-c3ccccc3)c2)c2cccc3c2sc2ccccc23)c1 ; Fc1cccc2c1c1c(-c3ccc4ccccc4c3)cccc1n2-c1ccc(N(c2cccc(-c3ccccc3)c2)c2ccc3c(c2)sc2ccccc23)cc1]

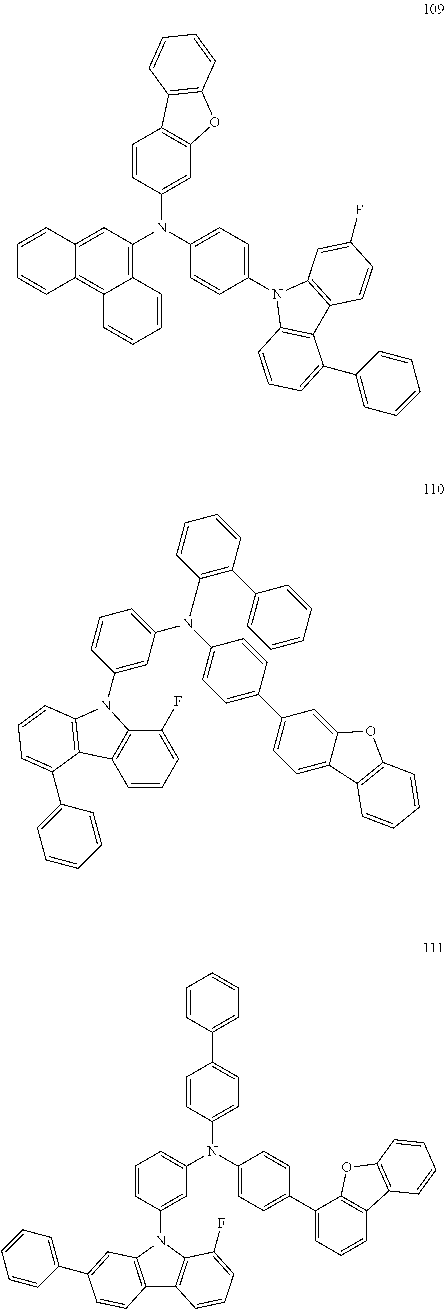 [SMILES: Fc1ccc2c3c(-c4ccccc4)cccc3n(-c3ccc(N(c4ccc5c(c4)oc4ccccc45)c4cc5ccccc5c5ccccc45)cc3)c2c1 ; Fc1cccc2c3c(-c4ccccc4)cccc3n(-c3cccc(N(c4ccc(-c5ccc6c(c5)oc5ccccc56)cc4)c4ccccc4-c4ccccc4)c3)c12 ; Fc1cccc2c3ccc(-c4ccccc4)cc3n(-c3cccc(N(c4ccc(-c5ccccc5)cc4)c4ccc(-c5cccc6c5oc5ccccc56)cc4)c3)c12]

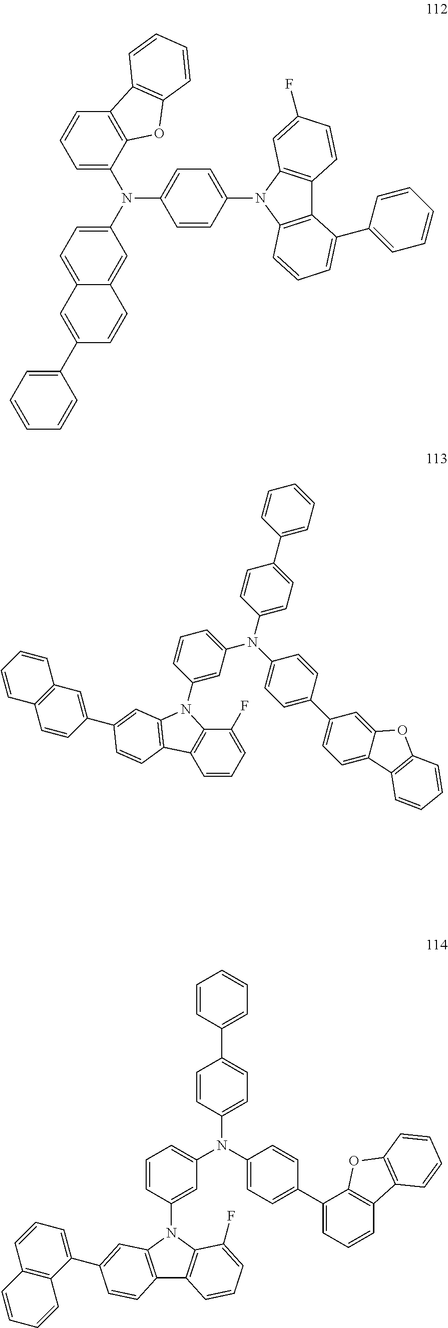 [SMILES: Fc1ccc2c3c(-c4ccccc4)cccc3n(-c3ccc(N(c4ccc5cc(-c6ccccc6)ccc5c4)c4cccc5c4oc4ccccc45)cc3)c2c1 ; Fc1cccc2c3ccc(-c4ccc5ccccc5c4)cc3n(-c3cccc(N(c4ccc(-c5ccccc5)cc4)c4ccc(-c5ccc6c(c5)oc5ccccc56)cc4)c3)c12 ; Fc1cccc2c3ccc(-c4cccc5ccccc45)cc3n(-c3cccc(N(c4ccc(-c5ccccc5)cc4)c4ccc(-c5cccc6c5oc5ccccc56)cc4)c3)c12]

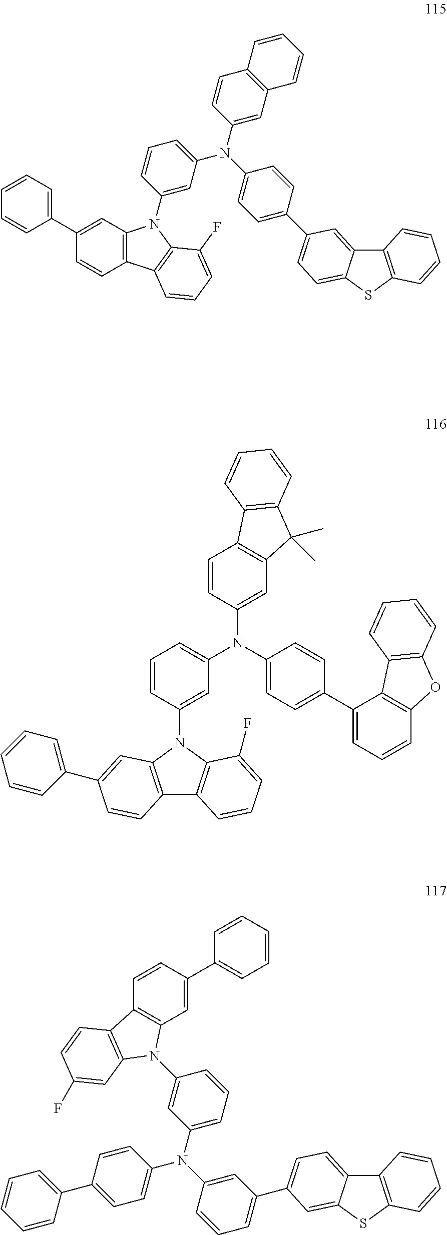 [SMILES: Fc1cccc2c3ccc(-c4ccccc4)cc3n(-c3cccc(N(c4ccc(-c5ccc6sc7ccccc7c6c5)cc4)c4ccc5ccccc5c4)c3)c12 ; CC1(C)c2ccccc2-c2ccc(N(c3ccc(-c4cccc5oc6ccccc6c45)cc3)c3cccc(-n4c5cc(-c6ccccc6)ccc5c5cccc(F)c54)c3)cc21 ; Fc1ccc2c3ccc(-c4ccccc4)cc3n(-c3cccc(N(c4ccc(-c5ccccc5)cc4)c4cccc(-c5ccc6c(c5)sc5ccccc56)c4)c3)c2c1]

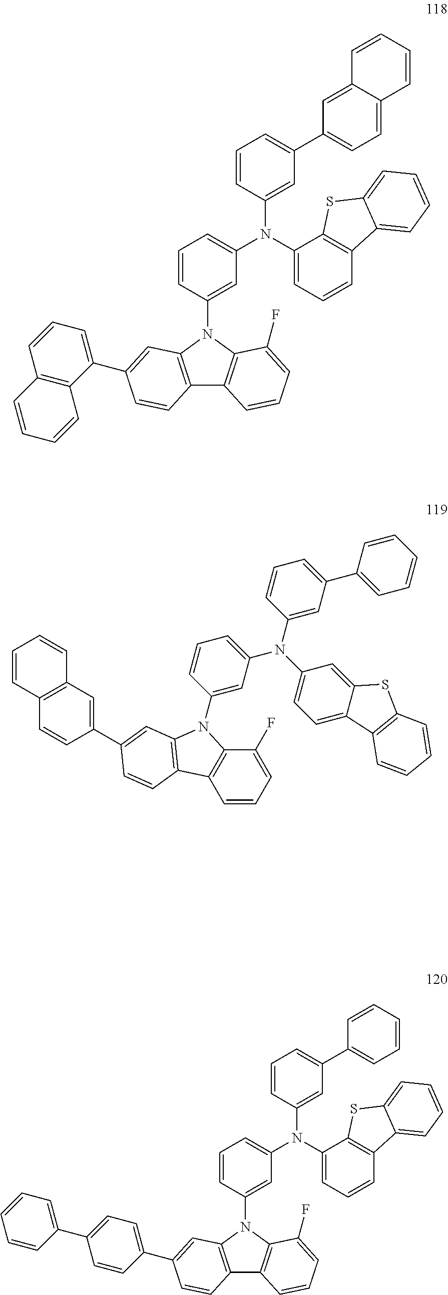 [SMILES: Fc1cccc2c3ccc(-c4cccc5ccccc45)cc3n(-c3cccc(N(c4cccc(-c5ccc6ccccc6c5)c4)c4cccc5c4sc4ccccc45)c3)c12 ; Fc1cccc2c3ccc(-c4ccc5ccccc5c4)cc3n(-c3cccc(N(c4cccc(-c5ccccc5)c4)c4ccc5c(c4)sc4ccccc45)c3)c12 ; Fc1cccc2c3ccc(-c4ccc(-c5ccccc5)cc4)cc3n(-c3cccc(N(c4cccc(-c5ccccc5)c4)c4cccc5c4sc4ccccc45)c3)c12]

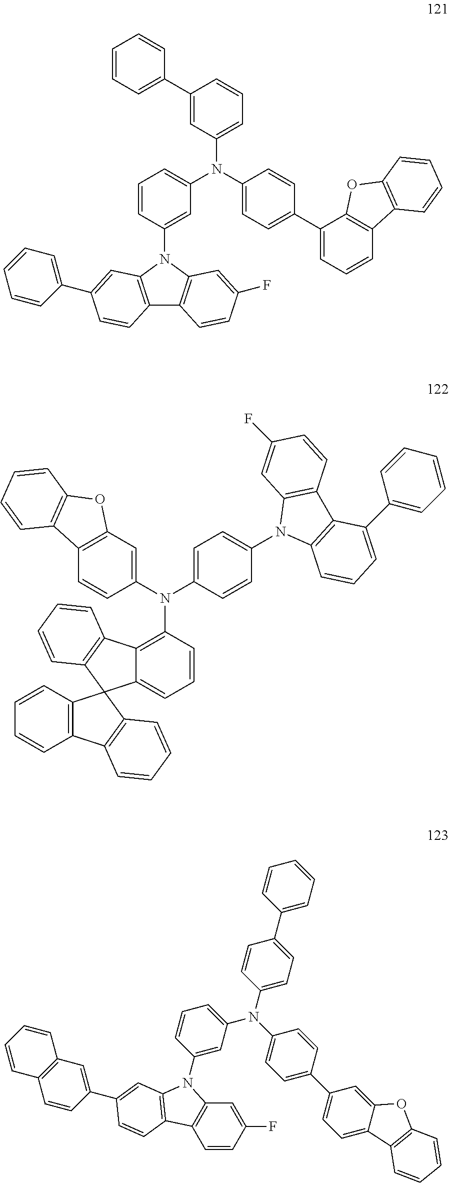 [SMILES: Fc1ccc2c3ccc(-c4ccccc4)cc3n(-c3cccc(N(c4ccc(-c5cccc6c5oc5ccccc56)cc4)c4cccc(-c5ccccc5)c4)c3)c2c1 ; Fc1ccc2c3c(-c4ccccc4)cccc3n(-c3ccc(N(c4ccc5c(c4)oc4ccccc45)c4cccc5c4-c4ccccc4C54c5ccccc5-c5ccccc54)cc3)c2c1 ; Fc1ccc2c3ccc(-c4ccc5ccccc5c4)cc3n(-c3cccc(N(c4ccc(-c5ccccc5)cc4)c4ccc(-c5ccc6c(c5)oc5ccccc56)cc4)c3)c2c1]

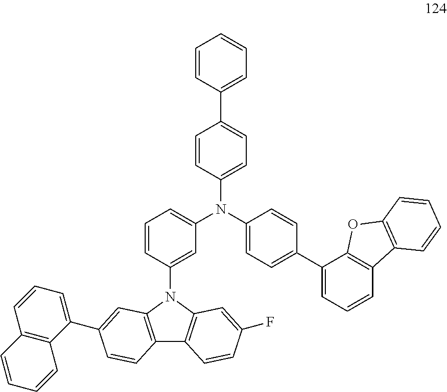 [SMILES: Fc1ccc2c3ccc(-c4cccc5ccccc45)cc3n(-c3cccc(N(c4ccc(-c5ccccc5)cc4)c4ccc(-c5cccc6c5oc5ccccc56)cc4)c3)c2c1]

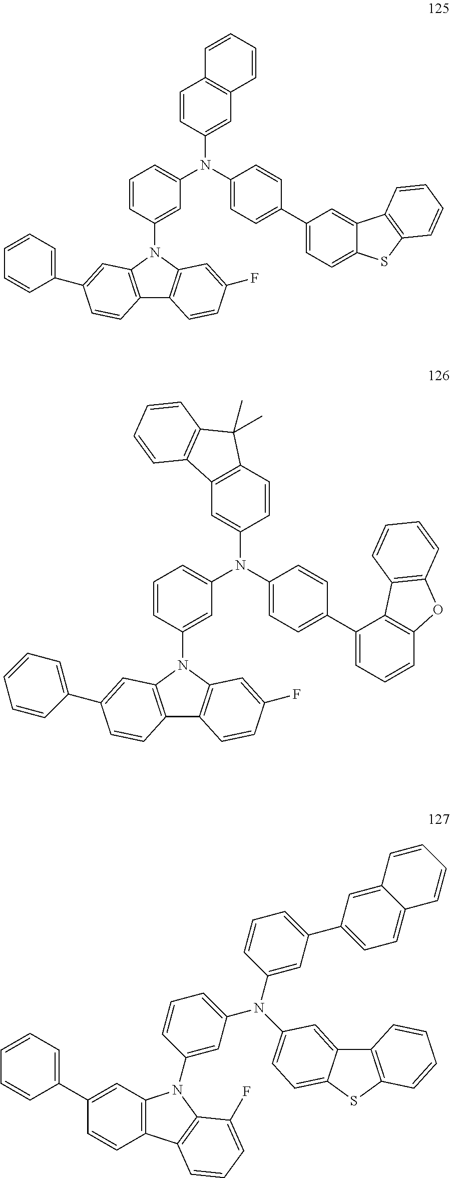 [SMILES: Fc1ccc2c3ccc(-c4ccccc4)cc3n(-c3cccc(N(c4ccc(-c5ccc6sc7ccccc7c6c5)cc4)c4ccc5ccccc5c4)c3)c2c1 ; CC1(C)c2ccccc2-c2cc(N(c3ccc(-c4cccc5oc6ccccc6c45)cc3)c3cccc(-n4c5cc(F)ccc5c5ccc(-c6ccccc6)cc54)c3)ccc21 ; Fc1cccc2c3ccc(-c4ccccc4)cc3n(-c3cccc(N(c4cccc(-c5ccc6ccccc6c5)c4)c4ccc5sc6ccccc6c5c4)c3)c12]

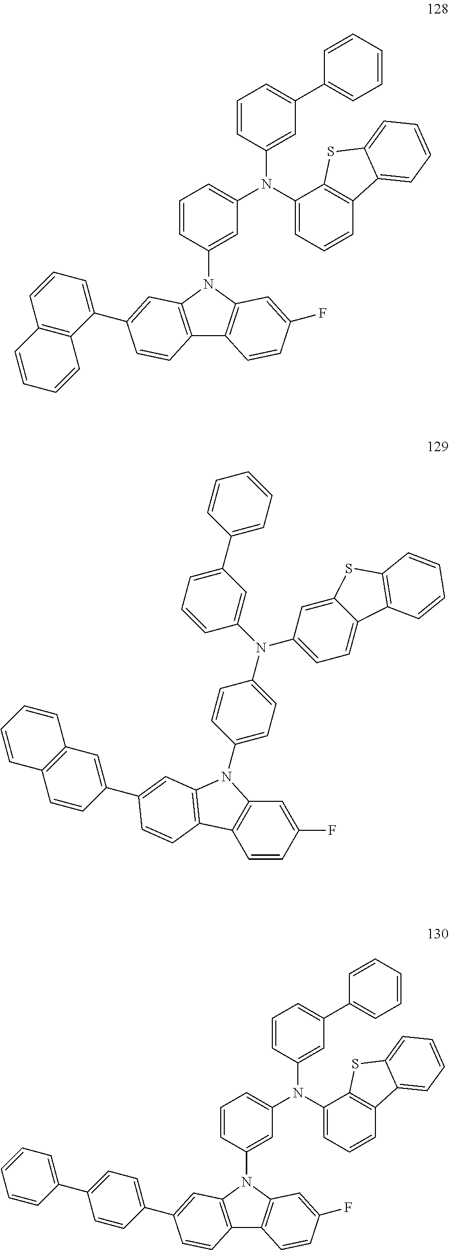 [SMILES: Fc1ccc2c3ccc(-c4cccc5ccccc45)cc3n(-c3cccc(N(c4cccc(-c5ccccc5)c4)c4cccc5c4sc4ccccc45)c3)c2c1 ; Fc1ccc2c3ccc(-c4ccc5ccccc5c4)cc3n(-c3ccc(N(c4cccc(-c5ccccc5)c4)c4ccc5c(c4)sc4ccccc45)cc3)c2c1 ; Fc1ccc2c3ccc(-c4ccc(-c5ccccc5)cc4)cc3n(-c3cccc(N(c4cccc(-c5ccccc5)c4)c4cccc5c4sc4ccccc45)c3)c2c1]

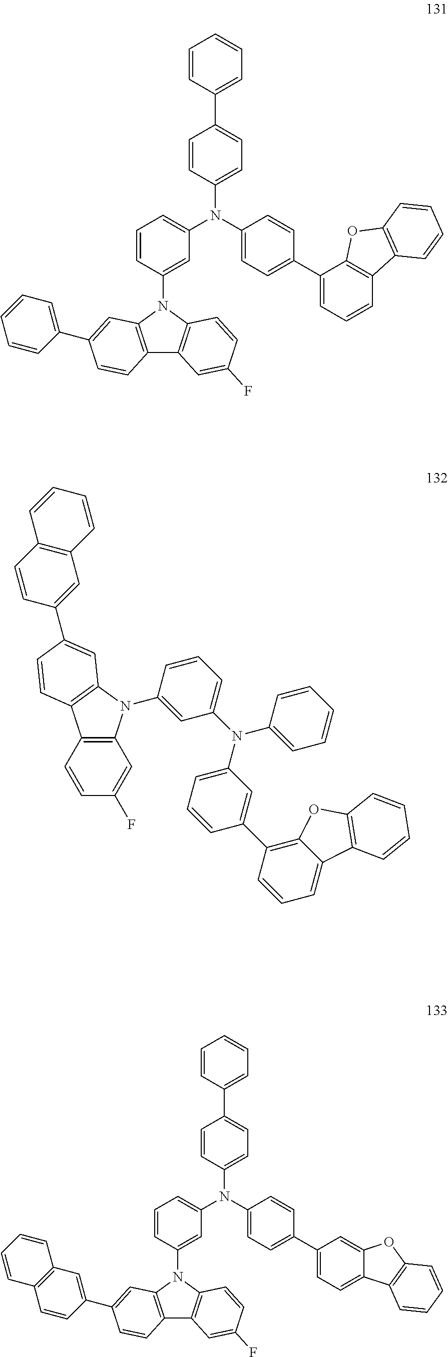 [SMILES: Fc1ccc2c(c1)c1ccc(-c3ccccc3)cc1n2-c1cccc(N(c2ccc(-c3ccccc3)cc2)c2ccc(-c3cccc4c3oc3ccccc34)cc2)c1 ; Fc1ccc2c3ccc(-c4ccc5ccccc5c4)cc3n(-c3cccc(N(c4ccccc4)c4cccc(-c5cccc6c5oc5ccccc56)c4)c3)c2c1 ; Fc1ccc2c(c1)c1ccc(-c3ccc4ccccc4c3)cc1n2-c1cccc(N(c2ccc(-c3ccccc3)cc2)c2ccc(-c3ccc4c(c3)oc3ccccc34)cc2)c1]

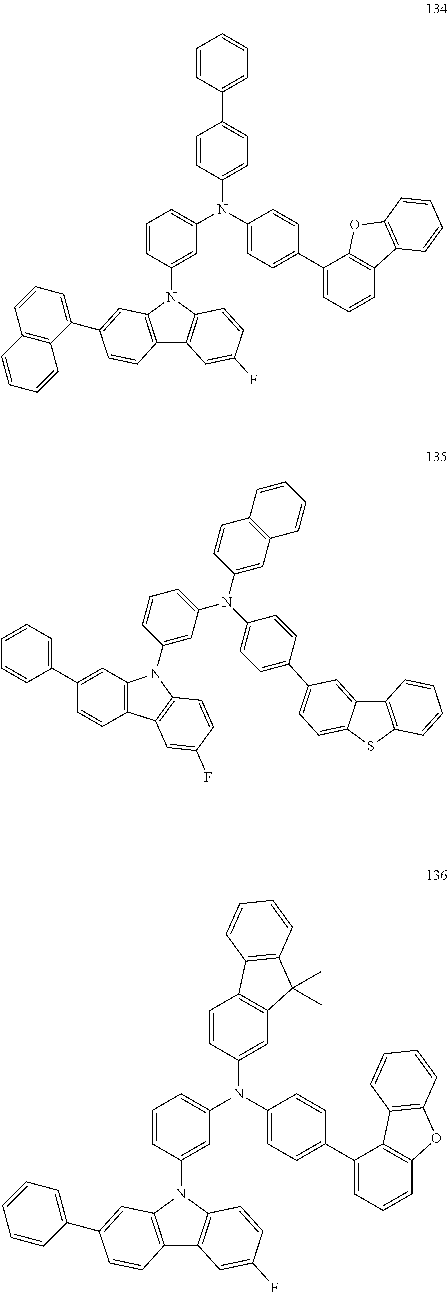 [SMILES: Fc1ccc2c(c1)c1ccc(-c3cccc4ccccc34)cc1n2-c1cccc(N(c2ccc(-c3ccccc3)cc2)c2ccc(-c3cccc4c3oc3ccccc34)cc2)c1 ; Fc1ccc2c(c1)c1ccc(-c3ccccc3)cc1n2-c1cccc(N(c2ccc(-c3ccc4sc5ccccc5c4c3)cc2)c2ccc3ccccc3c2)c1 ; CC1(C)c2ccccc2-c2ccc(N(c3ccc(-c4cccc5oc6ccccc6c45)cc3)c3cccc(-n4c5ccc(F)cc5c5ccc(-c6ccccc6)cc54)c3)cc21]

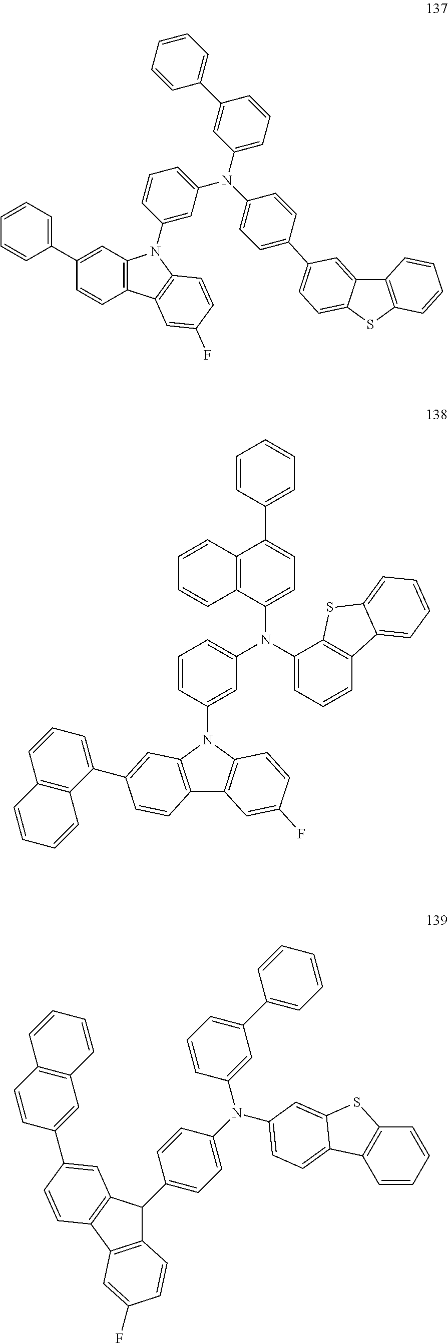 [SMILES: Fc1ccc2c(c1)c1ccc(-c3ccccc3)cc1n2-c1cccc(N(c2ccc(-c3ccc4sc5ccccc5c4c3)cc2)c2cccc(-c3ccccc3)c2)c1 ; Fc1ccc2c(c1)c1ccc(-c3cccc4ccccc34)cc1n2-c1cccc(N(c2ccc(-c3ccccc3)c3ccccc23)c2cccc3c2sc2ccccc23)c1 ; Fc1ccc2c(c1)-c1ccc(-c3ccc4ccccc4c3)cc1C2c1ccc(N(c2cccc(-c3ccccc3)c2)c2ccc3c(c2)sc2ccccc23)cc1]

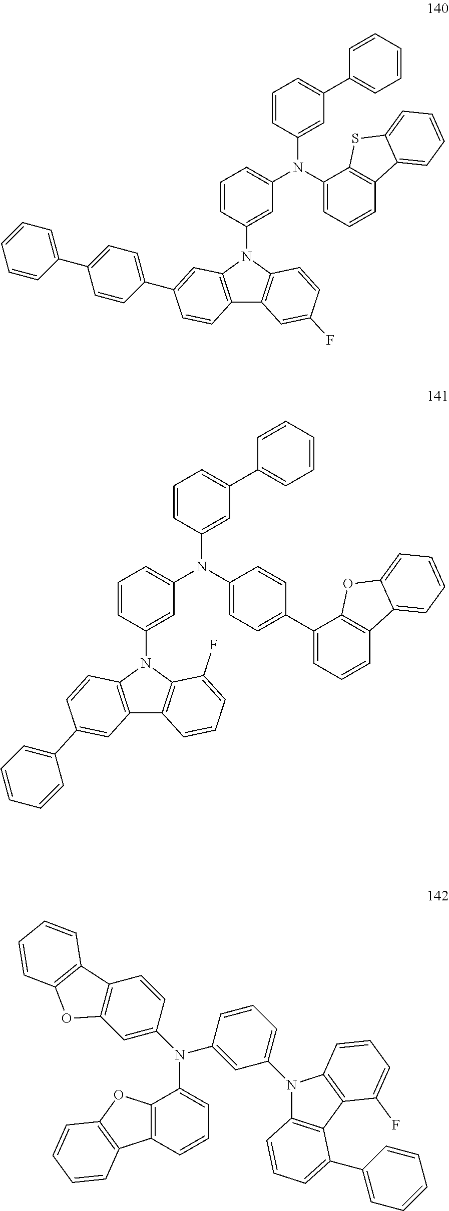 [SMILES: Fc1ccc2c(c1)c1ccc(-c3ccc(-c4ccccc4)cc3)cc1n2-c1cccc(N(c2cccc(-c3ccccc3)c2)c2cccc3c2sc2ccccc23)c1 ; Fc1cccc2c3cc(-c4ccccc4)ccc3n(-c3cccc(N(c4ccc(-c5cccc6c5oc5ccccc56)cc4)c4cccc(-c5ccccc5)c4)c3)c12 ; Fc1cccc2c1c1c(-c3ccccc3)cccc1n2-c1cccc(N(c2ccc3c(c2)oc2ccccc23)c2cccc3c2oc2ccccc23)c1]

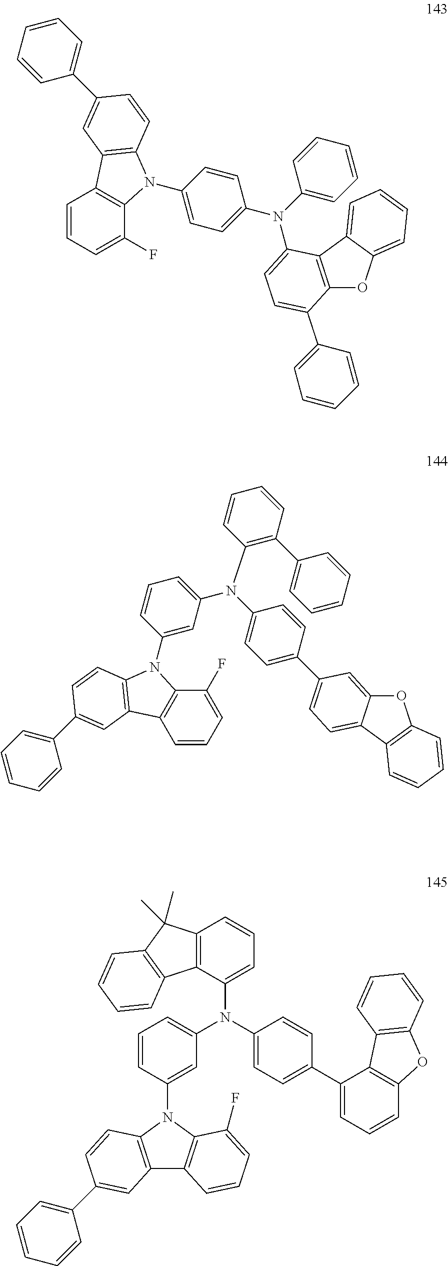 [SMILES: Fc1cccc2c3cc(-c4ccccc4)ccc3n(-c3ccc(N(c4ccccc4)c4ccc(-c5ccccc5)c5oc6ccccc6c45)cc3)c12 ; Fc1cccc2c3cc(-c4ccccc4)ccc3n(-c3cccc(N(c4ccc(-c5ccc6c(c5)oc5ccccc56)cc4)c4ccccc4-c4ccccc4)c3)c12 ; CC1(C)c2ccccc2-c2c(N(c3ccc(-c4cccc5oc6ccccc6c45)cc3)c3cccc(-n4c5ccc(-c6ccccc6)cc5c5cccc(F)c54)c3)cccc21]

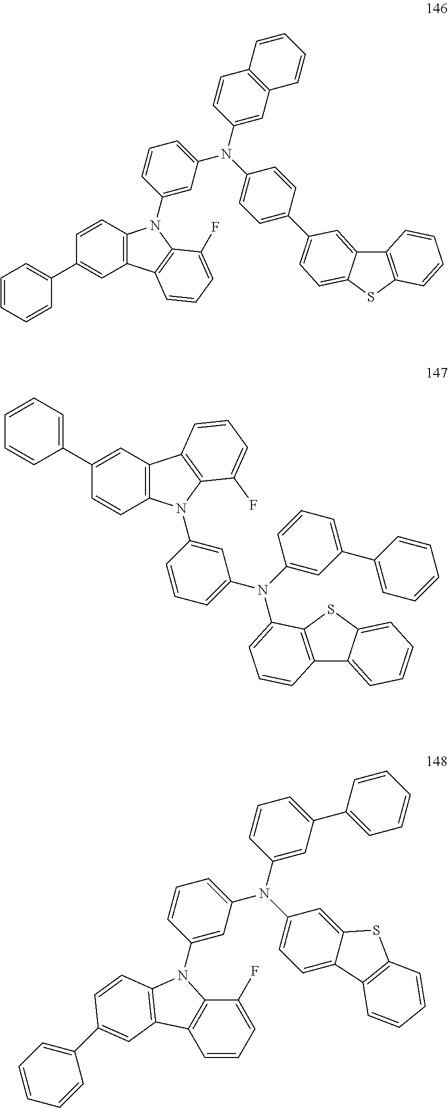 [SMILES: Fc1cccc2c3cc(-c4ccccc4)ccc3n(-c3cccc(N(c4ccc(-c5ccc6sc7ccccc7c6c5)cc4)c4ccc5ccccc5c4)c3)c12 ; Fc1cccc2c3cc(-c4ccccc4)ccc3n(-c3cccc(N(c4cccc(-c5ccccc5)c4)c4cccc5c4sc4ccccc45)c3)c12 ; Fc1cccc2c3cc(-c4ccccc4)ccc3n(-c3cccc(N(c4cccc(-c5ccccc5)c4)c4ccc5c(c4)sc4ccccc45)c3)c12]

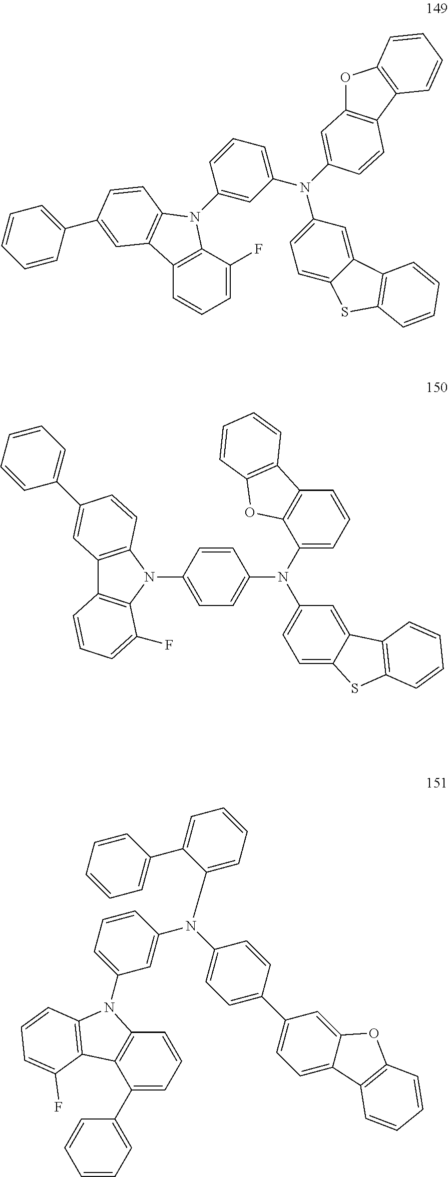 [SMILES: Fc1cccc2c3cc(-c4ccccc4)ccc3n(-c3cccc(N(c4ccc5c(c4)oc4ccccc45)c4ccc5sc6ccccc6c5c4)c3)c12 ; Fc1cccc2c3cc(-c4ccccc4)ccc3n(-c3ccc(N(c4ccc5sc6ccccc6c5c4)c4cccc5c4oc4ccccc45)cc3)c12 ; Fc1cccc2c1c1c(-c3ccccc3)cccc1n2-c1cccc(N(c2ccc(-c3ccc4c(c3)oc3ccccc34)cc2)c2ccccc2-c2ccccc2)c1]

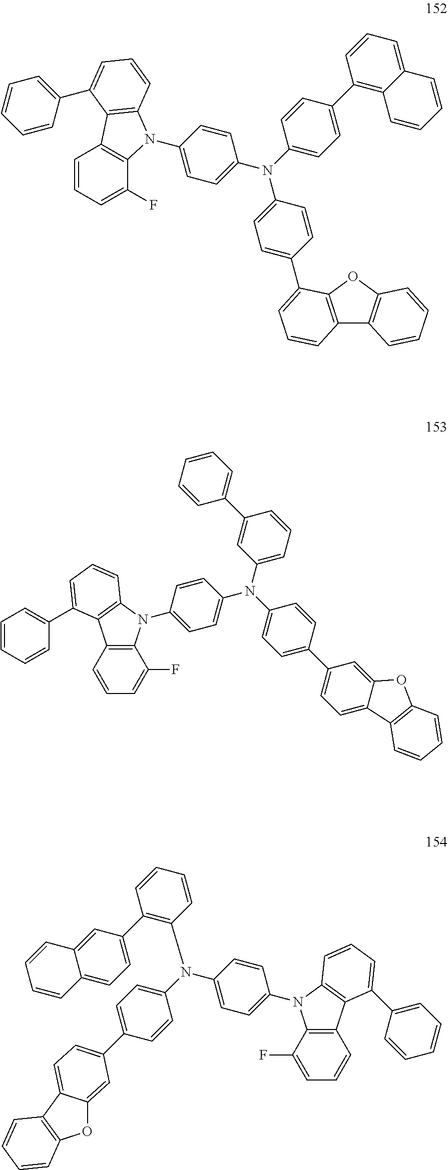 [SMILES: Fc1cccc2c3c(-c4ccccc4)cccc3n(-c3ccc(N(c4ccc(-c5cccc6ccccc56)cc4)c4ccc(-c5cccc6c5oc5ccccc56)cc4)cc3)c12 ; Fc1cccc2c3c(-c4ccccc4)cccc3n(-c3ccc(N(c4ccc(-c5ccc6c(c5)oc5ccccc56)cc4)c4cccc(-c5ccccc5)c4)cc3)c12 ; Fc1cccc2c3c(-c4ccccc4)cccc3n(-c3ccc(N(c4ccc(-c5ccc6c(c5)oc5ccccc56)cc4)c4ccccc4-c4ccc5ccccc5c4)cc3)c12]

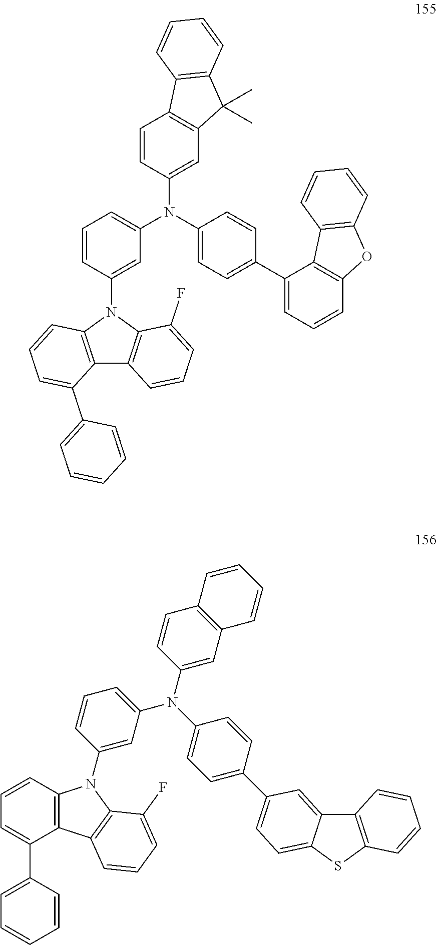 [SMILES: CC1(C)c2ccccc2-c2ccc(N(c3ccc(-c4cccc5oc6ccccc6c45)cc3)c3cccc(-n4c5cccc(-c6ccccc6)c5c5cccc(F)c54)c3)cc21 ; Fc1cccc2c3c(-c4ccccc4)cccc3n(-c3cccc(N(c4ccc(-c5ccc6sc7ccccc7c6c5)cc4)c4ccc5ccccc5c4)c3)c12]

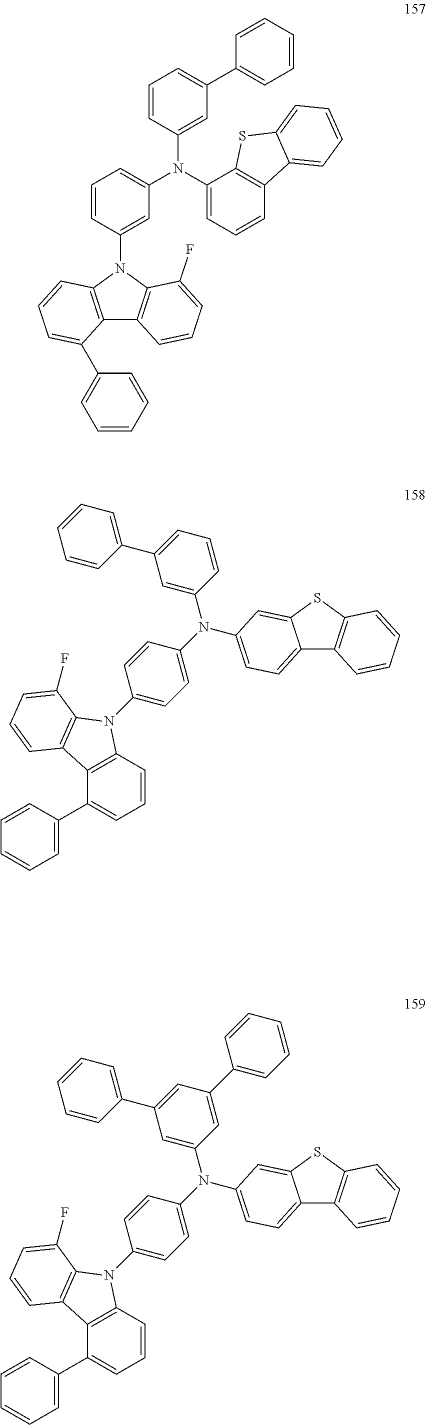 [SMILES: Fc1cccc2c3c(-c4ccccc4)cccc3n(-c3cccc(N(c4cccc(-c5ccccc5)c4)c4cccc5c4sc4ccccc45)c3)c12 ; Fc1cccc2c3c(-c4ccccc4)cccc3n(-c3ccc(N(c4cccc(-c5ccccc5)c4)c4ccc5c(c4)sc4ccccc45)cc3)c12 ; Fc1cccc2c3c(-c4ccccc4)cccc3n(-c3ccc(N(c4cc(-c5ccccc5)cc(-c5ccccc5)c4)c4ccc5c(c4)sc4ccccc45)cc3)c12]

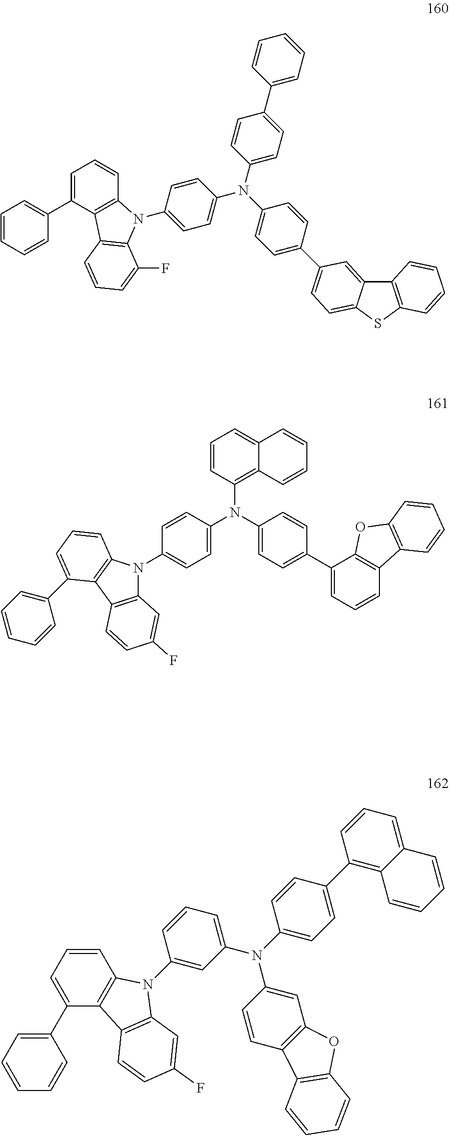 [SMILES: Fc1cccc2c3c(-c4ccccc4)cccc3n(-c3ccc(N(c4ccc(-c5ccccc5)cc4)c4ccc(-c5ccc6sc7ccccc7c6c5)cc4)cc3)c12 ; Fc1ccc2c3c(-c4ccccc4)cccc3n(-c3ccc(N(c4ccc(-c5cccc6c5oc5ccccc56)cc4)c4cccc5ccccc45)cc3)c2c1 ; Fc1ccc2c3c(-c4ccccc4)cccc3n(-c3cccc(N(c4ccc(-c5cccc6ccccc56)cc4)c4ccc5c(c4)oc4ccccc45)c3)c2c1]

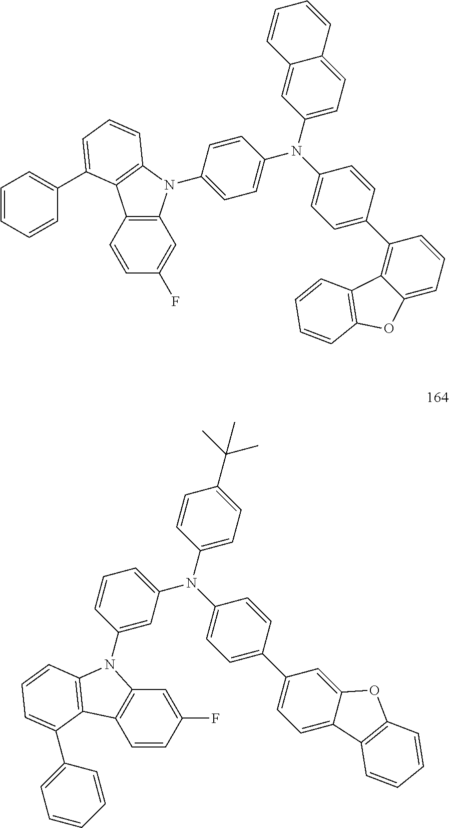 [SMILES: Fc1ccc2c3c(-c4ccccc4)cccc3n(-c3ccc(N(c4ccc(-c5cccc6oc7ccccc7c56)cc4)c4ccc5ccccc5c4)cc3)c2c1 ; CC(C)(C)c1ccc(N(c2ccc(-c3ccc4c(c3)oc3ccccc34)cc2)c2cccc(-n3c4cc(F)ccc4c4c(-c5ccccc5)cccc43)c2)cc1]

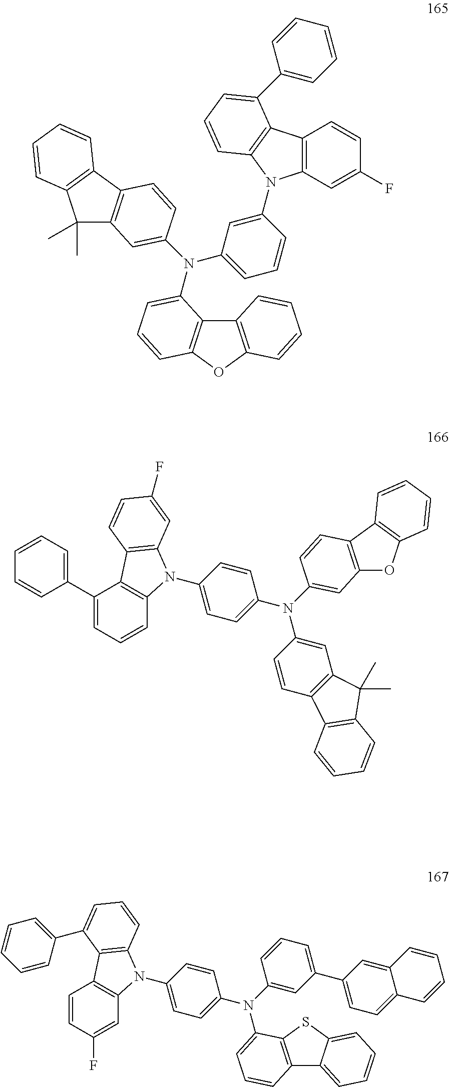 [SMILES: CC1(C)c2ccccc2-c2ccc(N(c3cccc(-n4c5cc(F)ccc5c5c(-c6ccccc6)cccc54)c3)c3cccc4oc5ccccc5c34)cc21 ; CC1(C)c2ccccc2-c2ccc(N(c3ccc(-n4c5cc(F)ccc5c5c(-c6ccccc6)cccc54)cc3)c3ccc4c(c3)oc3ccccc34)cc21 ; Fc1ccc2c3c(-c4ccccc4)cccc3n(-c3ccc(N(c4cccc(-c5ccc6ccccc6c5)c4)c4cccc5c4sc4ccccc45)cc3)c2c1]

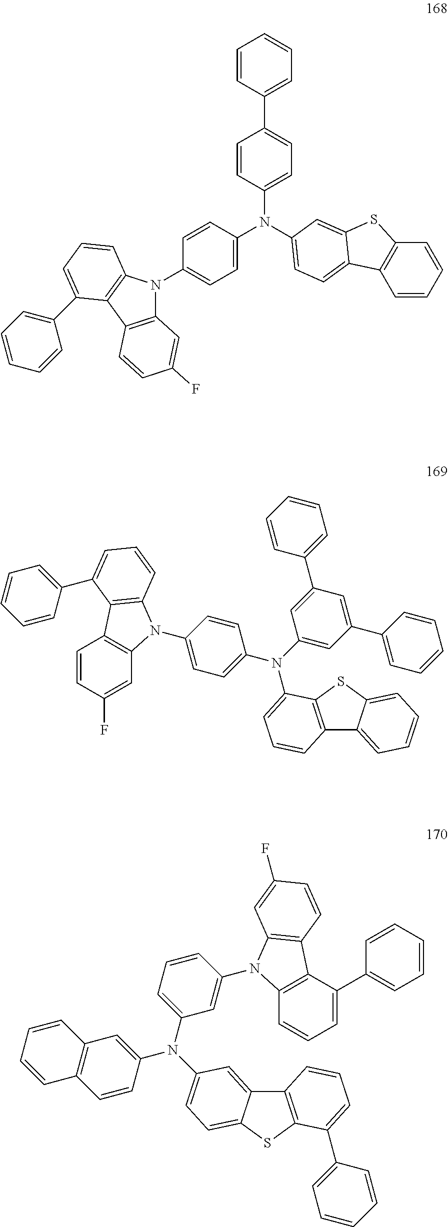 [SMILES: Fc1ccc2c3c(-c4ccccc4)cccc3n(-c3ccc(N(c4ccc(-c5ccccc5)cc4)c4ccc5c(c4)sc4ccccc45)cc3)c2c1 ; Fc1ccc2c3c(-c4ccccc4)cccc3n(-c3ccc(N(c4cc(-c5ccccc5)cc(-c5ccccc5)c4)c4cccc5c4sc4ccccc45)cc3)c2c1 ; Fc1ccc2c3c(-c4ccccc4)cccc3n(-c3cccc(N(c4ccc5ccccc5c4)c4ccc5sc6c(-c7ccccc7)cccc6c5c4)c3)c2c1]

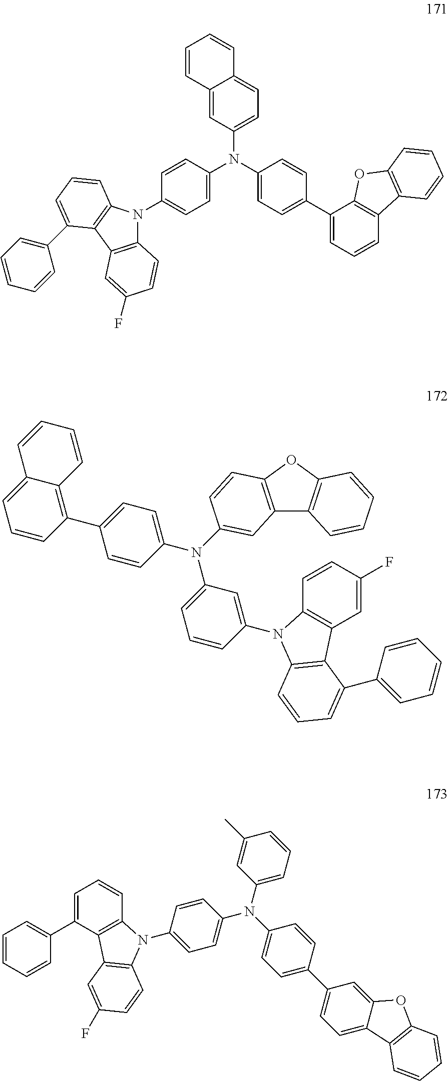 [SMILES: Fc1ccc2c(c1)c1c(-c3ccccc3)cccc1n2-c1ccc(N(c2ccc(-c3cccc4c3oc3ccccc34)cc2)c2ccc3ccccc3c2)cc1 ; Fc1ccc2c(c1)c1c(-c3ccccc3)cccc1n2-c1cccc(N(c2ccc(-c3cccc4ccccc34)cc2)c2ccc3oc4ccccc4c3c2)c1 ; Cc1cccc(N(c2ccc(-c3ccc4c(c3)oc3ccccc34)cc2)c2ccc(-n3c4ccc(F)cc4c4c(-c5ccccc5)cccc43)cc2)c1]

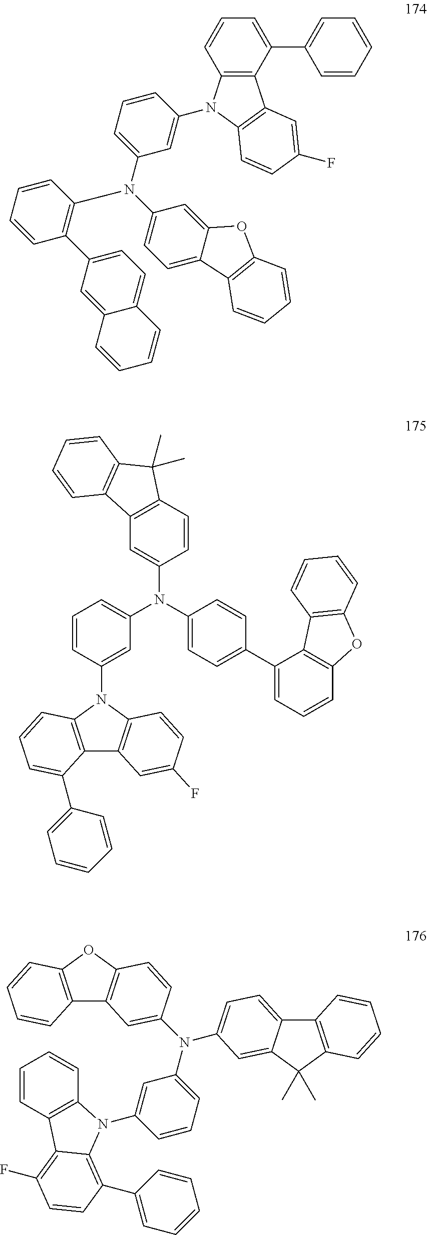 [SMILES: Fc1ccc2c(c1)c1c(-c3ccccc3)cccc1n2-c1cccc(N(c2ccc3c(c2)oc2ccccc23)c2ccccc2-c2ccc3ccccc3c2)c1 ; CC1(C)c2ccccc2-c2cc(N(c3ccc(-c4cccc5oc6ccccc6c45)cc3)c3cccc(-n4c5ccc(F)cc5c5c(-c6ccccc6)cccc54)c3)ccc21 ; CC1(C)c2ccccc2-c2ccc(N(c3cccc(-n4c5ccccc5c5c(F)ccc(-c6ccccc6)c54)c3)c3ccc4oc5ccccc5c4c3)cc21]

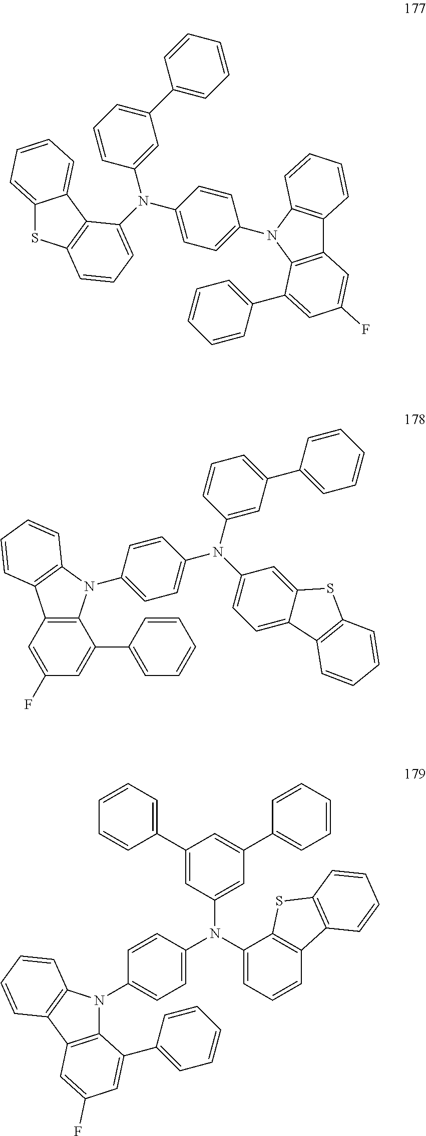 [SMILES: Fc1cc(-c2ccccc2)c2c(c1)c1ccccc1n2-c1ccc(N(c2cccc(-c3ccccc3)c2)c2cccc3sc4ccccc4c23)cc1 ; Fc1cc(-c2ccccc2)c2c(c1)c1ccccc1n2-c1ccc(N(c2cccc(-c3ccccc3)c2)c2ccc3c(c2)sc2ccccc23)cc1 ; Fc1cc(-c2ccccc2)c2c(c1)c1ccccc1n2-c1ccc(N(c2cc(-c3ccccc3)cc(-c3ccccc3)c2)c2cccc3c2sc2ccccc23)cc1]

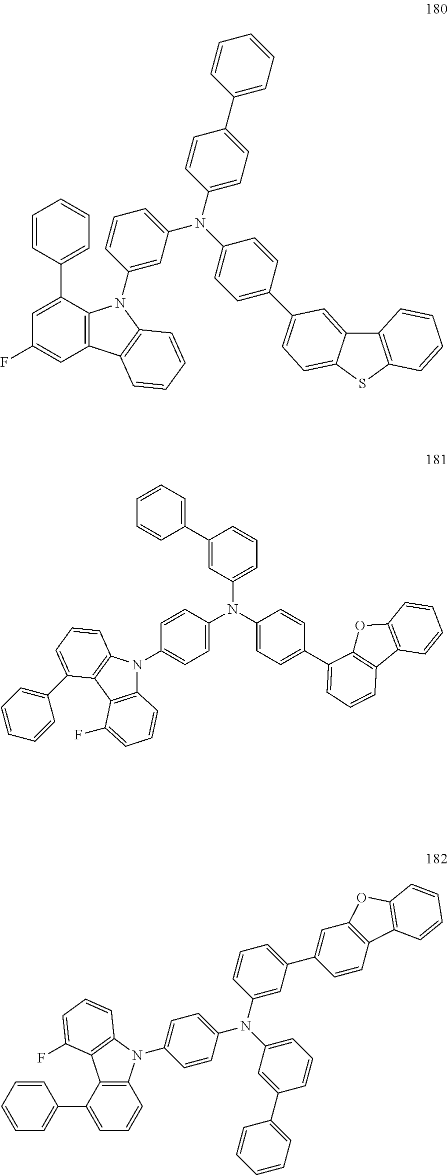 [SMILES: Fc1cc(-c2ccccc2)c2c(c1)c1ccccc1n2-c1cccc(N(c2ccc(-c3ccccc3)cc2)c2ccc(-c3ccc4sc5ccccc5c4c3)cc2)c1 ; Fc1cccc2c1c1c(-c3ccccc3)cccc1n2-c1ccc(N(c2ccc(-c3cccc4c3oc3ccccc34)cc2)c2cccc(-c3ccccc3)c2)cc1 ; Fc1cccc2c1c1c(-c3ccccc3)cccc1n2-c1ccc(N(c2cccc(-c3ccccc3)c2)c2cccc(-c3ccc4c(c3)oc3ccccc34)c2)cc1]

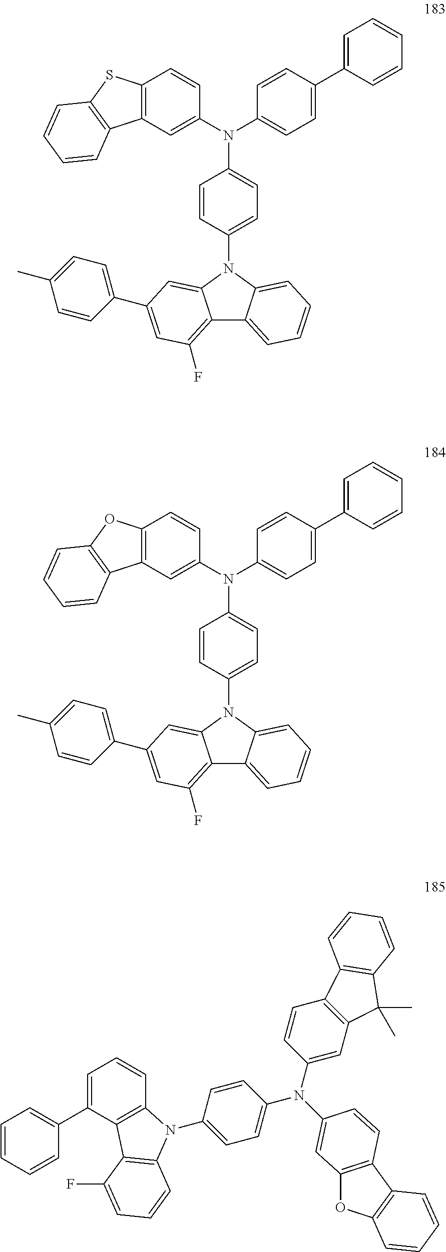 [SMILES: Cc1ccc(-c2cc(F)c3c4ccccc4n(-c4ccc(N(c5ccc(-c6ccccc6)cc5)c5ccc6sc7ccccc7c6c5)cc4)c3c2)cc1 ; Cc1ccc(-c2cc(F)c3c4ccccc4n(-c4ccc(N(c5ccc(-c6ccccc6)cc5)c5ccc6oc7ccccc7c6c5)cc4)c3c2)cc1 ; CC1(C)c2ccccc2-c2ccc(N(c3ccc(-n4c5cccc(F)c5c5c(-c6ccccc6)cccc54)cc3)c3ccc4c(c3)oc3ccccc34)cc21]

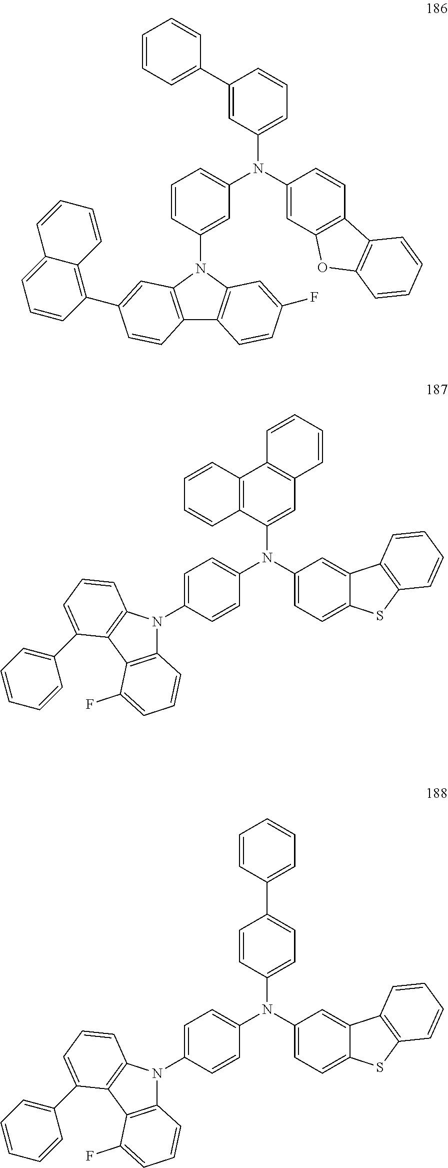 [SMILES: Fc1ccc2c3ccc(-c4cccc5ccccc45)cc3n(-c3cccc(N(c4cccc(-c5ccccc5)c4)c4ccc5c(c4)oc4ccccc45)c3)c2c1 ; Fc1cccc2c1c1c(-c3ccccc3)cccc1n2-c1ccc(N(c2ccc3sc4ccccc4c3c2)c2cc3ccccc3c3ccccc23)cc1 ; Fc1cccc2c1c1c(-c3ccccc3)cccc1n2-c1ccc(N(c2ccc(-c3ccccc3)cc2)c2ccc3sc4ccccc4c3c2)cc1]

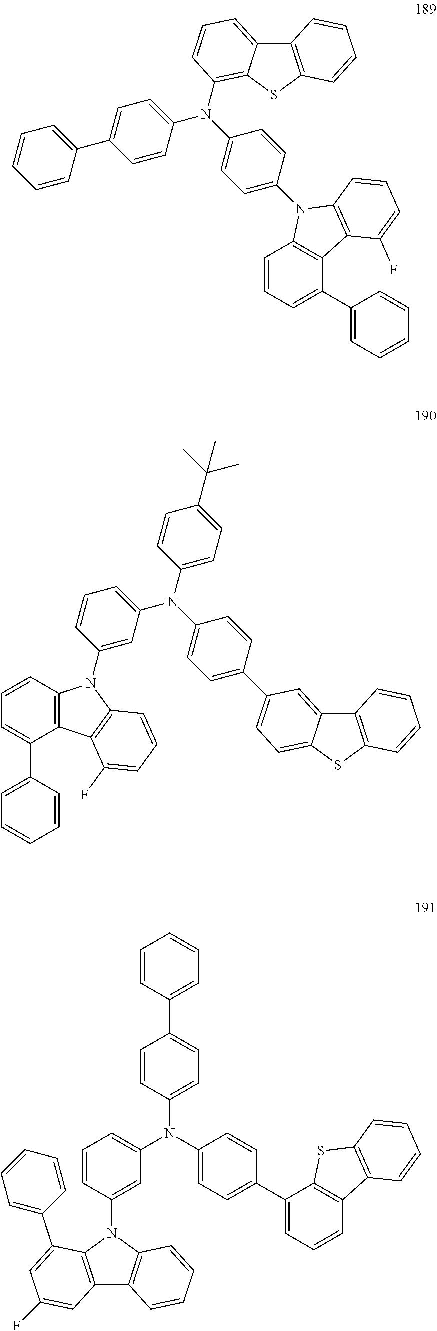 [SMILES: Fc1cccc2c1c1c(-c3ccccc3)cccc1n2-c1ccc(N(c2ccc(-c3ccccc3)cc2)c2cccc3c2sc2ccccc23)cc1 ; CC(C)(C)c1ccc(N(c2ccc(-c3ccc4sc5ccccc5c4c3)cc2)c2cccc(-n3c4cccc(F)c4c4c(-c5ccccc5)cccc43)c2)cc1 ; Fc1cc(-c2ccccc2)c2c(c1)c1ccccc1n2-c1cccc(N(c2ccc(-c3ccccc3)cc2)c2ccc(-c3cccc4c3sc3ccccc34)cc2)c1]

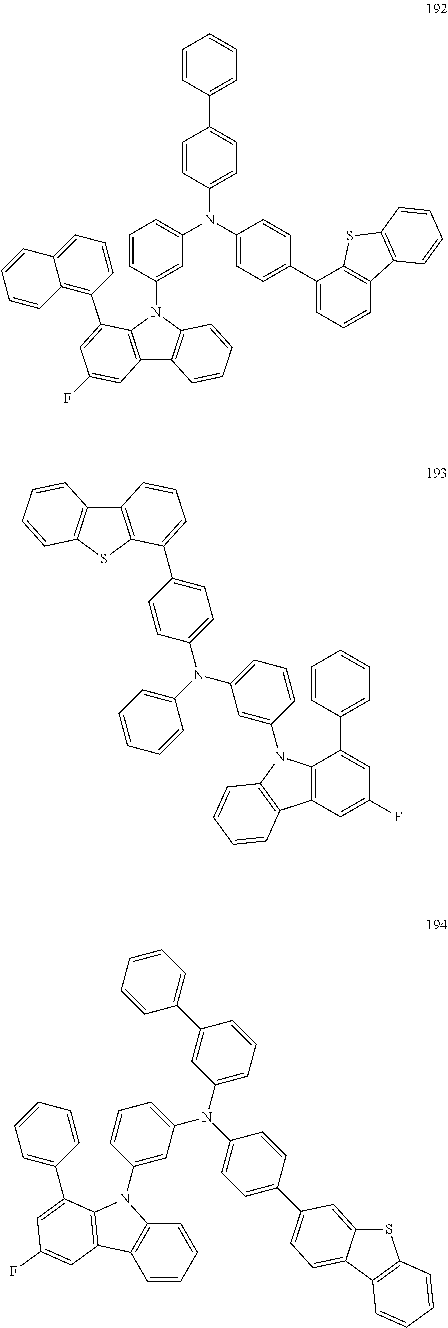 [SMILES: Fc1cc(-c2cccc3ccccc23)c2c(c1)c1ccccc1n2-c1cccc(N(c2ccc(-c3ccccc3)cc2)c2ccc(-c3cccc4c3sc3ccccc34)cc2)c1 ; Fc1cc(-c2ccccc2)c2c(c1)c1ccccc1n2-c1cccc(N(c2ccccc2)c2ccc(-c3cccc4c3sc3ccccc34)cc2)c1 ; Fc1cc(-c2ccccc2)c2c(c1)c1ccccc1n2-c1cccc(N(c2ccc(-c3ccc4c(c3)sc3ccccc34)cc2)c2cccc(-c3ccccc3)c2)c1]

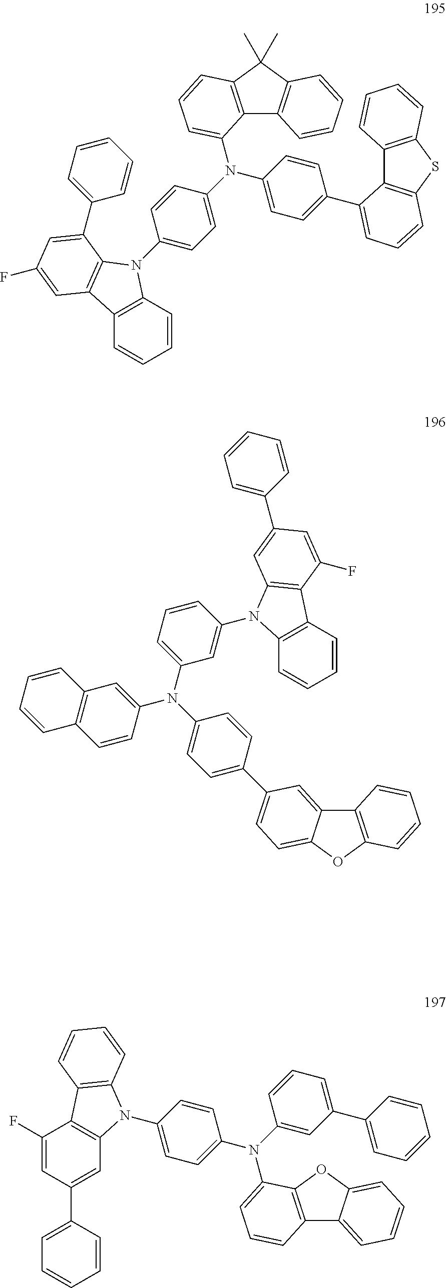 [SMILES: CC1(C)c2ccccc2-c2c(N(c3ccc(-c4cccc5sc6ccccc6c45)cc3)c3ccc(-n4c5ccccc5c5cc(F)cc(-c6ccccc6)c54)cc3)cccc21 ; Fc1cc(-c2ccccc2)cc2c1c1ccccc1n2-c1cccc(N(c2ccc(-c3ccc4oc5ccccc5c4c3)cc2)c2ccc3ccccc3c2)c1 ; Fc1cc(-c2ccccc2)cc2c1c1ccccc1n2-c1ccc(N(c2cccc(-c3ccccc3)c2)c2cccc3c2oc2ccccc23)cc1]

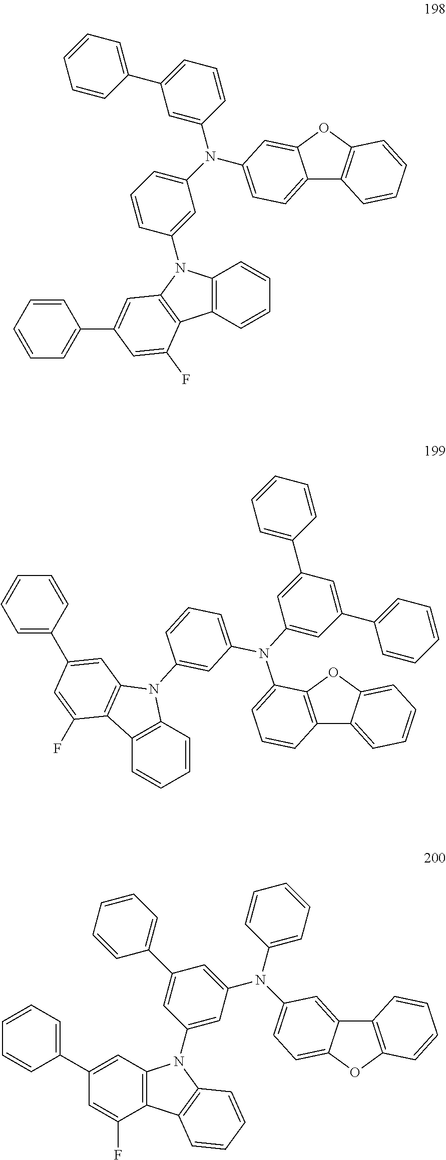 [SMILES: Fc1cc(-c2ccccc2)cc2c1c1ccccc1n2-c1cccc(N(c2cccc(-c3ccccc3)c2)c2ccc3c(c2)oc2ccccc23)c1 ; Fc1cc(-c2ccccc2)cc2c1c1ccccc1n2-c1cccc(N(c2cc(-c3ccccc3)cc(-c3ccccc3)c2)c2cccc3c2oc2ccccc23)c1 ; Fc1cc(-c2ccccc2)cc2c1c1ccccc1n2-c1cc(-c2ccccc2)cc(N(c2ccccc2)c2ccc3oc4ccccc4c3c2)c1]

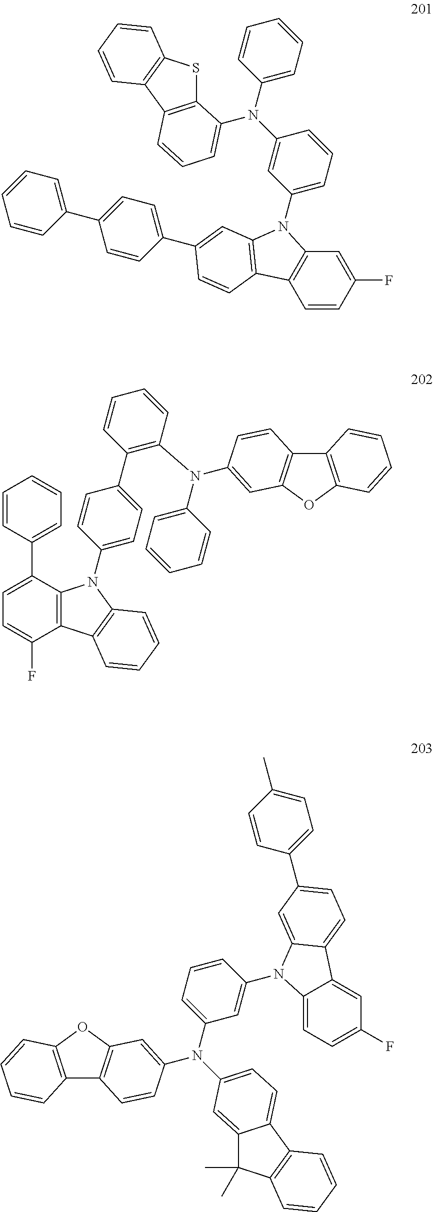 [SMILES: Fc1ccc2c3ccc(-c4ccc(-c5ccccc5)cc4)cc3n(-c3cccc(N(c4ccccc4)c4cccc5c4sc4ccccc45)c3)c2c1 ; Fc1ccc(-c2ccccc2)c2c1c1ccccc1n2-c1ccc(-c2ccccc2N(c2ccccc2)c2ccc3c(c2)oc2ccccc23)cc1 ; Cc1ccc(-c2ccc3c4cc(F)ccc4n(-c4cccc(N(c5ccc6c(c5)C(C)(C)c5ccccc5-6)c5ccc6c(c5)oc5ccccc56)c4)c3c2)cc1]

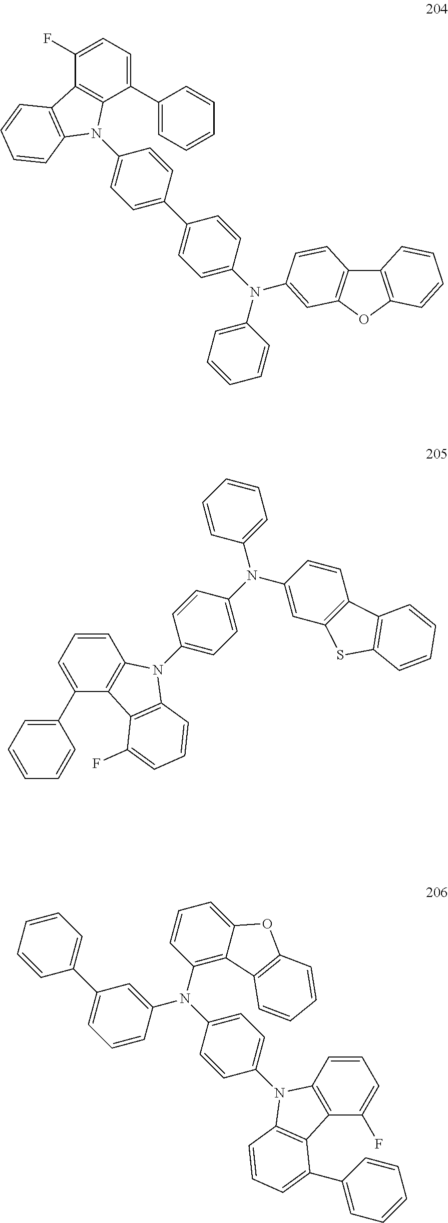 [SMILES: Fc1ccc(-c2ccccc2)c2c1c1ccccc1n2-c1ccc(-c2ccc(N(c3ccccc3)c3ccc4c(c3)oc3ccccc34)cc2)cc1 ; Fc1cccc2c1c1c(-c3ccccc3)cccc1n2-c1ccc(N(c2ccccc2)c2ccc3c(c2)sc2ccccc23)cc1 ; Fc1cccc2c1c1c(-c3ccccc3)cccc1n2-c1ccc(N(c2cccc(-c3ccccc3)c2)c2cccc3oc4ccccc4c23)cc1]

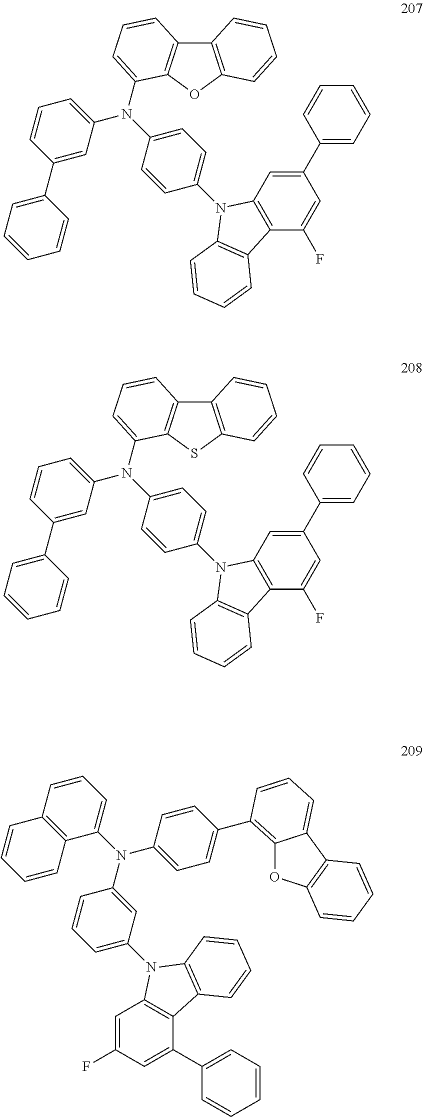 [SMILES: Fc1cc(-c2ccccc2)cc2c1c1ccccc1n2-c1ccc(N(c2cccc(-c3ccccc3)c2)c2cccc3c2oc2ccccc23)cc1 ; Fc1cc(-c2ccccc2)cc2c1c1ccccc1n2-c1ccc(N(c2cccc(-c3ccccc3)c2)c2cccc3c2sc2ccccc23)cc1 ; Fc1cc(-c2ccccc2)c2c3ccccc3n(-c3cccc(N(c4ccc(-c5cccc6c5oc5ccccc56)cc4)c4cccc5ccccc45)c3)c2c1]

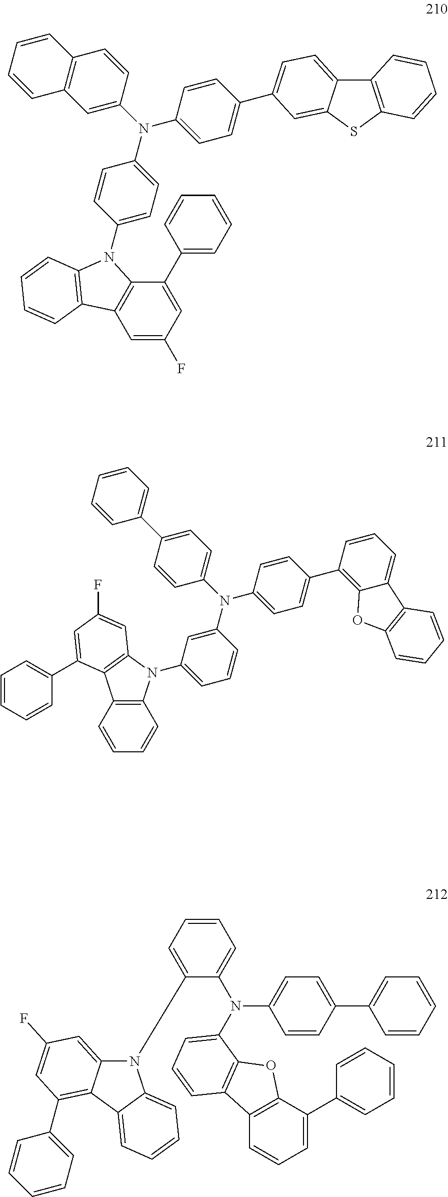 [SMILES: Fc1cc(-c2ccccc2)c2c(c1)c1ccccc1n2-c1ccc(N(c2ccc(-c3ccc4c(c3)sc3ccccc34)cc2)c2ccc3ccccc3c2)cc1 ; Fc1cc(-c2ccccc2)c2c3ccccc3n(-c3cccc(N(c4ccc(-c5ccccc5)cc4)c4ccc(-c5cccc6c5oc5ccccc56)cc4)c3)c2c1 ; Fc1cc(-c2ccccc2)c2c3ccccc3n(-c3ccccc3N(c3ccc(-c4ccccc4)cc3)c3cccc4c3oc3c(-c5ccccc5)cccc34)c2c1]

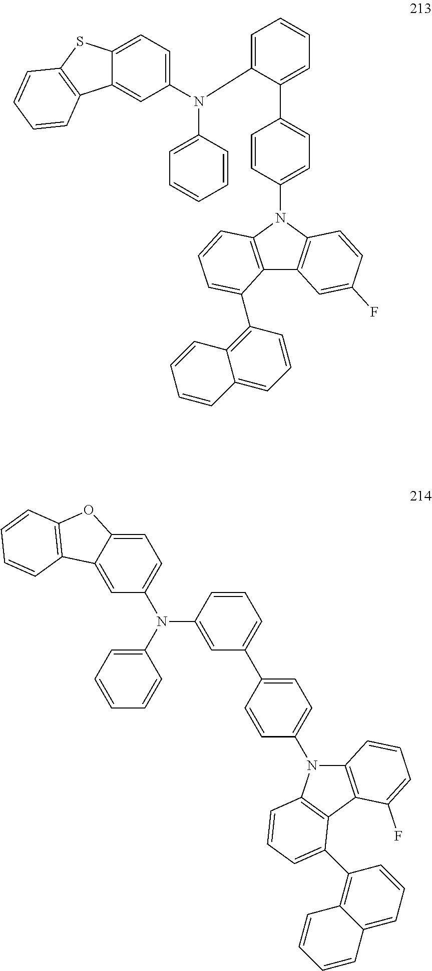 [SMILES: Fc1ccc2c(c1)c1c(-c3cccc4ccccc34)cccc1n2-c1ccc(-c2ccccc2N(c2ccccc2)c2ccc3sc4ccccc4c3c2)cc1 ; Fc1cccc2c1c1c(-c3cccc4ccccc34)cccc1n2-c1ccc(-c2cccc(N(c3ccccc3)c3ccc4oc5ccccc5c4c3)c2)cc1]

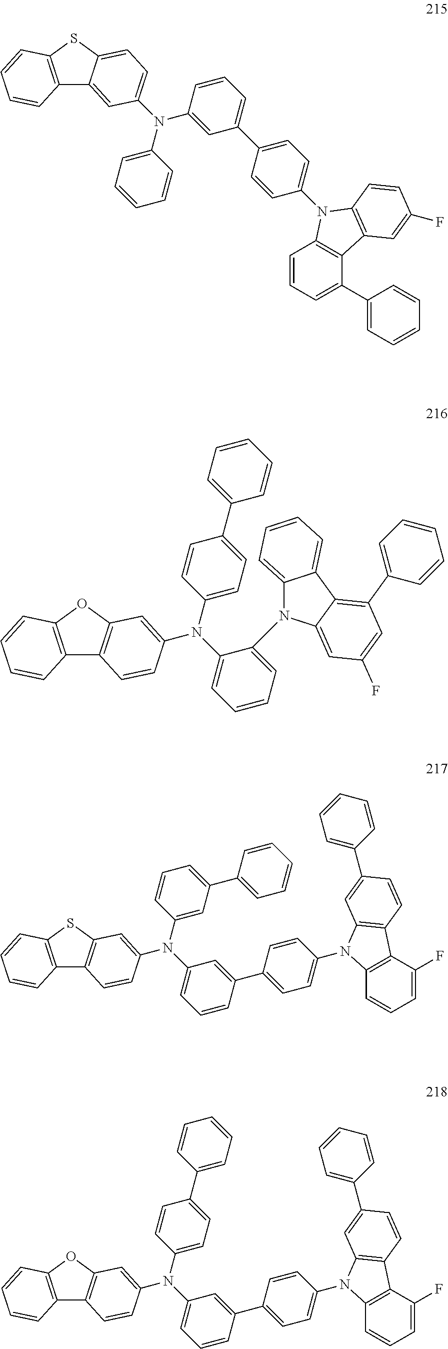 [SMILES: Fc1ccc2c(c1)c1c(-c3ccccc3)cccc1n2-c1ccc(-c2cccc(N(c3ccccc3)c3ccc4sc5ccccc5c4c3)c2)cc1 ; Fc1cc(-c2ccccc2)c2c3ccccc3n(-c3ccccc3N(c3ccc(-c4ccccc4)cc3)c3ccc4c(c3)oc3ccccc34)c2c1 ; Fc1cccc2c1c1ccc(-c3ccccc3)cc1n2-c1ccc(-c2cccc(N(c3cccc(-c4ccccc4)c3)c3ccc4c(c3)sc3ccccc34)c2)cc1 ; Fc1cccc2c1c1ccc(-c3ccccc3)cc1n2-c1ccc(-c2cccc(N(c3ccc(-c4ccccc4)cc3)c3ccc4c(c3)oc3ccccc34)c2)cc1]

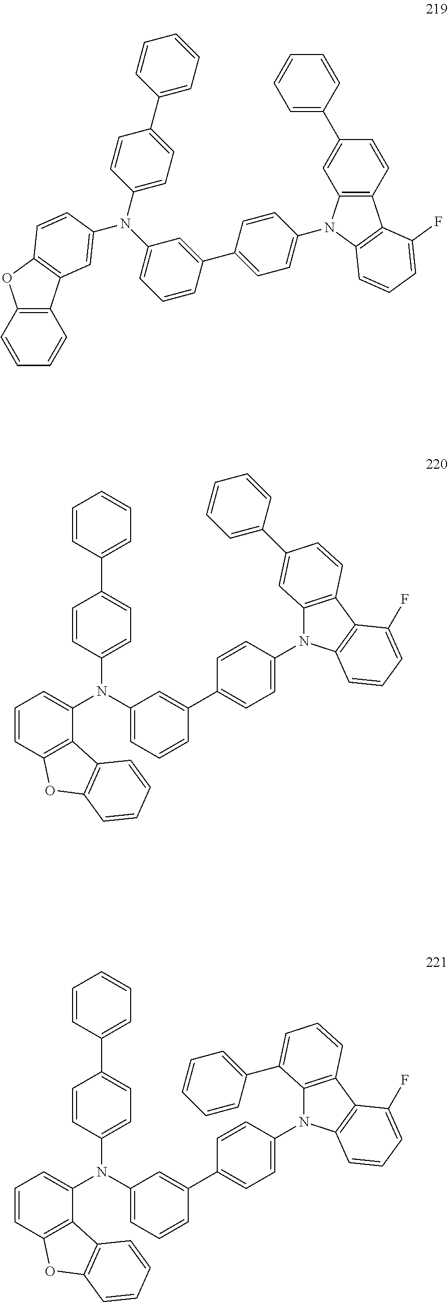 [SMILES: Fc1cccc2c1c1ccc(-c3ccccc3)cc1n2-c1ccc(-c2cccc(N(c3ccc(-c4ccccc4)cc3)c3ccc4oc5ccccc5c4c3)c2)cc1 ; Fc1cccc2c1c1ccc(-c3ccccc3)cc1n2-c1ccc(-c2cccc(N(c3ccc(-c4ccccc4)cc3)c3cccc4oc5ccccc5c34)c2)cc1 ; Fc1cccc2c1c1cccc(-c3ccccc3)c1n2-c1ccc(-c2cccc(N(c3ccc(-c4ccccc4)cc3)c3cccc4oc5ccccc5c34)c2)cc1]

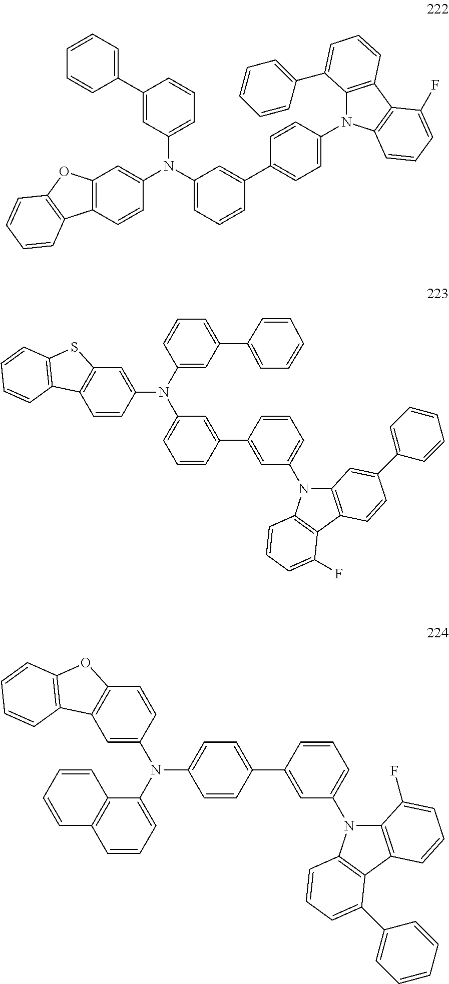 [SMILES: Fc1cccc2c1c1cccc(-c3ccccc3)c1n2-c1ccc(-c2cccc(N(c3cccc(-c4ccccc4)c3)c3ccc4c(c3)oc3ccccc34)c2)cc1 ; Fc1cccc2c1c1ccc(-c3ccccc3)cc1n2-c1cccc(-c2cccc(N(c3cccc(-c4ccccc4)c3)c3ccc4c(c3)sc3ccccc34)c2)c1 ; Fc1cccc2c3c(-c4ccccc4)cccc3n(-c3cccc(-c4ccc(N(c5ccc6oc7ccccc7c6c5)c5cccc6ccccc56)cc4)c3)c12]

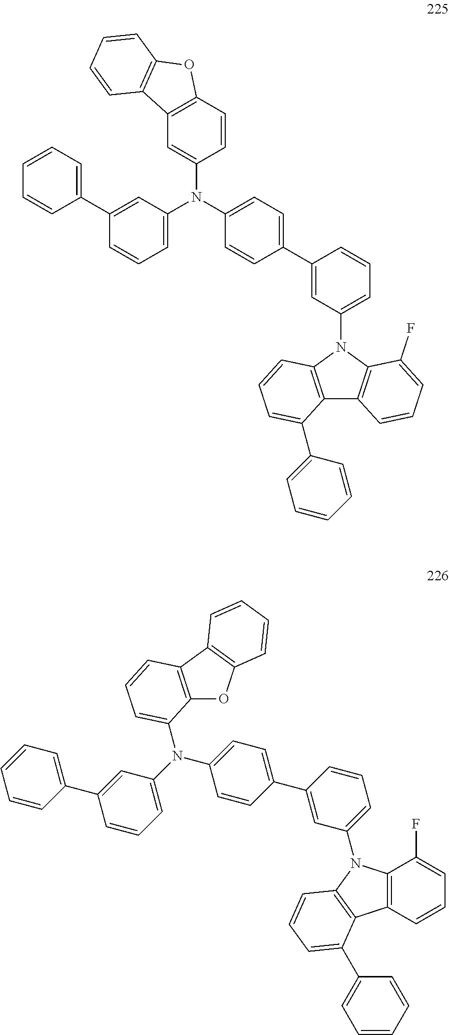 [SMILES: Fc1cccc2c3c(-c4ccccc4)cccc3n(-c3cccc(-c4ccc(N(c5cccc(-c6ccccc6)c5)c5ccc6oc7ccccc7c6c5)cc4)c3)c12 ; Fc1cccc2c3c(-c4ccccc4)cccc3n(-c3cccc(-c4ccc(N(c5cccc(-c6ccccc6)c5)c5cccc6c5oc5ccccc56)cc4)c3)c12]

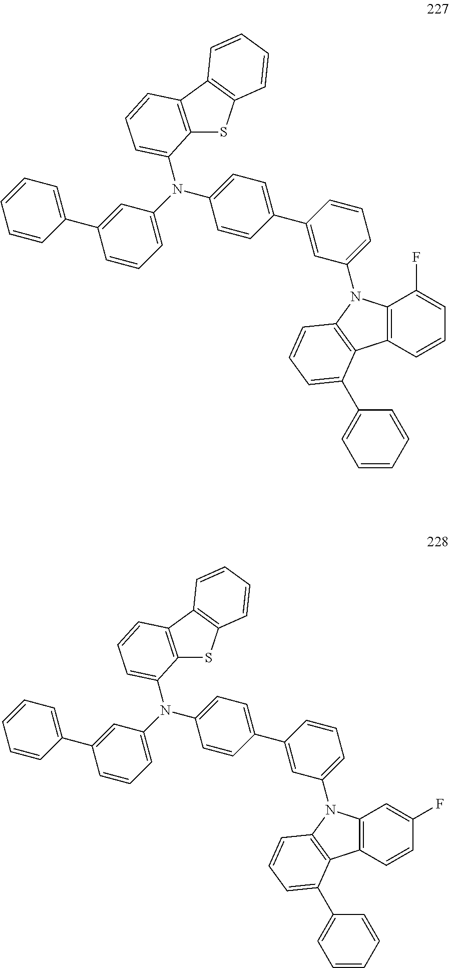 [SMILES: Fc1cccc2c3c(-c4ccccc4)cccc3n(-c3cccc(-c4ccc(N(c5cccc(-c6ccccc6)c5)c5cccc6c5sc5ccccc56)cc4)c3)c12 ; Fc1ccc2c3c(-c4ccccc4)cccc3n(-c3cccc(-c4ccc(N(c5cccc(-c6ccccc6)c5)c5cccc6c5sc5ccccc56)cc4)c3)c2c1]

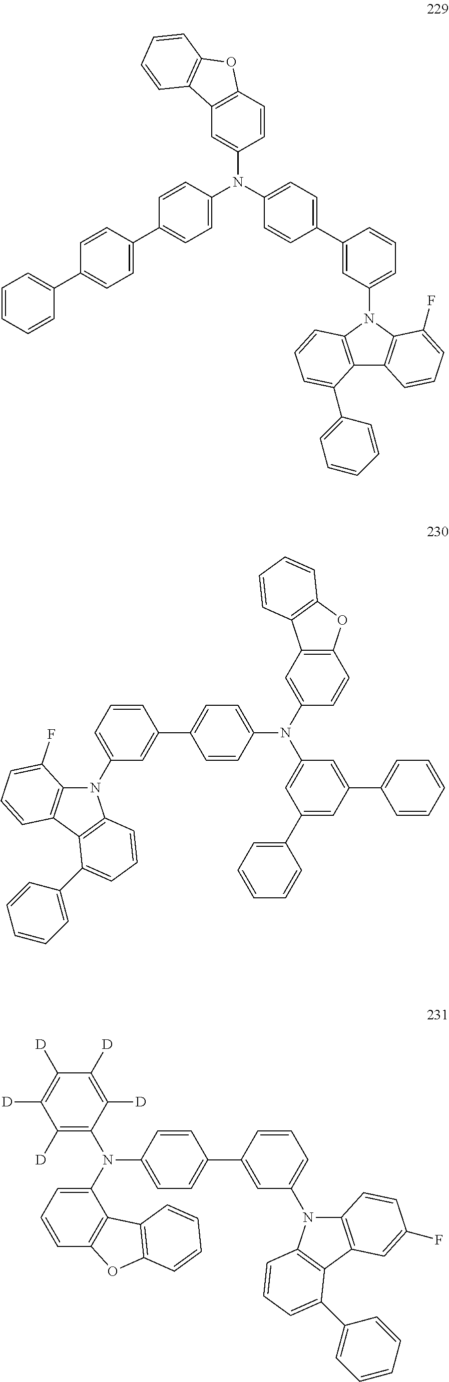 [SMILES: Fc1cccc2c3c(-c4ccccc4)cccc3n(-c3cccc(-c4ccc(N(c5ccc(-c6ccc(-c7ccccc7)cc6)cc5)c5ccc6oc7ccccc7c6c5)cc4)c3)c12 ; Fc1cccc2c3c(-c4ccccc4)cccc3n(-c3cccc(-c4ccc(N(c5cc(-c6ccccc6)cc(-c6ccccc6)c5)c5ccc6oc7ccccc7c6c5)cc4)c3)c12 ; [2H]c1c([2H])c([2H])c(N(c2ccc(-c3cccc(-n4c5ccc(F)cc5c5c(-c6ccccc6)cccc54)c3)cc2)c2cccc3oc4ccccc4c23)c([2H])c1[2H]]

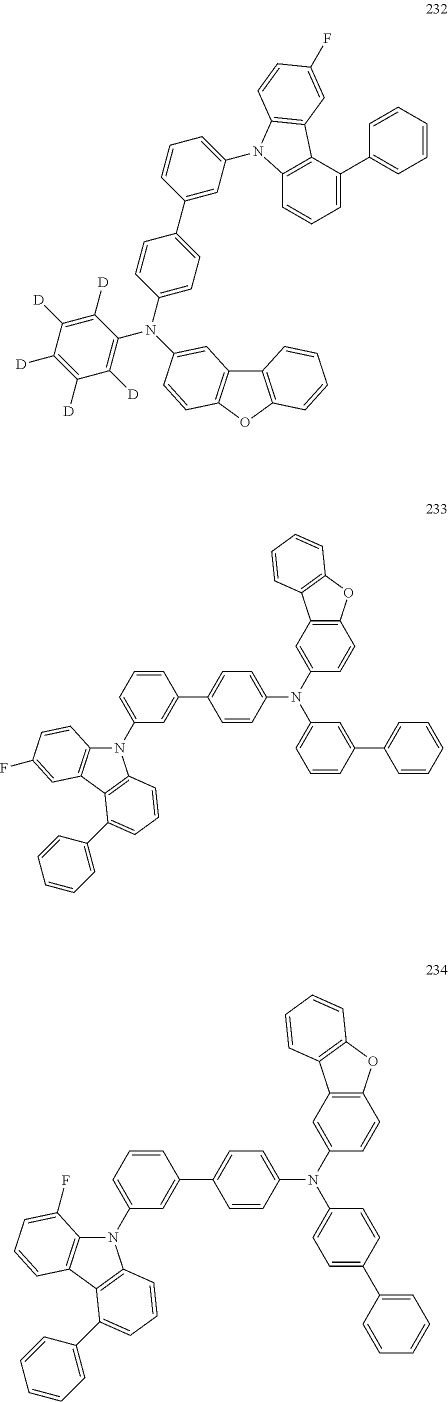 [SMILES: [2H]c1c([2H])c([2H])c(N(c2ccc(-c3cccc(-n4c5ccc(F)cc5c5c(-c6ccccc6)cccc54)c3)cc2)c2ccc3oc4ccccc4c3c2)c([2H])c1[2H] ; Fc1ccc2c(c1)c1c(-c3ccccc3)cccc1n2-c1cccc(-c2ccc(N(c3cccc(-c4ccccc4)c3)c3ccc4oc5ccccc5c4c3)cc2)c1 ; Fc1cccc2c3c(-c4ccccc4)cccc3n(-c3cccc(-c4ccc(N(c5ccc(-c6ccccc6)cc5)c5ccc6oc7ccccc7c6c5)cc4)c3)c12]

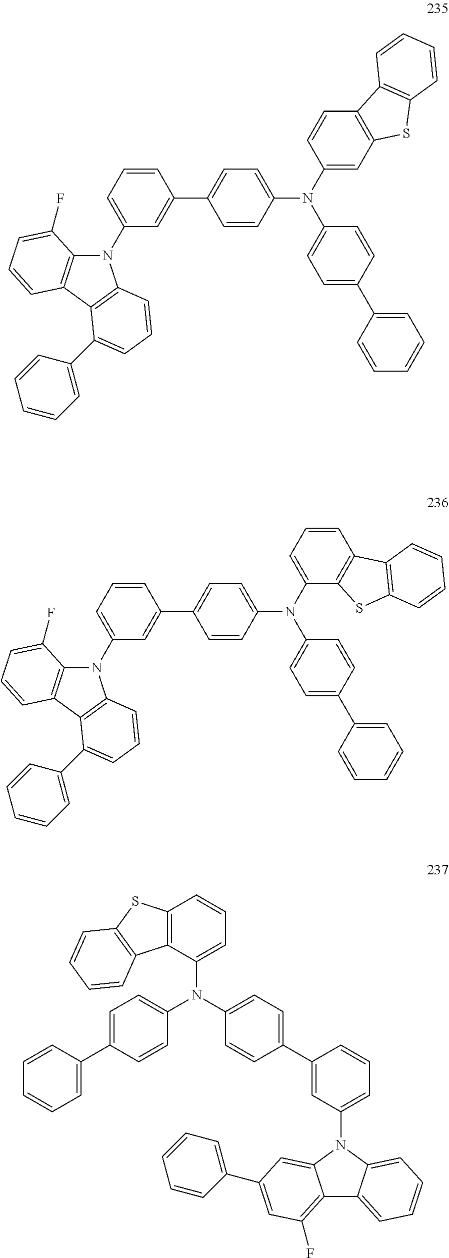 [SMILES: Fc1cccc2c3c(-c4ccccc4)cccc3n(-c3cccc(-c4ccc(N(c5ccc(-c6ccccc6)cc5)c5ccc6c(c5)sc5ccccc56)cc4)c3)c12 ; Fc1cccc2c3c(-c4ccccc4)cccc3n(-c3cccc(-c4ccc(N(c5ccc(-c6ccccc6)cc5)c5cccc6c5sc5ccccc56)cc4)c3)c12 ; Fc1cc(-c2ccccc2)cc2c1c1ccccc1n2-c1cccc(-c2ccc(N(c3ccc(-c4ccccc4)cc3)c3cccc4sc5ccccc5c34)cc2)c1]

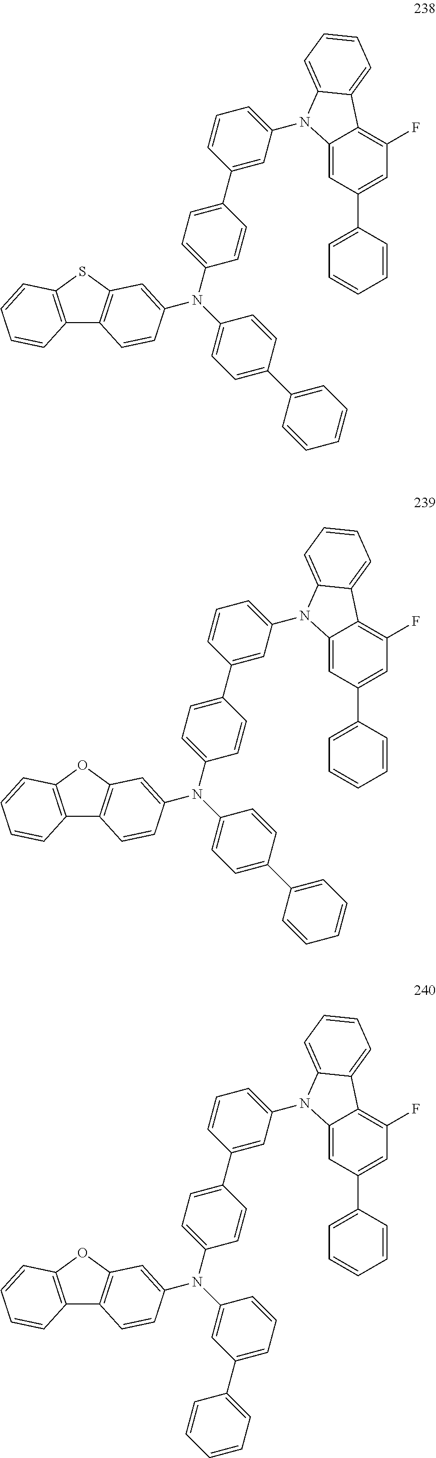 [SMILES: Fc1cc(-c2ccccc2)cc2c1c1ccccc1n2-c1cccc(-c2ccc(N(c3ccc(-c4ccccc4)cc3)c3ccc4c(c3)sc3ccccc34)cc2)c1 ; Fc1cc(-c2ccccc2)cc2c1c1ccccc1n2-c1cccc(-c2ccc(N(c3ccc(-c4ccccc4)cc3)c3ccc4c(c3)oc3ccccc34)cc2)c1 ; Fc1cc(-c2ccccc2)cc2c1c1ccccc1n2-c1cccc(-c2ccc(N(c3cccc(-c4ccccc4)c3)c3ccc4c(c3)oc3ccccc34)cc2)c1]

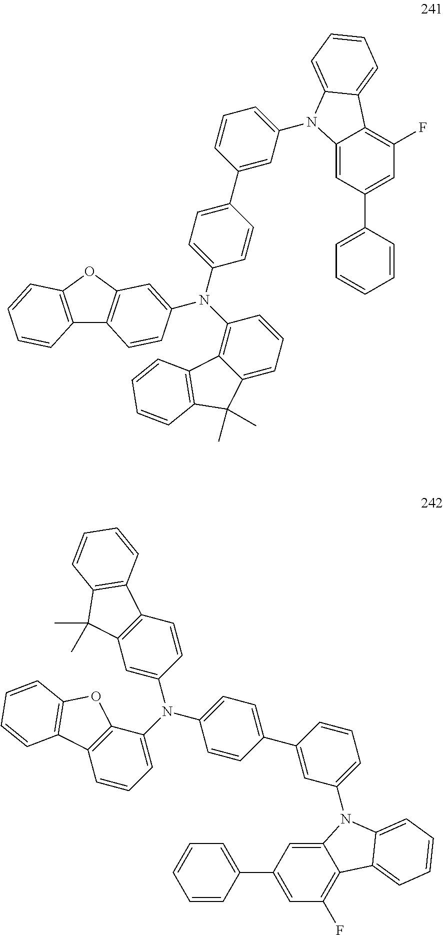 [SMILES: CC1(C)c2ccccc2-c2c(N(c3ccc(-c4cccc(-n5c6ccccc6c6c(F)cc(-c7ccccc7)cc65)c4)cc3)c3ccc4c(c3)oc3ccccc34)cccc21 ; CC1(C)c2ccccc2-c2ccc(N(c3ccc(-c4cccc(-n5c6ccccc6c6c(F)cc(-c7ccccc7)cc65)c4)cc3)c3cccc4c3oc3ccccc34)cc21]

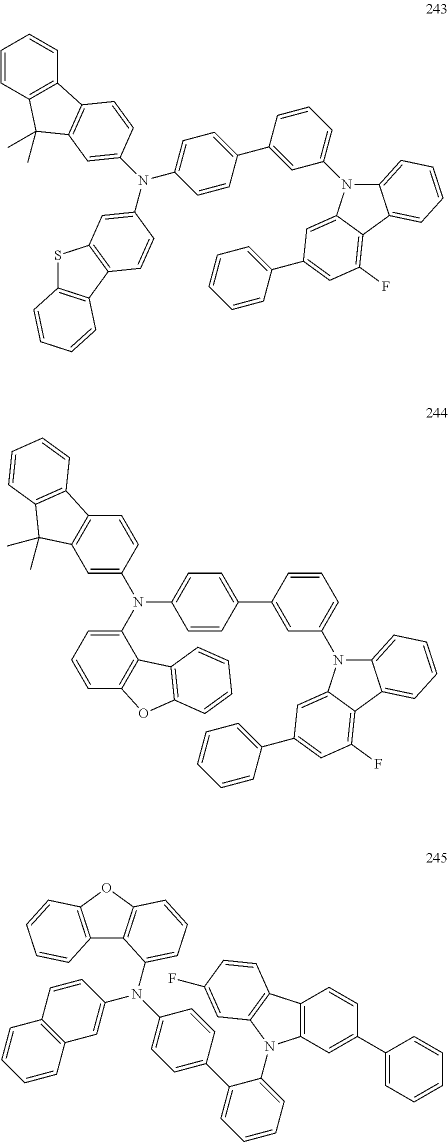 [SMILES: CC1(C)c2ccccc2-c2ccc(N(c3ccc(-c4cccc(-n5c6ccccc6c6c(F)cc(-c7ccccc7)cc65)c4)cc3)c3ccc4c(c3)sc3ccccc34)cc21 ; CC1(C)c2ccccc2-c2ccc(N(c3ccc(-c4cccc(-n5c6ccccc6c6c(F)cc(-c7ccccc7)cc65)c4)cc3)c3cccc4oc5ccccc5c34)cc21 ; Fc1ccc2c3ccc(-c4ccccc4)cc3n(-c3ccccc3-c3ccc(N(c4ccc5ccccc5c4)c4cccc5oc6ccccc6c45)cc3)c2c1]

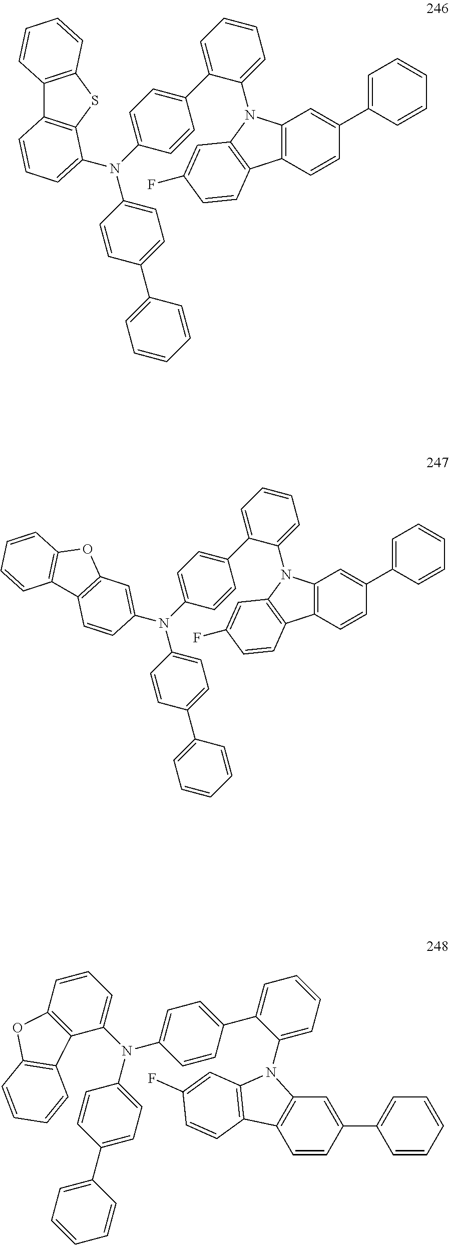 [SMILES: Fc1ccc2c3ccc(-c4ccccc4)cc3n(-c3ccccc3-c3ccc(N(c4ccc(-c5ccccc5)cc4)c4cccc5c4sc4ccccc45)cc3)c2c1 ; Fc1ccc2c3ccc(-c4ccccc4)cc3n(-c3ccccc3-c3ccc(N(c4ccc(-c5ccccc5)cc4)c4ccc5c(c4)oc4ccccc45)cc3)c2c1 ; Fc1ccc2c3ccc(-c4ccccc4)cc3n(-c3ccccc3-c3ccc(N(c4ccc(-c5ccccc5)cc4)c4cccc5oc6ccccc6c45)cc3)c2c1]

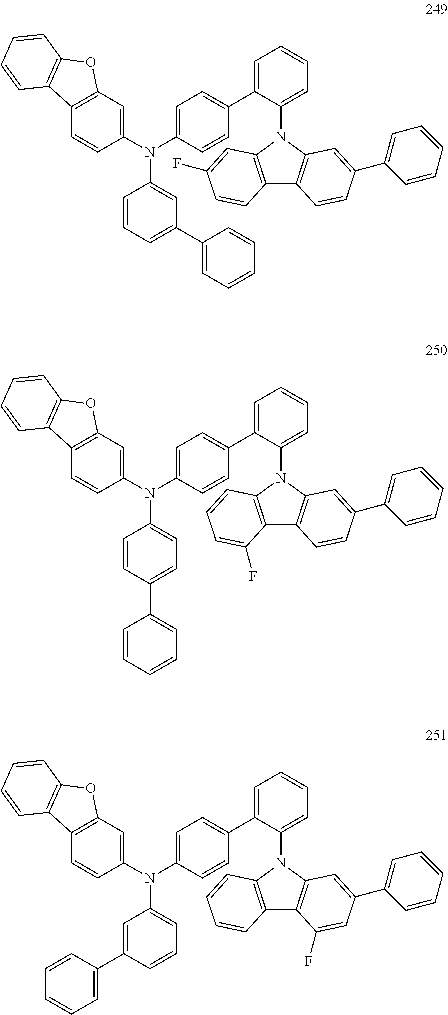 [SMILES: Fc1ccc2c3ccc(-c4ccccc4)cc3n(-c3ccccc3-c3ccc(N(c4cccc(-c5ccccc5)c4)c4ccc5c(c4)oc4ccccc45)cc3)c2c1 ; Fc1cccc2c1c1ccc(-c3ccccc3)cc1n2-c1ccccc1-c1ccc(N(c2ccc(-c3ccccc3)cc2)c2ccc3c(c2)oc2ccccc23)cc1 ; Fc1cc(-c2ccccc2)cc2c1c1ccccc1n2-c1ccccc1-c1ccc(N(c2cccc(-c3ccccc3)c2)c2ccc3c(c2)oc2ccccc23)cc1]

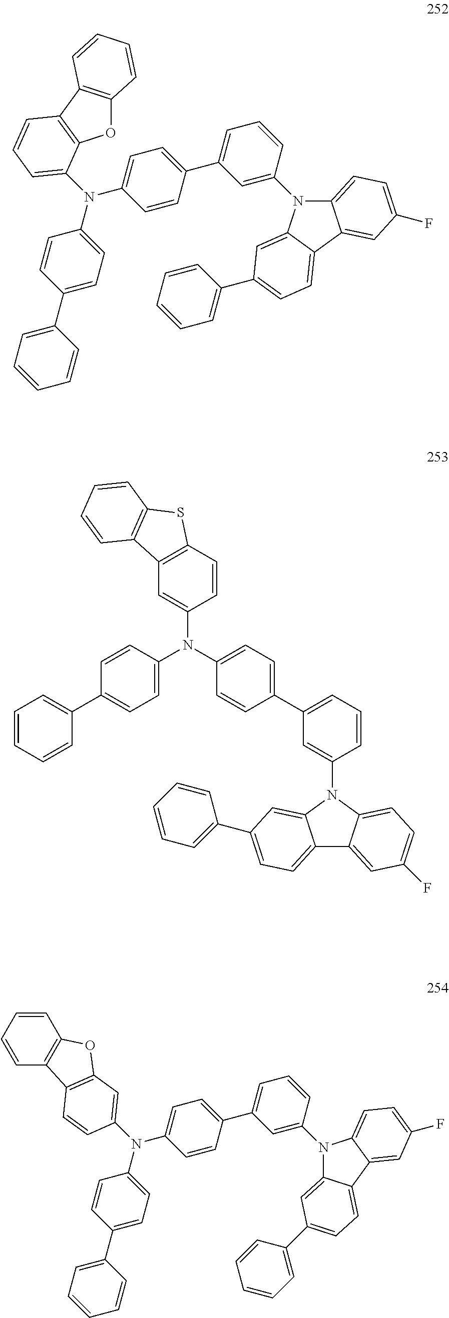 [SMILES: Fc1ccc2c(c1)c1ccc(-c3ccccc3)cc1n2-c1cccc(-c2ccc(N(c3ccc(-c4ccccc4)cc3)c3cccc4c3oc3ccccc34)cc2)c1 ; Fc1ccc2c(c1)c1ccc(-c3ccccc3)cc1n2-c1cccc(-c2ccc(N(c3ccc(-c4ccccc4)cc3)c3ccc4sc5ccccc5c4c3)cc2)c1 ; Fc1ccc2c(c1)c1ccc(-c3ccccc3)cc1n2-c1cccc(-c2ccc(N(c3ccc(-c4ccccc4)cc3)c3ccc4c(c3)oc3ccccc34)cc2)c1]

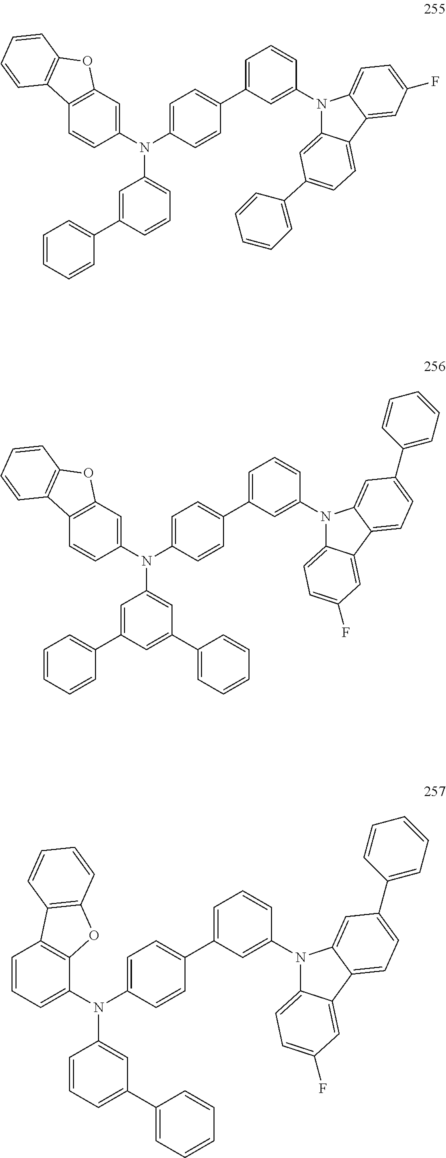 [SMILES: Fc1ccc2c(c1)c1ccc(-c3ccccc3)cc1n2-c1cccc(-c2ccc(N(c3cccc(-c4ccccc4)c3)c3ccc4c(c3)oc3ccccc34)cc2)c1 ; Fc1ccc2c(c1)c1ccc(-c3ccccc3)cc1n2-c1cccc(-c2ccc(N(c3cc(-c4ccccc4)cc(-c4ccccc4)c3)c3ccc4c(c3)oc3ccccc34)cc2)c1 ; Fc1ccc2c(c1)c1ccc(-c3ccccc3)cc1n2-c1cccc(-c2ccc(N(c3cccc(-c4ccccc4)c3)c3cccc4c3oc3ccccc34)cc2)c1]

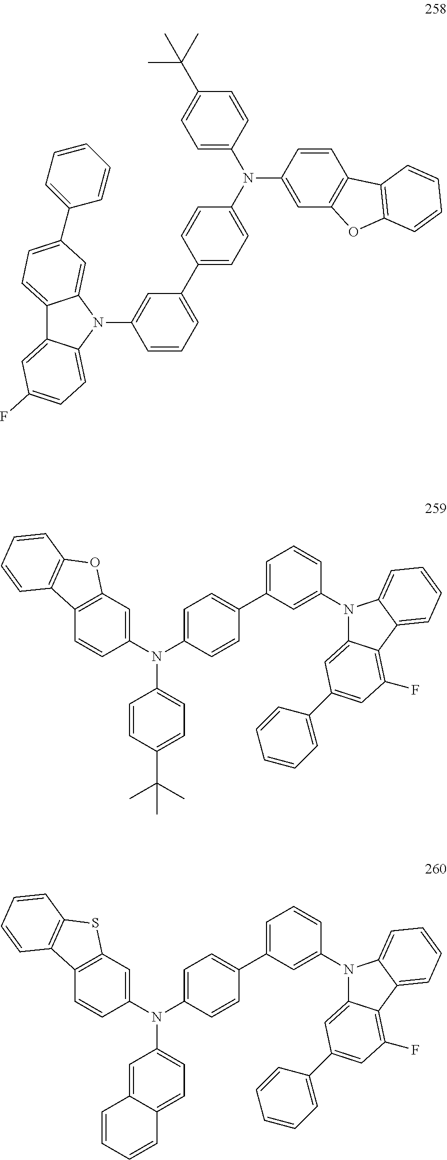 [SMILES: CC(C)(C)c1ccc(N(c2ccc(-c3cccc(-n4c5ccc(F)cc5c5ccc(-c6ccccc6)cc54)c3)cc2)c2ccc3c(c2)oc2ccccc23)cc1 ; CC(C)(C)c1ccc(N(c2ccc(-c3cccc(-n4c5ccccc5c5c(F)cc(-c6ccccc6)cc54)c3)cc2)c2ccc3c(c2)oc2ccccc23)cc1 ; Fc1cc(-c2ccccc2)cc2c1c1ccccc1n2-c1cccc(-c2ccc(N(c3ccc4ccccc4c3)c3ccc4c(c3)sc3ccccc34)cc2)c1]

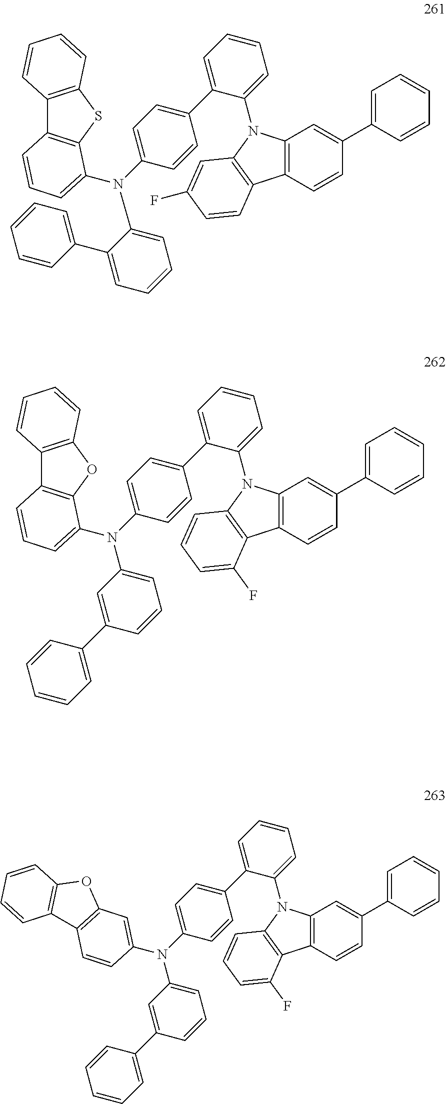 [SMILES: Fc1ccc2c3ccc(-c4ccccc4)cc3n(-c3ccccc3-c3ccc(N(c4ccccc4-c4ccccc4)c4cccc5c4sc4ccccc45)cc3)c2c1 ; Fc1cccc2c1c1ccc(-c3ccccc3)cc1n2-c1ccccc1-c1ccc(N(c2cccc(-c3ccccc3)c2)c2cccc3c2oc2ccccc23)cc1 ; Fc1cccc2c1c1ccc(-c3ccccc3)cc1n2-c1ccccc1-c1ccc(N(c2cccc(-c3ccccc3)c2)c2ccc3c(c2)oc2ccccc23)cc1]

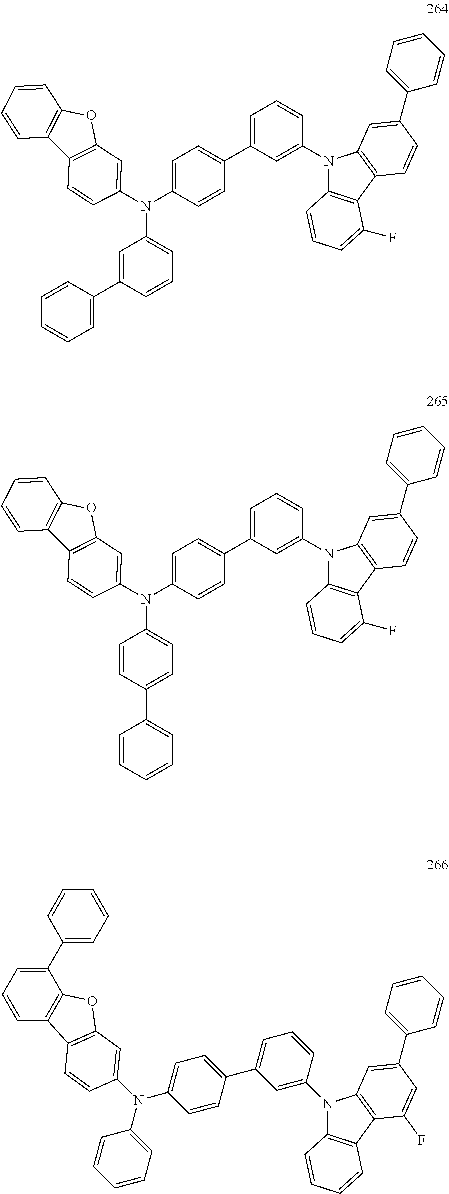 [SMILES: Fc1cccc2c1c1ccc(-c3ccccc3)cc1n2-c1cccc(-c2ccc(N(c3cccc(-c4ccccc4)c3)c3ccc4c(c3)oc3ccccc34)cc2)c1 ; Fc1cccc2c1c1ccc(-c3ccccc3)cc1n2-c1cccc(-c2ccc(N(c3ccc(-c4ccccc4)cc3)c3ccc4c(c3)oc3ccccc34)cc2)c1 ; Fc1cc(-c2ccccc2)cc2c1c1ccccc1n2-c1cccc(-c2ccc(N(c3ccccc3)c3ccc4c(c3)oc3c(-c5ccccc5)cccc34)cc2)c1]

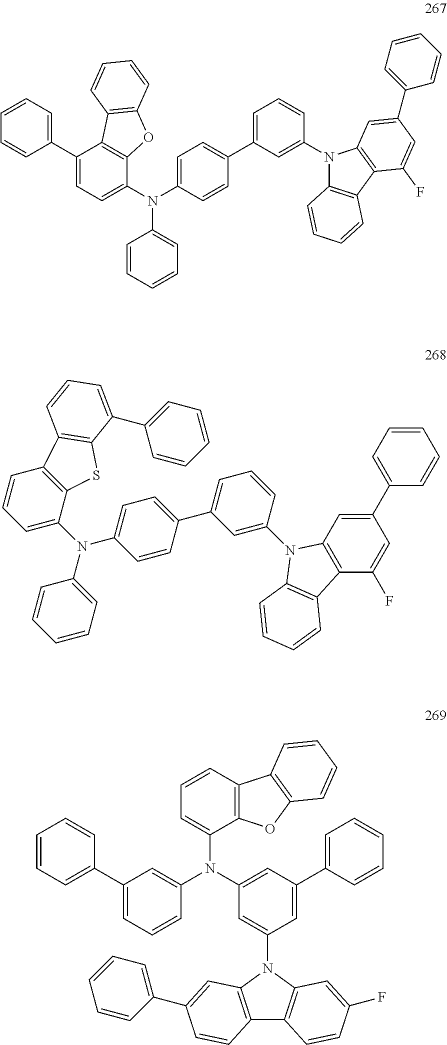 [SMILES: Fc1cc(-c2ccccc2)cc2c1c1ccccc1n2-c1cccc(-c2ccc(N(c3ccccc3)c3ccc(-c4ccccc4)c4c3oc3ccccc34)cc2)c1 ; Fc1cc(-c2ccccc2)cc2c1c1ccccc1n2-c1cccc(-c2ccc(N(c3ccccc3)c3cccc4c3sc3c(-c5ccccc5)cccc34)cc2)c1 ; Fc1ccc2c3ccc(-c4ccccc4)cc3n(-c3cc(-c4ccccc4)cc(N(c4cccc(-c5ccccc5)c4)c4cccc5c4oc4ccccc45)c3)c2c1]

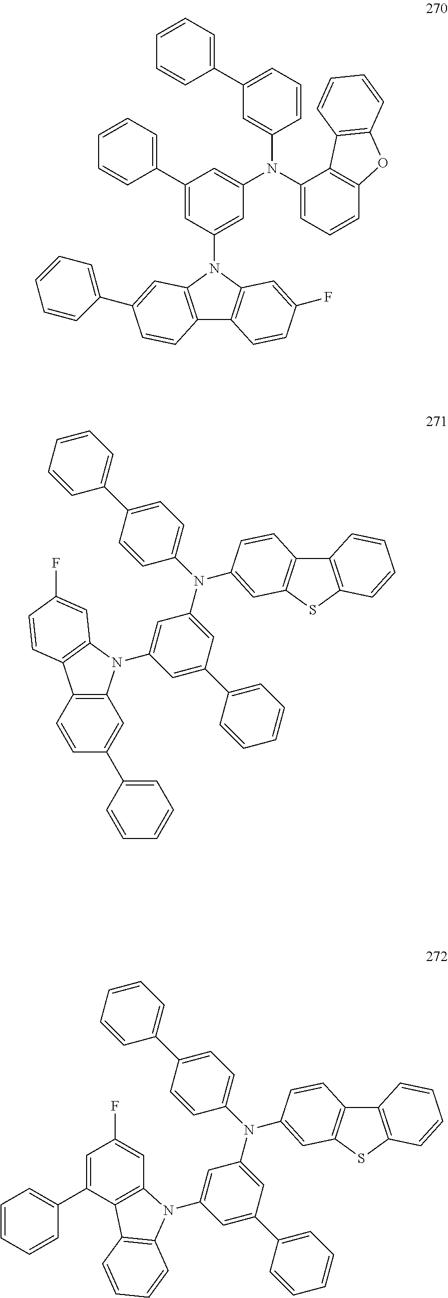 [SMILES: Fc1ccc2c3ccc(-c4ccccc4)cc3n(-c3cc(-c4ccccc4)cc(N(c4cccc(-c5ccccc5)c4)c4cccc5oc6ccccc6c45)c3)c2c1 ; Fc1ccc2c3ccc(-c4ccccc4)cc3n(-c3cc(-c4ccccc4)cc(N(c4ccc(-c5ccccc5)cc4)c4ccc5c(c4)sc4ccccc45)c3)c2c1 ; Fc1cc(-c2ccccc2)c2c3ccccc3n(-c3cc(-c4ccccc4)cc(N(c4ccc(-c5ccccc5)cc4)c4ccc5c(c4)sc4ccccc45)c3)c2c1]

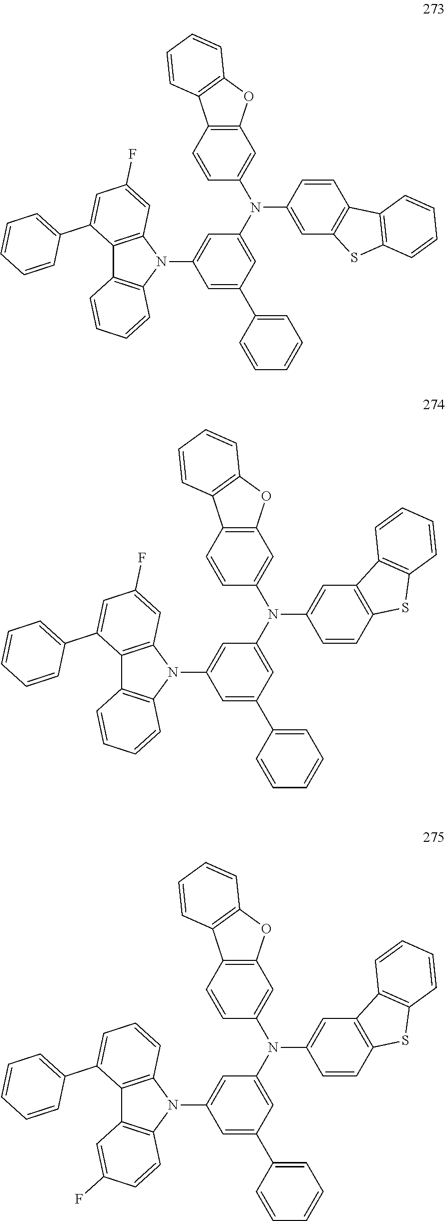 [SMILES: Fc1cc(-c2ccccc2)c2c3ccccc3n(-c3cc(-c4ccccc4)cc(N(c4ccc5c(c4)oc4ccccc45)c4ccc5c(c4)sc4ccccc45)c3)c2c1 ; Fc1cc(-c2ccccc2)c2c3ccccc3n(-c3cc(-c4ccccc4)cc(N(c4ccc5c(c4)oc4ccccc45)c4ccc5sc6ccccc6c5c4)c3)c2c1 ; Fc1ccc2c(c1)c1c(-c3ccccc3)cccc1n2-c1cc(-c2ccccc2)cc(N(c2ccc3c(c2)oc2ccccc23)c2ccc3sc4ccccc4c3c2)c1]

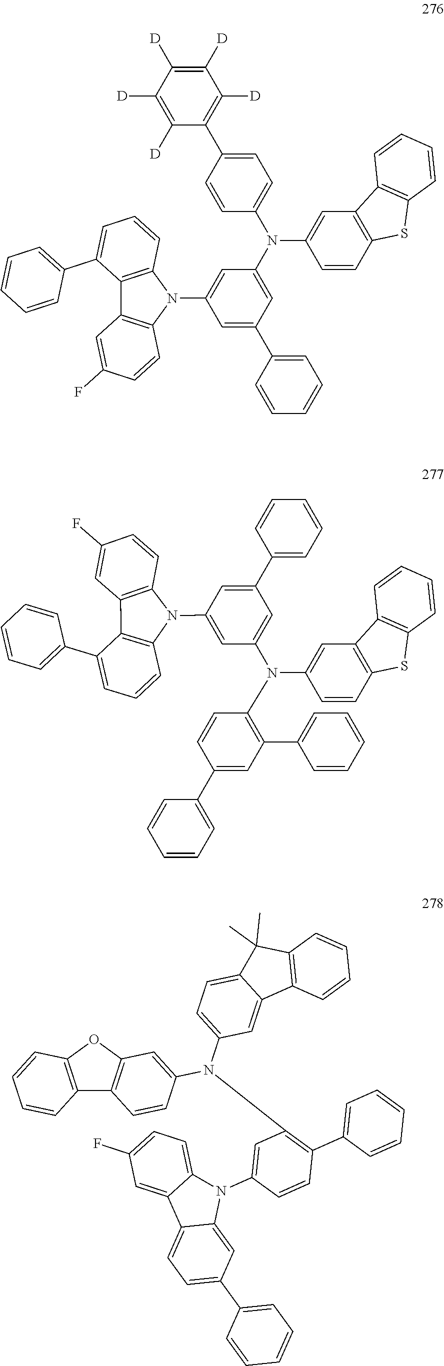 [SMILES: [2H]c1c([2H])c([2H])c(-c2ccc(N(c3cc(-c4ccccc4)cc(-n4c5ccc(F)cc5c5c(-c6ccccc6)cccc54)c3)c3ccc4sc5ccccc5c4c3)cc2)c([2H])c1[2H] ; Fc1ccc2c(c1)c1c(-c3ccccc3)cccc1n2-c1cc(-c2ccccc2)cc(N(c2ccc3sc4ccccc4c3c2)c2ccc(-c3ccccc3)cc2-c2ccccc2)c1 ; CC1(C)c2ccccc2-c2cc(N(c3ccc4c(c3)oc3ccccc34)c3cc(-n4c5ccc(F)cc5c5ccc(-c6ccccc6)cc54)ccc3-c3ccccc3)ccc21]

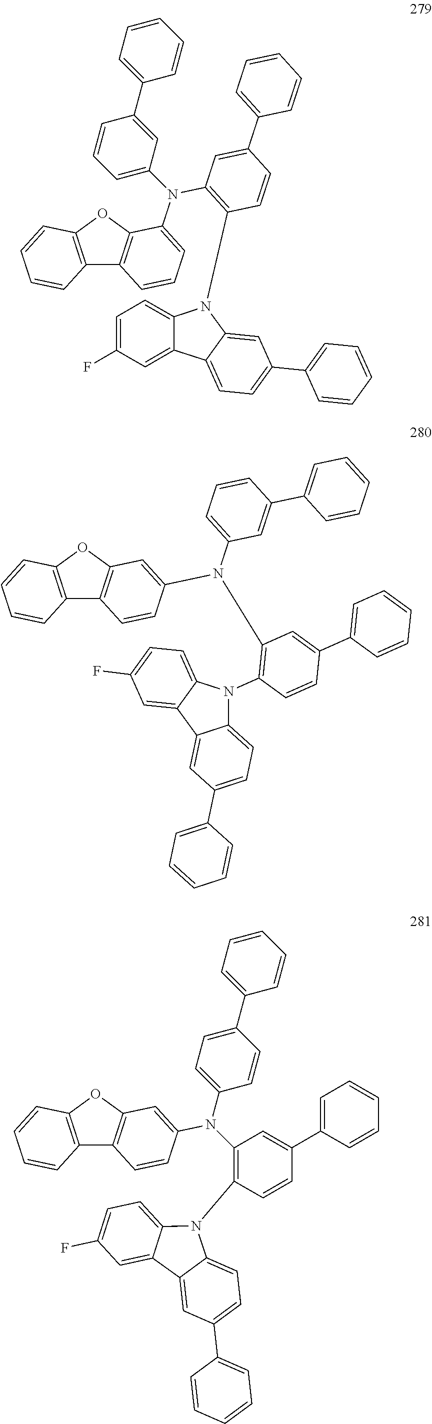 [SMILES: Fc1ccc2c(c1)c1ccc(-c3ccccc3)cc1n2-c1ccc(-c2ccccc2)cc1N(c1cccc(-c2ccccc2)c1)c1cccc2c1oc1ccccc12 ; Fc1ccc2c(c1)c1cc(-c3ccccc3)ccc1n2-c1ccc(-c2ccccc2)cc1N(c1cccc(-c2ccccc2)c1)c1ccc2c(c1)oc1ccccc12 ; Fc1ccc2c(c1)c1cc(-c3ccccc3)ccc1n2-c1ccc(-c2ccccc2)cc1N(c1ccc(-c2ccccc2)cc1)c1ccc2c(c1)oc1ccccc12]

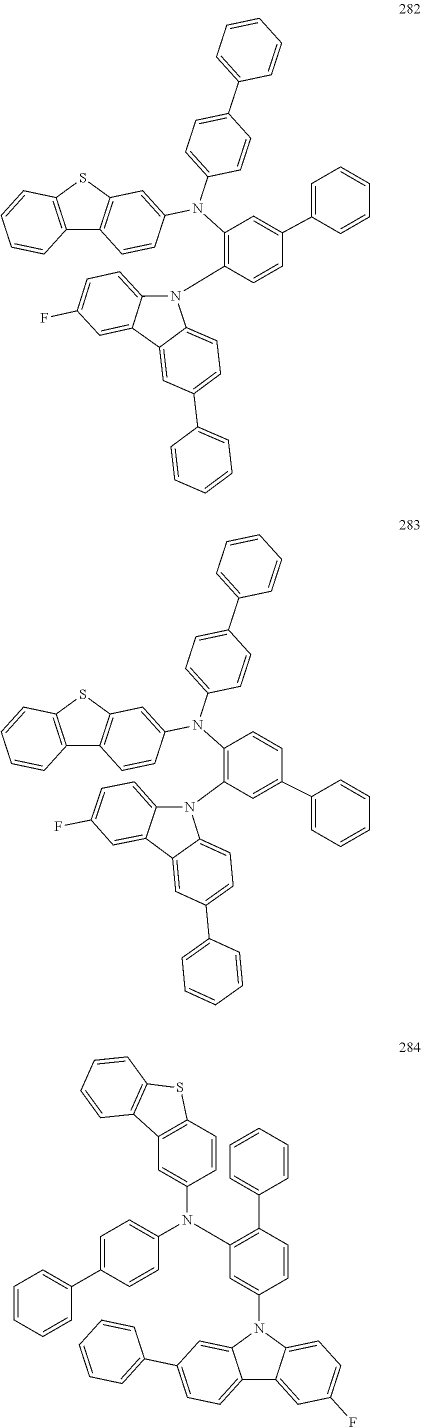 [SMILES: Fc1ccc2c(c1)c1cc(-c3ccccc3)ccc1n2-c1ccc(-c2ccccc2)cc1N(c1ccc(-c2ccccc2)cc1)c1ccc2c(c1)sc1ccccc12 ; Fc1ccc2c(c1)c1cc(-c3ccccc3)ccc1n2-c1cc(-c2ccccc2)ccc1N(c1ccc(-c2ccccc2)cc1)c1ccc2c(c1)sc1ccccc12 ; Fc1ccc2c(c1)c1ccc(-c3ccccc3)cc1n2-c1ccc(-c2ccccc2)c(N(c2ccc(-c3ccccc3)cc2)c2ccc3sc4ccccc4c3c2)c1]

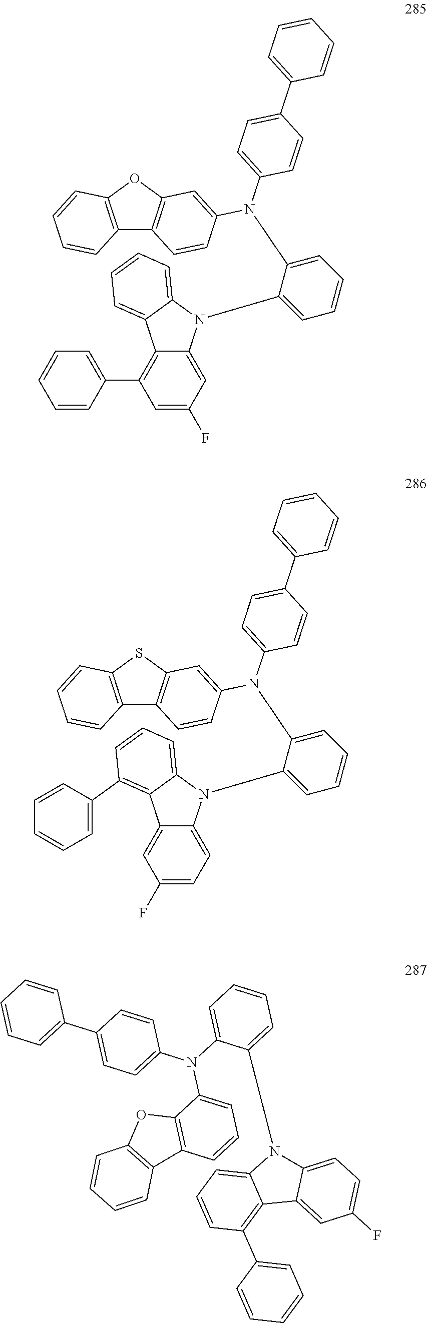 [SMILES: Fc1cc(-c2ccccc2)c2c3ccccc3n(-c3ccccc3N(c3ccc(-c4ccccc4)cc3)c3ccc4c(c3)oc3ccccc34)c2c1 ; Fc1ccc2c(c1)c1c(-c3ccccc3)cccc1n2-c1ccccc1N(c1ccc(-c2ccccc2)cc1)c1ccc2c(c1)sc1ccccc12 ; Fc1ccc2c(c1)c1c(-c3ccccc3)cccc1n2-c1ccccc1N(c1ccc(-c2ccccc2)cc1)c1cccc2c1oc1ccccc12]

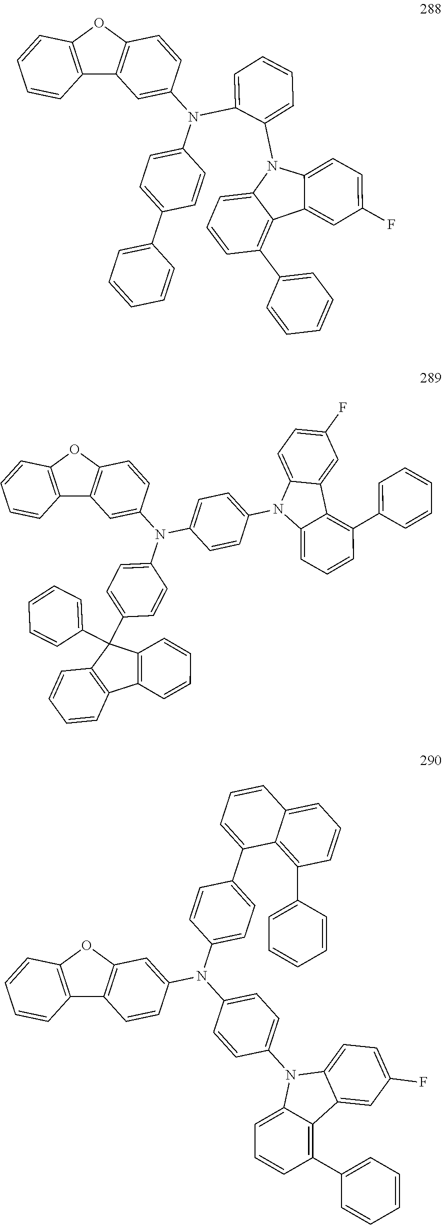 [SMILES: Fc1ccc2c(c1)c1c(-c3ccccc3)cccc1n2-c1ccccc1N(c1ccc(-c2ccccc2)cc1)c1ccc2oc3ccccc3c2c1 ; Fc1ccc2c(c1)c1c(-c3ccccc3)cccc1n2-c1ccc(N(c2ccc(C3(c4ccccc4)c4ccccc4-c4ccccc43)cc2)c2ccc3oc4ccccc4c3c2)cc1 ; Fc1ccc2c(c1)c1c(-c3ccccc3)cccc1n2-c1ccc(N(c2ccc(-c3cccc4cccc(-c5ccccc5)c34)cc2)c2ccc3c(c2)oc2ccccc23)cc1]

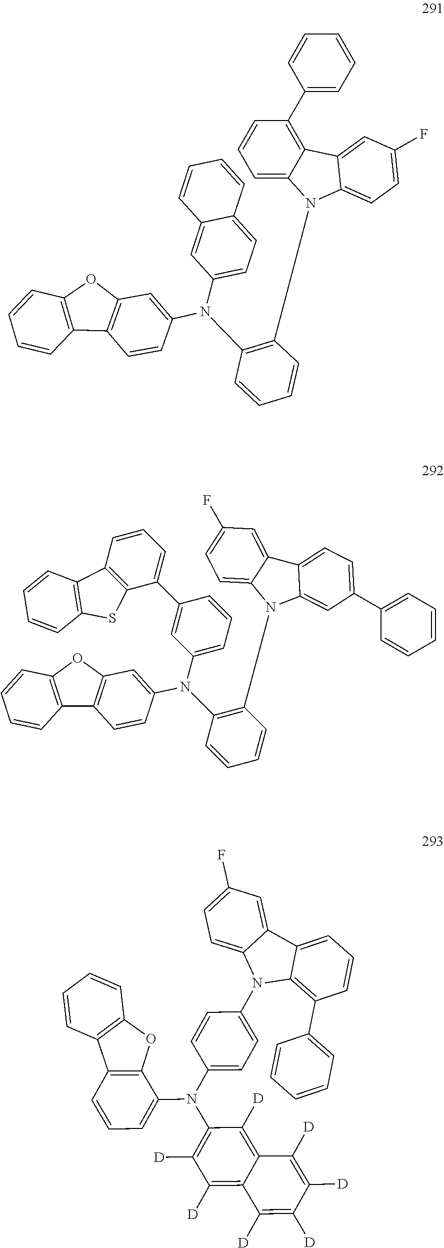 [SMILES: Fc1ccc2c(c1)c1c(-c3ccccc3)cccc1n2-c1ccccc1N(c1ccc2ccccc2c1)c1ccc2c(c1)oc1ccccc12 ; Fc1ccc2c(c1)c1ccc(-c3ccccc3)cc1n2-c1ccccc1N(c1cccc(-c2cccc3c2sc2ccccc23)c1)c1ccc2c(c1)oc1ccccc12 ; [2H]c1c([2H])c([2H])c2c([2H])c(N(c3ccc(-n4c5ccc(F)cc5c5cccc(-c6ccccc6)c54)cc3)c3cccc4c3oc3ccccc34)c([2H])c([2H])c2c1[2H]]

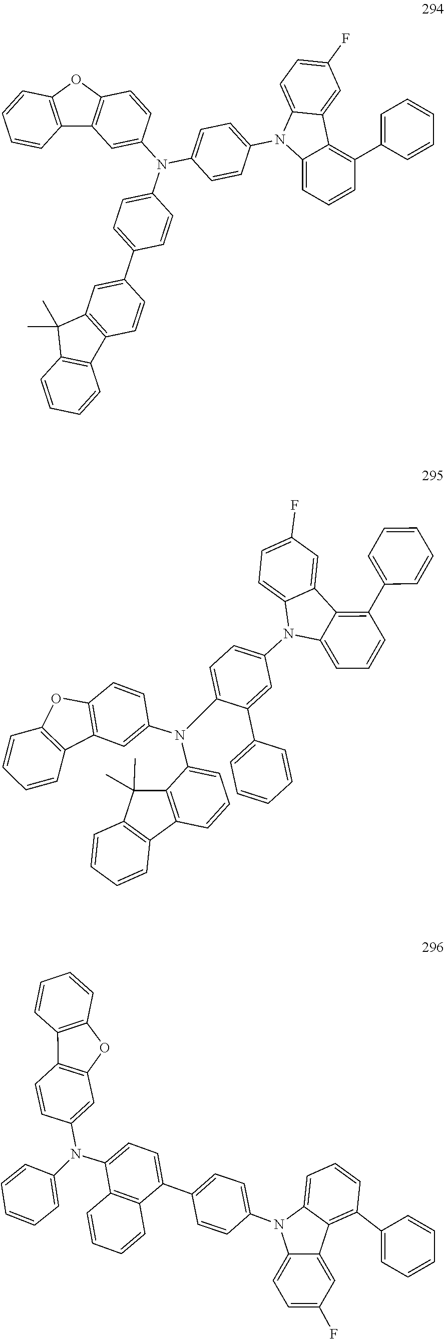 [SMILES: CC1(C)c2ccccc2-c2ccc(-c3ccc(N(c4ccc(-n5c6ccc(F)cc6c6c(-c7ccccc7)cccc65)cc4)c4ccc5oc6ccccc6c5c4)cc3)cc21 ; CC1(C)c2ccccc2-c2cccc(N(c3ccc4oc5ccccc5c4c3)c3ccc(-n4c5ccc(F)cc5c5c(-c6ccccc6)cccc54)cc3-c3ccccc3)c21 ; Fc1ccc2c(c1)c1c(-c3ccccc3)cccc1n2-c1ccc(-c2ccc(N(c3ccccc3)c3ccc4c(c3)oc3ccccc34)c3ccccc23)cc1]

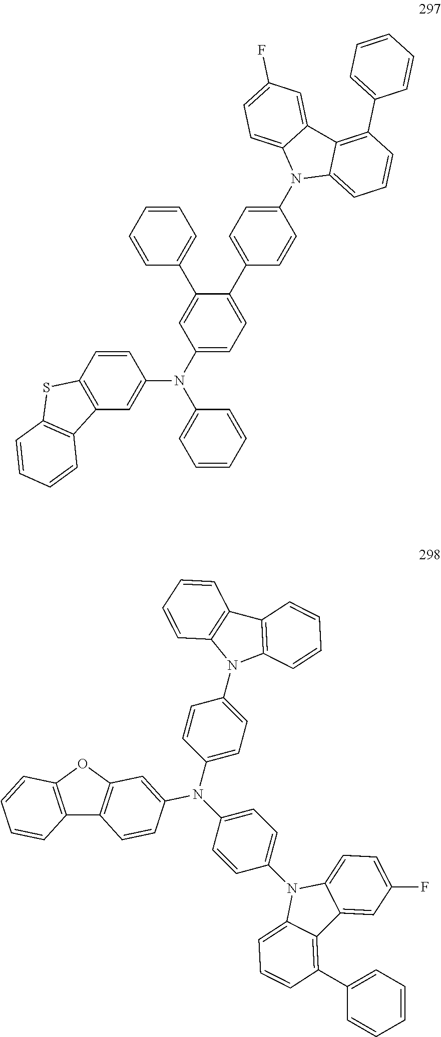 [SMILES: Fc1ccc2c(c1)c1c(-c3ccccc3)cccc1n2-c1ccc(-c2ccc(N(c3ccccc3)c3ccc4sc5ccccc5c4c3)cc2-c2ccccc2)cc1 ; Fc1ccc2c(c1)c1c(-c3ccccc3)cccc1n2-c1ccc(N(c2ccc(-n3c4ccccc4c4ccccc43)cc2)c2ccc3c(c2)oc2ccccc23)cc1]

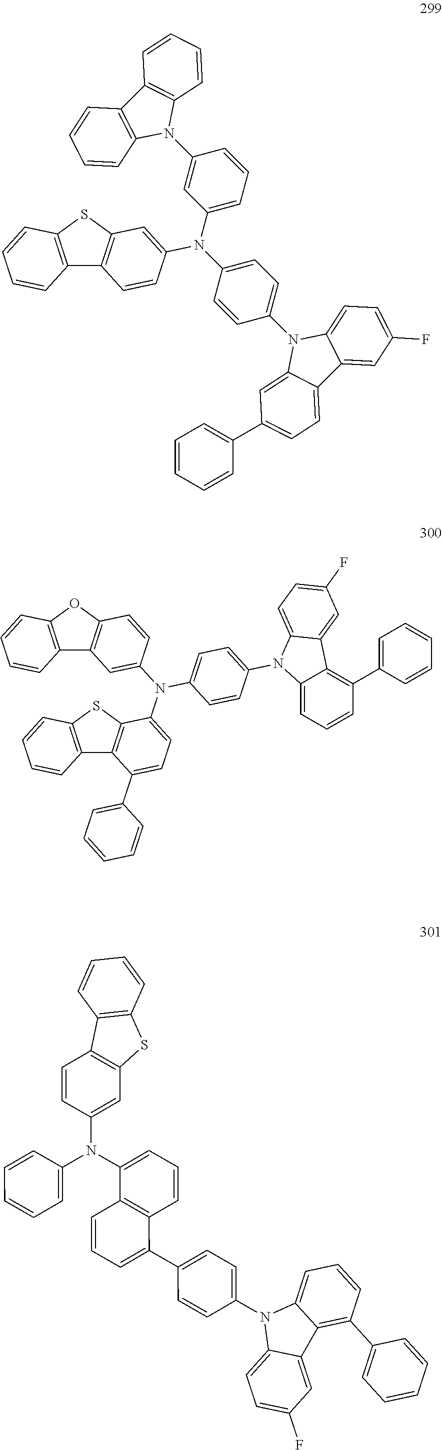 [SMILES: Fc1ccc2c(c1)c1ccc(-c3ccccc3)cc1n2-c1ccc(N(c2cccc(-n3c4ccccc4c4ccccc43)c2)c2ccc3c(c2)sc2ccccc23)cc1 ; Fc1ccc2c(c1)c1c(-c3ccccc3)cccc1n2-c1ccc(N(c2ccc3oc4ccccc4c3c2)c2ccc(-c3ccccc3)c3c2sc2ccccc23)cc1 ; Fc1ccc2c(c1)c1c(-c3ccccc3)cccc1n2-c1ccc(-c2cccc3c(N(c4ccccc4)c4ccc5c(c4)sc4ccccc45)cccc23)cc1]

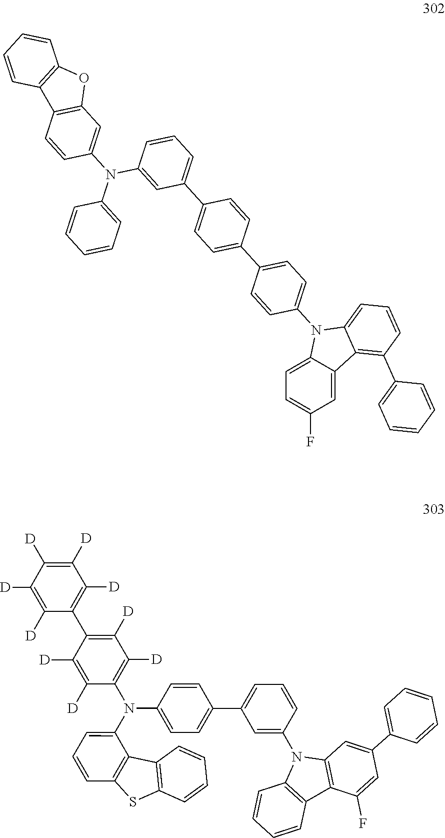 [SMILES: Fc1ccc2c(c1)c1c(-c3ccccc3)cccc1n2-c1ccc(-c2ccc(-c3cccc(N(c4ccccc4)c4ccc5c(c4)oc4ccccc45)c3)cc2)cc1 ; [2H]c1c([2H])c([2H])c(-c2c([2H])c([2H])c(N(c3ccc(-c4cccc(-n5c6ccccc6c6c(F)cc(-c7ccccc7)cc65)c4)cc3)c3cccc4sc5ccccc5c34)c([2H])c2[2H])c([2H])c1[2H]]

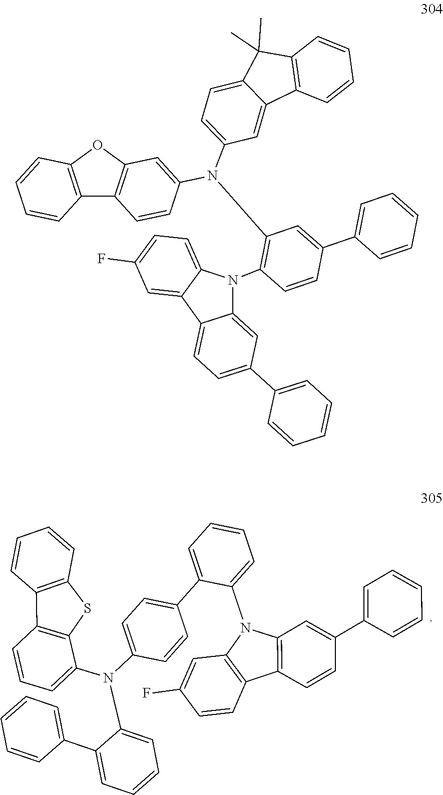 [SMILES: CC1(C)c2ccccc2-c2cc(N(c3ccc4c(c3)oc3ccccc34)c3cc(-c4ccccc4)ccc3-n3c4ccc(F)cc4c4ccc(-c5ccccc5)cc43)ccc21 ; Fc1ccc2c3ccc(-c4ccccc4)cc3n(-c3ccccc3-c3ccc(N(c4ccccc4-c4ccccc4)c4cccc5c4sc4ccccc45)cc3)c2c1]

In a second aspect, the present disclosure provides an organic electroluminescent device comprising an anode and a cathode, and a functional layer disposed between the anode and the cathode, wherein the functional layer comprises the organic compound described in the first aspect of the present disclosure. The organic compound provided in the present disclosure can be used to form at least one organic film layer in the functional layer, to improve performances of the organic electroluminescent device such as lifetime.

Optionally, the functional layer comprises a hole transport layer, which comprises the organic compound of the present disclosure.

Further optionally, the hole transport layer comprises a first hole transport layer and a second hole transport layer (also known as an “electron blocking layer” or a “light emitting auxiliary layer”), and the first hole transport layer is closer to the anode than the second hole transport layer, wherein the second hole transport layer comprises the organic compound.

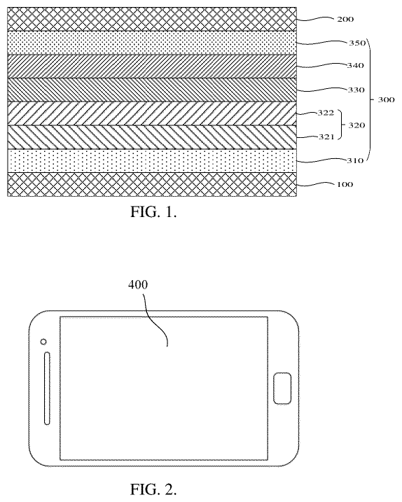

According to a specific embodiment, as shown in FIG. 1, the organic electroluminescent device comprises an anode 100, a first hole transport layer 321, a second hole transport layer 322, an organic light emitting layer 330 as an energy conversion layer, an electron transport layer 340, and a cathode 200 stacked in sequence. The first hole transport layer 321 and the second hole transport layer 322 constitute the hole transport layer 320.

Optionally, the anode 100 comprises an anode material as follows, which is preferably a large work function material contributing to injection of holes into the functional layer 300.

Specific examples of the anode material include: metals such as nickel, platinum, vanadium, chromium, copper, zinc and gold, or alloys thereof; metal oxides such as zinc oxide, indium oxide, indium tin oxide (ITO), and indium zinc oxide (IZO); combinations of metals and oxides, such as ZnO: Al or SnO₂: Sb; and conductive polymers such as poly(3-methylthiophene), poly [3, 4-(ethylene-1,2-dioxy)thiophene] (PEDT), polypyrrole, and polyaniline, but are not limited threto. Preferably, a transparent electrode comprising indium tin (ITO) as the anode is included.

Optionally, the first hole transport layer 321 may include one or more hole transport material(s). The hole transport materials may be selected from carbazole polymers, carbazole-connected triarylamine compounds, and other types of compounds. It is not particularly limited in the present disclosure. The specific examples of the hole transport material include but are not limited to:

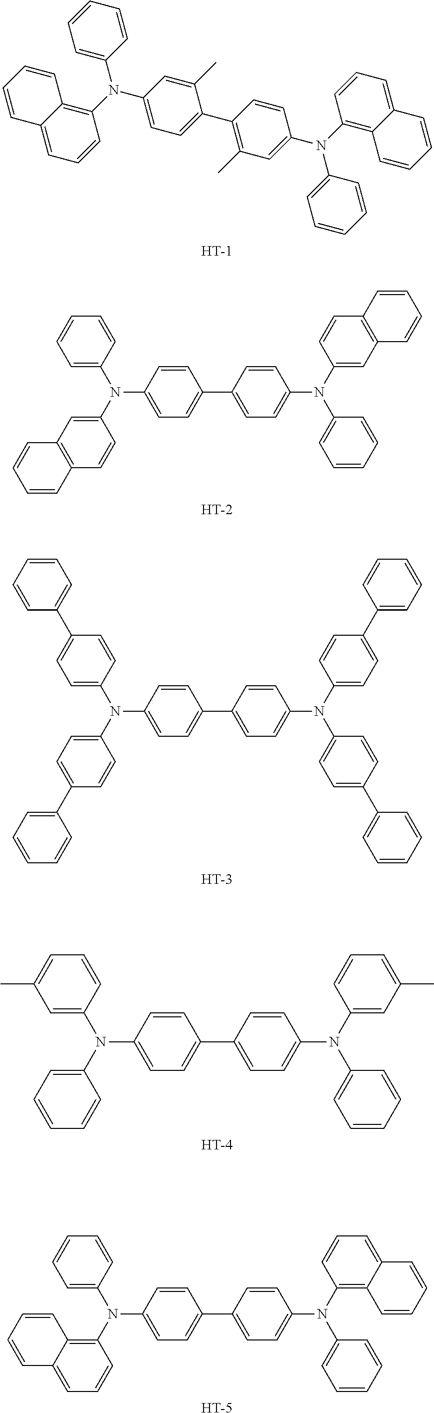

In a specific embodiment, the material of the first hole transport layer 321 is HT-4.

Optionally, the material of the second hole transport layer 322 is selected from the organic compounds of the present disclosure.

In the present disclosure, the organic light emitting layer 330 may be composed of a single light emitting material or may include a host material and a guest material. Preferably, the organic light emitting layer 330 is composed of a host material and a guest material. The holes injected into the organic light emitting layer 330 and the electrons injected into the organic light emitting layer 330 can recombine in the organic light emitting layer 330 to form excitons. The excitons transmit energy to the host material, and the host material transmits the energy to the guest material, thereby enabling the guest material to emit light.

The host material of the organic light emitting layer 330 may be a metal chelating compound, a stilbene-based derivative, an arylamine derivative, a dibenzofuran derivative, and other types of materials. The specific examples of the host material include but are not limited to the following compounds:

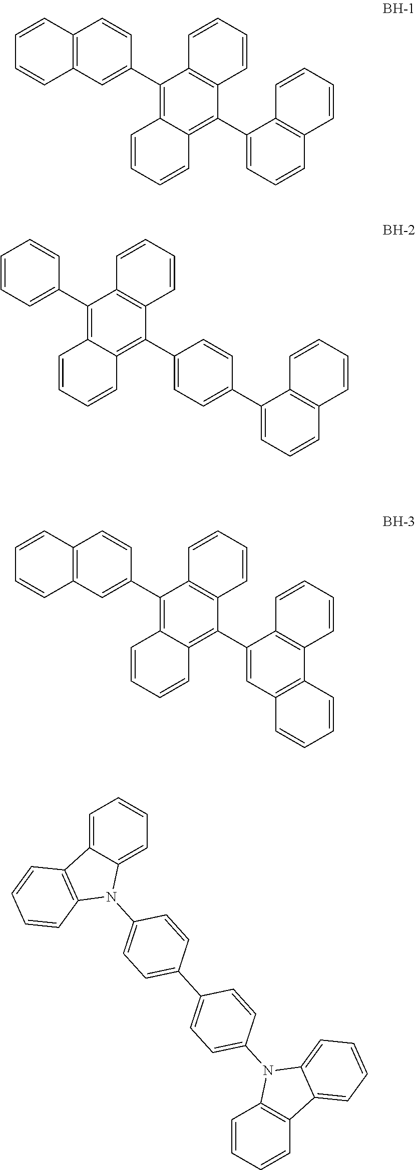

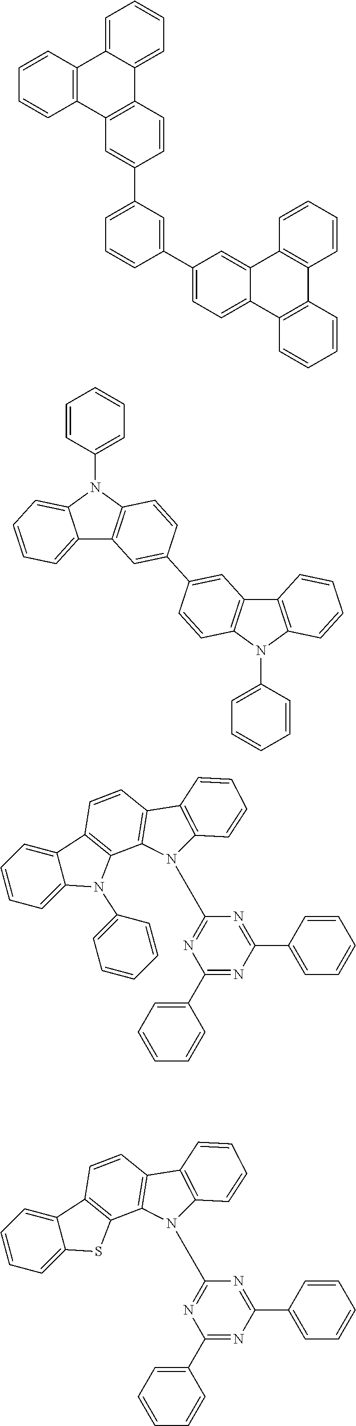

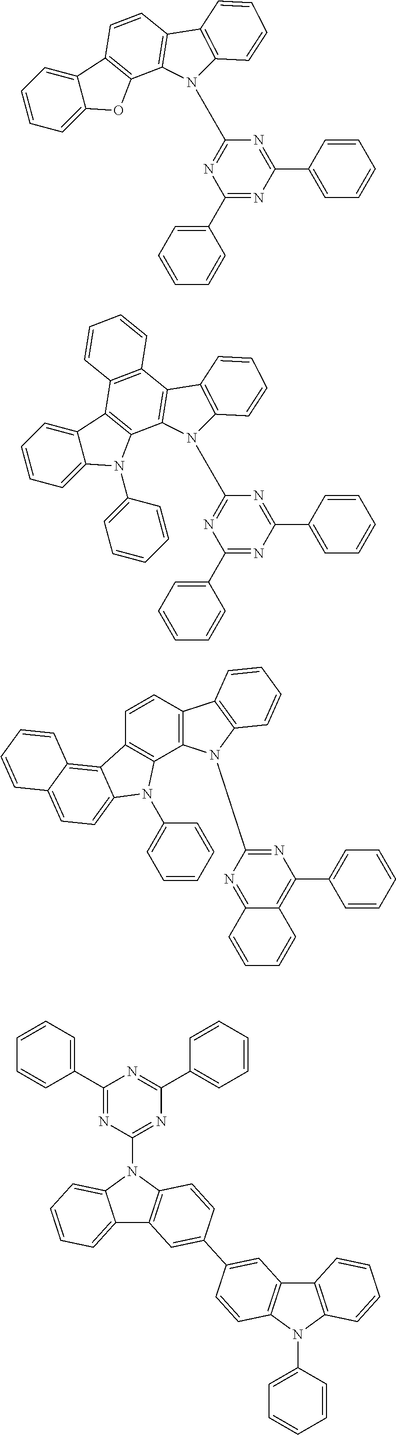

In a specific embodiment, the host material is BH-2.

The guest material of the organic light emitting layer 330 may be a compound having a condensed aryl ring or its derivative, a compound having a heteroaryl ring or its derivative, an arylamine derivative, and other materials. It is not particularly limited in the present disclosure. For example, the guest material is selected from at least one of the following compounds:

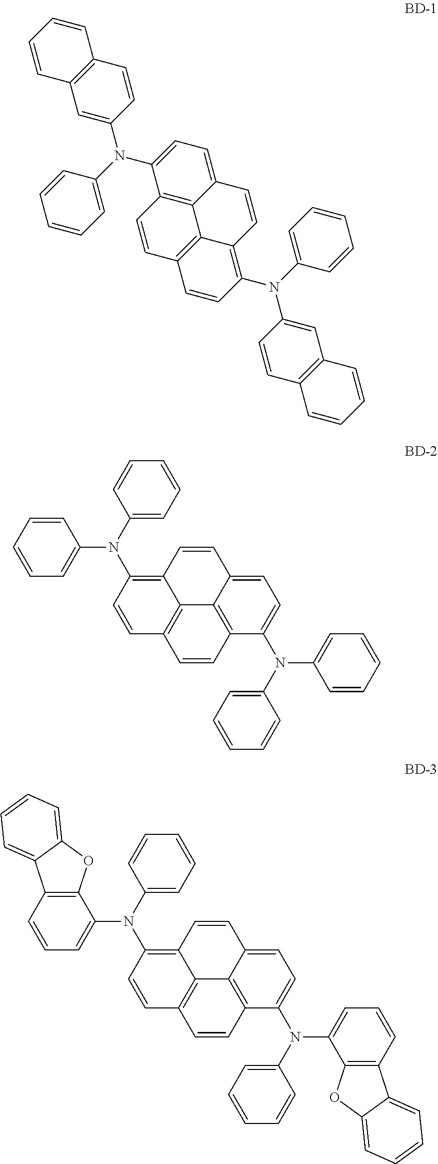 [SMILES: c1ccc(N(c2ccc3ccccc3c2)c2ccc3ccc4c(N(c5ccccc5)c5ccc6ccccc6c5)ccc5ccc2c3c54)cc1 ; c1ccc(N(c2ccccc2)c2ccc3ccc4c(N(c5ccccc5)c5ccccc5)ccc5ccc2c3c54)cc1 ; c1ccc(N(c2ccc3ccc4c(N(c5ccccc5)c5cccc6c5oc5ccccc56)ccc5ccc2c3c54)c2cccc3c2oc2ccccc23)cc1]

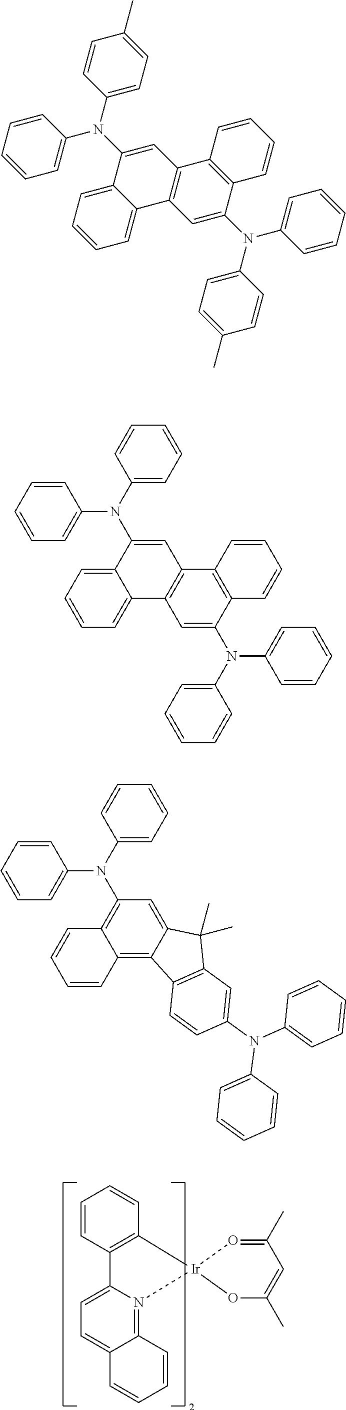

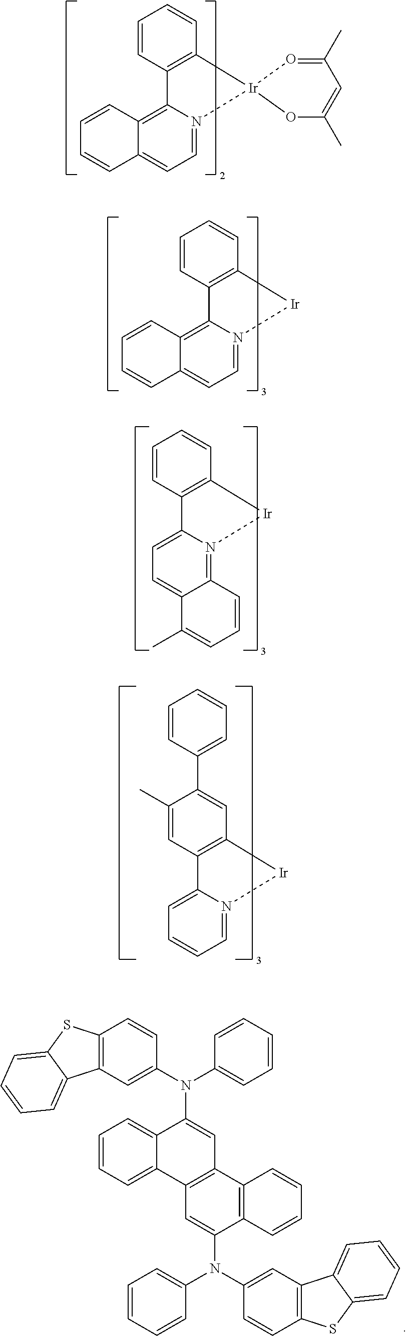

In a specific embodiment, the guest material is BD-3.

In the present disclosure, the electron transport layer 340 can be a single-layer structure or a multi-layer structure, which can include one or more electron transport material(s). The electron transport material is selected from, but not limited to, benzimidazole derivatives, oxadiazole derivatives, quinoxaline derivatives, and other electron transport materials. For example, the electron transport layer material is selected from LiQ and the group consisting the following compounds:

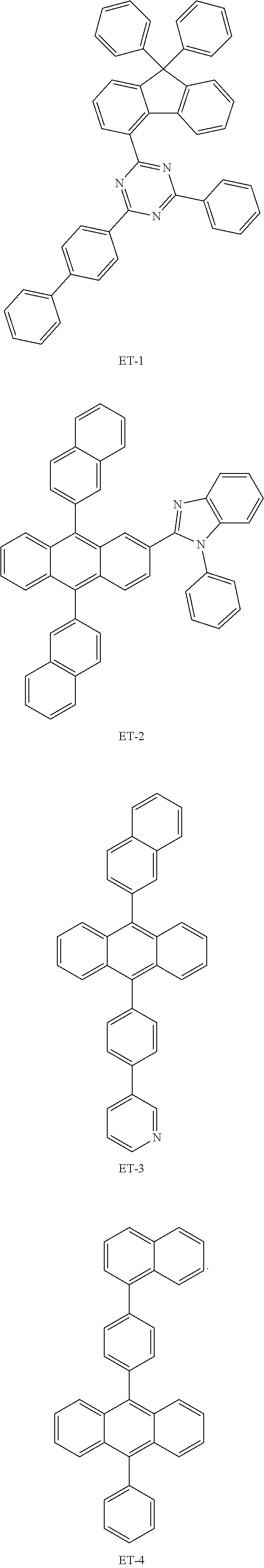

In a specific embodiment, the material of the electron transport layer 340 is composed of LiQ and ET-2.

In the present disclosure, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, and alloys thereof; or multilayer materials such as LiF/Al, Liq/Al, LiO₂/Al, LiF/Ca, LiF/Al, and BaF₂/Ca. Preferably, a metal electrode comprising magnesium and silver is included as the cathode.

Optionally, as shown in FIG. 1, a hole injection layer 310 may be further provided between the anode 100 and the first hole transport layer 321 to enhance the ability to inject holes into the first hole transport layer 321. The hole injection layer 310 may choose to use benzidine derivatives, starburst arylamine-based compounds, phthalocyanine based compounds, and other materials. It is not particularly limited in the present disclosure. For example, the hole injection layer 310 is selected from one or more of the following compounds:

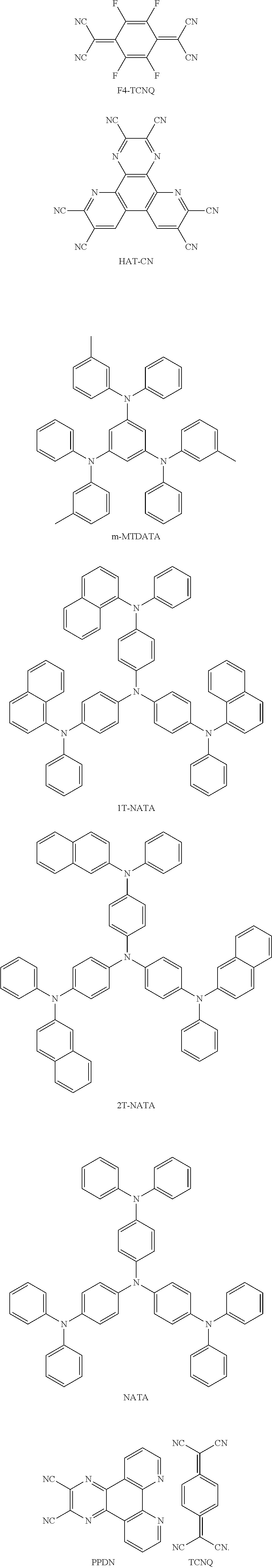

In a specific embodiment, the material of the hole injection layer 310 is F4-TCNQ.

Optionally, as shown in FIG. 1, an electron injection layer 350 is further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and an organic compound. For example, the electron injection layer 350 comprises LiQ or Yb.

In a third aspect, the present disclosure provides an electronic apparatus, comprising the organic electroluminescent device described in the second aspect of the present disclosure.

According to an embodiment, as shown in FIG. 2, the electronic apparatus is an electronic apparatus 400 comprising the above-described organic electroluminescent device. The electronic apparatus 400 may be for example, a display apparatus, a lighting apparatus, an optical communication apparatus, and other type of electronic devices, examples of which may include, but are not limited to, computer screens, mobile phone screens, televisions, electronic paper, emergency lamps, and optical modules, etc.

The following examples are provided to further illustrate the present disclosure, but the present disclosure is not limited thereto. Compounds for which synthesis methods are not mentioned in the present disclosure can be obtained through commercial sources.

### 1. Synthesis of IMAX

The synthesis of IM AX is illustrated by means of an example of IM A1.

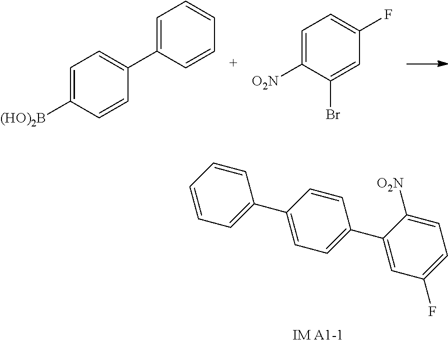 [SMILES: OB(O)c1ccc(-c2ccccc2)cc1 ; O=[N+]([O-])c1ccc(F)cc1Br ; O=[N+]([O-])c1ccc(F)cc1-c1ccc(-c2ccccc2)cc1]

(1) 4-biphenylboronic acid (19.8 g, 100.00 mmol), 2-bromo-4-fluoronitrobenzene (22.00 g, 100.00 mmol), tetrakistriphenylphosphine palladium (2.31 g, 2.00 mmol), tetrabutylammonium bromide (3.22 g, 10.00 mmol) and potassium carbonate (27.6 g, 200 mmol) were added into a mixed solvent of toluene (80 mL), ethanol (40 mL) and water (20 mL), and the resulting mixture was heated to 72° C. under nitrogen protection and stirred for 15 hours of reaction. Then the reaction solution was cooled to room temperature, and washed with water followed by drying over magnesium sulfate, and after filtration, the filtrate was concentrated under reduced pressure to remove the solvent, to obtain a crude product. The crude product was purified by recrystallization from toluene (1 g crude product: 3 mL toluene) to obtain IM A1-1 as a light-yellow product (21.09 g, yield: 72%).

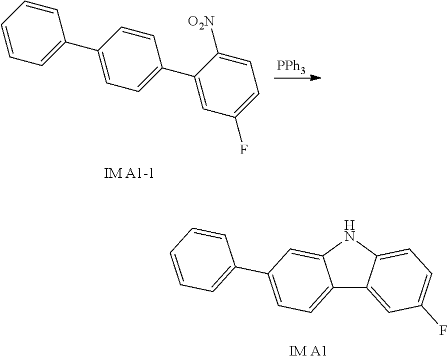

(2) To a 250 mL three-necked flask was added IM A1-1 (14.66 g, 50.00 mmol), triphenylphosphine (39.34 g, 150.00 mmol), and o-dichlorobenzene (150 mL) and the resulting mixture was heated to 160° C. under nitrogen protection, and stirred for 18 hours of reaction. Then the reaction solution was cooled to room temperature, and washed with water and extracted with toluene. The extracted solution was then dried over magnesium sulfate, and after filtration, the filtrate was concentrated under reduced pressure to remove the solvent, to obtain a crude product. The crude product was purified by recrystallization from n-heptane and dichloromethane, to obtain IM A1 (9.93 g, yield: 76%).

Other IM AX listed in Table 1 were synthesized by reference to the method of synthesizing IM A1, except that Raw Material 1 was used instead of 4-biphenylboronic acid, and Raw Material 2 was used instead of 2-bromo-4-fluoronitrobenzene. The main raw materials used, the synthesized IM AX, and their yields are presented in Table 1.

TABLE 1

| Raw material 1 | Raw material 2 | IM AX | Yield/% |
|---|---|---|---|
| 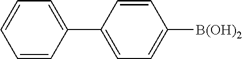 [SMILES: OB(O)c1ccc(-c2ccccc2)cc1] | 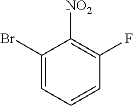 [SMILES: O=[N+]([O-])c1c(F)cccc1Br] | 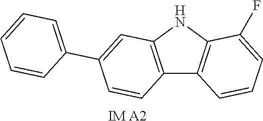 | 43 |
| 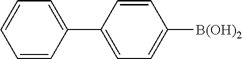 [SMILES: OB(O)c1ccc(-c2ccccc2)cc1] | 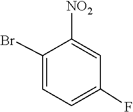 [SMILES: O=[N+]([O-])c1cc(F)ccc1Br] | 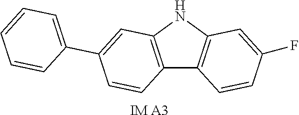 | 50 |
| 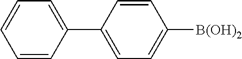 [SMILES: OB(O)c1ccc(-c2ccccc2)cc1] | 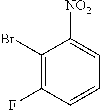 [SMILES: O=[N+]([O-])c1cccc(F)c1Br] | 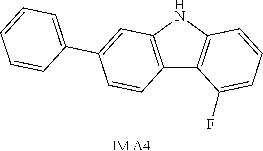 | 45 |
| 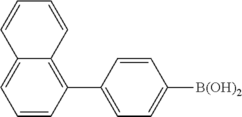 [SMILES: OB(O)c1ccc(-c2cccc3ccccc23)cc1] | 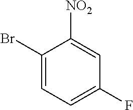 [SMILES: O=[N+]([O-])c1cc(F)ccc1Br] | 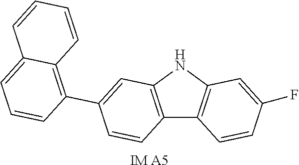 | 39 |
| 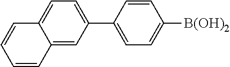 [SMILES: OB(O)c1ccc(-c2ccc3ccccc3c2)cc1] | 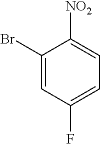 [SMILES: O=[N+]([O-])c1ccc(F)cc1Br] | 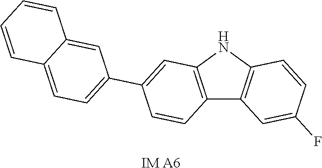 | 42 |
| 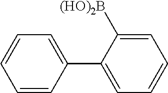 [SMILES: OB(O)c1ccccc1-c1ccccc1] | 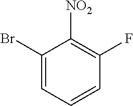 [SMILES: O=[N+]([O-])c1c(F)cccc1Br] | 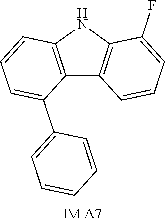 | 38 |
| 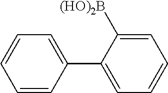 [SMILES: OB(O)c1ccccc1-c1ccccc1] | 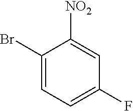 [SMILES: O=[N+]([O-])c1cc(F)ccc1Br] | 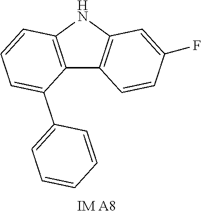 | 40 |
| 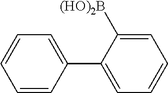 [SMILES: OB(O)c1ccccc1-c1ccccc1] | 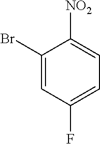 [SMILES: O=[N+]([O-])c1ccc(F)cc1Br] | 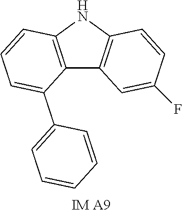 | 39 |
| 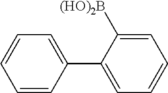 [SMILES: OB(O)c1ccccc1-c1ccccc1] | 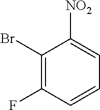 [SMILES: O=[N+]([O-])c1cccc(F)c1Br] | 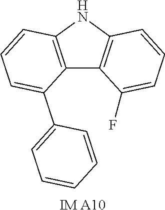 | 42 |
| 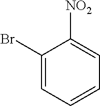 | 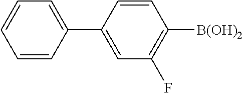 [SMILES: OB(O)c1ccc(-c2ccccc2)cc1F] | 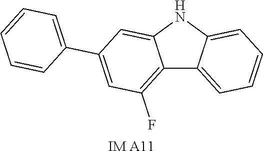 | 43 |
| 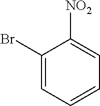 | 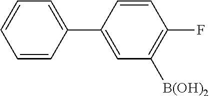 [SMILES: OB(O)c1cc(-c2ccccc2)ccc1F] | 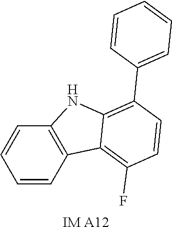 | 46 |
| 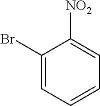 | 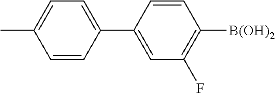 [SMILES: Cc1ccc(-c2ccc(B(O)O)c(F)c2)cc1] | 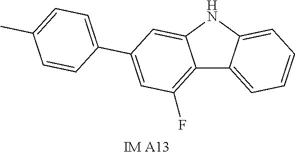 | 48 |
| 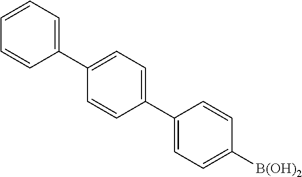 [SMILES: OB(O)c1ccc(-c2ccc(-c3ccccc3)cc2)cc1] | 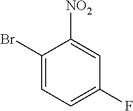 [SMILES: O=[N+]([O-])c1cc(F)ccc1Br] | 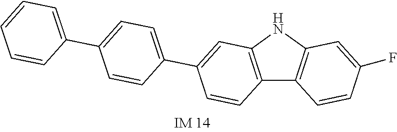 | 42 |
| 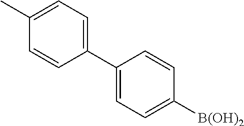 [SMILES: Cc1ccc(-c2ccc(B(O)O)cc2)cc1] | 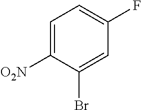 [SMILES: O=[N+]([O-])c1ccc(F)cc1Br] | 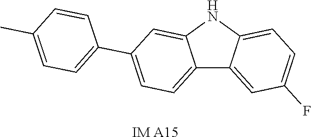 | 45 |
| 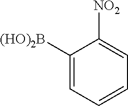 [SMILES: O=[N+]([O-])c1ccccc1B(O)O] | 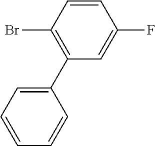 [SMILES: Fc1ccc(Br)c(-c2ccccc2)c1] | 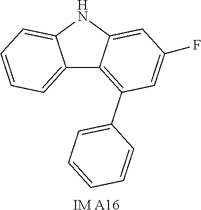 | 44 |

### 2. Synthesis of IM BX

The synthesis of IM BX is illustrated by means of an example of IM B1.

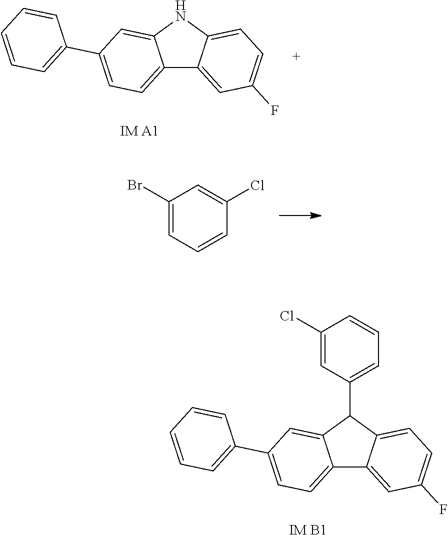

IM A1 (6.53 g, 25.00 mmol), 3-chloro-1-bromobenzene (4.78 g, 25.00 mmol), tris(dibenzylidene acetone) dipalladium (0.23 g, 0.25 mmol), 2-dicyclohexylphosphino-2′,4′,6′-triisopropylbiphenyl (0.24 g, 0.50 mmol), and sodium tert-butoxide (4.8 g, 50.0 mmol) were added into toluene (50 mL). The resulting mixture was heated to 108° C. under nitrogen protection and stirred for 3 hours of reaction. Then the reaction solution was cooled to room temperature, and washed with water. The separated organic phase was then dried over magnesium sulfate, and after filtration, the filtrate was concentrated under reduced pressure to remove the solvent, to obtain a crude product. The crude product was purified by recrystallization from toluene (1 g crude product: 6 mL toluene), to obtain IM B1 as a white product (4.83 g, yield: 52.0%).

Other IM BX listed in Table 2 were synthesized by reference to the method of synthesizing IM B1, except that Raw material 3 was used instead of IM A1, and Raw material 4 was used instead of 3-chloro-1-bromobenzene. The synthesized IM BX and its yield are presented in Table 2.

TABLE 2

| Raw material 3 | Raw material 4 | IM BX | Yield/% |
|---|---|---|---|
| 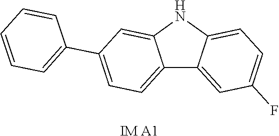 | 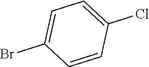 | 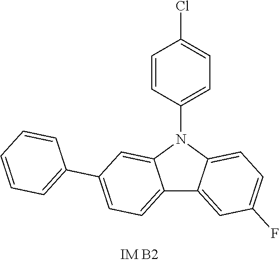 | 44 |
| 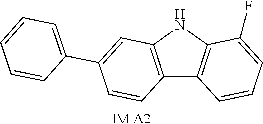 | 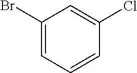 | 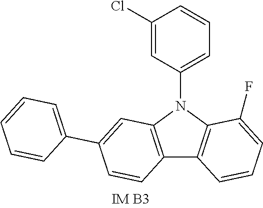 | 45 |
| 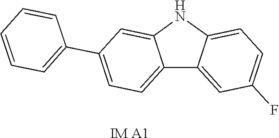 | 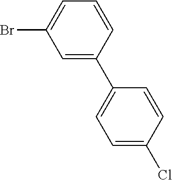 [SMILES: Clc1ccc(-c2cccc(Br)c2)cc1] | 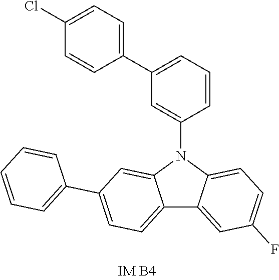 | 48 |
| 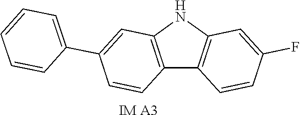 | 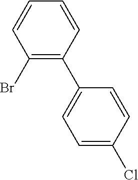 [SMILES: Clc1ccc(-c2ccccc2Br)cc1] | 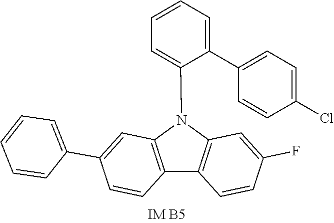 | 43 |
| 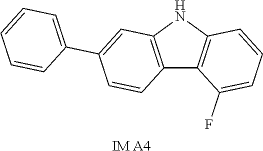 | 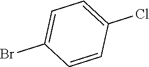 | 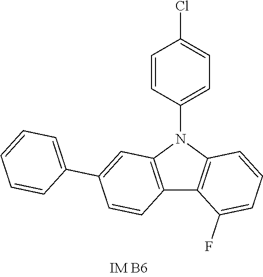 | 44 |
| 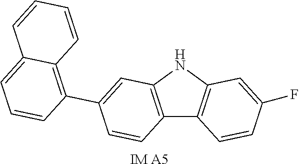 | 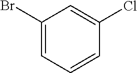 | 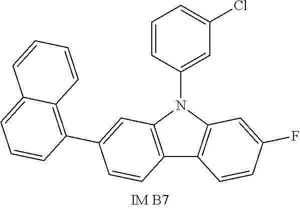 | 39 |
| 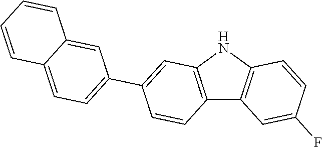 [SMILES: Fc1ccc2[nH]c3cc(-c4ccc5ccccc5c4)ccc3c2c1] IM A6 | 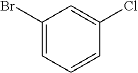 | 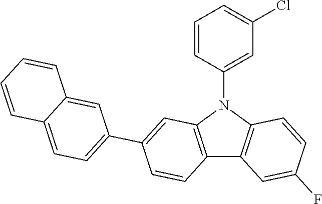 IM B8 | 41 |
| 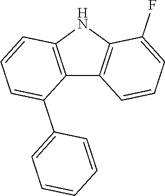 [SMILES: Fc1cccc2c1[nH]c1cccc(-c3ccccc3)c12] IM A7 | 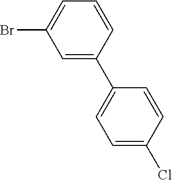 [SMILES: Clc1ccc(-c2cccc(Br)c2)cc1] | 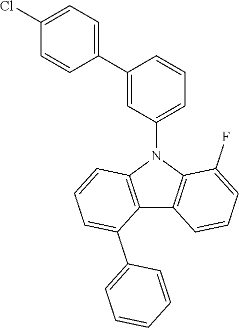 IM B9 | 47 |
| 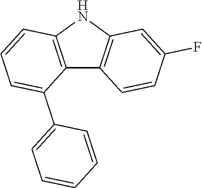 [SMILES: Fc1ccc2c(c1)[nH]c1cccc(-c3ccccc3)c12] IM A8 | 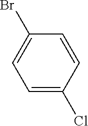 | 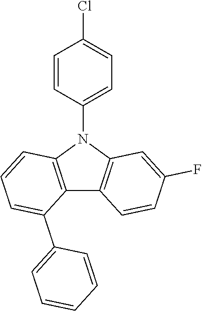 IM B10 | 48 |
| 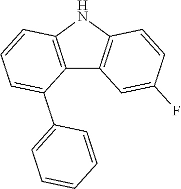 [SMILES: Fc1ccc2[nH]c3cccc(-c4ccccc4)c3c2c1] IM A9 | 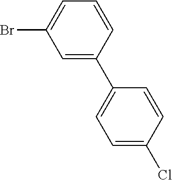 [SMILES: Clc1ccc(-c2cccc(Br)c2)cc1] | 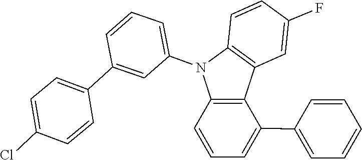 IM B11 | 50 |
| 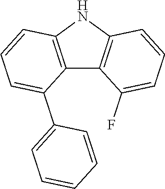 [SMILES: Fc1cccc2[nH]c3cccc(-c4ccccc4)c3c12] IM A10 | 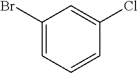 | 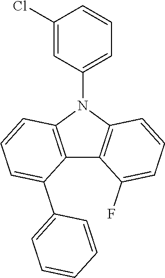 IM B12 | 48 |
| 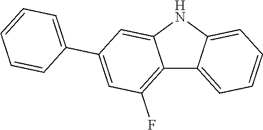 [SMILES: Fc1cc(-c2ccccc2)cc2[nH]c3ccccc3c12] IM A11 | 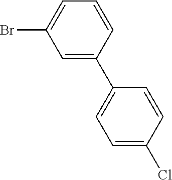 [SMILES: Clc1ccc(-c2cccc(Br)c2)cc1] | 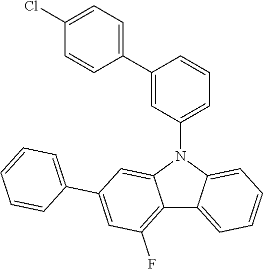 IM B13 | 46 |
| 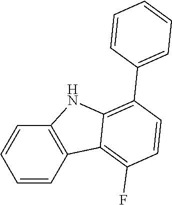 [SMILES: Fc1ccc(-c2ccccc2)c2[nH]c3ccccc3c12] IM A12 | 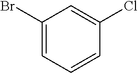 | 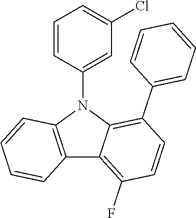 IM B14 | 41 |
| 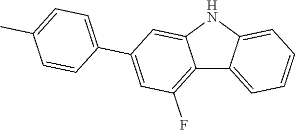 [SMILES: Cc1ccc(-c2cc(F)c3c(c2)[nH]c2ccccc23)cc1] IM A13 | 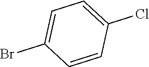 | 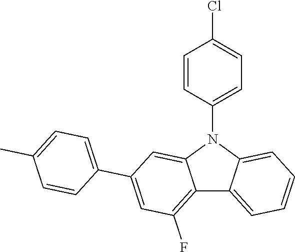 IM B15 | 39 |
| 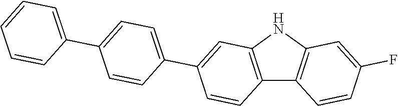 [SMILES: Fc1ccc2c(c1)[nH]c1cc(-c3ccc(-c4ccccc4)cc3)ccc12] IM A14 | 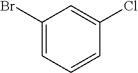 | 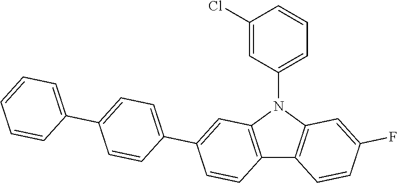 IM B16 | 43 |
| 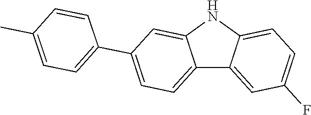 [SMILES: Cc1ccc(-c2ccc3c(c2)[nH]c2ccc(F)cc23)cc1] IM A15 | 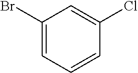 | 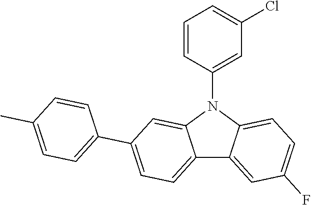 IM B17 | 41 |
| 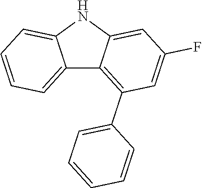 [SMILES: Fc1cc(-c2ccccc2)c2c(c1)[nH]c1ccccc12] IM A16 | 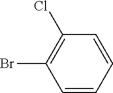 | 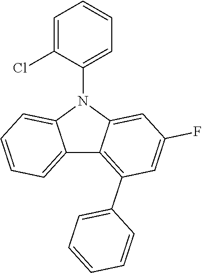 IM B18 | 45 |
| 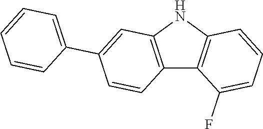 [SMILES: Fc1cccc2[nH]c3cc(-c4ccccc4)ccc3c12] IM A4 | 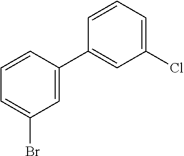 [SMILES: Clc1cccc(-c2cccc(Br)c2)c1] | 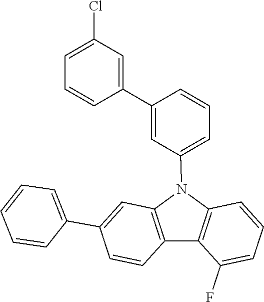 IM B19 | 49 |
| 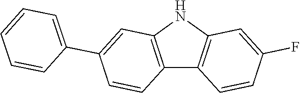 [SMILES: Fc1ccc2c(c1)[nH]c1cc(-c3ccccc3)ccc12] IM A3 | 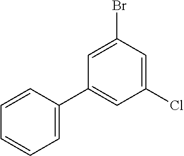 [SMILES: Clc1cc(Br)cc(-c2ccccc2)c1] | 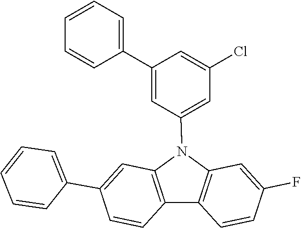 IM B20 | 42 |
| 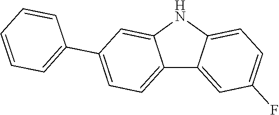 [SMILES: Fc1ccc2[nH]c3cc(-c4ccccc4)ccc3c2c1] IM A1 | 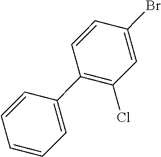 [SMILES: Clc1cc(Br)ccc1-c1ccccc1] | 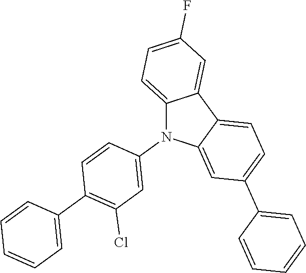 IM B21 | 46 |

### Synthesis Example 1

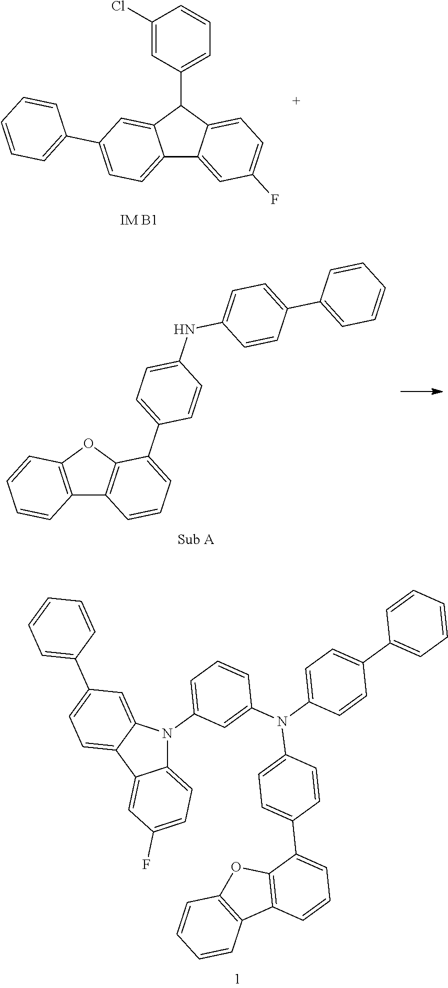

IM B1 (7.43 g, 20.00 mmol), Raw material Sub A (8.23 g, 20.00 mmol), tris(dibenzylidene acetone) dipalladium (0.18 g, 0.20 mmol), 2-dicyclohexylphosphino-2′,6′-dimethoxybiphenyl (0.16 g, 0.40 mmol), and sodium tert-butoxide (3.85 g, 40.00 mmol) were added into toluene (100 mL). The resulting mixture was heated to 105° C. under nitrogen protection and stirred for 4 hours. Then the reaction solution was cooled to room temperature, and washed with water, and the separated organic phase was dried over magnesium sulfate. After filtration, the filtrate was concentrated under reduced pressure to remove the solvent, to obtain a crude product. The crude product was purified by recrystallization from toluene (1 g crude product: 12 mL toluene) to obtain Compound 1 as a white solid (10.91 g, yield: 73%), with a mass spectrum (m/z)=747.3 [M+H]⁺.

### Synthesis Examples 2 to 37

Other compounds listed in Table 3 were synthesized by reference to the method of synthesizing Compound 1, except that Raw material 5 was used instead of IM B1, and Raw material 6 was used instead of Sub A. The main raw materials used, the synthesized compounds, their yields, and the mass spectral results are presented in Table 3.

TABLE 3

| Syn- | | | | | Mass |
|---|---|---|---|---|---|
| thesis | | | | | Spectra/ |
| Exam- | | | | Yield/ | [M + |
| ple | Raw material 5 | Raw material 6 | Compound | % | H]⁺ |
| 2 | 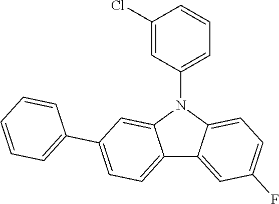 [SMILES: Fc1ccc2c(c1)c1ccc(-c3ccccc3)cc1n2-c1cccc(Cl)c1] IM B1 | 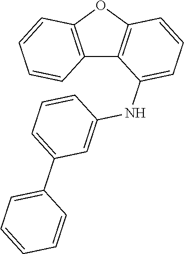 [SMILES: c1ccc(-c2cccc(Nc3cccc4oc5ccccc5c34)c2)cc1] | 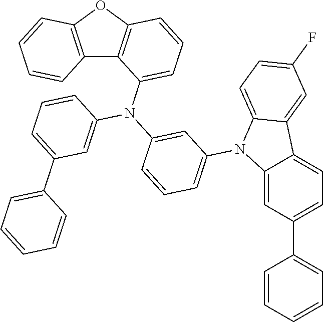 [SMILES: Fc1ccc2c(c1)c1ccc(-c3ccccc3)cc1n2-c1cccc(N(c2cccc(-c3ccccc3)c2)c2cccc3oc4ccccc4c23)c1] 7 | 66 | 671.2 |
| 3 | 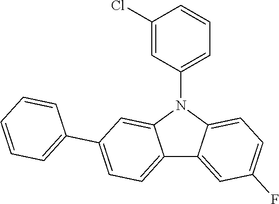 [SMILES: Fc1ccc2c(c1)c1ccc(-c3ccccc3)cc1n2-c1cccc(Cl)c1] IM B1 | 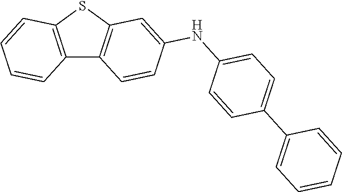 [SMILES: c1ccc(-c2ccc(Nc3ccc4c(c3)sc3ccccc34)cc2)cc1] | 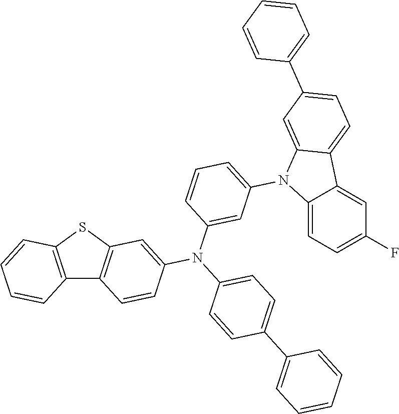 [SMILES: Fc1ccc2c(c1)c1ccc(-c3ccccc3)cc1n2-c1cccc(N(c2ccc(-c3ccccc3)cc2)c2ccc3c(c2)sc2ccccc23)c1] 12 | 70 | 687.2 |
| 4 | 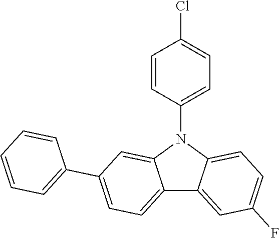 [SMILES: Fc1ccc2c(c1)c1ccc(-c3ccccc3)cc1n2-c1ccc(Cl)cc1] IM B2 | 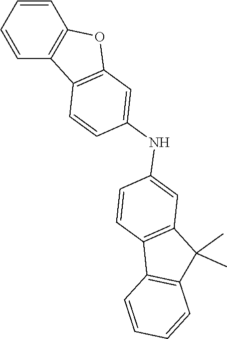 [SMILES: CC1(C)c2ccccc2-c2ccc(Nc3ccc4c(c3)oc3ccccc34)cc21] | 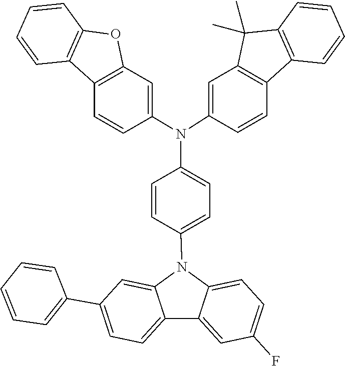 [SMILES: CC1(C)c2ccccc2-c2ccc(N(c3ccc(-n4c5ccc(F)cc5c5ccc(-c6ccccc6)cc54)cc3)c3ccc4c(c3)oc3ccccc34)cc21] 15 | 69 | 711.3 |
| 5 | 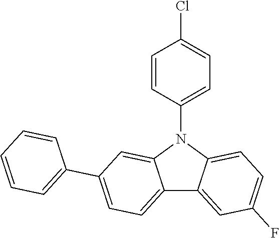 [SMILES: Fc1ccc2c(c1)c1ccc(-c3ccccc3)cc1n2-c1ccc(Cl)cc1] IM B2 | 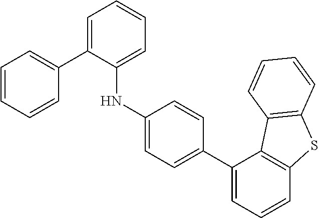 [SMILES: c1ccc(-c2ccccc2Nc2ccc(-c3cccc4sc5ccccc5c34)cc2)cc1] | 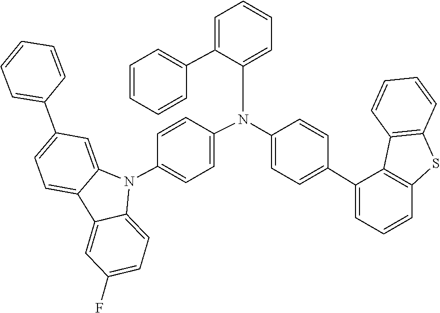 [SMILES: Fc1ccc2c(c1)c1ccc(-c3ccccc3)cc1n2-c1ccc(N(c2ccc(-c3cccc4sc5ccccc5c34)cc2)c2ccccc2-c2ccccc2)cc1] 20 | 71 | 763.3 |
| 6 | 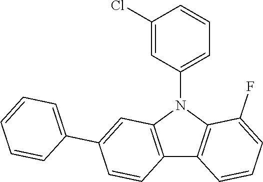 [SMILES: Fc1cccc2c3ccc(-c4ccccc4)cc3n(-c3cccc(Cl)c3)c12] IM B3 | 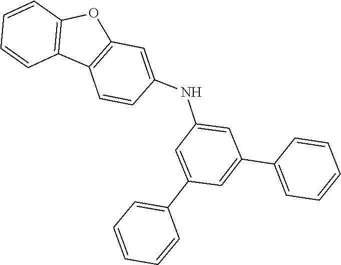 [SMILES: c1ccc(-c2cc(Nc3ccc4c(c3)oc3ccccc34)cc(-c3ccccc3)c2)cc1] | 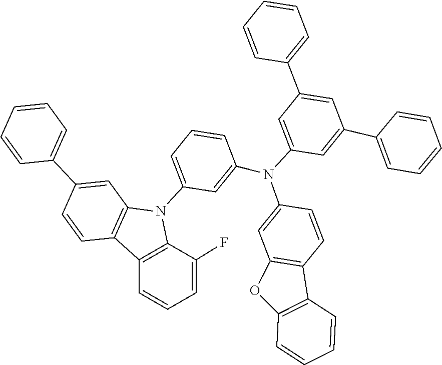 [SMILES: Fc1cccc2c3ccc(-c4ccccc4)cc3n(-c3cccc(N(c4cc(-c5ccccc5)cc(-c5ccccc5)c4)c4ccc5c(c4)oc4ccccc45)c3)c12] 25 | 67 | 747.3 |
| 7 | 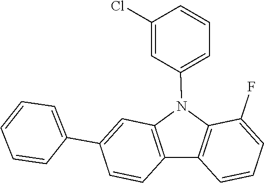 [SMILES: Fc1cccc2c3ccc(-c4ccccc4)cc3n(-c3cccc(Cl)c3)c12] IM B3 | 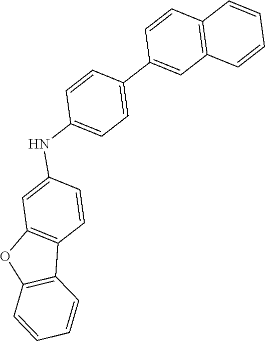 [SMILES: c1ccc2cc(-c3ccc(Nc4ccc5c(c4)oc4ccccc45)cc3)ccc2c1] | 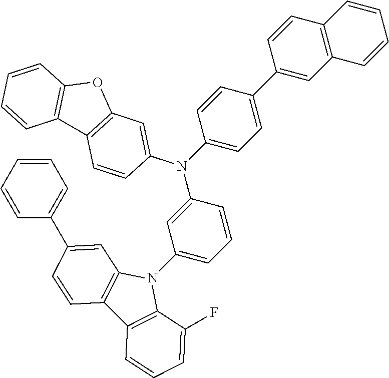 [SMILES: Fc1cccc2c3ccc(-c4ccccc4)cc3n(-c3cccc(N(c4ccc(-c5ccc6ccccc6c5)cc4)c4ccc5c(c4)oc4ccccc45)c3)c12] 41 | 79 | 721.3 |
| 8 | 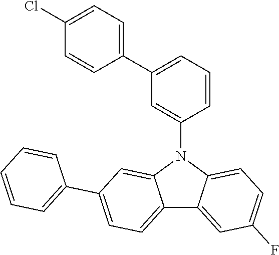 [SMILES: Fc1ccc2c(c1)c1ccc(-c3ccccc3)cc1n2-c1cccc(-c2ccc(Cl)cc2)c1] IM B4 | 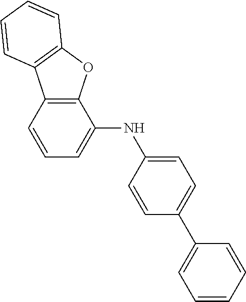 [SMILES: c1ccc(-c2ccc(Nc3cccc4c3oc3ccccc34)cc2)cc1] | 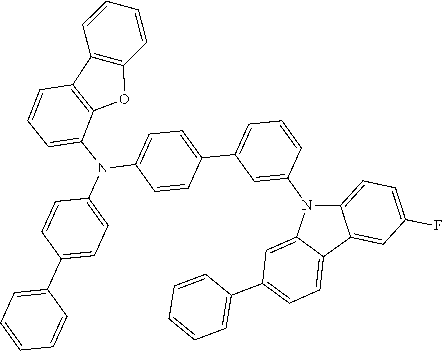 [SMILES: Fc1ccc2c(c1)c1ccc(-c3ccccc3)cc1n2-c1cccc(-c2ccc(N(c3ccc(-c4ccccc4)cc3)c3cccc4c3oc3ccccc34)cc2)c1] 252 | 73 | 747.3 |
| 9 | 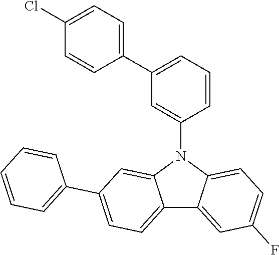 [SMILES: Fc1ccc2c(c1)c1ccc(-c3ccccc3)cc1n2-c1cccc(-c2ccc(Cl)cc2)c1] IM B4 | 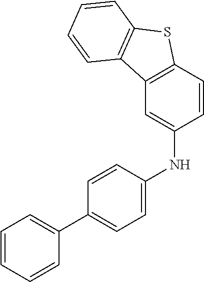 [SMILES: c1ccc(-c2ccc(Nc3ccc4sc5ccccc5c4c3)cc2)cc1] | 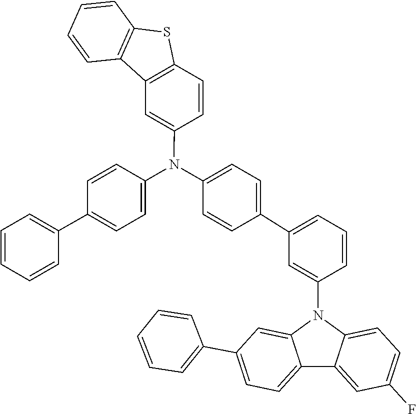 [SMILES: Fc1ccc2c(c1)c1ccc(-c3ccccc3)cc1n2-c1cccc(-c2ccc(N(c3ccc(-c4ccccc4)cc3)c3ccc4sc5ccccc5c4c3)cc2)c1] 253 | 74 | 763.3 |
| 10 | 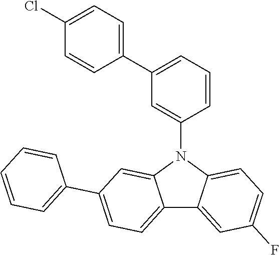 [SMILES: Fc1ccc2c(c1)c1ccc(-c3ccccc3)cc1n2-c1cccc(-c2ccc(Cl)cc2)c1] IM B4 | 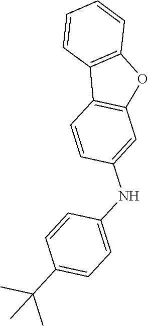 [SMILES: CC(C)(C)c1ccc(Nc2ccc3c(c2)oc2ccccc23)cc1] | 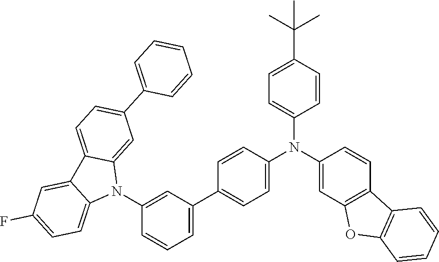 [SMILES: CC(C)(C)c1ccc(N(c2ccc(-c3cccc(-n4c5ccc(F)cc5c5ccc(-c6ccccc6)cc54)c3)cc2)c2ccc3c(c2)oc2ccccc23)cc1] 258 | 71 | 727.3 |
| 11 | 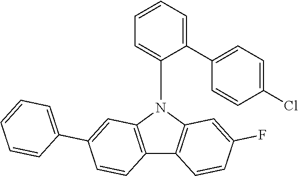 [SMILES: Fc1ccc2c3ccc(-c4ccccc4)cc3n(-c3ccccc3-c3ccc(Cl)cc3)c2c1] IM B5 | 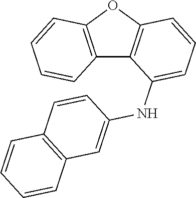 [SMILES: c1ccc2cc(Nc3cccc4oc5ccccc5c34)ccc2c1] | 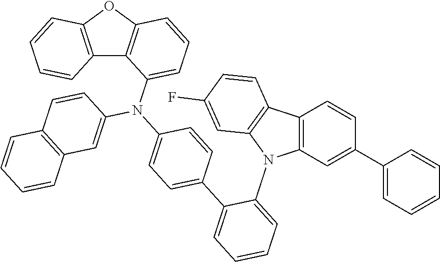 [SMILES: Fc1ccc2c3ccc(-c4ccccc4)cc3n(-c3ccccc3-c3ccc(N(c4ccc5ccccc5c4)c4cccc5oc6ccccc6c45)cc3)c2c1] 245 | 74 | 721.3 |
| 12 | 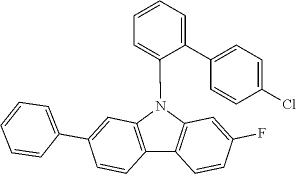 [SMILES: Fc1ccc2c3ccc(-c4ccccc4)cc3n(-c3ccccc3-c3ccc(Cl)cc3)c2c1] IM B5 | 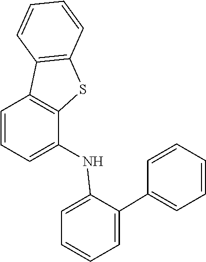 [SMILES: c1ccc(-c2ccccc2Nc2cccc3c2sc2ccccc23)cc1] | 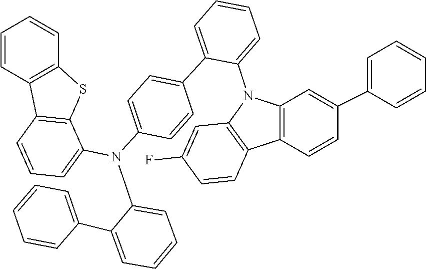 [SMILES: Fc1ccc2c3ccc(-c4ccccc4)cc3n(-c3ccccc3-c3ccc(N(c4ccccc4-c4ccccc4)c4cccc5c4sc4ccccc45)cc3)c2c1] 261 | 67 | 763.3 |
| 13 | 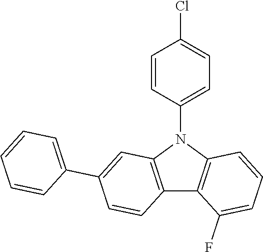 [SMILES: Fc1cccc2c1c1ccc(-c3ccccc3)cc1n2-c1ccc(Cl)cc1] IM B6 | 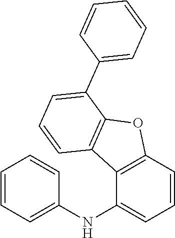 [SMILES: c1ccc(Nc2cccc3oc4c(-c5ccccc5)cccc4c23)cc1] | 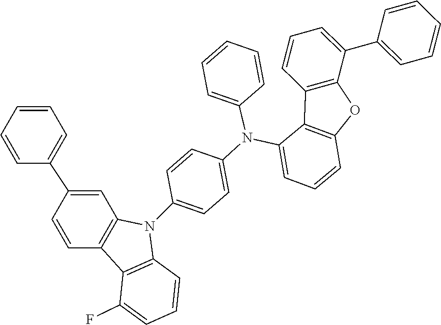 [SMILES: Fc1cccc2c1c1ccc(-c3ccccc3)cc1n2-c1ccc(N(c2ccccc2)c2cccc3oc4c(-c5ccccc5)cccc4c23)cc1] 73 | 68 | 671.2 |
| 14 | 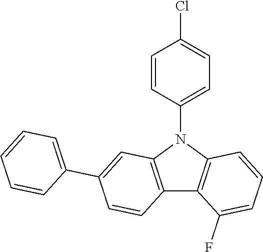 [SMILES: Fc1cccc2c1c1ccc(-c3ccccc3)cc1n2-c1ccc(Cl)cc1] IM B6 | 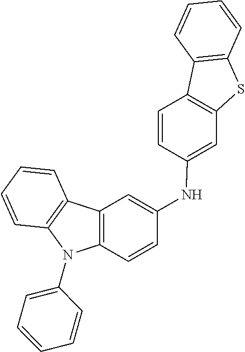 [SMILES: c1ccc(-n2c3ccccc3c3cc(Nc4ccc5c(c4)sc4ccccc45)ccc32)cc1] | 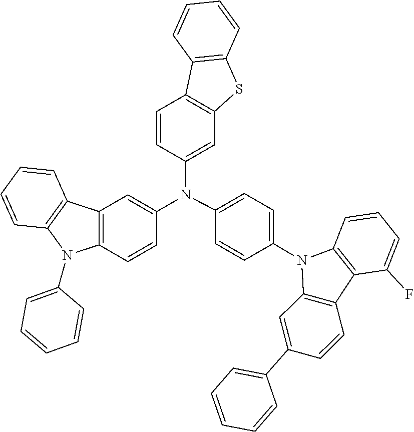 [SMILES: Fc1cccc2c1c1ccc(-c3ccccc3)cc1n2-c1ccc(N(c2ccc3c(c2)sc2ccccc23)c2ccc3c(c2)c2ccccc2n3-c2ccccc2)cc1] 81 | 70 | 776.3 |
| 15 | 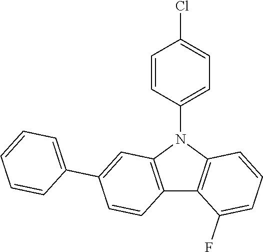 [SMILES: Fc1cccc2c1c1ccc(-c3ccccc3)cc1n2-c1ccc(Cl)cc1] IM B6 | 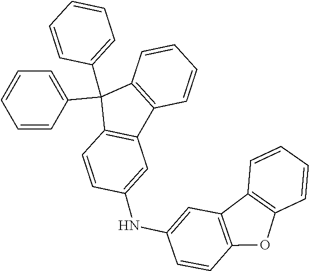 [SMILES: c1ccc(C2(c3ccccc3)c3ccccc3-c3cc(Nc4ccc5oc6ccccc6c5c4)ccc32)cc1] | 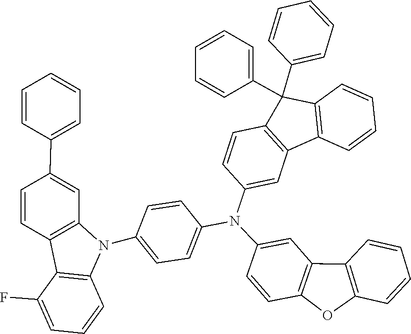 [SMILES: Fc1cccc2c1c1ccc(-c3ccccc3)cc1n2-c1ccc(N(c2ccc3c(c2)-c2ccccc2C3(c2ccccc2)c2ccccc2)c2ccc3oc4ccccc4c3c2)cc1] 90 | 64 | 835.3 |
| 16 | 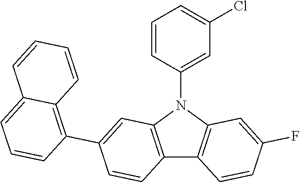 [SMILES: Fc1ccc2c3ccc(-c4cccc5ccccc45)cc3n(-c3cccc(Cl)c3)c2c1] IM B7 | 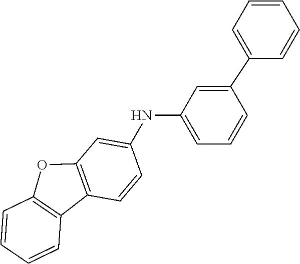 [SMILES: c1ccc(-c2cccc(Nc3ccc4c(c3)oc3ccccc34)c2)cc1] | 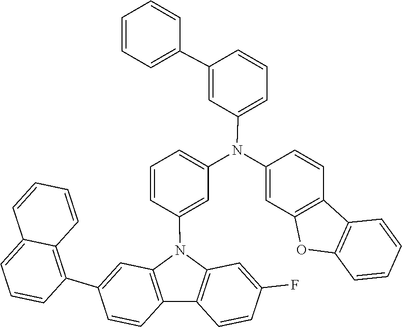 [SMILES: Fc1ccc2c3ccc(-c4cccc5ccccc45)cc3n(-c3cccc(N(c4cccc(-c5ccccc5)c4)c4ccc5c(c4)oc4ccccc45)c3)c2c1] 186 | 71 | 721.3 |
| 17 | 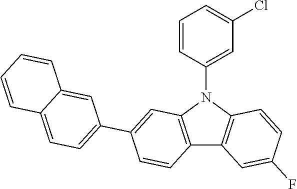 [SMILES: Fc1ccc2c(c1)c1ccc(-c3ccc4ccccc4c3)cc1n2-c1cccc(Cl)c1] IM B8 | 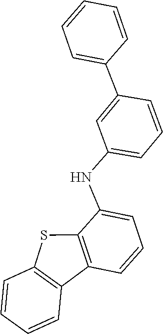 [SMILES: c1ccc(-c2cccc(Nc3cccc4c3sc3ccccc34)c2)cc1] | 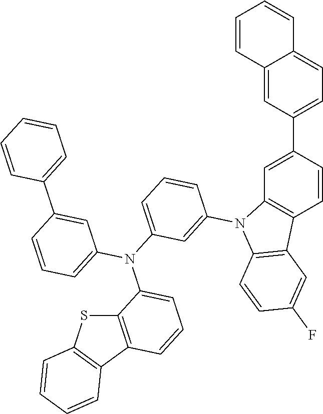 [SMILES: Fc1ccc2c(c1)c1ccc(-c3ccc4ccccc4c3)cc1n2-c1cccc(N(c2cccc(-c3ccccc3)c2)c2cccc3c2sc2ccccc23)c1] 99 | 60 | 737.2 |
| 18 | 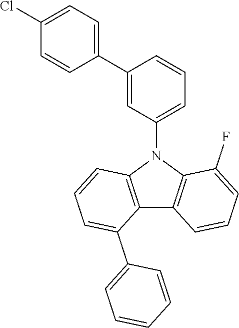 [SMILES: Fc1cccc2c3c(-c4ccccc4)cccc3n(-c3cccc(-c4ccc(Cl)cc4)c3)c12] IM B9 | 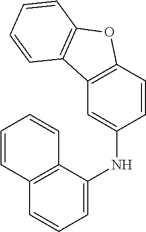 [SMILES: c1ccc2c(Nc3ccc4oc5ccccc5c4c3)cccc2c1] | 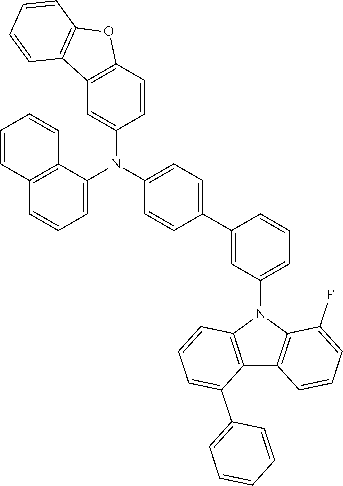 [SMILES: Fc1cccc2c3c(-c4ccccc4)cccc3n(-c3cccc(-c4ccc(N(c5ccc6oc7ccccc7c6c5)c5cccc6ccccc56)cc4)c3)c12] 224 | 66 | 721.3 |
| 19 | 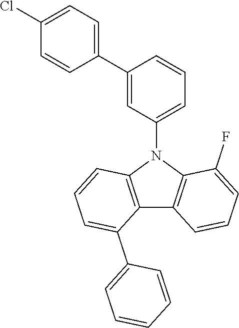 [SMILES: Fc1cccc2c3c(-c4ccccc4)cccc3n(-c3cccc(-c4ccc(Cl)cc4)c3)c12] IM B9 | 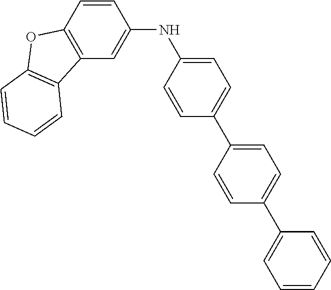 [SMILES: c1ccc(-c2ccc(-c3ccc(Nc4ccc5oc6ccccc6c5c4)cc3)cc2)cc1] | 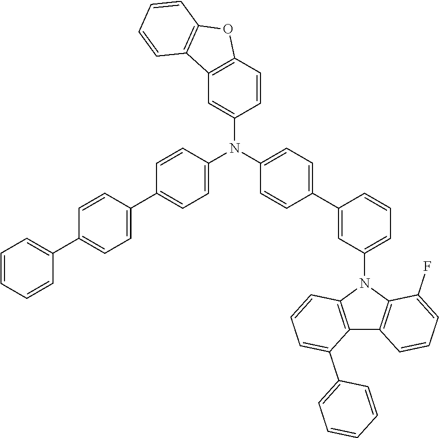 [SMILES: Fc1cccc2c3c(-c4ccccc4)cccc3n(-c3cccc(-c4ccc(N(c5ccc(-c6ccc(-c7ccccc7)cc6)cc5)c5ccc6oc7ccccc7c6c5)cc4)c3)c12] 229 | 77 | 823.3 |
| 20 | 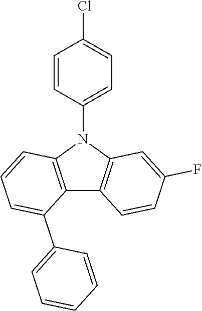 [SMILES: Fc1ccc2c3c(-c4ccccc4)cccc3n(-c3ccc(Cl)cc3)c2c1] IM B10 | 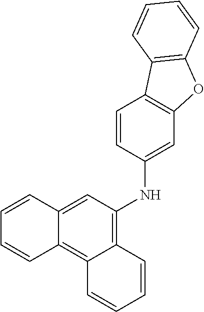 [SMILES: c1ccc2c(c1)cc(Nc1ccc3c(c1)oc1ccccc13)c1ccccc12] | 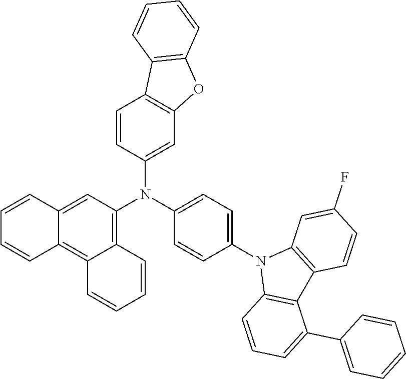 [SMILES: Fc1ccc2c3c(-c4ccccc4)cccc3n(-c3ccc(N(c4ccc5c(c4)oc4ccccc45)c4cc5ccccc5c5ccccc45)cc3)c2c1] 109 | 72 | 695.2 |
| 21 | 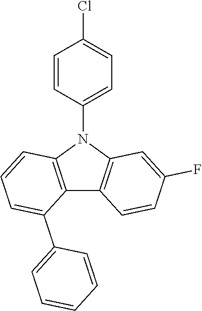 [SMILES: Fc1ccc2c3c(-c4ccccc4)cccc3n(-c3ccc(Cl)cc3)c2c1] IM B10 | 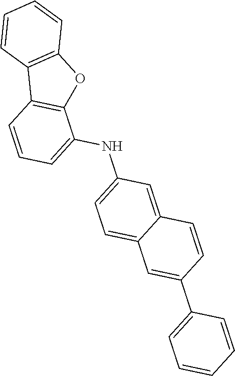 [SMILES: c1ccc(-c2ccc3cc(Nc4cccc5c4oc4ccccc45)ccc3c2)cc1] | 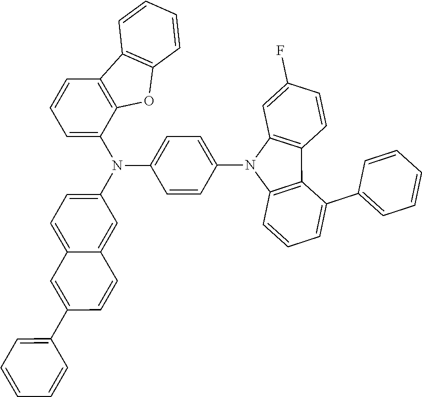 [SMILES: Fc1ccc2c3c(-c4ccccc4)cccc3n(-c3ccc(N(c4ccc5cc(-c6ccccc6)ccc5c4)c4cccc5c4oc4ccccc45)cc3)c2c1] 112 | 69 | 721.3 |
| 22 | 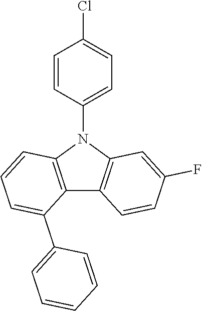 [SMILES: Fc1ccc2c3c(-c4ccccc4)cccc3n(-c3ccc(Cl)cc3)c2c1] IM B10 | 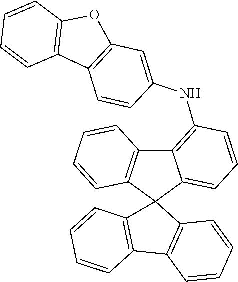 [SMILES: c1ccc2c(c1)-c1ccccc1C21c2ccccc2-c2c(Nc3ccc4c(c3)oc3ccccc34)cccc21] | 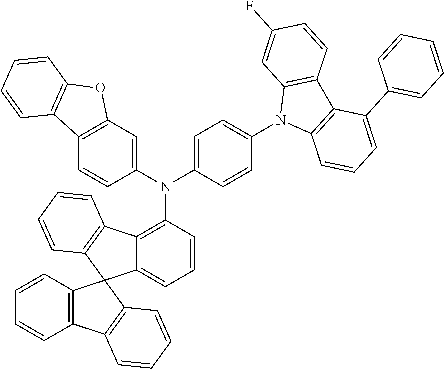 [SMILES: Fc1ccc2c3c(-c4ccccc4)cccc3n(-c3ccc(N(c4ccc5c(c4)oc4ccccc45)c4cccc5c4-c4ccccc4C54c5ccccc5-c5ccccc54)cc3)c2c1] 122 | 72 | 833.3 |
| 23 | 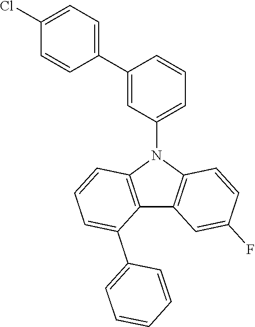 [SMILES: Fc1ccc2c(c1)c1c(-c3ccccc3)cccc1n2-c1cccc(-c2ccc(Cl)cc2)c1] IM B11 | 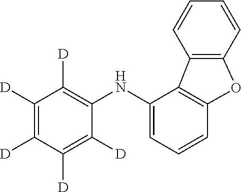 [SMILES: [2H]c1c([2H])c([2H])c(Nc2cccc3oc4ccccc4c23)c([2H])c1[2H]] | 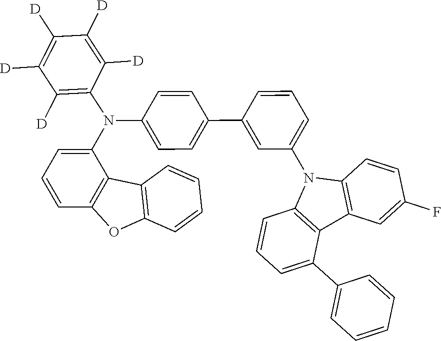 [SMILES: [2H]c1c([2H])c([2H])c(N(c2ccc(-c3cccc(-n4c5ccc(F)cc5c5c(-c6ccccc6)cccc54)c3)cc2)c2cccc3oc4ccccc4c23)c([2H])c1[2H]] 231 | 71 | 676.3 |
| 24 | 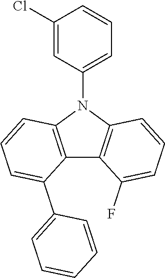 [SMILES: Fc1cccc2c1c1c(-c3ccccc3)cccc1n2-c1cccc(Cl)c1] IM B12 | 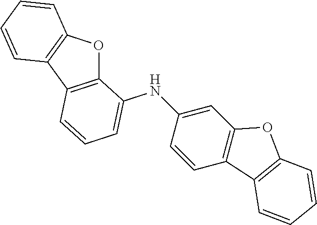 [SMILES: c1ccc2c(c1)oc1cc(Nc3cccc4c3oc3ccccc34)ccc12] | 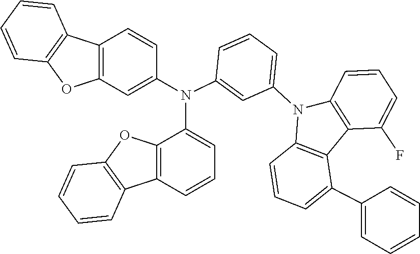 [SMILES: Fc1cccc2c1c1c(-c3ccccc3)cccc1n2-c1cccc(N(c2ccc3c(c2)oc2ccccc23)c2cccc3c2oc2ccccc23)c1] 142 | 74 | 685.2 |
| 25 | 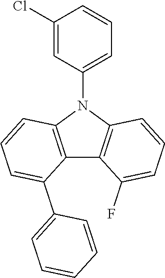 [SMILES: Fc1cccc2c1c1c(-c3ccccc3)cccc1n2-c1cccc(Cl)c1] IM B12 | 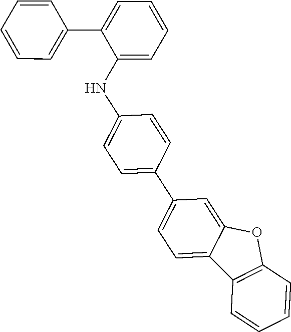 [SMILES: c1ccc(-c2ccccc2Nc2ccc(-c3ccc4c(c3)oc3ccccc34)cc2)cc1] | 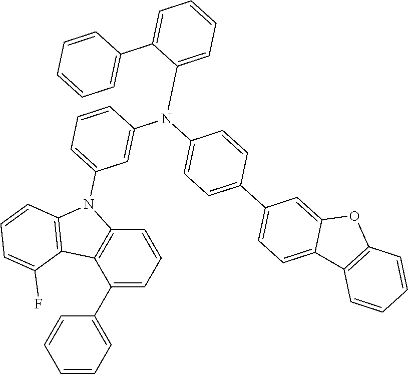 [SMILES: Fc1cccc2c1c1c(-c3ccccc3)cccc1n2-c1cccc(N(c2ccc(-c3ccc4c(c3)oc3ccccc34)cc2)c2ccccc2-c2ccccc2)c1] 151 | 66 | 747.3 |
| 26 | 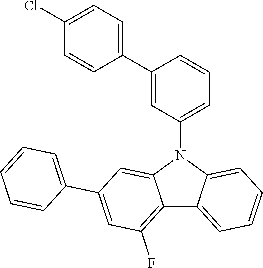 [SMILES: Fc1cc(-c2ccccc2)cc2c1c1ccccc1n2-c1cccc(-c2ccc(Cl)cc2)c1] IM B13 | 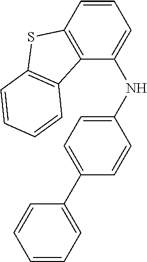 [SMILES: c1ccc(-c2ccc(Nc3cccc4sc5ccccc5c34)cc2)cc1] | 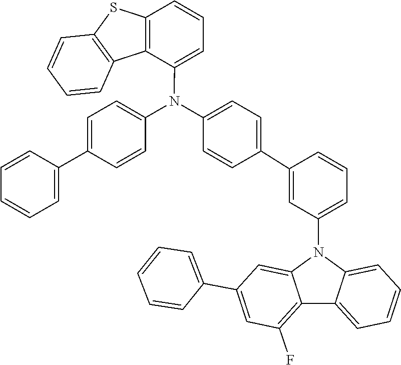 [SMILES: Fc1cc(-c2ccccc2)cc2c1c1ccccc1n2-c1cccc(-c2ccc(N(c3ccc(-c4ccccc4)cc3)c3cccc4sc5ccccc5c34)cc2)c1] 237 | 70 | 763.3 |
| 27 | 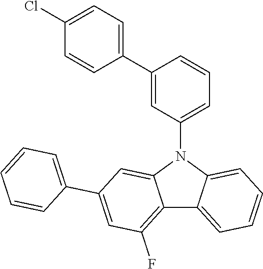 [SMILES: Fc1cc(-c2ccccc2)cc2c1c1ccccc1n2-c1cccc(-c2ccc(Cl)cc2)c1] IM B13 | 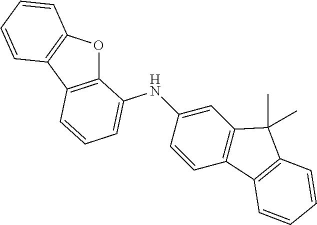 [SMILES: CC1(C)c2ccccc2-c2ccc(Nc3cccc4c3oc3ccccc34)cc21] | 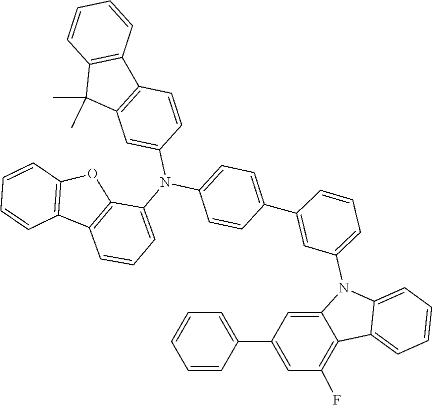 [SMILES: CC1(C)c2ccccc2-c2ccc(N(c3ccc(-c4cccc(-n5c6ccccc6c6c(F)cc(-c7ccccc7)cc65)c4)cc3)c3cccc4c3oc3ccccc34)cc21] 242 | 61 | 787.3 |
| 28 | 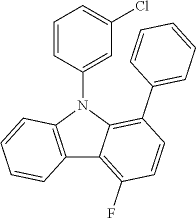 [SMILES: Fc1ccc(-c2ccccc2)c2c1c1ccccc1n2-c1cccc(Cl)c1] IM B14 | 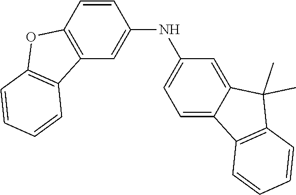 [SMILES: CC1(C)c2ccccc2-c2ccc(Nc3ccc4oc5ccccc5c4c3)cc21] | 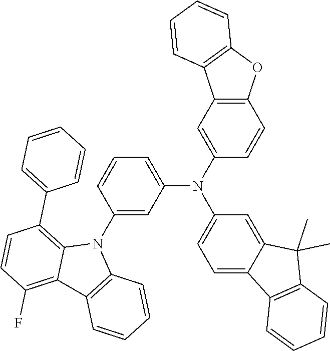 [SMILES: CC1(C)c2ccccc2-c2ccc(N(c3cccc(-n4c5ccccc5c5c(F)ccc(-c6ccccc6)c54)c3)c3ccc4oc5ccccc5c4c3)cc21] 176 | 72 | 711.3 |
| 29 | 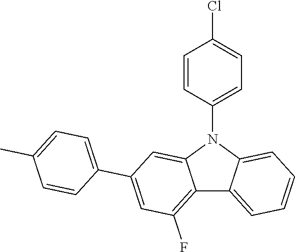 [SMILES: Cc1ccc(-c2cc(F)c3c4ccccc4n(-c4ccc(Cl)cc4)c3c2)cc1] IM B15 | 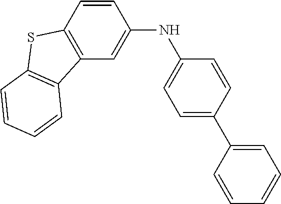 [SMILES: c1ccc(-c2ccc(Nc3ccc4sc5ccccc5c4c3)cc2)cc1] | 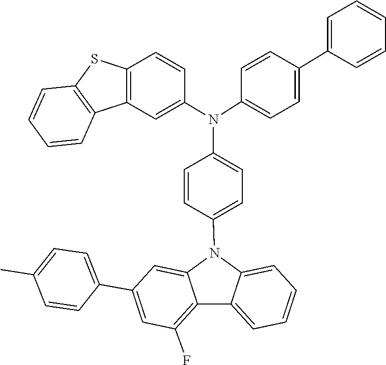 [SMILES: Cc1ccc(-c2cc(F)c3c4ccccc4n(-c4ccc(N(c5ccc(-c6ccccc6)cc5)c5ccc6sc7ccccc7c6c5)cc4)c3c2)cc1] 183 | 74 | 701.2 |
| 30 | 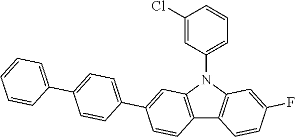 [SMILES: Fc1ccc2c3ccc(-c4ccc(-c5ccccc5)cc4)cc3n(-c3cccc(Cl)c3)c2c1] IM B16 | 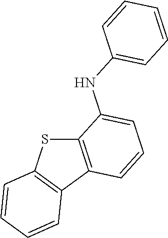 [SMILES: c1ccc(Nc2cccc3c2sc2ccccc23)cc1] | 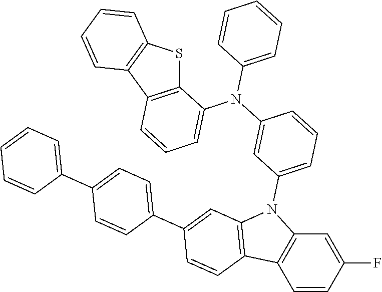 [SMILES: Fc1ccc2c3ccc(-c4ccc(-c5ccccc5)cc4)cc3n(-c3cccc(N(c4ccccc4)c4cccc5c4sc4ccccc45)c3)c2c1] 201 | 79 | 687.2 |
| 31 | 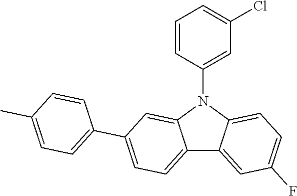 [SMILES: Cc1ccc(-c2ccc3c4cc(F)ccc4n(-c4cccc(Cl)c4)c3c2)cc1] IM B17 | 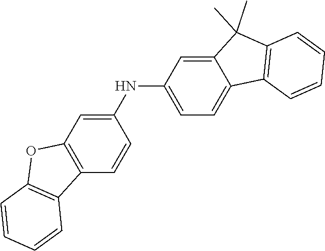 [SMILES: CC1(C)c2ccccc2-c2ccc(Nc3ccc4c(c3)oc3ccccc34)cc21] | 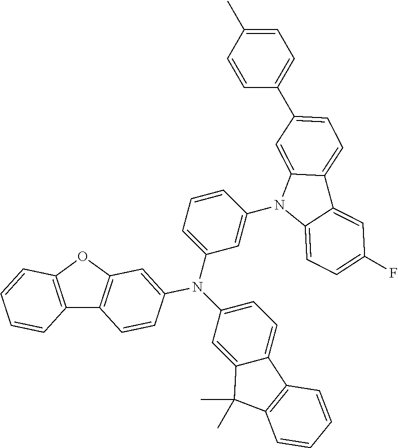 [SMILES: Cc1ccc(-c2ccc3c4cc(F)ccc4n(-c4cccc(N(c5ccc6c(c5)C(C)(C)c5ccccc5-6)c5ccc6c(c5)oc5ccccc56)c4)c3c2)cc1] 203 | 71 | 725.3 |
| 32 | 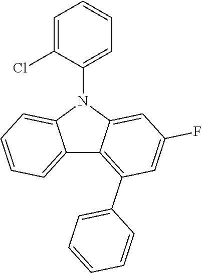 [SMILES: Fc1cc(-c2ccccc2)c2c3ccccc3n(-c3ccccc3Cl)c2c1] IM B18 | 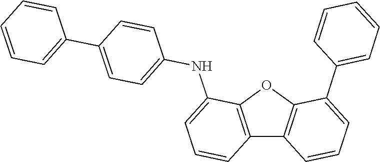 [SMILES: c1ccc(-c2ccc(Nc3cccc4c3oc3c(-c5ccccc5)cccc34)cc2)cc1] | 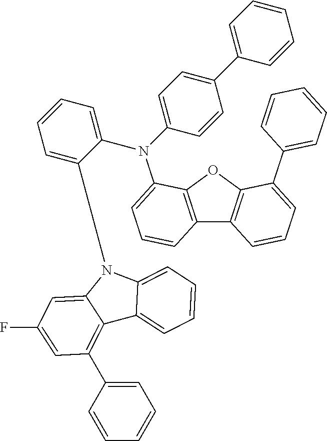 [SMILES: Fc1cc(-c2ccccc2)c2c3ccccc3n(-c3ccccc3N(c3ccc(-c4ccccc4)cc3)c3cccc4c3oc3c(-c5ccccc5)cccc34)c2c1] 212 | 71 | 747.3 |
| 33 | 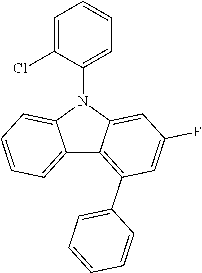 [SMILES: Fc1cc(-c2ccccc2)c2c3ccccc3n(-c3ccccc3Cl)c2c1] IM B18 | 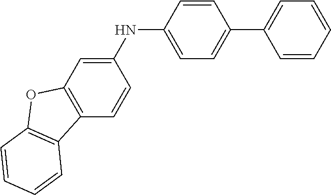 [SMILES: c1ccc(-c2ccc(Nc3ccc4c(c3)oc3ccccc34)cc2)cc1] | 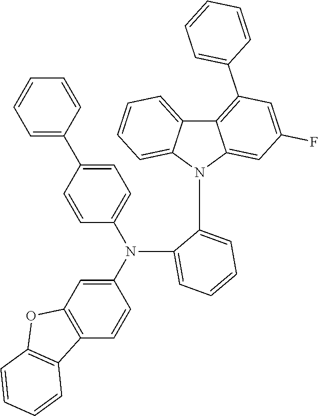 [SMILES: Fc1cc(-c2ccccc2)c2c3ccccc3n(-c3ccccc3N(c3ccc(-c4ccccc4)cc3)c3ccc4c(c3)oc3ccccc34)c2c1] 216 | 67 | 671.2 |
| 34 | 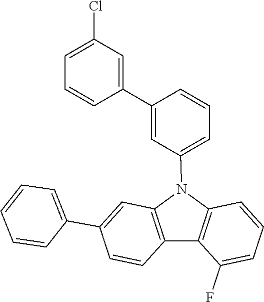 [SMILES: Fc1cccc2c1c1ccc(-c3ccccc3)cc1n2-c1cccc(-c2cccc(Cl)c2)c1] IM B19 | 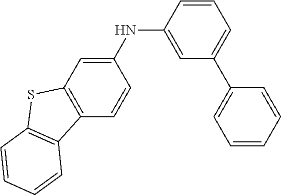 [SMILES: c1ccc(-c2cccc(Nc3ccc4c(c3)sc3ccccc34)c2)cc1] | 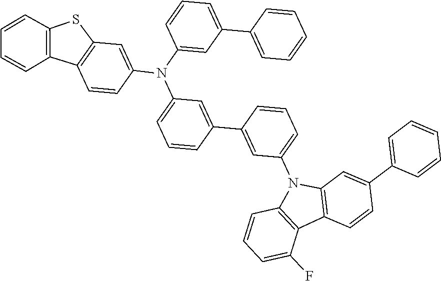 [SMILES: Fc1cccc2c1c1ccc(-c3ccccc3)cc1n2-c1cccc(-c2cccc(N(c3cccc(-c4ccccc4)c3)c3ccc4c(c3)sc3ccccc34)c2)c1] 223 | 74 | 763.3 |
| 35 | 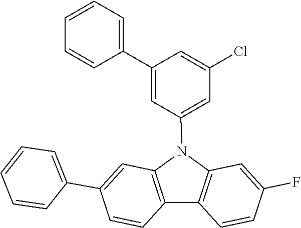 [SMILES: Fc1ccc2c3ccc(-c4ccccc4)cc3n(-c3cc(Cl)cc(-c4ccccc4)c3)c2c1] IM B20 | 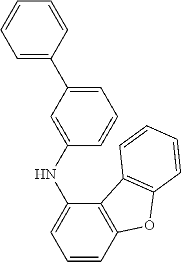 [SMILES: c1ccc(-c2cccc(Nc3cccc4oc5ccccc5c34)c2)cc1] | 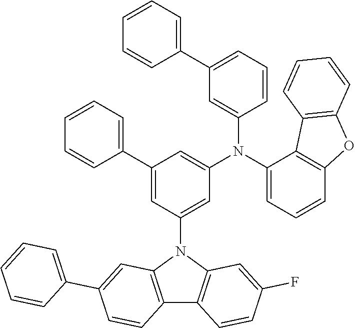 [SMILES: Fc1ccc2c3ccc(-c4ccccc4)cc3n(-c3cc(-c4ccccc4)cc(N(c4cccc(-c5ccccc5)c4)c4cccc5oc6ccccc6c45)c3)c2c1] 270 | 68 | 747.3 |
| 36 | 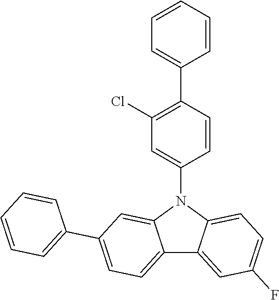 [SMILES: Fc1ccc2c(c1)c1ccc(-c3ccccc3)cc1n2-c1ccc(-c2ccccc2)c(Cl)c1] IM B21 | 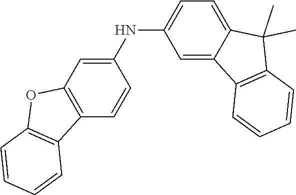 [SMILES: CC1(C)c2ccccc2-c2cc(Nc3ccc4c(c3)oc3ccccc34)ccc21] | 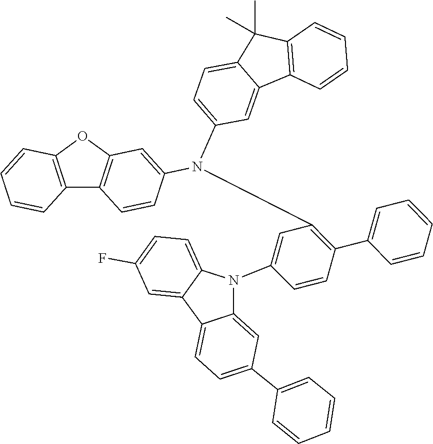 [SMILES: CC1(C)c2ccccc2-c2cc(N(c3ccc4c(c3)oc3ccccc34)c3cc(-n4c5ccc(F)cc5c5ccc(-c6ccccc6)cc54)ccc3-c3ccccc3)ccc21] 278 | 68 | 787.3 |
| 37 | 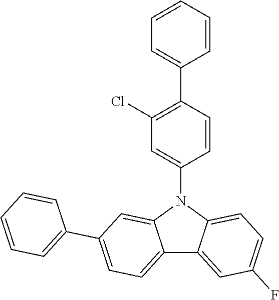 [SMILES: Fc1ccc2c(c1)c1ccc(-c3ccccc3)cc1n2-c1ccc(-c2ccccc2)c(Cl)c1] IM B21 | 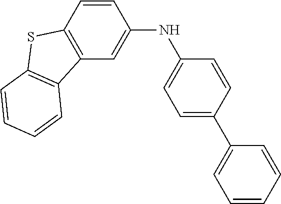 [SMILES: c1ccc(-c2ccc(Nc3ccc4sc5ccccc5c4c3)cc2)cc1] | 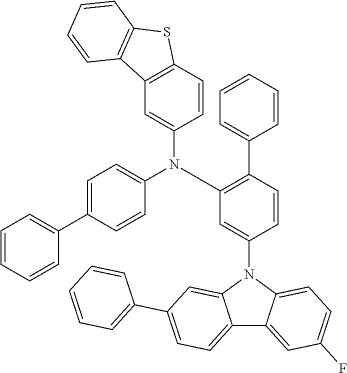 [SMILES: Fc1ccc2c(c1)c1ccc(-c3ccccc3)cc1n2-c1ccc(-c2ccccc2)c(N(c2ccc(-c3ccccc3)cc2)c2ccc3sc4ccccc4c3c2)c1] 284 | 62 | 763.3 |

NMR data of Compound 216: 1H-NMR (400 MHz, CD₂Cl₂) δ ppm: 8.22(d, 1H), 7.97(d, 1H), 7.93(d, 1H), 7.76-7.72(m, 2H), 7.62(d, 2H), 7.55-7.51(m, 4H), 7.50-7.46(m, 3H), 7.45-7.31(m, 8H), 7.23(d, 1H), 7.21(s, 1H), 7.10(s, 1H), 7.03(d, 1H), 6.98(d, 1H), 6.92(d, 1H), 6.82(s, 1H), 6.72(d, 2H).

### Fabrication and Evaluation of Organic Electroluminescent Device

### Example 1: Blue Organic Electroluminescent Device

An organic electroluminescent device was fabricated through the following process: a ITO/Ag/ITO substrate with thicknesses of 100 Å, 1100 Å, and 100 Å in sequence was cut to dimensions of 40 mm (length)×40 mm (width)×0.7 mm (thickness), and then fabricated, by a photoetching process, into an experimental substrate with a cathode lap region, an anode, and patterns of an insulation layer. Surface treatment was conducted using ultraviolet ozone and O₂: N₂ plasma to enhance the work function of the anode, and the substrate surface was cleaned using an organic solvent to remove impurities and oil stains on the substrate surface.

On the experimental substrate (the anode), F4-TCNQ was deposited by vacuum evaporation to form a hole injection layer (HIL) with a thickness of 120 Å, and HT-4 was deposited by vacuum evaporation on the hole injection layer to form a first hole transport layer with a thickness of 1150 Å.

On the first hole transport layer, Compound 1 was deposited by vacuum evaporation to form a second hole transport layer with a thickness of 150 Å.

On the second hole transport layer, BH-2 used as the host was co-deposited with simultaneously doped BD-3 at a film thickness ratio of 100:3, to form an organic light emitting layer (EML) with a thickness of 240 Å.

On the organic light emitting layer, ET-2 and LiQ were co-deposited at a film thickness ratio of 1:1 to form an electron transport layer (ETL) with a thickness of 280 Å. Subsequently, Yb was deposited on the electron transport layer to form an electron injection layer (EIL) with a thickness of 10 Å. Then, magnesium (Mg) and silver (Ag) were mixed at a vapor deposition rate of 1:9 and deposited by vacuum evaporation on the electron injection layer to form a cathode with a thickness of 115 Å.

Additionally, on the above cathode, CP-1 was deposited to form an organic capping layer (CPL) with a thickness of 680 Å, thus completing the fabrication of the organic electroluminescent device.

### Examples 2 to 37

Organic electroluminescent devices were fabricated using the same method as in Example 1, except that other compounds as indicated in Table 4 (column labeled “Second Hole Transport Layer”) were used instead of Compound 1 during the formation of the second hole transport layer.

### Comparative Examples 1 to 3

Organic electroluminescent devices were fabricated using the same method as in Example 1, except that Compound A, Compound B, and Compound C were used instead of Compound 1 during the formation of the second hole transport layer.

The main material structures used in the above examples and comparative examples are as follows:

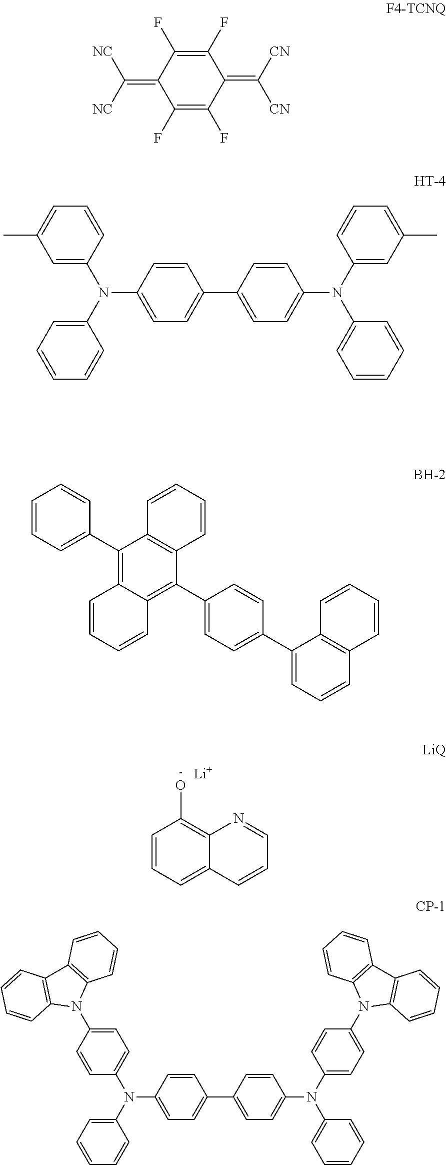

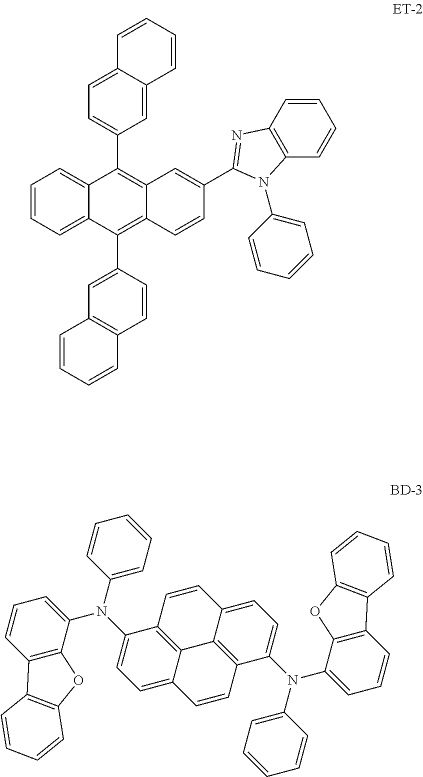 [SMILES: c1ccc(-n2c(-c3ccc4c(-c5ccc6ccccc6c5)c5ccccc5c(-c5ccc6ccccc6c5)c4c3)nc3ccccc32)cc1 ; c1ccc(N(c2ccc3ccc4c(N(c5ccccc5)c5cccc6c5oc5ccccc56)ccc5ccc2c3c54)c2cccc3c2oc2ccccc23)cc1]

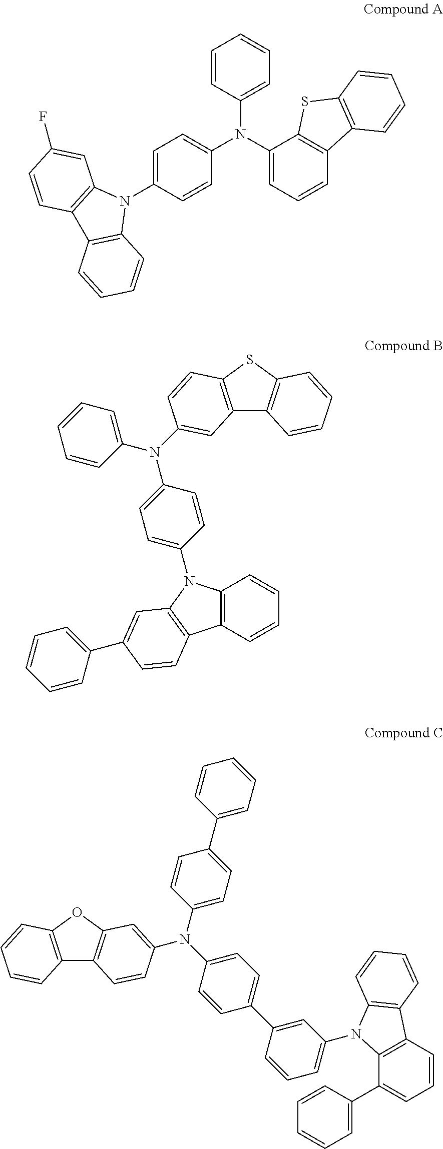 [SMILES: Fc1ccc2c3ccccc3n(-c3ccc(N(c4ccccc4)c4cccc5c4sc4ccccc45)cc3)c2c1 ; c1ccc(-c2ccc3c4ccccc4n(-c4ccc(N(c5ccccc5)c5ccc6sc7ccccc7c6c5)cc4)c3c2)cc1 ; c1ccc(-c2ccc(N(c3ccc(-c4cccc(-n5c6ccccc6c6cccc(-c7ccccc7)c65)c4)cc3)c3ccc4c(c3)oc3ccccc34)cc2)cc1]

The performances of the organic electroluminescent devices prepared in the above examples and comparative examples were analyzed. Specifically, the IVL performances (voltage, color coordinates, and efficiency) of the devices were tested at a current density of 10 mA/cm², and the T95 lifetime of the devices was tested at a current density of 20 mA/cm². The results are presented in Table 4.

TABLE 4

| | Second Hole | Operating | Current | | | |
|---|---|---|---|---|---|---|
| | Transport | Voltage | Efficiency | | | T95(hrs) |
| No. | Layer | (V) | (Cd/A) | CIE-x | CIE-y | @20 mA/cm² |
| Example 1 | Compound 1 | 3.84 | 6.31 | 0.135 | 0.047 | 176 |
| Example 2 | Compound 7 | 3.88 | 6.42 | 0.135 | 0.048 | 175 |
| Example 3 | Compound 12 | 3.90 | 6.35 | 0.135 | 0.049 | 172 |
| Example 4 | Compound 15 | 3.87 | 6.19 | 0.136 | 0.048 | 180 |
| Example 5 | Compound 20 | 3.86 | 6.15 | 0.134 | 0.047 | 178 |
| Example 6 | Compound 25 | 3.91 | 6.23 | 0.136 | 0.048 | 174 |
| Example 7 | Compound 41 | 3.85 | 6.41 | 0.135 | 0.047 | 176 |
| Example 8 | Compound | 3.99 | 6.22 | 0.133 | 0.049 | 160 |
| | 252 | | | | | |
| Example 9 | Compound | 4.03 | 6.32 | 0.136 | 0.050 | 155 |
| | 253 | | | | | |
| Example 10 | Compound | 3.97 | 6.19 | 0.134 | 0.051 | 158 |
| | 258 | | | | | |
| Example 11 | Compound | 3.98 | 6.11 | 0.133 | 0.048 | 157 |
| | 245 | | | | | |
| Example 12 | Compound | 3.96 | 6.35 | 0.136 | 0.047 | 156 |
| | 261 | | | | | |
| Example 13 | Compound 73 | 3.85 | 6.16 | 0.135 | 0.049 | 175 |
| Example 14 | Compound 81 | 3.88 | 6.22 | 0.132 | 0.050 | 181 |
| Example 15 | Compound 90 | 3.90 | 6.19 | 0.133 | 0.048 | 176 |
| Example 16 | Compound186 | 4.04 | 6.26 | 0.136 | 0.048 | 183 |
| Example 17 | Compound 99 | 4.02 | 6.14 | 0.137 | 0.050 | 174 |
| Example 18 | Compound | 4.06 | 6.18 | 0.135 | 0.050 | 158 |
| | 224 | | | | | |
| Example 19 | Compound | 3.94 | 6.24 | 0.133 | 0.049 | 162 |
| | 229 | | | | | |
| Example 20 | Compound | 3.86 | 6.40 | 0.136 | 0.048 | 177 |
| | 109 | | | | | |
| Example 21 | Compound | 3.87 | 6.24 | 0.136 | 0.049 | 179 |
| | 112 | | | | | |
| Example 22 | Compound | 3.89 | 6.29 | 0.133 | 0.047 | 174 |
| | 122 | | | | | |
| Example 23 | Compound | 3.95 | 6.15 | 0.135 | 0.050 | 160 |
| | 231 | | | | | |
| Example 24 | Compound | 3.88 | 6.26 | 0.134 | 0.047 | 182 |
| | 142 | | | | | |
| Example 25 | Compound | 3.85 | 6.38 | 0.135 | 0.047 | 179 |
| | 151 | | | | | |
| Example 26 | Compound | 3.94 | 6.25 | 0.136 | 0.050 | 156 |
| | 237 | | | | | |
| Example 27 | Compound | 3.89 | 6.41 | 0.133 | 0.050 | 158 |
| | 242 | | | | | |
| Example 28 | Compound | 3.94 | 6.23 | 0.135 | 0.049 | 161 |
| | 176 | | | | | |
| Example 29 | Compound | 3.97 | 6.21 | 0.135 | 0.049 | 156 |
| | 183 | | | | | |
| Example 30 | Compound | 4.03 | 6.34 | 0.135 | 0.050 | 178 |
| | 201 | | | | | |
| Example 31 | Compound | 4.05 | 6.18 | 0.134 | 0.047 | 182 |
| | 203 | | | | | |
| Example 32 | Compound | 3.89 | 6.37 | 0.136 | 0.048 | 161 |
| | 212 | | | | | |
| Example 33 | Compound | 4.03 | 6.42 | 0.135 | 0.050 | 163 |
| | 216 | | | | | |
| Example 34 | Compound | 3.93 | 6.28 | 0.136 | 0.049 | 159 |
| | 223 | | | | | |
| Example 35 | Compound | 3.87 | 6.19 | 0.135 | 0.047 | 184 |
| | 270 | | | | | |
| Example 36 | Compound | 3.91 | 6.43 | 0.134 | 0.050 | 179 |
| | 278 | | | | | |
| Example 37 | Compound | 3.86 | 6.37 | 0.135 | 0.048 | 182 |
| | 284 | | | | | |
| Comparative | Compound A | 4.02 | 5.24 | 0.136 | 0.047 | 129 |
| Example 1 | | | | | | |
| Comparative | Compound B | 4.14 | 5.43 | 0.135 | 0.050 | 133 |
| Example 2 | | | | | | |
| Comparative | Compound C | 4.08 | 5.36 | 0.134 | 0.047 | 140 |
| Example 3 | | | | | | |

In conjunction with the results presented in Table 4, it is observed that when Examples 1 to 37 are compared to Comparative Examples 1 to 3, the organic electroluminescent devices, in which the compounds of the present disclosure are used as the second hole transport layer (i.e., the electron blocking layer) have exhibited an improvement in current efficiency of at least 12.5% and a T95 lifetime increase of at least 10.7% while maintaining a lower operating voltage. It can be seen that, the incorporation of the compounds of the present disclosure as the second hole transport layer in an organic electroluminescent device, while maintaining a lower operating voltage, can further improve the luminous efficiency and service life of the devices.

The preferred embodiments of the present disclosure are described in detail above in conjunction with the accompanying drawings. However, the present disclosure is not limited to the specific details of the above embodiments. Within the scope of the technical concept of the present disclosure, various simple modifications can be made to the technical solutions of the present disclosure, and all these simple modifications fall within the protection scope of the present disclosure. In addition, various embodiments of the present disclosure can also be combined arbitrarily, as long as these do not violate the concept of the present disclosure, these should also be regarded as the content disclosed in the present disclosure.

1. An organic compound, having a structure shown in Formula 1: 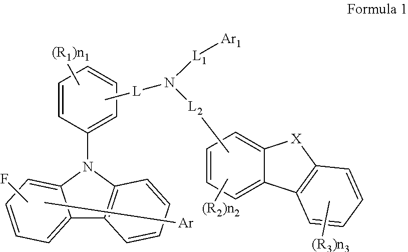
wherein X is selected from O and S;
Ar is selected from a substituted or unsubstituted aryl having 6 to 15 carbon atoms, and substituent(s) in Ar are each independently selected from a deuterium, a cyano, and an alkyl having 1 to 4 carbon atoms;
L, L₁, and L₂ are the same or different, and are each independently selected from a single bond, a substituted or unsubstituted arylene having 6 to 30 carbon atoms, and a substituted or unsubstituted heteroarylene having 5 to 30 carbon atoms;
Ar₁ is selected from a substituted or unsubstituted aryl having 6 to 40 carbon atoms, and a substituted or unsubstituted heteroaryl having 5 to 40 carbon atoms;
substituent(s) in L, L₁, L₂, and Ar₁, as well as R₁, R₂, and R₃, are the same or different, and are each independently selected from a deuterium, a cyano, an aryl having 6 to 18 carbon atoms, a heteroaryl having 5 to 18 carbon atoms, a trialkylsilyl having 3 to 12 carbon atoms, an alkyl having 1 to 10 carbon atoms, a deuteroalkyl having 1 to 10 carbon atoms, a cycloalkyl having 3 to 10 carbon atoms, and a triphenylsilyl; optionally, any two adjacent substituents in Ar₁ form a 3-to 15-membered saturated or unsaturated ring;
n₁ represents the number of R₁, and is selected from 0, 1, 2, 3, and 4; when n₁ is greater than 1, each R₁ is the same or different;
n₂ represents the number of R₂, and is selected from 0, 1, 2, and 3; when n₂ is greater than 1, each R₂ is the same or different;
n₃ represents the number of R₃, and is selected from 0, 1, 2, 3, and 4; when n₃ is greater than 1, each R₃ is the same or different.

2. The organic compound according to claim 1, wherein Ar is selected from a substituted or unsubstituted phenyl, a substituted or unsubstituted naphthyl, and a substituted or unsubstituted biphenyl, and substituent(s) in Ar are each independently selected from a deuterium, a cyano, a methyl, an ethyl, an isopropyl, and a tert-butyl.

3. The organic compound according to claim 1, wherein Ar is selected from the group consisting of the following groups:

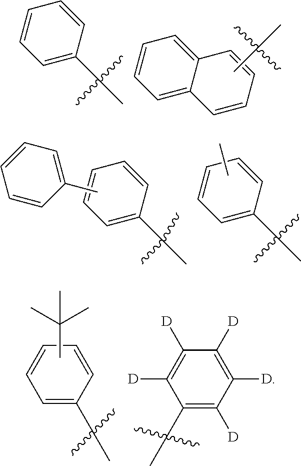

4. The organic compound according to claim 1, wherein L, L₁, and L₂ are the same or different, and are each independently selected from a single bond, a substituted or unsubstituted arylene having 6 to 18 carbon atoms, and a substituted or unsubstituted heteroarylene having 12 to 18 carbon atoms;
optionally, substituent(s) in L, L₁ and L₂ are each independently selected from a deuterium, a cyano, an aryl having 6 to 12 carbon atoms, a heteroaryl having 5 to 12 carbon atoms, a trialkylsilyl having 3 to 7 carbon atoms, and an alkyl having 1 to 4 carbon atoms.

5. The organic compound according to claim 1, wherein L, L₁ and L₂ are the same or different, and each independently selected from a single bond, a substituted or unsubstituted group Z, and the unsubstituted group Z is selected from the group consisting of the following groups: 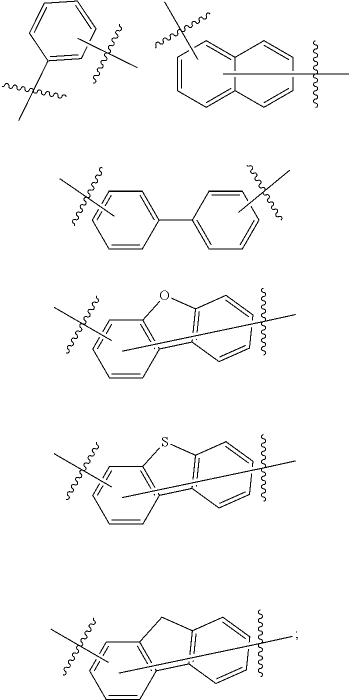
substituent(s) in the substituent group Z are each independently selected from a deuterium, a cyano, a methyl, an ethyl, an isopropyl, a tert-butyl, a trimethylsilyl, a phenyl, and a naphthyl, when the number of the substituents is greater than 1, each substituent is the same or different.

6. The organic compound according to claim 1, wherein L and L₂ are each independently selected from a single bond and the group consisting of the following groups: 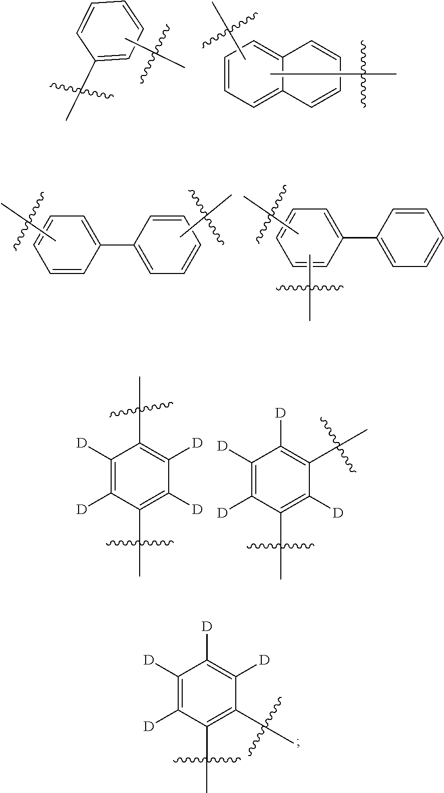
optionally, L₁ is selected from a single bond, and the group consisting of the following groups:

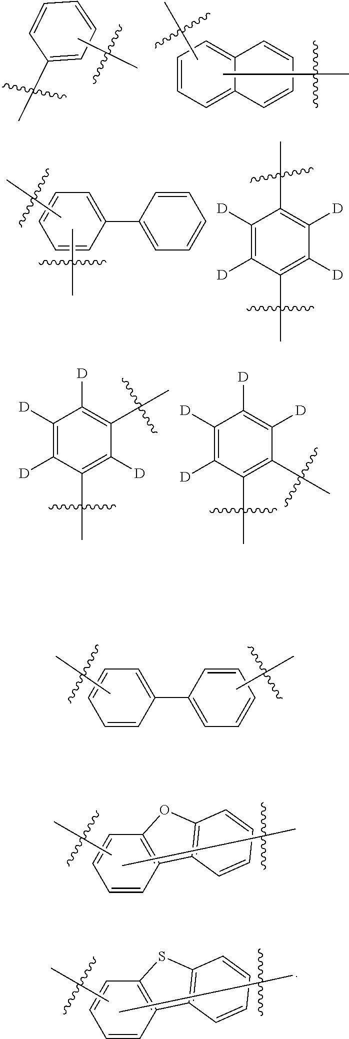

7. The organic compound according to claim 1, wherein Ar₁ is selected from a substituted or unsubstituted aryl having 6 to 25 carbon atoms, and a substituted or unsubstituted heteroaryl having 12 to 25 carbon atoms;
optionally, substituent(s) in Ar₁ are each independently selected from a deuterium, a cyano, an aryl having 6 to 12 carbon atoms, a heteroaryl having 5 to 12 carbon atoms, a trialkylsilyl having 3 to 7 carbon atoms, an alkyl having 1 to 4 carbon atoms, a deuteroalkyl having 1 to 4 carbon atoms, and a cycloalkyl having 5 to 10 carbon atoms; optionally, any two adjacent substituents in Ar₁ form a saturated or unsaturated 5- to 15-membered ring.

8. The organic compound according to claim 1, wherein Ar₁ is selected from a substituted or unsubstituted group W, and the unsubstituted group W is selected from the group consisting of the following groups: 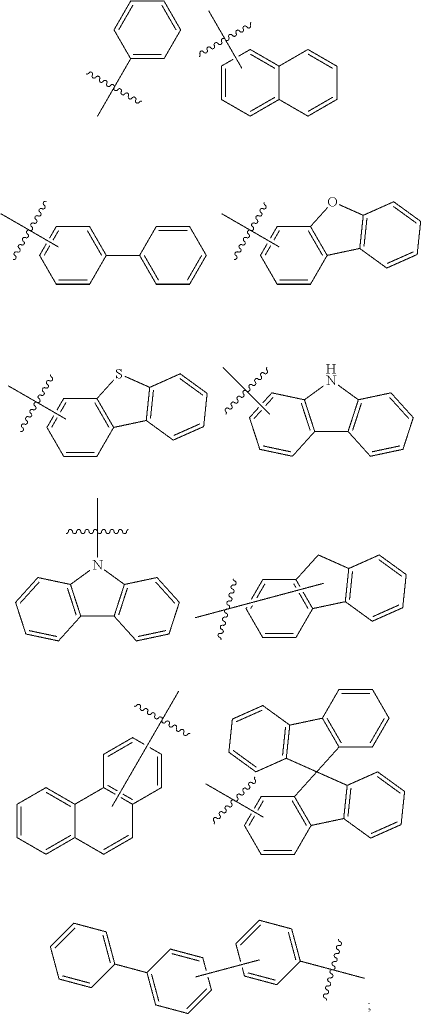
substituent(s) in the substituted group W are each independently selected from a deuterium, a cyano, a methyl, an ethyl, an isopropyl, a tert-butyl, a trideuteromethyl, a trimethylsilyl, a phenyl, and a naphthyl; and when the number of substituents is greater than 1, each substituent is the same or different.

9. The organic compound according to claim 1, wherein Ar₁ is selected from the group consisting of the following groups:

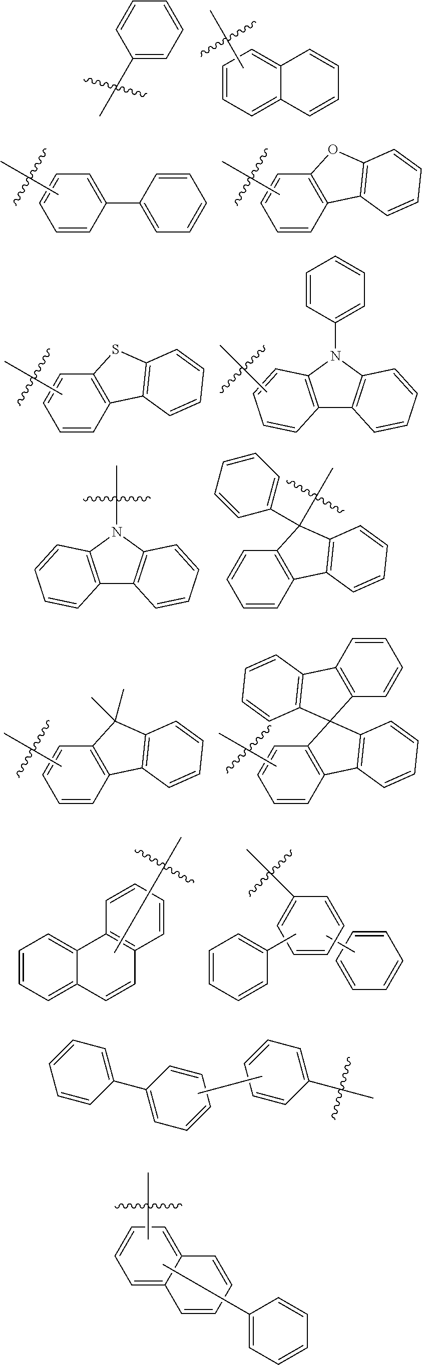

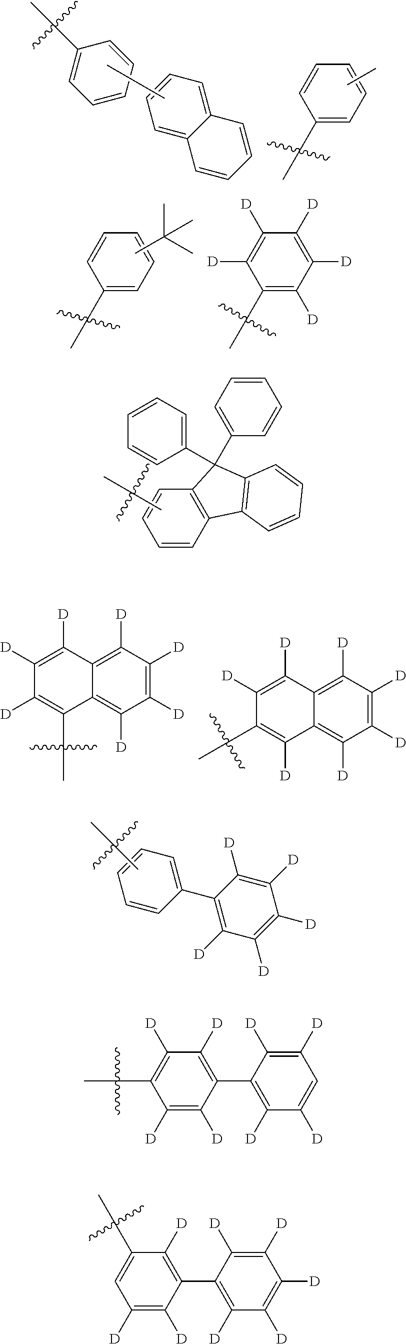

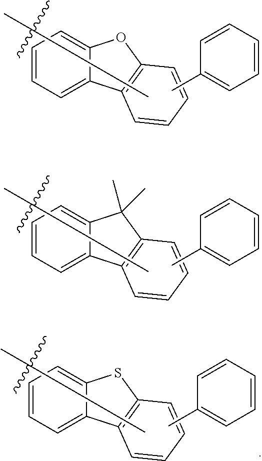

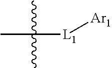

10. The organic compound according to claim 1, wherein is selected from the group consisting of the following groups:

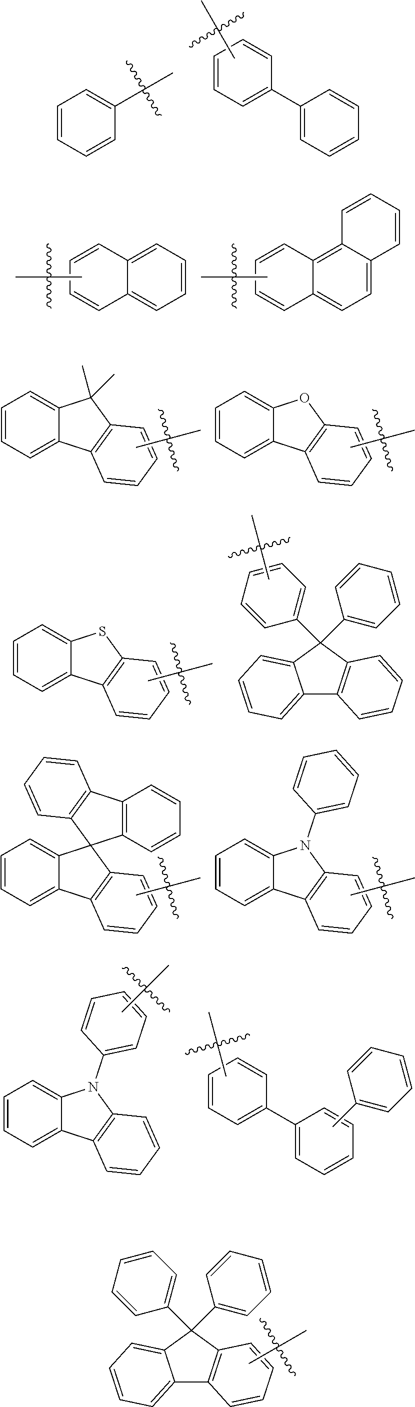

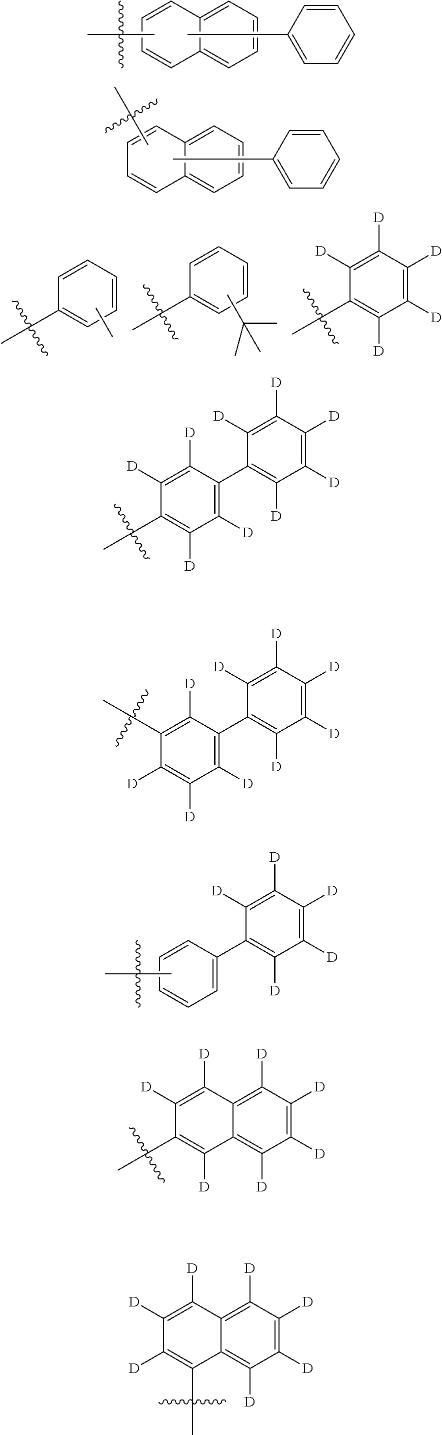

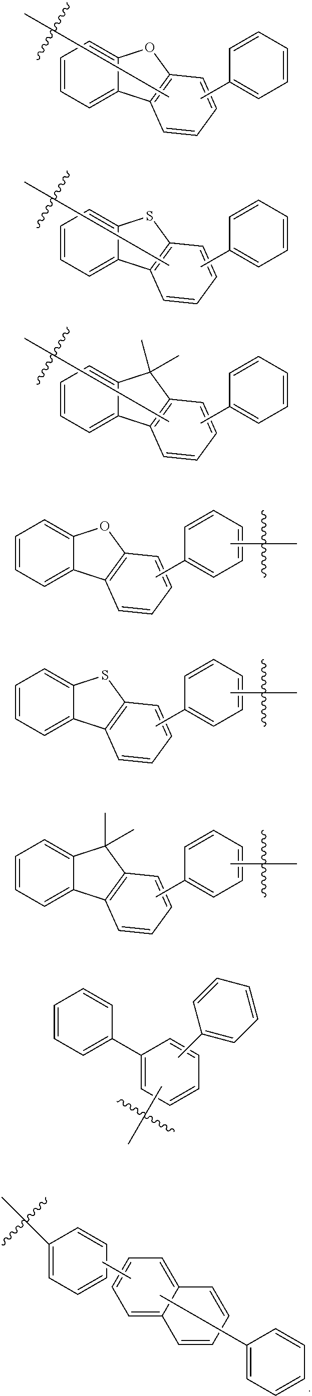

11. The organic compound according to claim 1, wherein, R₁, R₂ and R₃ are the same or different and are each independently selected from a deuterium, a cyano, a methyl, an ethyl, an isopropyl, a tert-butyl, a trideuteromethyl, a phenyl, and a naphthyl.

12. The organic compound according to claim 1, wherein the organic compound is selected from the group consisting of the following compounds:

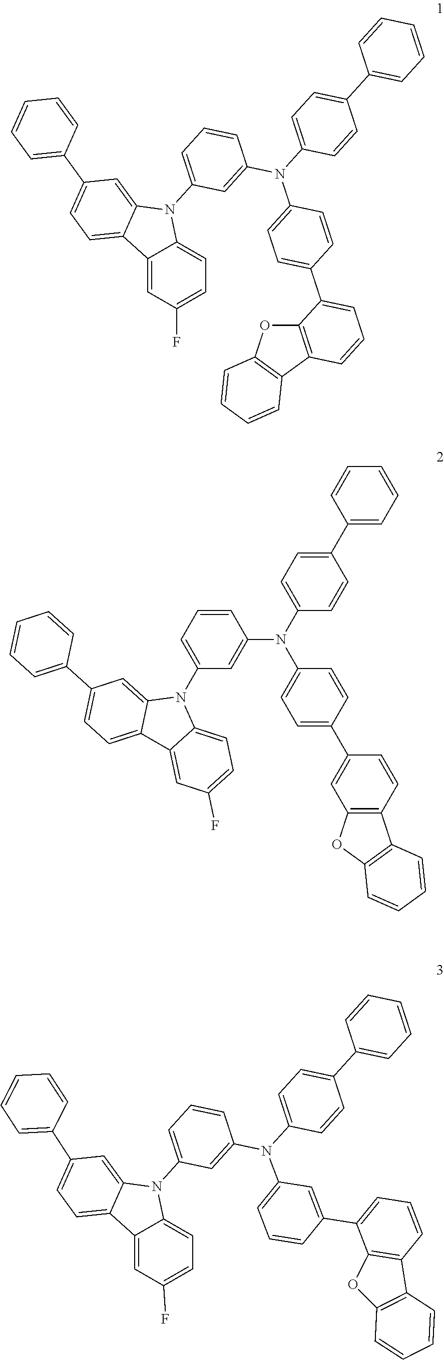 [SMILES: Fc1ccc2c(c1)c1ccc(-c3ccccc3)cc1n2-c1cccc(N(c2ccc(-c3ccccc3)cc2)c2ccc(-c3cccc4c3oc3ccccc34)cc2)c1 ; Fc1ccc2c(c1)c1ccc(-c3ccccc3)cc1n2-c1cccc(N(c2ccc(-c3ccccc3)cc2)c2ccc(-c3ccc4c(c3)oc3ccccc34)cc2)c1 ; Fc1ccc2c(c1)c1ccc(-c3ccccc3)cc1n2-c1cccc(N(c2ccc(-c3ccccc3)cc2)c2cccc(-c3cccc4c3oc3ccccc34)c2)c1]

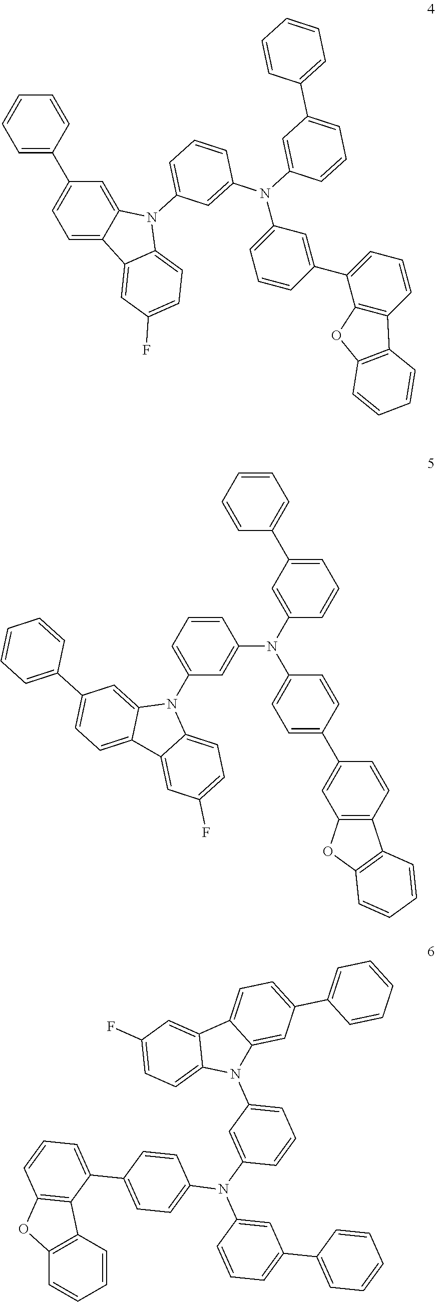 [SMILES: Fc1ccc2c(c1)c1ccc(-c3ccccc3)cc1n2-c1cccc(N(c2cccc(-c3ccccc3)c2)c2cccc(-c3cccc4c3oc3ccccc34)c2)c1 ; Fc1ccc2c(c1)c1ccc(-c3ccccc3)cc1n2-c1cccc(N(c2ccc(-c3ccc4c(c3)oc3ccccc34)cc2)c2cccc(-c3ccccc3)c2)c1 ; Fc1ccc2c(c1)c1ccc(-c3ccccc3)cc1n2-c1cccc(N(c2ccc(-c3cccc4oc5ccccc5c34)cc2)c2cccc(-c3ccccc3)c2)c1]

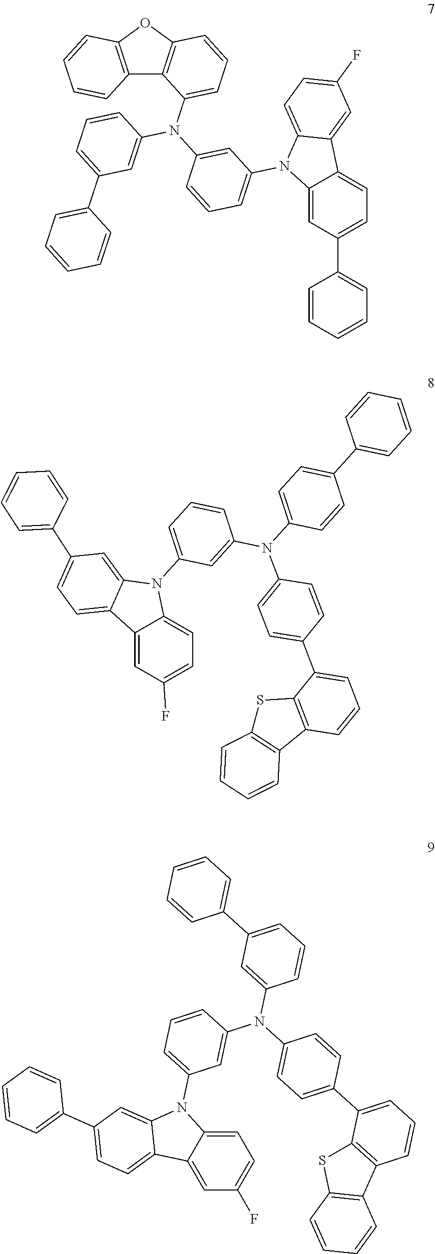 [SMILES: Fc1ccc2c(c1)c1ccc(-c3ccccc3)cc1n2-c1cccc(N(c2cccc(-c3ccccc3)c2)c2cccc3oc4ccccc4c23)c1 ; Fc1ccc2c(c1)c1ccc(-c3ccccc3)cc1n2-c1cccc(N(c2ccc(-c3ccccc3)cc2)c2ccc(-c3cccc4c3sc3ccccc34)cc2)c1 ; Fc1ccc2c(c1)c1ccc(-c3ccccc3)cc1n2-c1cccc(N(c2ccc(-c3cccc4c3sc3ccccc34)cc2)c2cccc(-c3ccccc3)c2)c1]

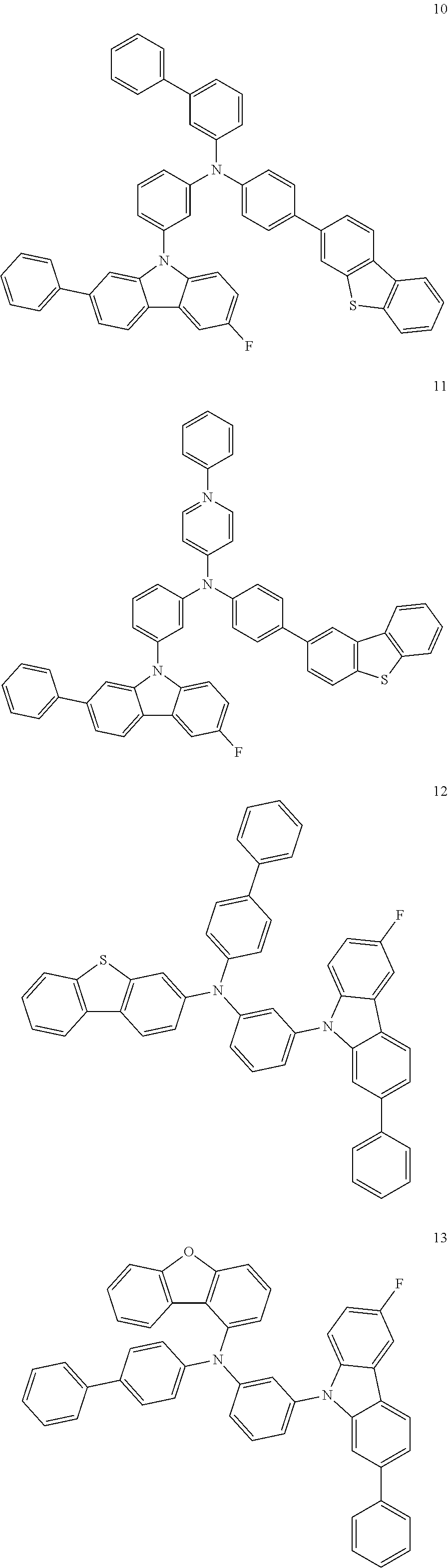

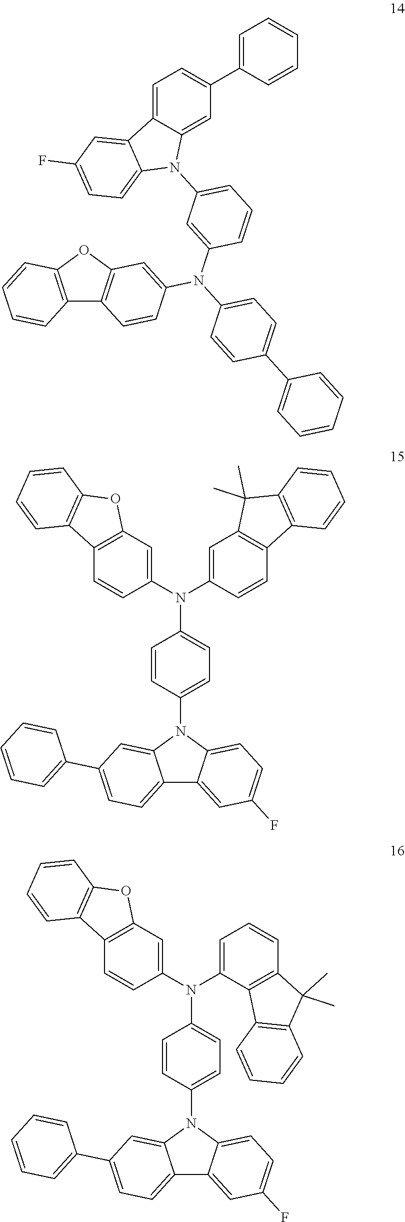

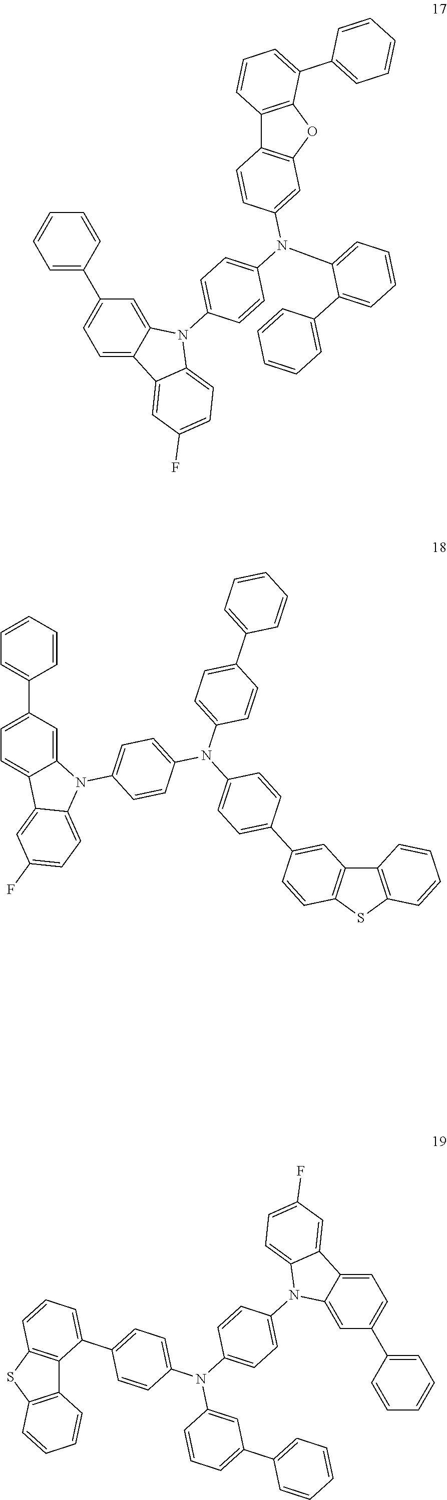 [SMILES: Fc1ccc2c(c1)c1ccc(-c3ccccc3)cc1n2-c1ccc(N(c2ccc3c(c2)oc2c(-c4ccccc4)cccc23)c2ccccc2-c2ccccc2)cc1 ; Fc1ccc2c(c1)c1ccc(-c3ccccc3)cc1n2-c1ccc(N(c2ccc(-c3ccccc3)cc2)c2ccc(-c3ccc4sc5ccccc5c4c3)cc2)cc1 ; Fc1ccc2c(c1)c1ccc(-c3ccccc3)cc1n2-c1ccc(N(c2ccc(-c3cccc4sc5ccccc5c34)cc2)c2cccc(-c3ccccc3)c2)cc1]

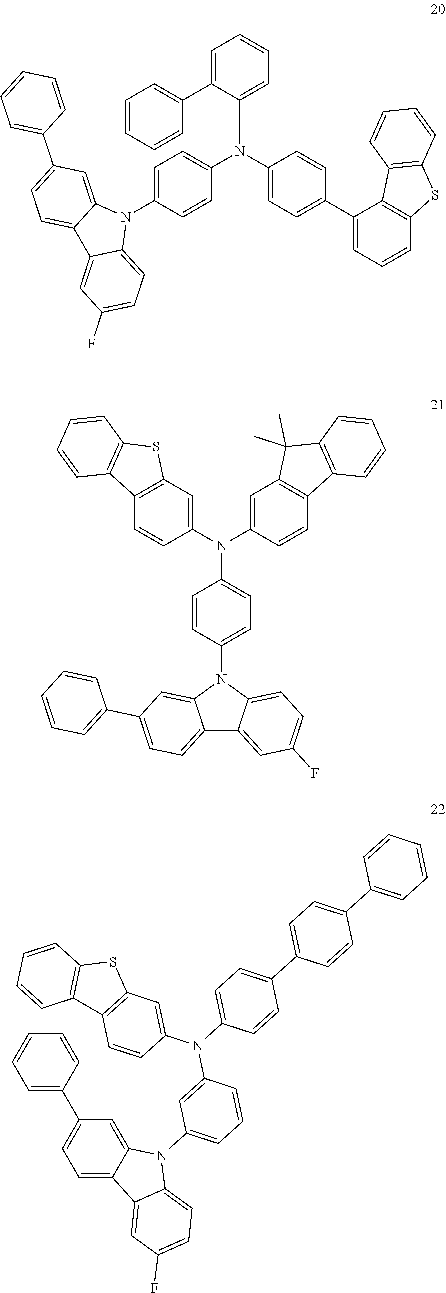 [SMILES: Fc1ccc2c(c1)c1ccc(-c3ccccc3)cc1n2-c1ccc(N(c2ccc(-c3cccc4sc5ccccc5c34)cc2)c2ccccc2-c2ccccc2)cc1 ; CC1(C)c2ccccc2-c2ccc(N(c3ccc(-n4c5ccc(F)cc5c5ccc(-c6ccccc6)cc54)cc3)c3ccc4c(c3)sc3ccccc34)cc21 ; Fc1ccc2c(c1)c1ccc(-c3ccccc3)cc1n2-c1cccc(N(c2ccc(-c3ccc(-c4ccccc4)cc3)cc2)c2ccc3c(c2)sc2ccccc23)c1]

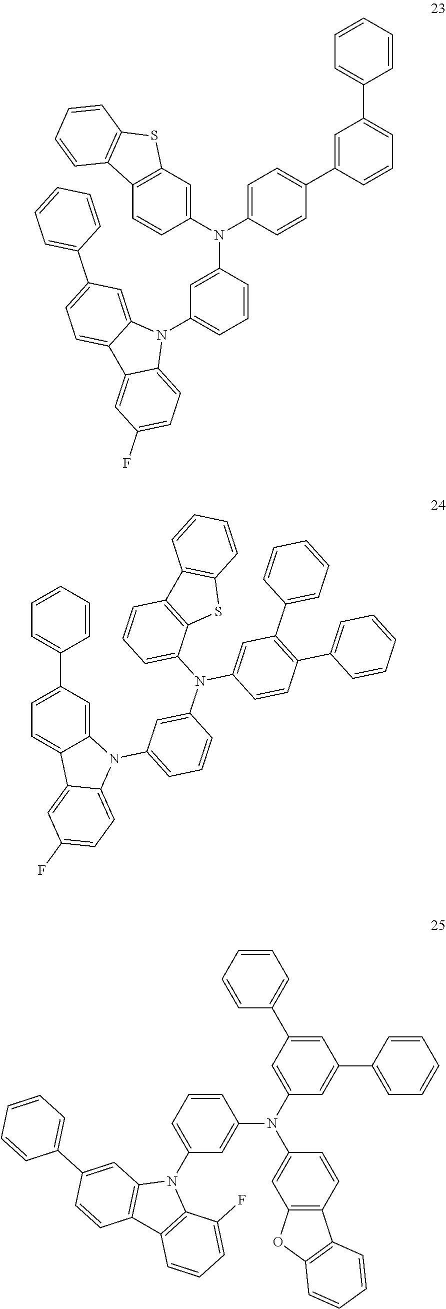 [SMILES: Fc1ccc2c(c1)c1ccc(-c3ccccc3)cc1n2-c1cccc(N(c2ccc(-c3cccc(-c4ccccc4)c3)cc2)c2ccc3c(c2)sc2ccccc23)c1 ; Fc1ccc2c(c1)c1ccc(-c3ccccc3)cc1n2-c1cccc(N(c2ccc(-c3ccccc3)c(-c3ccccc3)c2)c2cccc3c2sc2ccccc23)c1 ; Fc1cccc2c3ccc(-c4ccccc4)cc3n(-c3cccc(N(c4cc(-c5ccccc5)cc(-c5ccccc5)c4)c4ccc5c(c4)oc4ccccc45)c3)c12]

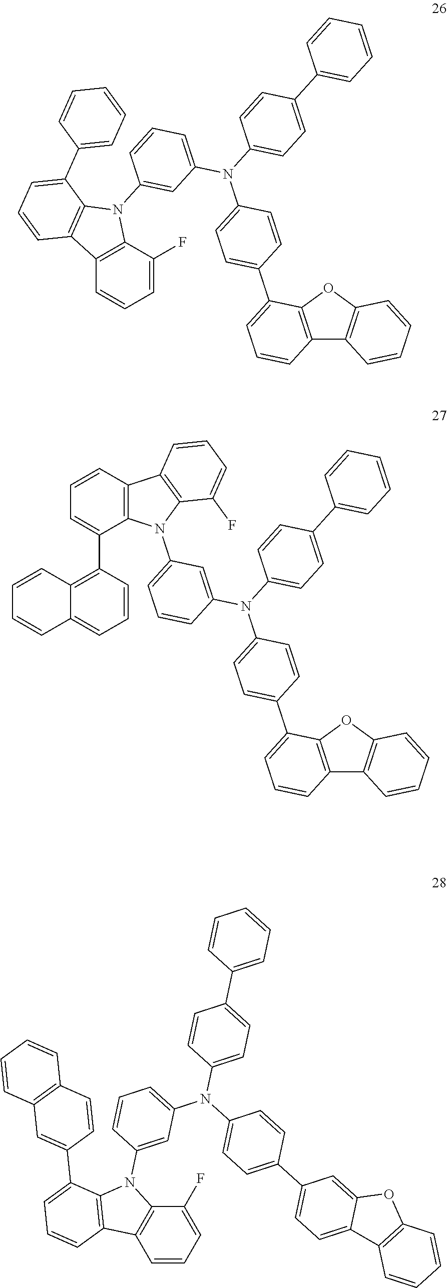 [SMILES: Fc1cccc2c3cccc(-c4ccccc4)c3n(-c3cccc(N(c4ccc(-c5ccccc5)cc4)c4ccc(-c5cccc6c5oc5ccccc56)cc4)c3)c12 ; Fc1cccc2c3cccc(-c4cccc5ccccc45)c3n(-c3cccc(N(c4ccc(-c5ccccc5)cc4)c4ccc(-c5cccc6c5oc5ccccc56)cc4)c3)c12 ; Fc1cccc2c3cccc(-c4ccc5ccccc5c4)c3n(-c3cccc(N(c4ccc(-c5ccccc5)cc4)c4ccc(-c5ccc6c(c5)oc5ccccc56)cc4)c3)c12]

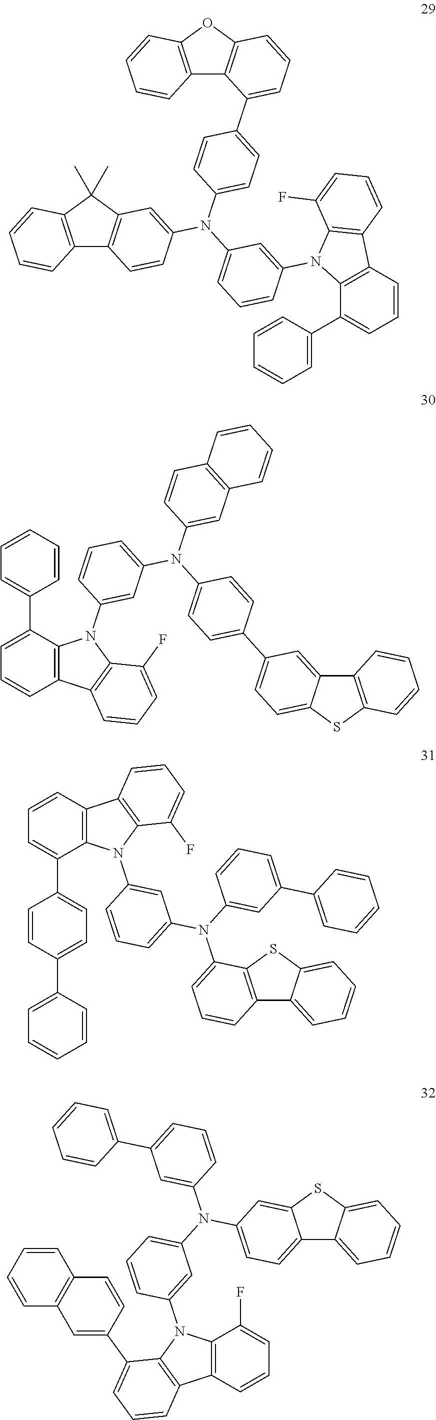

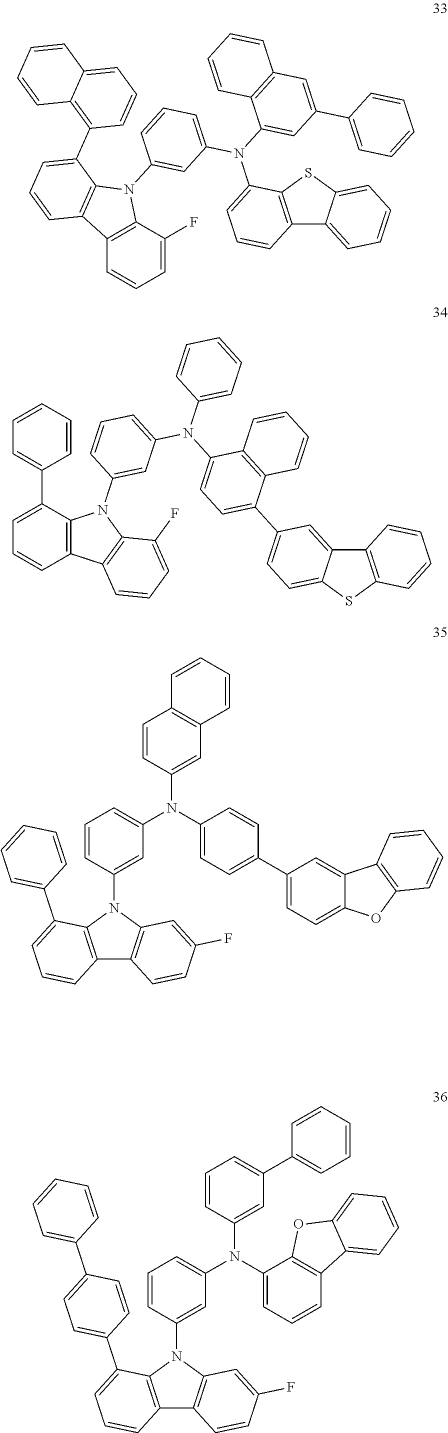 [SMILES: Fc1cccc2c3cccc(-c4cccc5ccccc45)c3n(-c3cccc(N(c4cc(-c5ccccc5)cc5ccccc45)c4cccc5c4sc4ccccc45)c3)c12 ; Fc1cccc2c3cccc(-c4ccccc4)c3n(-c3cccc(N(c4ccccc4)c4ccc(-c5ccc6sc7ccccc7c6c5)c5ccccc45)c3)c12 ; Fc1ccc2c3cccc(-c4ccccc4)c3n(-c3cccc(N(c4ccc(-c5ccc6oc7ccccc7c6c5)cc4)c4ccc5ccccc5c4)c3)c2c1 ; Fc1ccc2c3cccc(-c4ccc(-c5ccccc5)cc4)c3n(-c3cccc(N(c4cccc(-c5ccccc5)c4)c4cccc5c4oc4ccccc45)c3)c2c1]

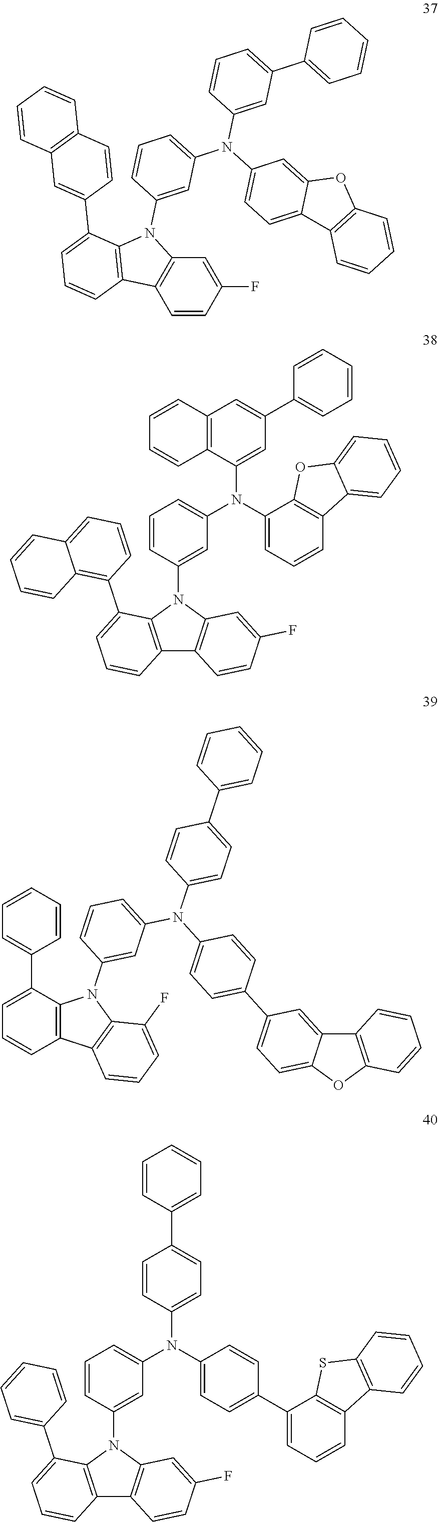 [SMILES: Fc1ccc2c3cccc(-c4ccc5ccccc5c4)c3n(-c3cccc(N(c4cccc(-c5ccccc5)c4)c4ccc5c(c4)oc4ccccc45)c3)c2c1 ; Fc1ccc2c3cccc(-c4cccc5ccccc45)c3n(-c3cccc(N(c4cc(-c5ccccc5)cc5ccccc45)c4cccc5c4oc4ccccc45)c3)c2c1 ; Fc1cccc2c3cccc(-c4ccccc4)c3n(-c3cccc(N(c4ccc(-c5ccccc5)cc4)c4ccc(-c5ccc6oc7ccccc7c6c5)cc4)c3)c12 ; Fc1ccc2c3cccc(-c4ccccc4)c3n(-c3cccc(N(c4ccc(-c5ccccc5)cc4)c4ccc(-c5cccc6c5sc5ccccc56)cc4)c3)c2c1]

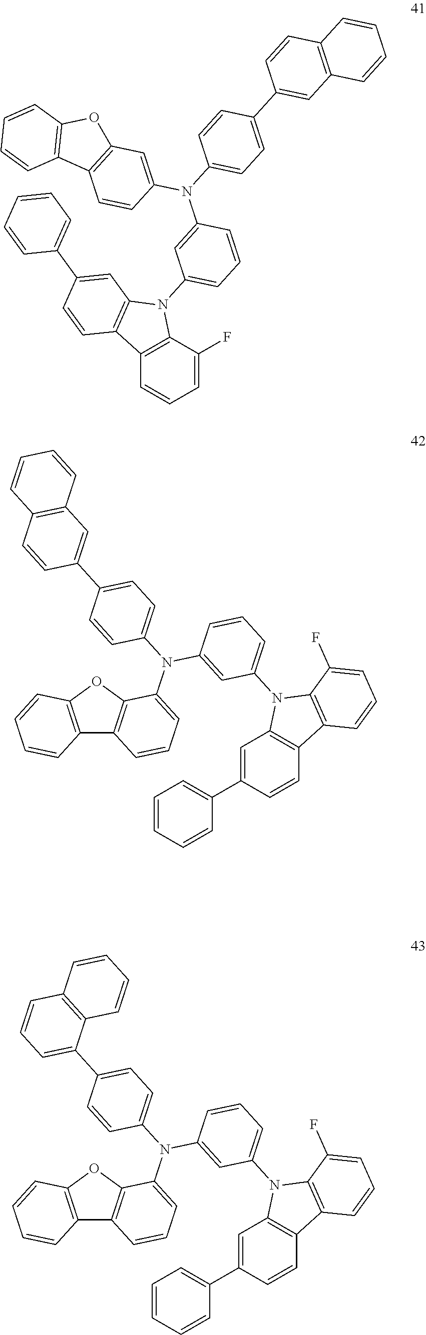 [SMILES: Fc1cccc2c3ccc(-c4ccccc4)cc3n(-c3cccc(N(c4ccc(-c5ccc6ccccc6c5)cc4)c4ccc5c(c4)oc4ccccc45)c3)c12 ; Fc1cccc2c3ccc(-c4ccccc4)cc3n(-c3cccc(N(c4ccc(-c5ccc6ccccc6c5)cc4)c4cccc5c4oc4ccccc45)c3)c12 ; Fc1cccc2c3ccc(-c4ccccc4)cc3n(-c3cccc(N(c4ccc(-c5cccc6ccccc56)cc4)c4cccc5c4oc4ccccc45)c3)c12]

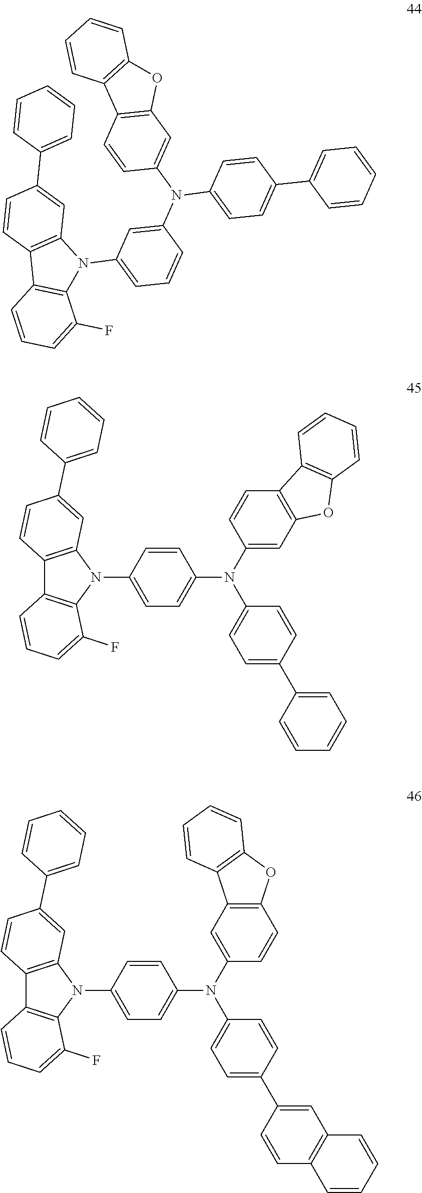 [SMILES: Fc1cccc2c3ccc(-c4ccccc4)cc3n(-c3cccc(N(c4ccc(-c5ccccc5)cc4)c4ccc5c(c4)oc4ccccc45)c3)c12 ; Fc1cccc2c3ccc(-c4ccccc4)cc3n(-c3ccc(N(c4ccc(-c5ccccc5)cc4)c4ccc5c(c4)oc4ccccc45)cc3)c12 ; Fc1cccc2c3ccc(-c4ccccc4)cc3n(-c3ccc(N(c4ccc(-c5ccc6ccccc6c5)cc4)c4ccc5oc6ccccc6c5c4)cc3)c12]

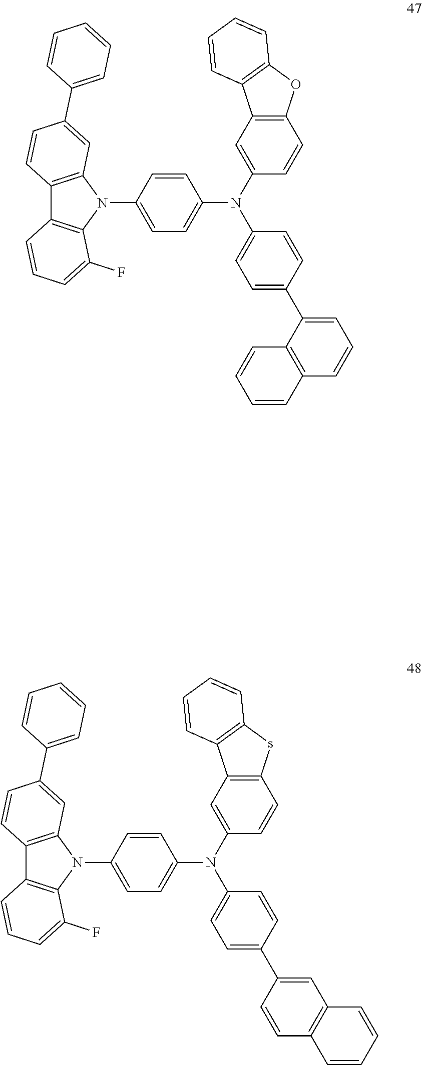

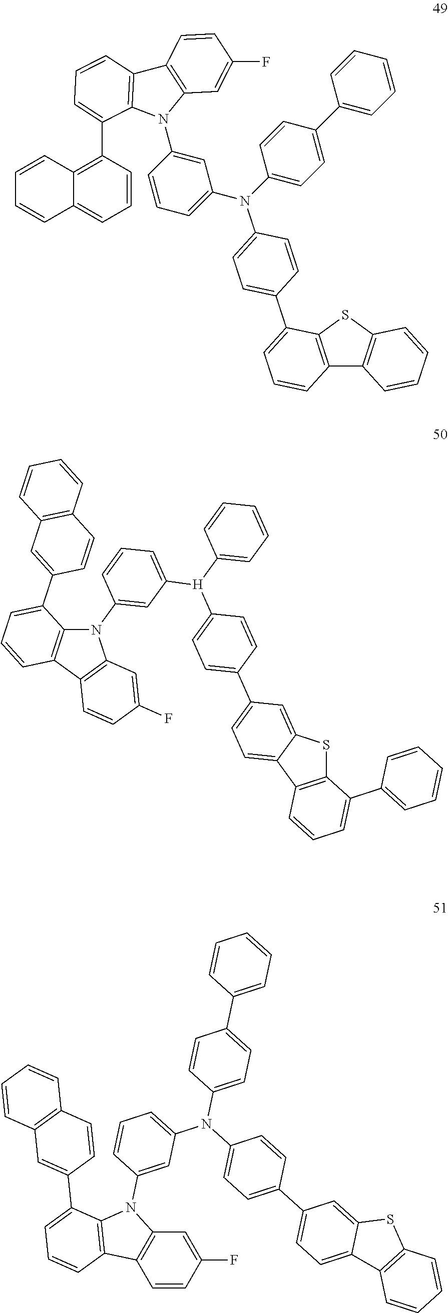

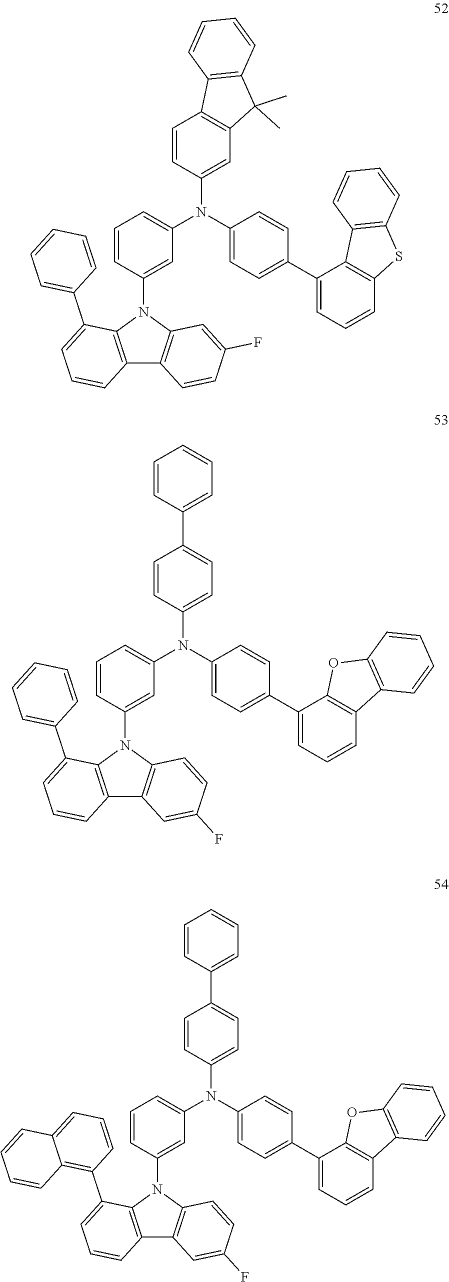 [SMILES: CC1(C)c2ccccc2-c2ccc(N(c3ccc(-c4cccc5sc6ccccc6c45)cc3)c3cccc(-n4c5cc(F)ccc5c5cccc(-c6ccccc6)c54)c3)cc21 ; Fc1ccc2c(c1)c1cccc(-c3ccccc3)c1n2-c1cccc(N(c2ccc(-c3ccccc3)cc2)c2ccc(-c3cccc4c3oc3ccccc34)cc2)c1 ; Fc1ccc2c(c1)c1cccc(-c3cccc4ccccc34)c1n2-c1cccc(N(c2ccc(-c3ccccc3)cc2)c2ccc(-c3cccc4c3oc3ccccc34)cc2)c1]

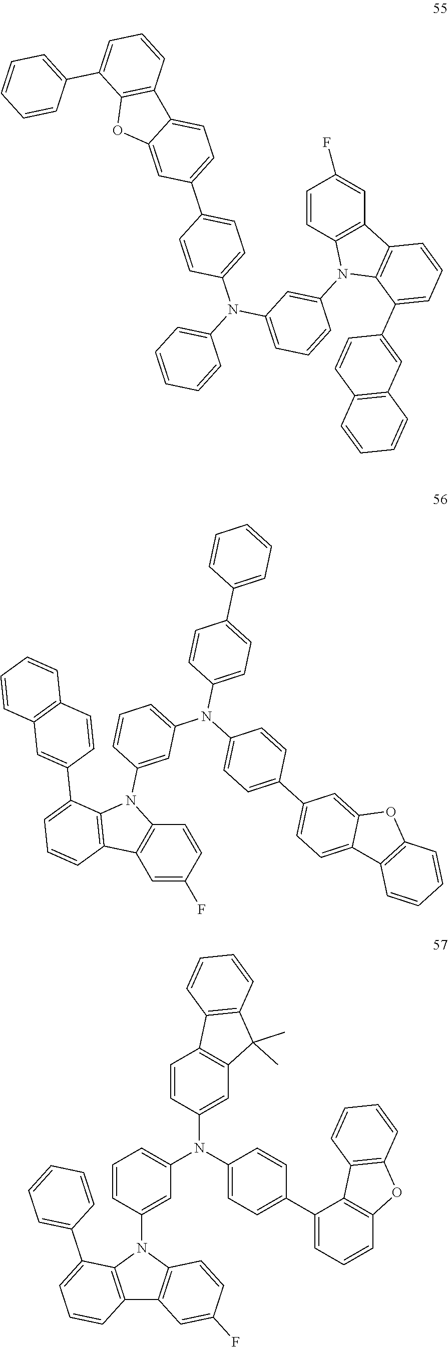 [SMILES: Fc1ccc2c(c1)c1cccc(-c3ccc4ccccc4c3)c1n2-c1cccc(N(c2ccccc2)c2ccc(-c3ccc4c(c3)oc3c(-c5ccccc5)cccc34)cc2)c1 ; Fc1ccc2c(c1)c1cccc(-c3ccc4ccccc4c3)c1n2-c1cccc(N(c2ccc(-c3ccccc3)cc2)c2ccc(-c3ccc4c(c3)oc3ccccc34)cc2)c1 ; CC1(C)c2ccccc2-c2ccc(N(c3ccc(-c4cccc5oc6ccccc6c45)cc3)c3cccc(-n4c5ccc(F)cc5c5cccc(-c6ccccc6)c54)c3)cc21]

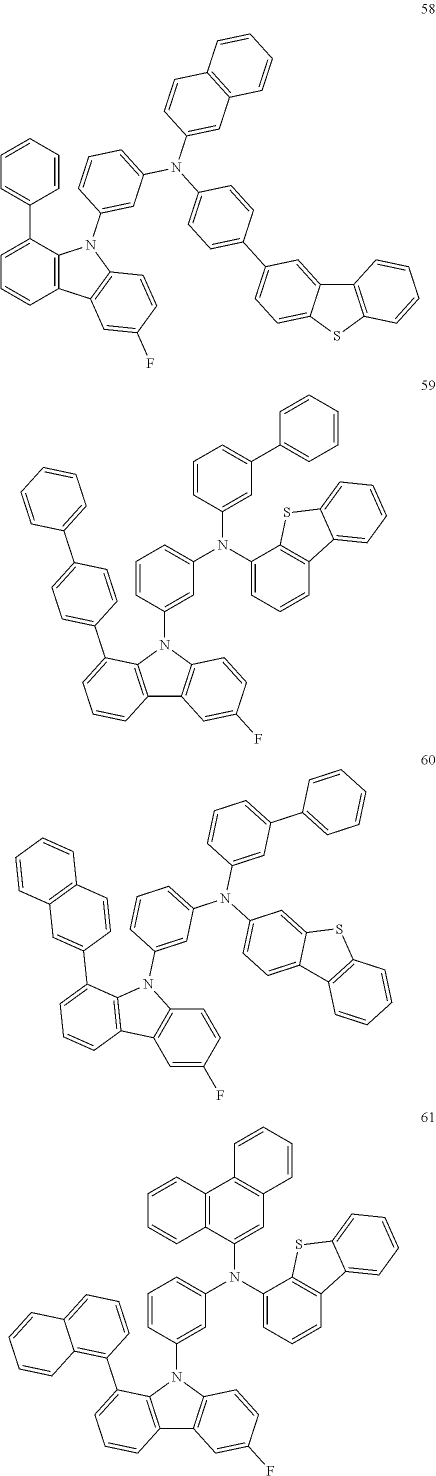 [SMILES: Fc1ccc2c(c1)c1cccc(-c3ccccc3)c1n2-c1cccc(N(c2ccc(-c3ccc4sc5ccccc5c4c3)cc2)c2ccc3ccccc3c2)c1 ; Fc1ccc2c(c1)c1cccc(-c3ccc(-c4ccccc4)cc3)c1n2-c1cccc(N(c2cccc(-c3ccccc3)c2)c2cccc3c2sc2ccccc23)c1 ; Fc1ccc2c(c1)c1cccc(-c3ccc4ccccc4c3)c1n2-c1cccc(N(c2cccc(-c3ccccc3)c2)c2ccc3c(c2)sc2ccccc23)c1 ; Fc1ccc2c(c1)c1cccc(-c3cccc4ccccc34)c1n2-c1cccc(N(c2cc3ccccc3c3ccccc23)c2cccc3c2sc2ccccc23)c1]

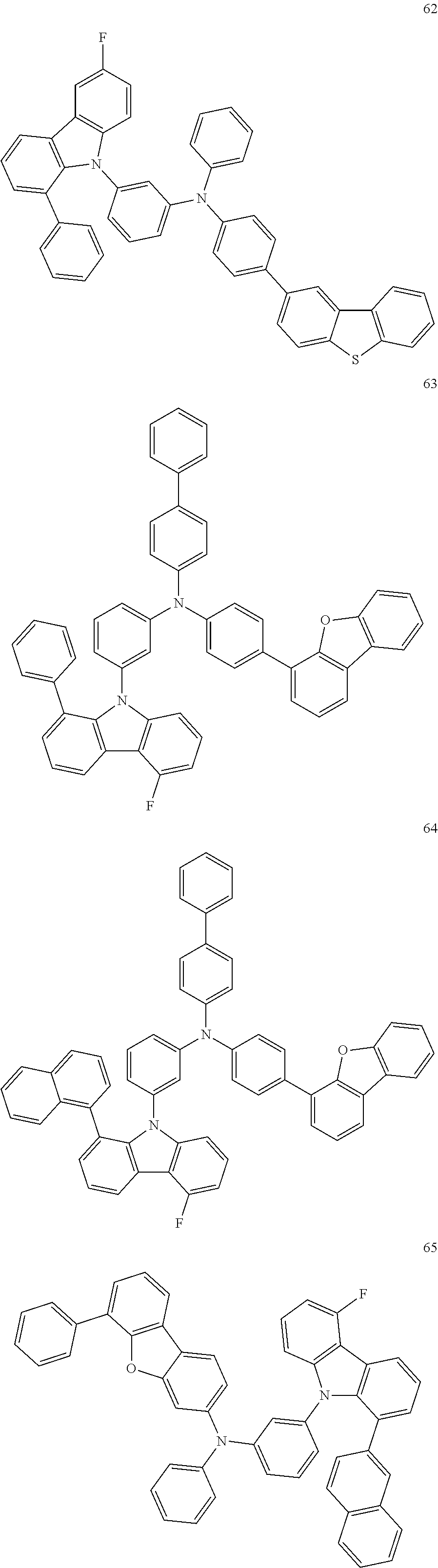 [SMILES: Fc1ccc2c(c1)c1cccc(-c3ccccc3)c1n2-c1cccc(N(c2ccccc2)c2ccc(-c3ccc4sc5ccccc5c4c3)cc2)c1 ; Fc1cccc2c1c1cccc(-c3ccccc3)c1n2-c1cccc(N(c2ccc(-c3ccccc3)cc2)c2ccc(-c3cccc4c3oc3ccccc34)cc2)c1 ; Fc1cccc2c1c1cccc(-c3cccc4ccccc34)c1n2-c1cccc(N(c2ccc(-c3ccccc3)cc2)c2ccc(-c3cccc4c3oc3ccccc34)cc2)c1 ; Fc1cccc2c1c1cccc(-c3ccc4ccccc4c3)c1n2-c1cccc(N(c2ccccc2)c2ccc3c(c2)oc2c(-c4ccccc4)cccc23)c1]

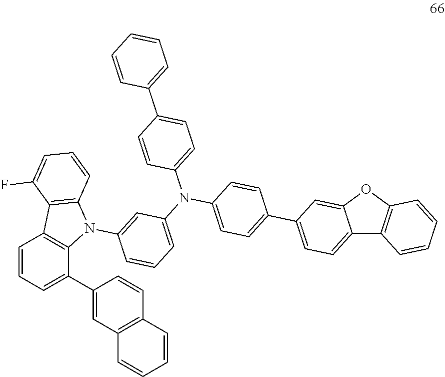 [SMILES: Fc1cccc2c1c1cccc(-c3ccc4ccccc4c3)c1n2-c1cccc(N(c2ccc(-c3ccccc3)cc2)c2ccc(-c3ccc4c(c3)oc3ccccc34)cc2)c1]

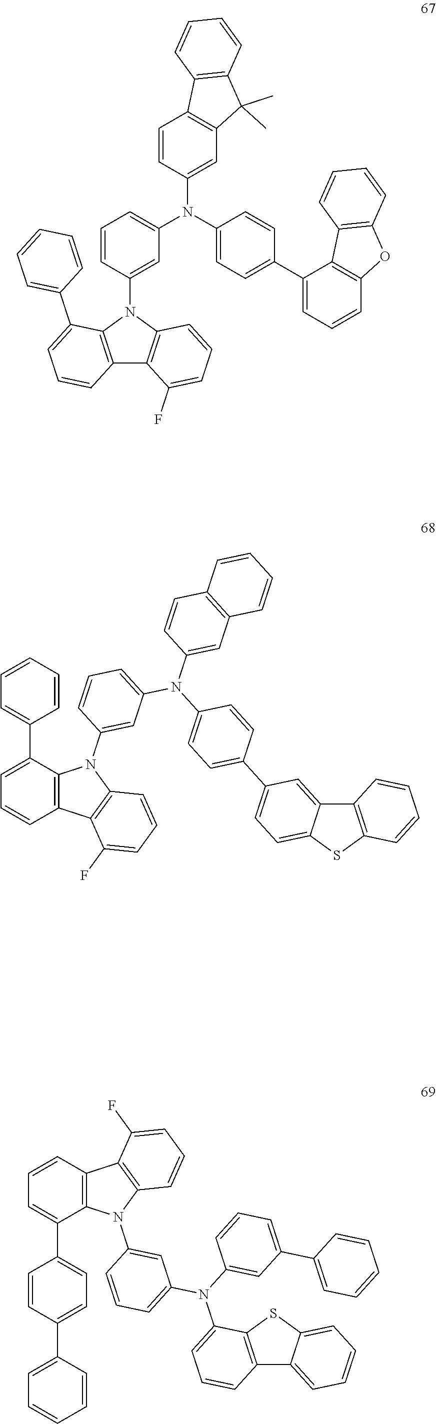 [SMILES: CC1(C)c2ccccc2-c2ccc(N(c3ccc(-c4cccc5oc6ccccc6c45)cc3)c3cccc(-n4c5cccc(F)c5c5cccc(-c6ccccc6)c54)c3)cc21 ; Fc1cccc2c1c1cccc(-c3ccccc3)c1n2-c1cccc(N(c2ccc(-c3ccc4sc5ccccc5c4c3)cc2)c2ccc3ccccc3c2)c1 ; Fc1cccc2c1c1cccc(-c3ccc(-c4ccccc4)cc3)c1n2-c1cccc(N(c2cccc(-c3ccccc3)c2)c2cccc3c2sc2ccccc23)c1]

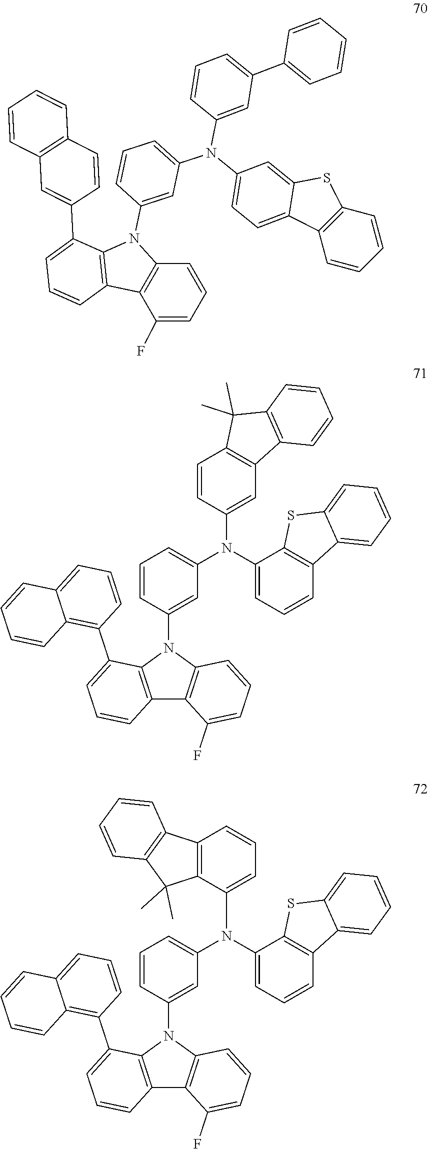 [SMILES: Fc1cccc2c1c1cccc(-c3ccc4ccccc4c3)c1n2-c1cccc(N(c2cccc(-c3ccccc3)c2)c2ccc3c(c2)sc2ccccc23)c1 ; CC1(C)c2ccccc2-c2cc(N(c3cccc(-n4c5cccc(F)c5c5cccc(-c6cccc7ccccc67)c54)c3)c3cccc4c3sc3ccccc34)ccc21 ; CC1(C)c2ccccc2-c2cccc(N(c3cccc(-n4c5cccc(F)c5c5cccc(-c6cccc7ccccc67)c54)c3)c3cccc4c3sc3ccccc34)c21]

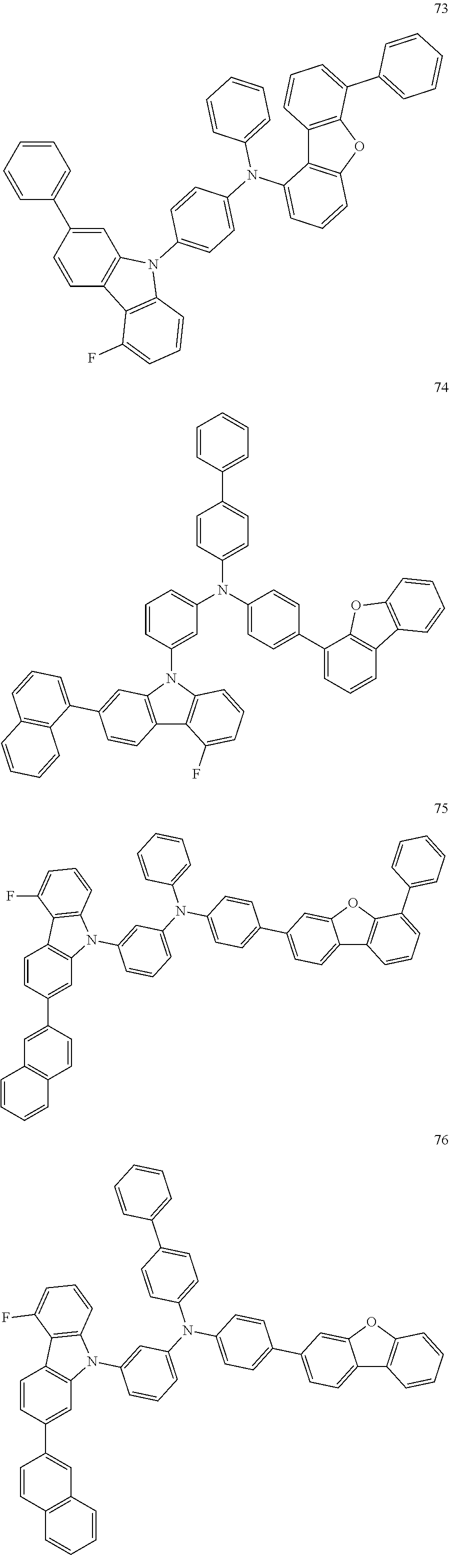 [SMILES: Fc1cccc2c1c1ccc(-c3ccccc3)cc1n2-c1ccc(N(c2ccccc2)c2cccc3oc4c(-c5ccccc5)cccc4c23)cc1 ; Fc1cccc2c1c1ccc(-c3cccc4ccccc34)cc1n2-c1cccc(N(c2ccc(-c3ccccc3)cc2)c2ccc(-c3cccc4c3oc3ccccc34)cc2)c1 ; Fc1cccc2c1c1ccc(-c3ccc4ccccc4c3)cc1n2-c1cccc(N(c2ccccc2)c2ccc(-c3ccc4c(c3)oc3c(-c5ccccc5)cccc34)cc2)c1 ; Fc1cccc2c1c1ccc(-c3ccc4ccccc4c3)cc1n2-c1cccc(N(c2ccc(-c3ccccc3)cc2)c2ccc(-c3ccc4c(c3)oc3ccccc34)cc2)c1]

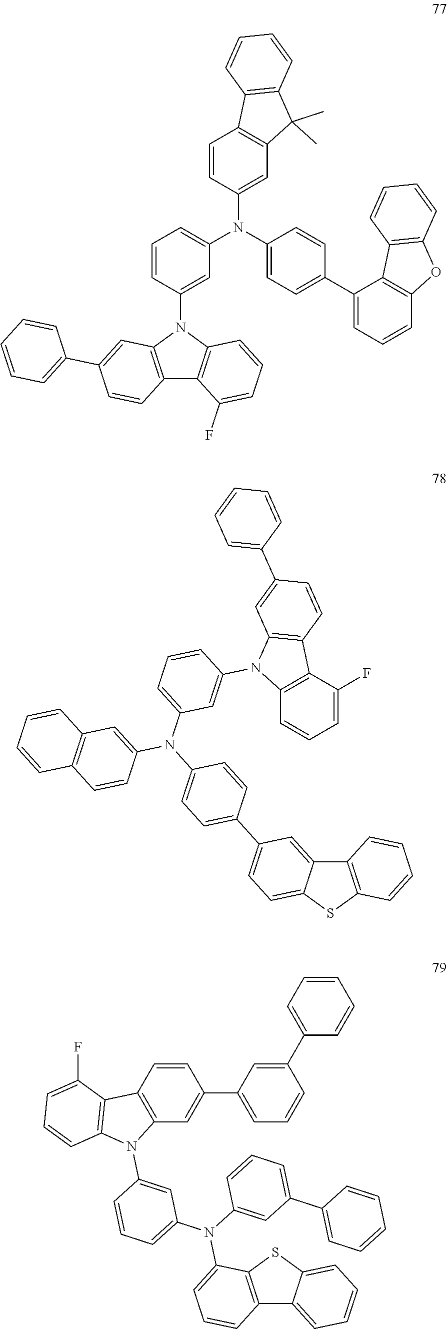 [SMILES: CC1(C)c2ccccc2-c2ccc(N(c3ccc(-c4cccc5oc6ccccc6c45)cc3)c3cccc(-n4c5cc(-c6ccccc6)ccc5c5c(F)cccc54)c3)cc21 ; Fc1cccc2c1c1ccc(-c3ccccc3)cc1n2-c1cccc(N(c2ccc(-c3ccc4sc5ccccc5c4c3)cc2)c2ccc3ccccc3c2)c1 ; Fc1cccc2c1c1ccc(-c3cccc(-c4ccccc4)c3)cc1n2-c1cccc(N(c2cccc(-c3ccccc3)c2)c2cccc3c2sc2ccccc23)c1]

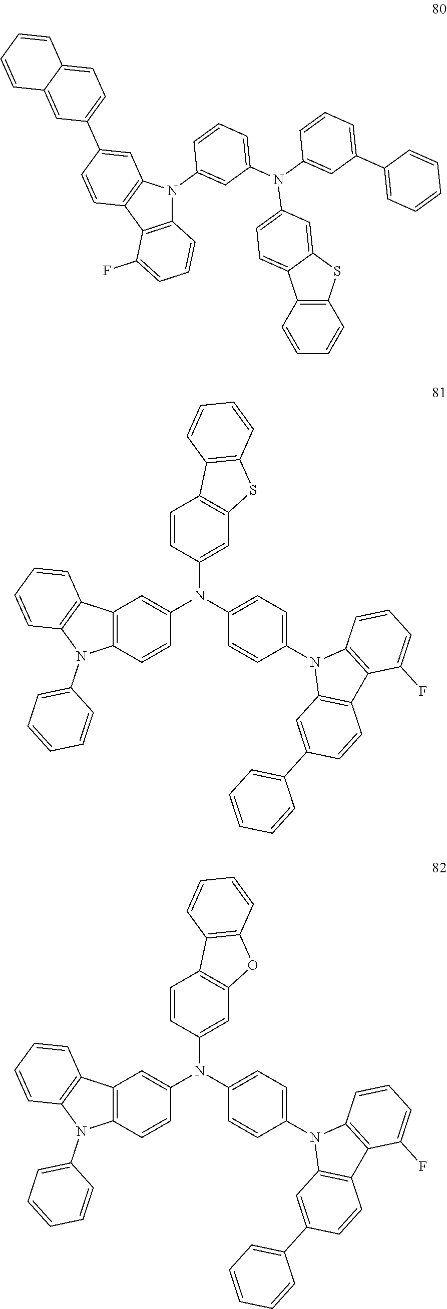 [SMILES: Fc1cccc2c1c1ccc(-c3ccc4ccccc4c3)cc1n2-c1cccc(N(c2cccc(-c3ccccc3)c2)c2ccc3c(c2)sc2ccccc23)c1 ; Fc1cccc2c1c1ccc(-c3ccccc3)cc1n2-c1ccc(N(c2ccc3c(c2)sc2ccccc23)c2ccc3c(c2)c2ccccc2n3-c2ccccc2)cc1 ; Fc1cccc2c1c1ccc(-c3ccccc3)cc1n2-c1ccc(N(c2ccc3c(c2)oc2ccccc23)c2ccc3c(c2)c2ccccc2n3-c2ccccc2)cc1]

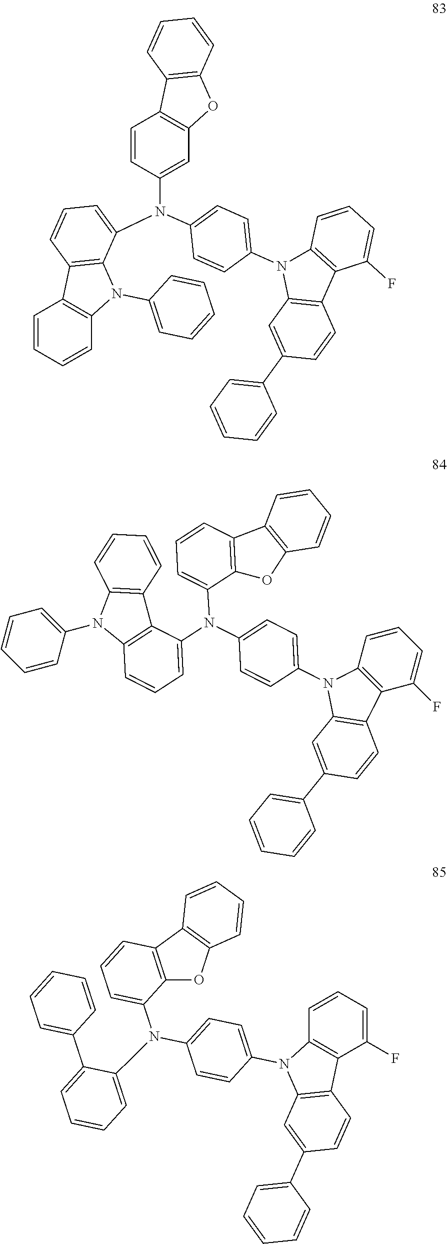 [SMILES: Fc1cccc2c1c1ccc(-c3ccccc3)cc1n2-c1ccc(N(c2ccc3c(c2)oc2ccccc23)c2cccc3c4ccccc4n(-c4ccccc4)c23)cc1 ; Fc1cccc2c1c1ccc(-c3ccccc3)cc1n2-c1ccc(N(c2cccc3c2oc2ccccc23)c2cccc3c2c2ccccc2n3-c2ccccc2)cc1 ; Fc1cccc2c1c1ccc(-c3ccccc3)cc1n2-c1ccc(N(c2ccccc2-c2ccccc2)c2cccc3c2oc2ccccc23)cc1]

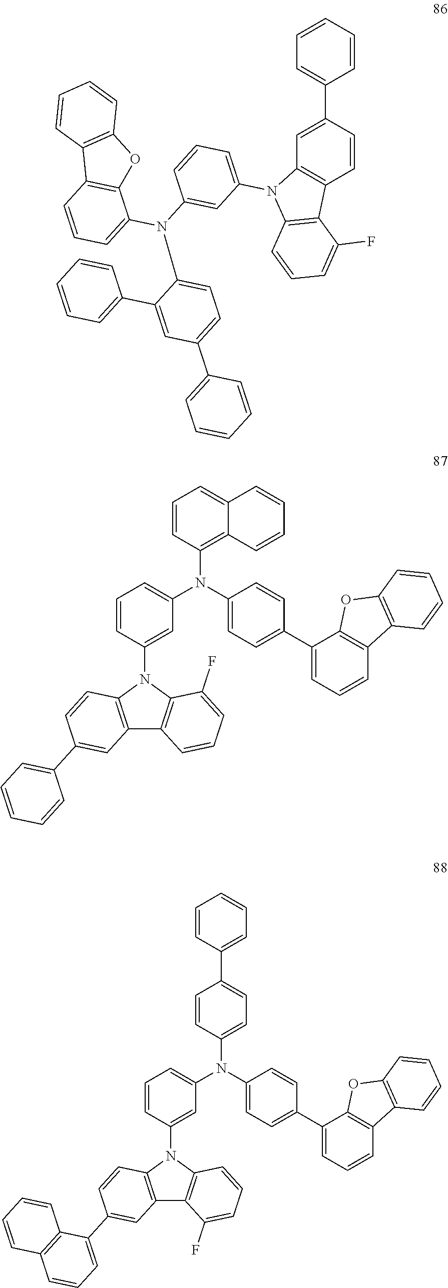 [SMILES: Fc1cccc2c1c1ccc(-c3ccccc3)cc1n2-c1cccc(N(c2ccc(-c3ccccc3)cc2-c2ccccc2)c2cccc3c2oc2ccccc23)c1 ; Fc1cccc2c3cc(-c4ccccc4)ccc3n(-c3cccc(N(c4ccc(-c5cccc6c5oc5ccccc56)cc4)c4cccc5ccccc45)c3)c12 ; Fc1cccc2c1c1cc(-c3cccc4ccccc34)ccc1n2-c1cccc(N(c2ccc(-c3ccccc3)cc2)c2ccc(-c3cccc4c3oc3ccccc34)cc2)c1]

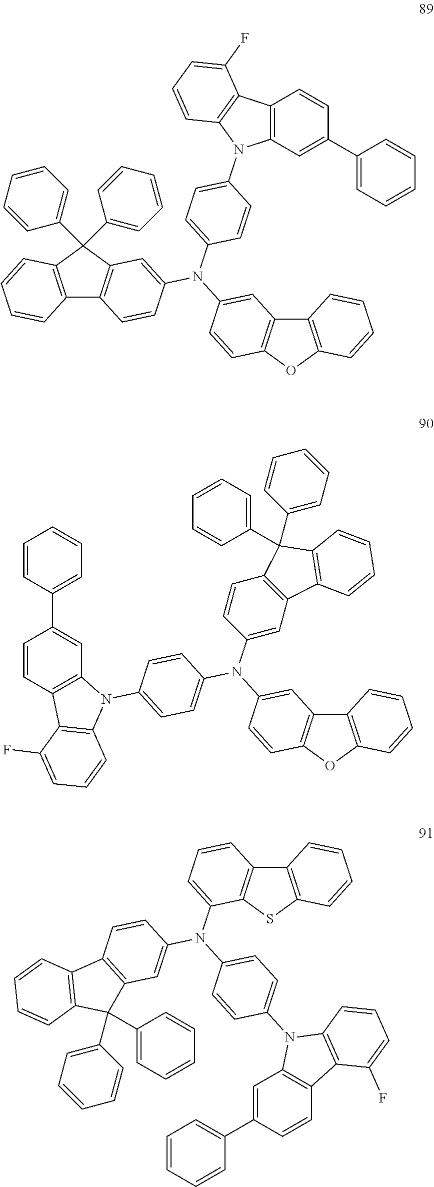 [SMILES: Fc1cccc2c1c1ccc(-c3ccccc3)cc1n2-c1ccc(N(c2ccc3c(c2)C(c2ccccc2)(c2ccccc2)c2ccccc2-3)c2ccc3oc4ccccc4c3c2)cc1 ; Fc1cccc2c1c1ccc(-c3ccccc3)cc1n2-c1ccc(N(c2ccc3c(c2)-c2ccccc2C3(c2ccccc2)c2ccccc2)c2ccc3oc4ccccc4c3c2)cc1 ; Fc1cccc2c1c1ccc(-c3ccccc3)cc1n2-c1ccc(N(c2ccc3c(c2)C(c2ccccc2)(c2ccccc2)c2ccccc2-3)c2cccc3c2sc2ccccc23)cc1]

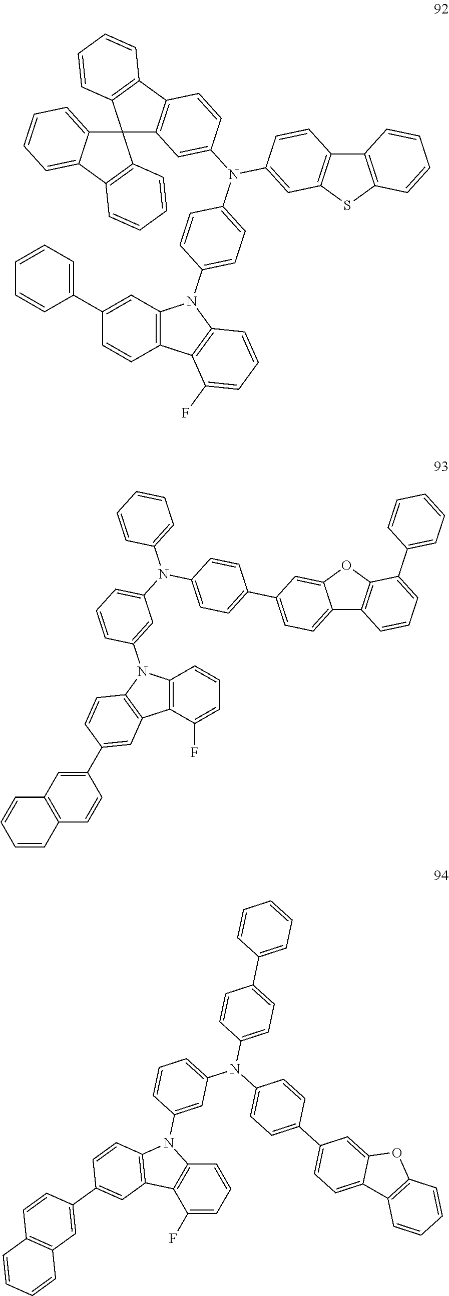 [SMILES: Fc1cccc2c1c1ccc(-c3ccccc3)cc1n2-c1ccc(N(c2ccc3c(c2)C2(c4ccccc4-c4ccccc42)c2ccccc2-3)c2ccc3c(c2)sc2ccccc23)cc1 ; Fc1cccc2c1c1cc(-c3ccc4ccccc4c3)ccc1n2-c1cccc(N(c2ccccc2)c2ccc(-c3ccc4c(c3)oc3c(-c5ccccc5)cccc34)cc2)c1 ; Fc1cccc2c1c1cc(-c3ccc4ccccc4c3)ccc1n2-c1cccc(N(c2ccc(-c3ccccc3)cc2)c2ccc(-c3ccc4c(c3)oc3ccccc34)cc2)c1]

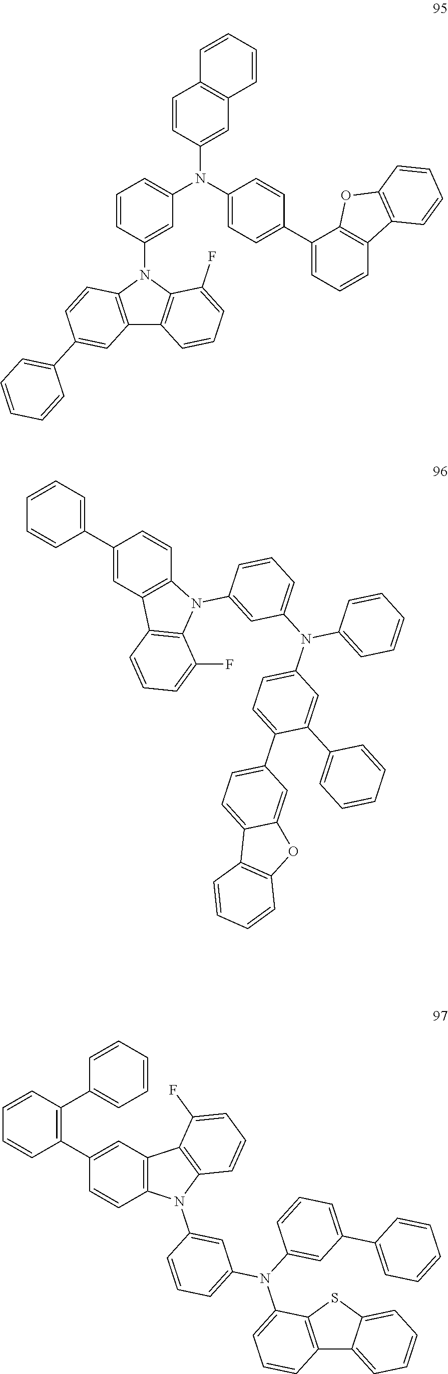 [SMILES: Fc1cccc2c3cc(-c4ccccc4)ccc3n(-c3cccc(N(c4ccc(-c5cccc6c5oc5ccccc56)cc4)c4ccc5ccccc5c4)c3)c12 ; Fc1cccc2c3cc(-c4ccccc4)ccc3n(-c3cccc(N(c4ccccc4)c4ccc(-c5ccc6c(c5)oc5ccccc56)c(-c5ccccc5)c4)c3)c12 ; Fc1cccc2c1c1cc(-c3ccccc3-c3ccccc3)ccc1n2-c1cccc(N(c2cccc(-c3ccccc3)c2)c2cccc3c2sc2ccccc23)c1]

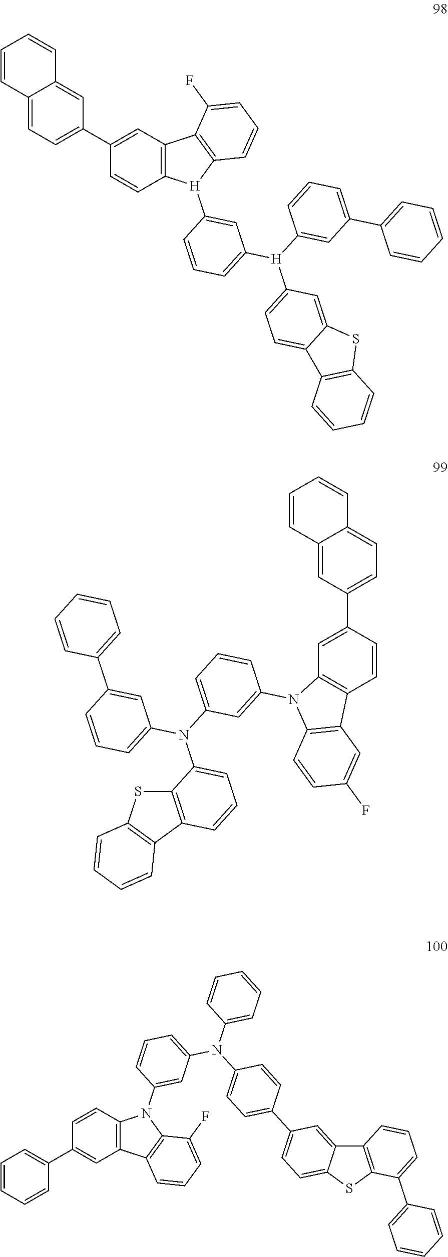

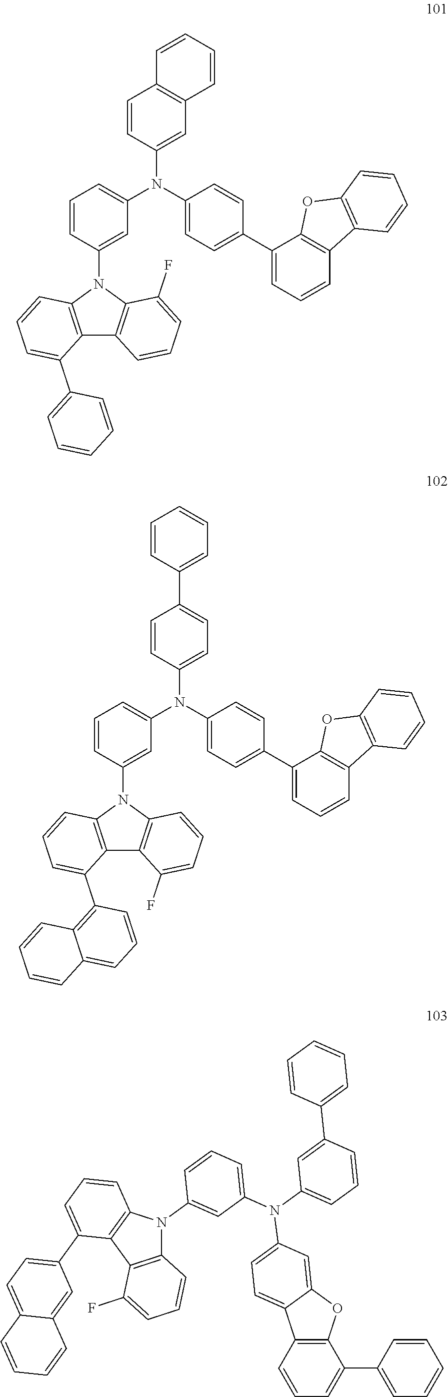 [SMILES: Fc1cccc2c3c(-c4ccccc4)cccc3n(-c3cccc(N(c4ccc(-c5cccc6c5oc5ccccc56)cc4)c4ccc5ccccc5c4)c3)c12 ; Fc1cccc2c1c1c(-c3cccc4ccccc34)cccc1n2-c1cccc(N(c2ccc(-c3ccccc3)cc2)c2ccc(-c3cccc4c3oc3ccccc34)cc2)c1 ; Fc1cccc2c1c1c(-c3ccc4ccccc4c3)cccc1n2-c1cccc(N(c2cccc(-c3ccccc3)c2)c2ccc3c(c2)oc2c(-c4ccccc4)cccc23)c1]

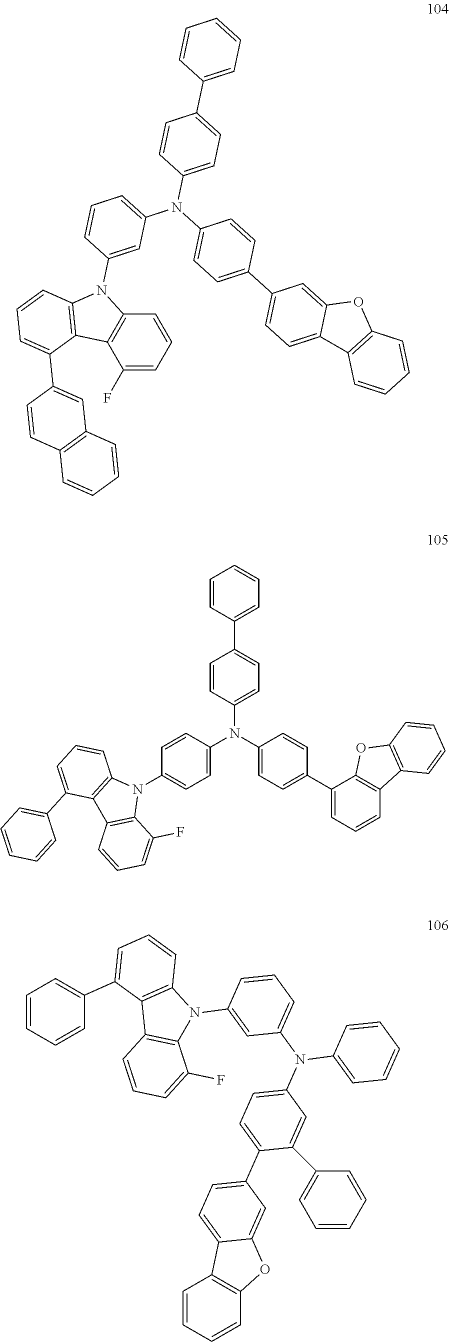 [SMILES: Fc1cccc2c1c1c(-c3ccc4ccccc4c3)cccc1n2-c1cccc(N(c2ccc(-c3ccccc3)cc2)c2ccc(-c3ccc4c(c3)oc3ccccc34)cc2)c1 ; Fc1cccc2c3c(-c4ccccc4)cccc3n(-c3ccc(N(c4ccc(-c5ccccc5)cc4)c4ccc(-c5cccc6c5oc5ccccc56)cc4)cc3)c12 ; Fc1cccc2c3c(-c4ccccc4)cccc3n(-c3cccc(N(c4ccccc4)c4ccc(-c5ccc6c(c5)oc5ccccc56)c(-c5ccccc5)c4)c3)c12]

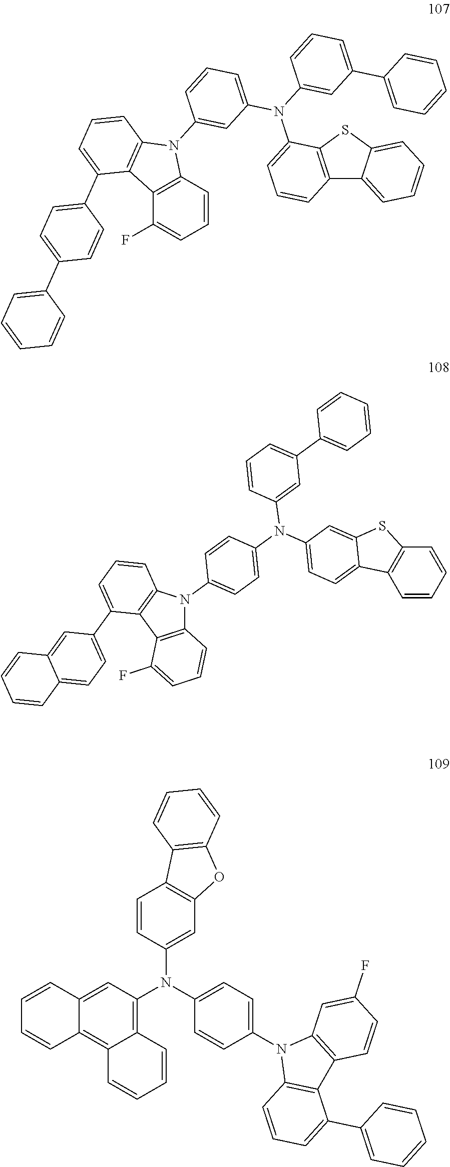 [SMILES: Fc1cccc2c1c1c(-c3ccc(-c4ccccc4)cc3)cccc1n2-c1cccc(N(c2cccc(-c3ccccc3)c2)c2cccc3c2sc2ccccc23)c1 ; Fc1cccc2c1c1c(-c3ccc4ccccc4c3)cccc1n2-c1ccc(N(c2cccc(-c3ccccc3)c2)c2ccc3c(c2)sc2ccccc23)cc1 ; Fc1ccc2c3c(-c4ccccc4)cccc3n(-c3ccc(N(c4ccc5c(c4)oc4ccccc45)c4cc5ccccc5c5ccccc45)cc3)c2c1]

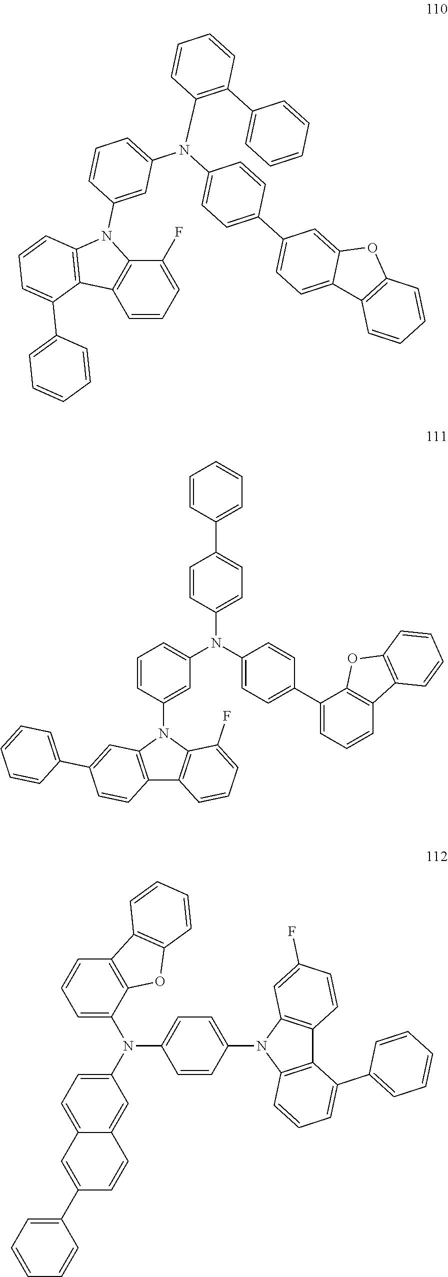 [SMILES: Fc1cccc2c3c(-c4ccccc4)cccc3n(-c3cccc(N(c4ccc(-c5ccc6c(c5)oc5ccccc56)cc4)c4ccccc4-c4ccccc4)c3)c12 ; Fc1cccc2c3ccc(-c4ccccc4)cc3n(-c3cccc(N(c4ccc(-c5ccccc5)cc4)c4ccc(-c5cccc6c5oc5ccccc56)cc4)c3)c12 ; Fc1ccc2c3c(-c4ccccc4)cccc3n(-c3ccc(N(c4ccc5cc(-c6ccccc6)ccc5c4)c4cccc5c4oc4ccccc45)cc3)c2c1]

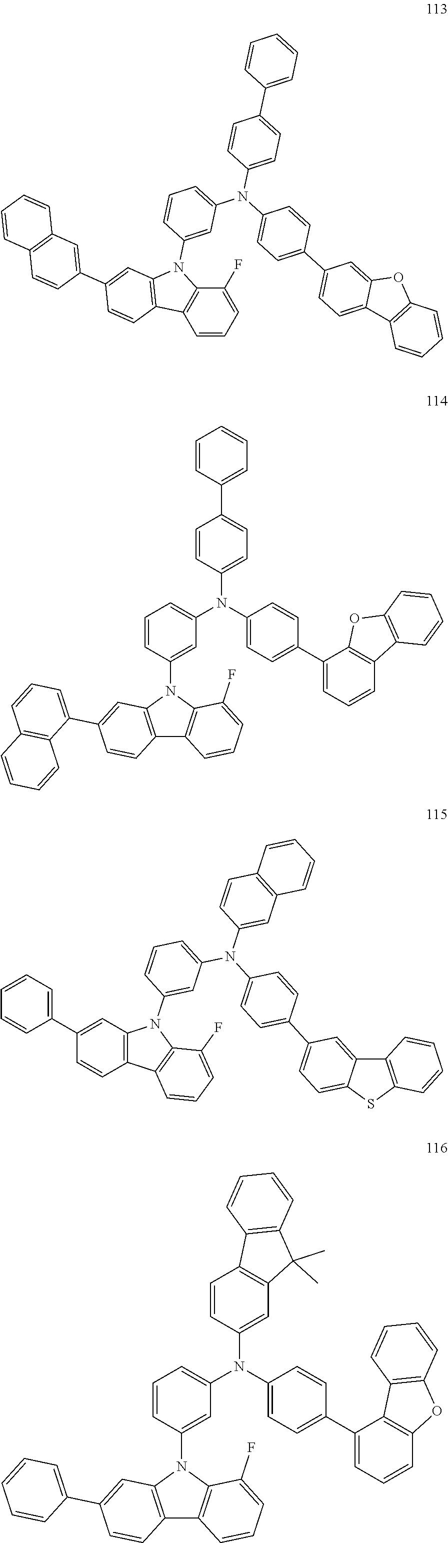 [SMILES: Fc1cccc2c3ccc(-c4ccc5ccccc5c4)cc3n(-c3cccc(N(c4ccc(-c5ccccc5)cc4)c4ccc(-c5ccc6c(c5)oc5ccccc56)cc4)c3)c12 ; Fc1cccc2c3ccc(-c4cccc5ccccc45)cc3n(-c3cccc(N(c4ccc(-c5ccccc5)cc4)c4ccc(-c5cccc6c5oc5ccccc56)cc4)c3)c12 ; Fc1cccc2c3ccc(-c4ccccc4)cc3n(-c3cccc(N(c4ccc(-c5ccc6sc7ccccc7c6c5)cc4)c4ccc5ccccc5c4)c3)c12 ; CC1(C)c2ccccc2-c2ccc(N(c3ccc(-c4cccc5oc6ccccc6c45)cc3)c3cccc(-n4c5cc(-c6ccccc6)ccc5c5cccc(F)c54)c3)cc21]

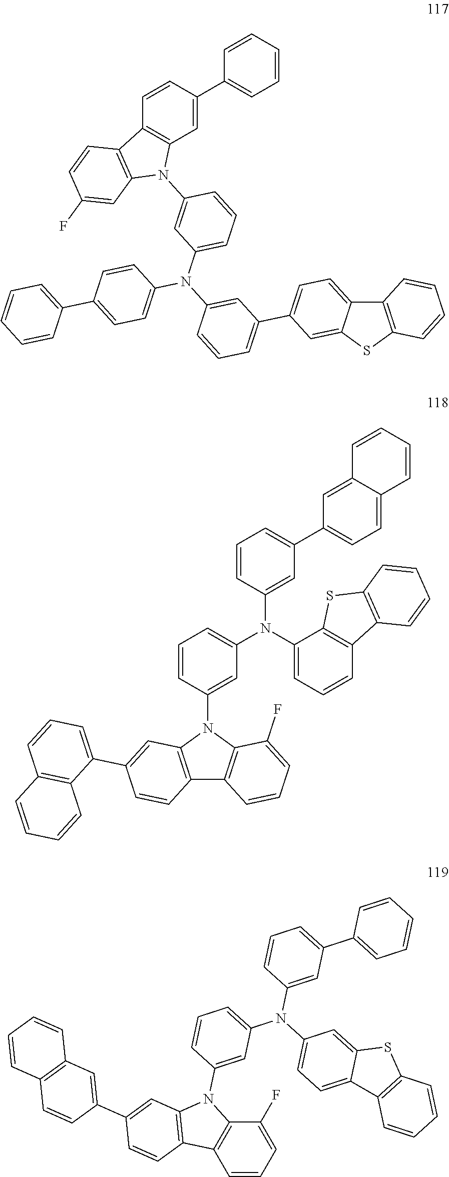 [SMILES: Fc1ccc2c3ccc(-c4ccccc4)cc3n(-c3cccc(N(c4ccc(-c5ccccc5)cc4)c4cccc(-c5ccc6c(c5)sc5ccccc56)c4)c3)c2c1 ; Fc1cccc2c3ccc(-c4cccc5ccccc45)cc3n(-c3cccc(N(c4cccc(-c5ccc6ccccc6c5)c4)c4cccc5c4sc4ccccc45)c3)c12 ; Fc1cccc2c3ccc(-c4ccc5ccccc5c4)cc3n(-c3cccc(N(c4cccc(-c5ccccc5)c4)c4ccc5c(c4)sc4ccccc45)c3)c12]

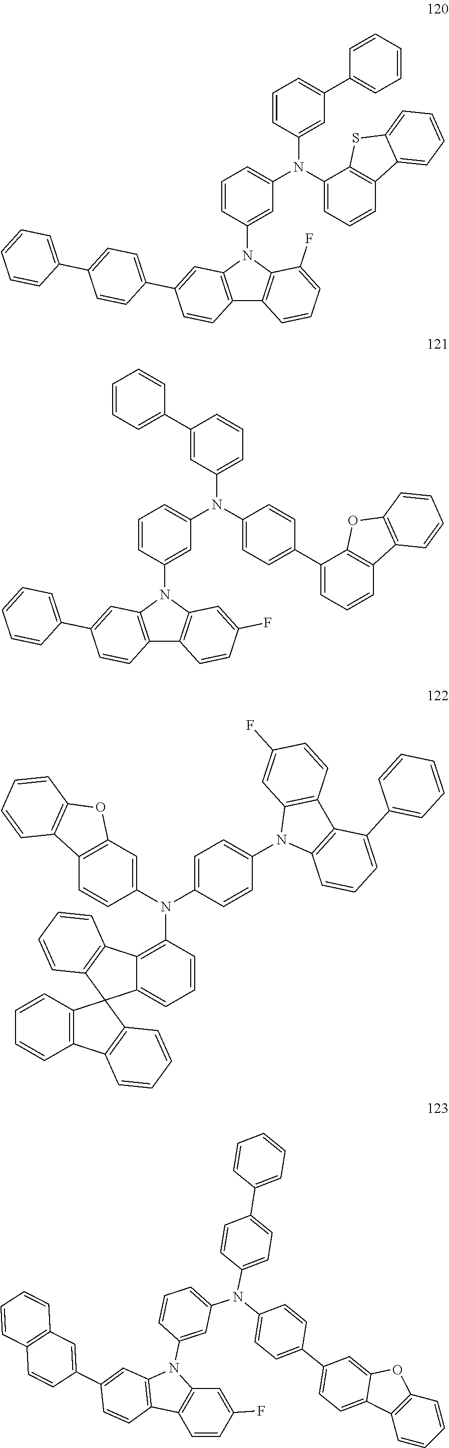 [SMILES: Fc1cccc2c3ccc(-c4ccc(-c5ccccc5)cc4)cc3n(-c3cccc(N(c4cccc(-c5ccccc5)c4)c4cccc5c4sc4ccccc45)c3)c12 ; Fc1ccc2c3ccc(-c4ccccc4)cc3n(-c3cccc(N(c4ccc(-c5cccc6c5oc5ccccc56)cc4)c4cccc(-c5ccccc5)c4)c3)c2c1 ; Fc1ccc2c3c(-c4ccccc4)cccc3n(-c3ccc(N(c4ccc5c(c4)oc4ccccc45)c4cccc5c4-c4ccccc4C54c5ccccc5-c5ccccc54)cc3)c2c1 ; Fc1ccc2c3ccc(-c4ccc5ccccc5c4)cc3n(-c3cccc(N(c4ccc(-c5ccccc5)cc4)c4ccc(-c5ccc6c(c5)oc5ccccc56)cc4)c3)c2c1]

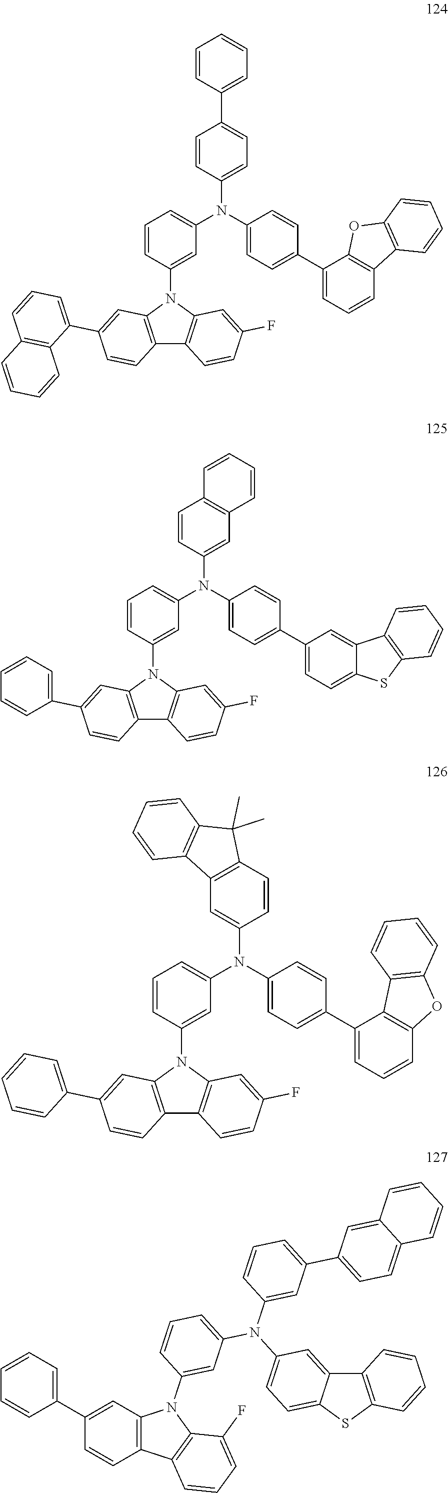 [SMILES: Fc1ccc2c3ccc(-c4cccc5ccccc45)cc3n(-c3cccc(N(c4ccc(-c5ccccc5)cc4)c4ccc(-c5cccc6c5oc5ccccc56)cc4)c3)c2c1 ; Fc1ccc2c3ccc(-c4ccccc4)cc3n(-c3cccc(N(c4ccc(-c5ccc6sc7ccccc7c6c5)cc4)c4ccc5ccccc5c4)c3)c2c1 ; CC1(C)c2ccccc2-c2cc(N(c3ccc(-c4cccc5oc6ccccc6c45)cc3)c3cccc(-n4c5cc(F)ccc5c5ccc(-c6ccccc6)cc54)c3)ccc21 ; Fc1cccc2c3ccc(-c4ccccc4)cc3n(-c3cccc(N(c4cccc(-c5ccc6ccccc6c5)c4)c4ccc5sc6ccccc6c5c4)c3)c12]

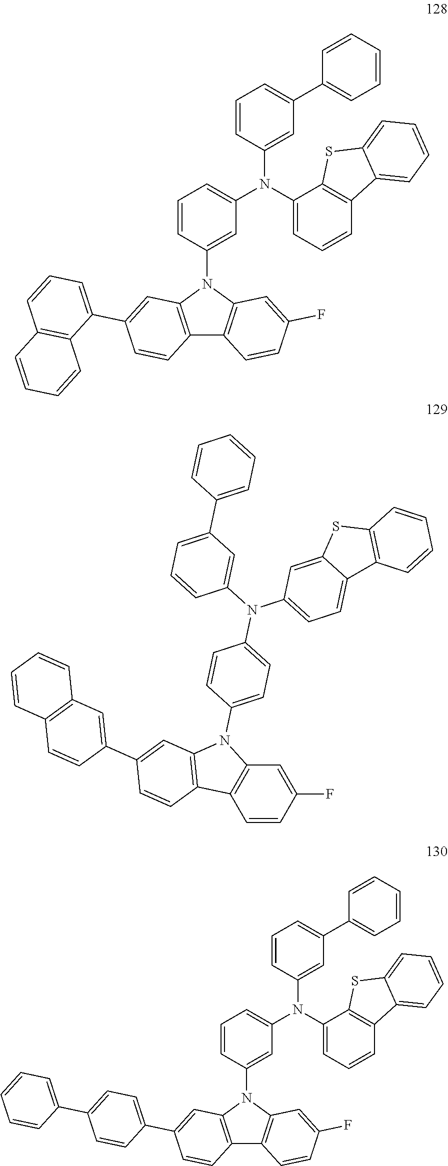 [SMILES: Fc1ccc2c3ccc(-c4cccc5ccccc45)cc3n(-c3cccc(N(c4cccc(-c5ccccc5)c4)c4cccc5c4sc4ccccc45)c3)c2c1 ; Fc1ccc2c3ccc(-c4ccc5ccccc5c4)cc3n(-c3ccc(N(c4cccc(-c5ccccc5)c4)c4ccc5c(c4)sc4ccccc45)cc3)c2c1 ; Fc1ccc2c3ccc(-c4ccc(-c5ccccc5)cc4)cc3n(-c3cccc(N(c4cccc(-c5ccccc5)c4)c4cccc5c4sc4ccccc45)c3)c2c1]

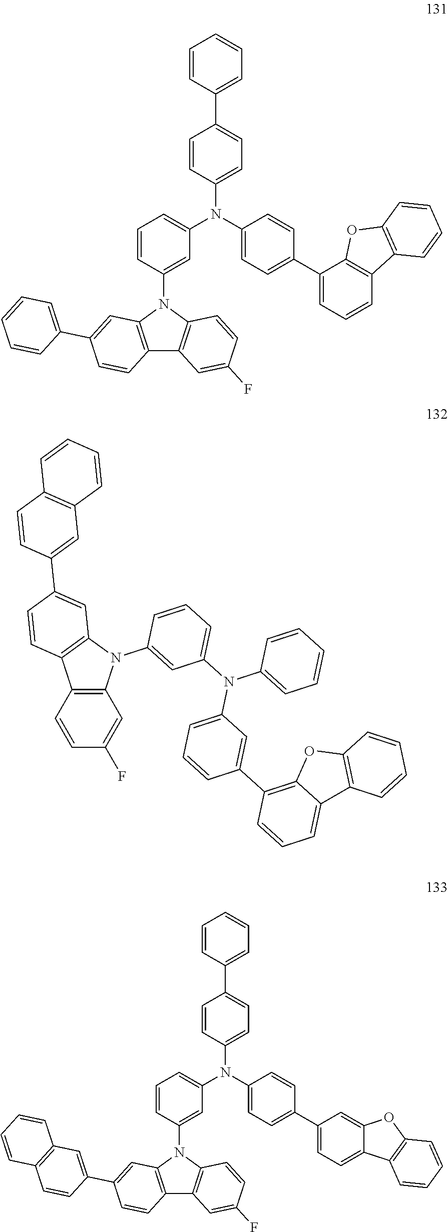 [SMILES: Fc1ccc2c(c1)c1ccc(-c3ccccc3)cc1n2-c1cccc(N(c2ccc(-c3ccccc3)cc2)c2ccc(-c3cccc4c3oc3ccccc34)cc2)c1 ; Fc1ccc2c3ccc(-c4ccc5ccccc5c4)cc3n(-c3cccc(N(c4ccccc4)c4cccc(-c5cccc6c5oc5ccccc56)c4)c3)c2c1 ; Fc1ccc2c(c1)c1ccc(-c3ccc4ccccc4c3)cc1n2-c1cccc(N(c2ccc(-c3ccccc3)cc2)c2ccc(-c3ccc4c(c3)oc3ccccc34)cc2)c1]

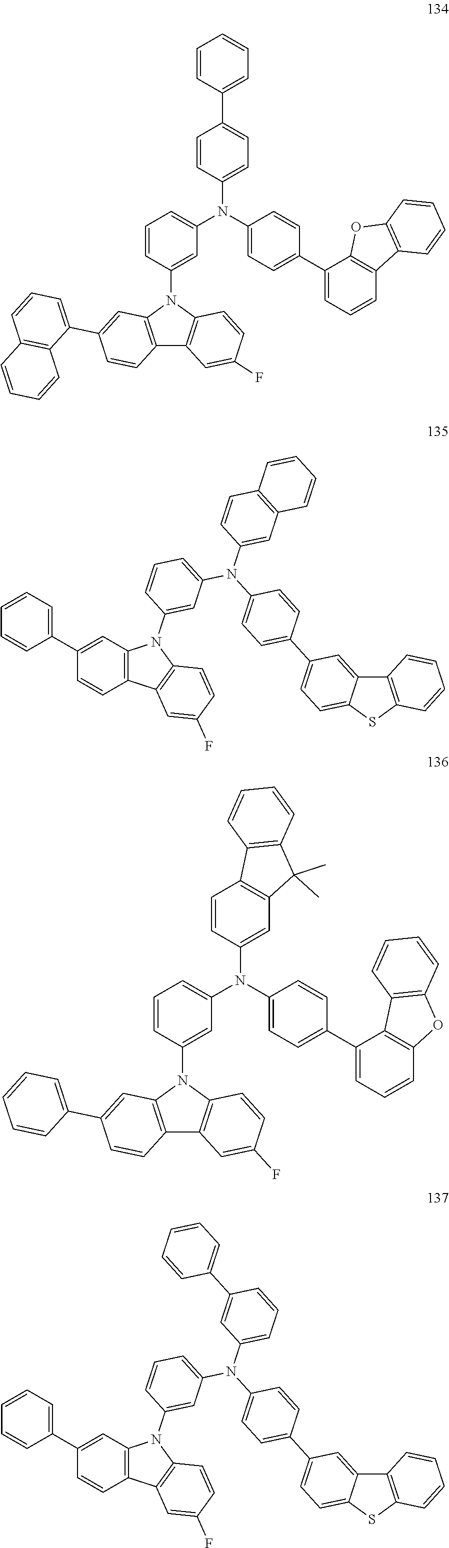 [SMILES: Fc1ccc2c(c1)c1ccc(-c3cccc4ccccc34)cc1n2-c1cccc(N(c2ccc(-c3ccccc3)cc2)c2ccc(-c3cccc4c3oc3ccccc34)cc2)c1 ; Fc1ccc2c(c1)c1ccc(-c3ccccc3)cc1n2-c1cccc(N(c2ccc(-c3ccc4sc5ccccc5c4c3)cc2)c2ccc3ccccc3c2)c1 ; CC1(C)c2ccccc2-c2ccc(N(c3ccc(-c4cccc5oc6ccccc6c45)cc3)c3cccc(-n4c5ccc(F)cc5c5ccc(-c6ccccc6)cc54)c3)cc21 ; Fc1ccc2c(c1)c1ccc(-c3ccccc3)cc1n2-c1cccc(N(c2ccc(-c3ccc4sc5ccccc5c4c3)cc2)c2cccc(-c3ccccc3)c2)c1]

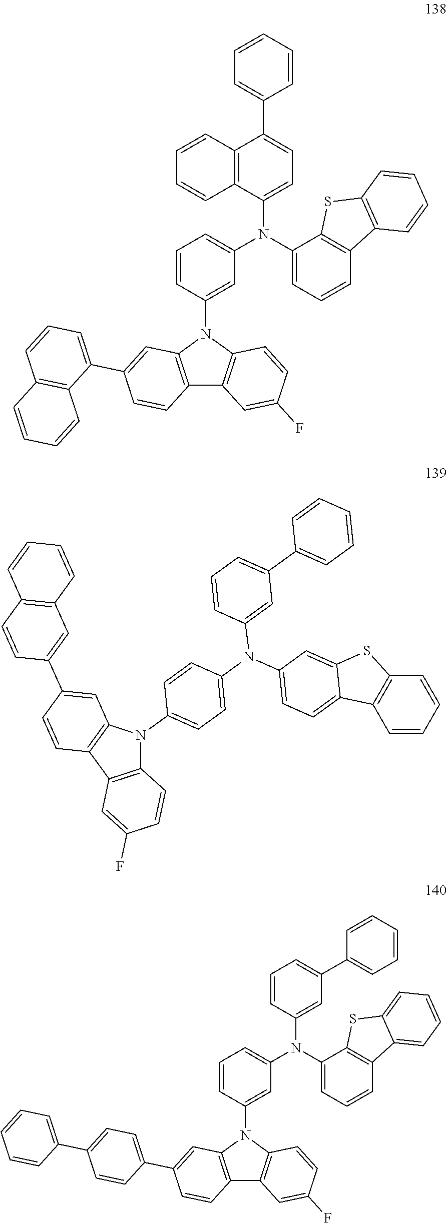 [SMILES: Fc1ccc2c(c1)c1ccc(-c3cccc4ccccc34)cc1n2-c1cccc(N(c2ccc(-c3ccccc3)c3ccccc23)c2cccc3c2sc2ccccc23)c1 ; Fc1ccc2c(c1)c1ccc(-c3ccc4ccccc4c3)cc1n2-c1ccc(N(c2cccc(-c3ccccc3)c2)c2ccc3c(c2)sc2ccccc23)cc1 ; Fc1ccc2c(c1)c1ccc(-c3ccc(-c4ccccc4)cc3)cc1n2-c1cccc(N(c2cccc(-c3ccccc3)c2)c2cccc3c2sc2ccccc23)c1]

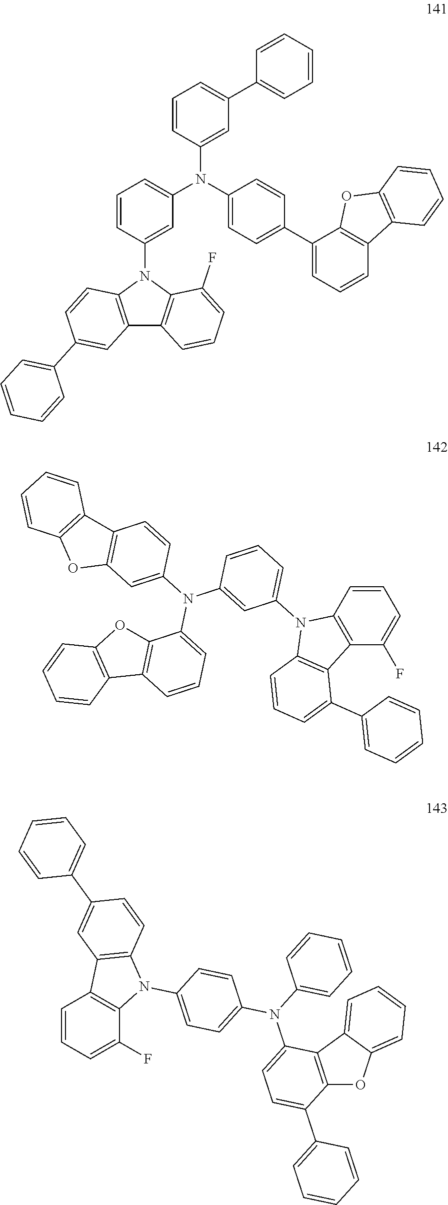 [SMILES: Fc1cccc2c3cc(-c4ccccc4)ccc3n(-c3cccc(N(c4ccc(-c5cccc6c5oc5ccccc56)cc4)c4cccc(-c5ccccc5)c4)c3)c12 ; Fc1cccc2c1c1c(-c3ccccc3)cccc1n2-c1cccc(N(c2ccc3c(c2)oc2ccccc23)c2cccc3c2oc2ccccc23)c1 ; Fc1cccc2c3cc(-c4ccccc4)ccc3n(-c3ccc(N(c4ccccc4)c4ccc(-c5ccccc5)c5oc6ccccc6c45)cc3)c12]

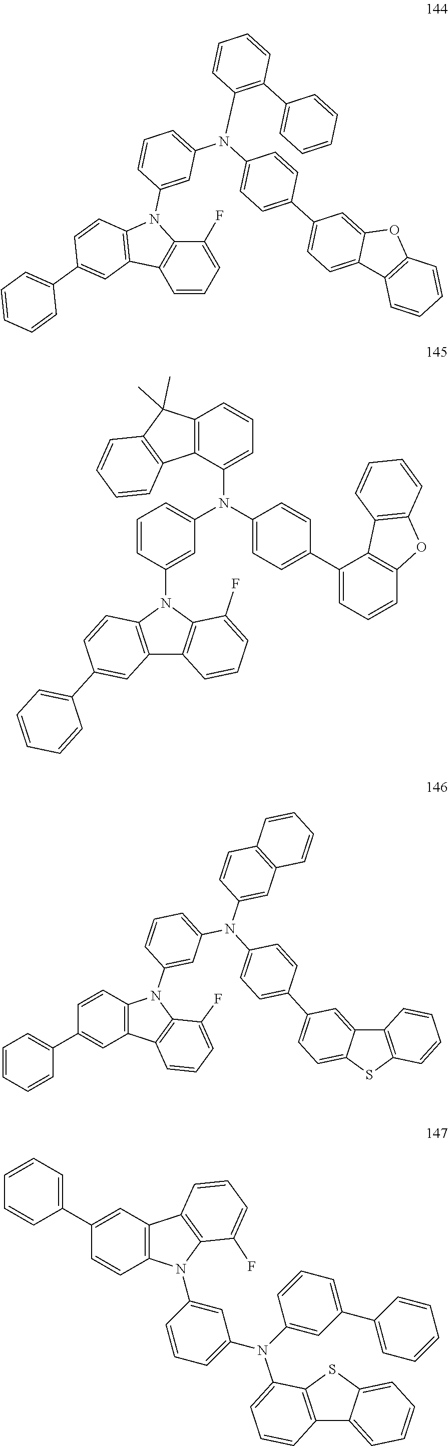 [SMILES: Fc1cccc2c3cc(-c4ccccc4)ccc3n(-c3cccc(N(c4ccc(-c5ccc6c(c5)oc5ccccc56)cc4)c4ccccc4-c4ccccc4)c3)c12 ; CC1(C)c2ccccc2-c2c(N(c3ccc(-c4cccc5oc6ccccc6c45)cc3)c3cccc(-n4c5ccc(-c6ccccc6)cc5c5cccc(F)c54)c3)cccc21 ; Fc1cccc2c3cc(-c4ccccc4)ccc3n(-c3cccc(N(c4ccc(-c5ccc6sc7ccccc7c6c5)cc4)c4ccc5ccccc5c4)c3)c12 ; Fc1cccc2c3cc(-c4ccccc4)ccc3n(-c3cccc(N(c4cccc(-c5ccccc5)c4)c4cccc5c4sc4ccccc45)c3)c12]

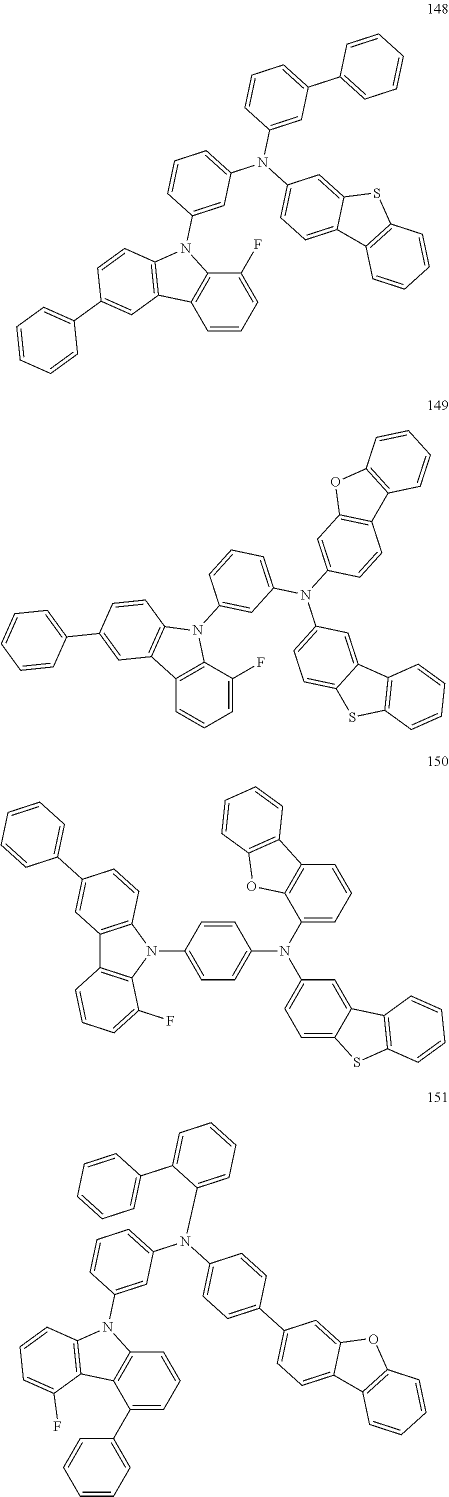 [SMILES: Fc1cccc2c3cc(-c4ccccc4)ccc3n(-c3cccc(N(c4cccc(-c5ccccc5)c4)c4ccc5c(c4)sc4ccccc45)c3)c12 ; Fc1cccc2c3cc(-c4ccccc4)ccc3n(-c3cccc(N(c4ccc5c(c4)oc4ccccc45)c4ccc5sc6ccccc6c5c4)c3)c12 ; Fc1cccc2c3cc(-c4ccccc4)ccc3n(-c3ccc(N(c4ccc5sc6ccccc6c5c4)c4cccc5c4oc4ccccc45)cc3)c12 ; Fc1cccc2c1c1c(-c3ccccc3)cccc1n2-c1cccc(N(c2ccc(-c3ccc4c(c3)oc3ccccc34)cc2)c2ccccc2-c2ccccc2)c1]

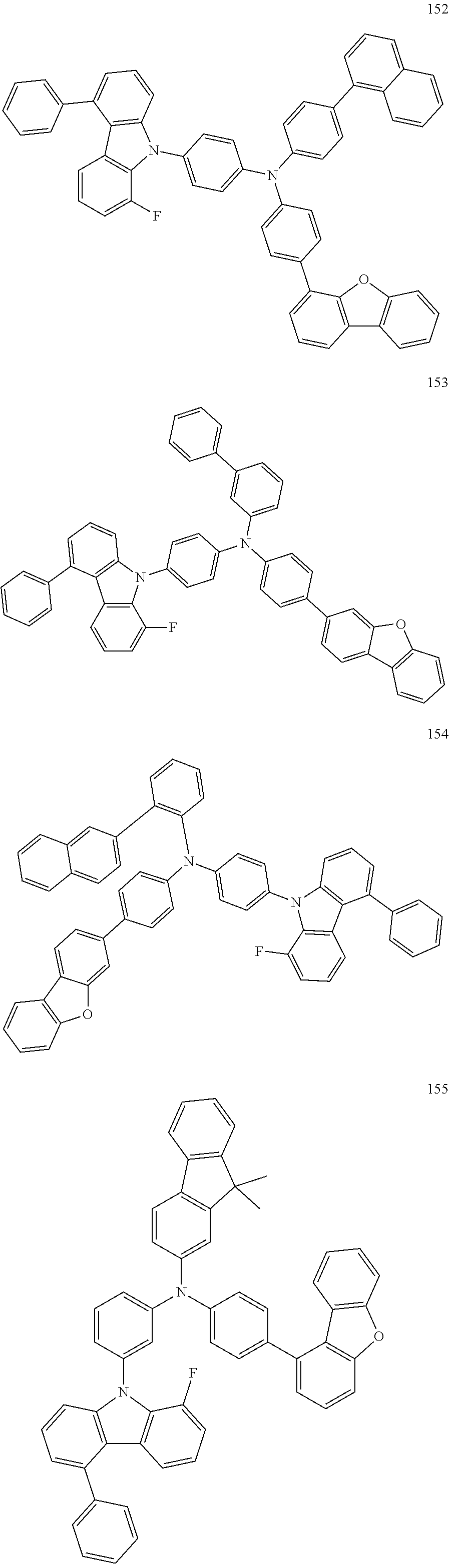 [SMILES: Fc1cccc2c3c(-c4ccccc4)cccc3n(-c3ccc(N(c4ccc(-c5cccc6ccccc56)cc4)c4ccc(-c5cccc6c5oc5ccccc56)cc4)cc3)c12 ; Fc1cccc2c3c(-c4ccccc4)cccc3n(-c3ccc(N(c4ccc(-c5ccc6c(c5)oc5ccccc56)cc4)c4cccc(-c5ccccc5)c4)cc3)c12 ; Fc1cccc2c3c(-c4ccccc4)cccc3n(-c3ccc(N(c4ccc(-c5ccc6c(c5)oc5ccccc56)cc4)c4ccccc4-c4ccc5ccccc5c4)cc3)c12 ; CC1(C)c2ccccc2-c2ccc(N(c3ccc(-c4cccc5oc6ccccc6c45)cc3)c3cccc(-n4c5cccc(-c6ccccc6)c5c5cccc(F)c54)c3)cc21]

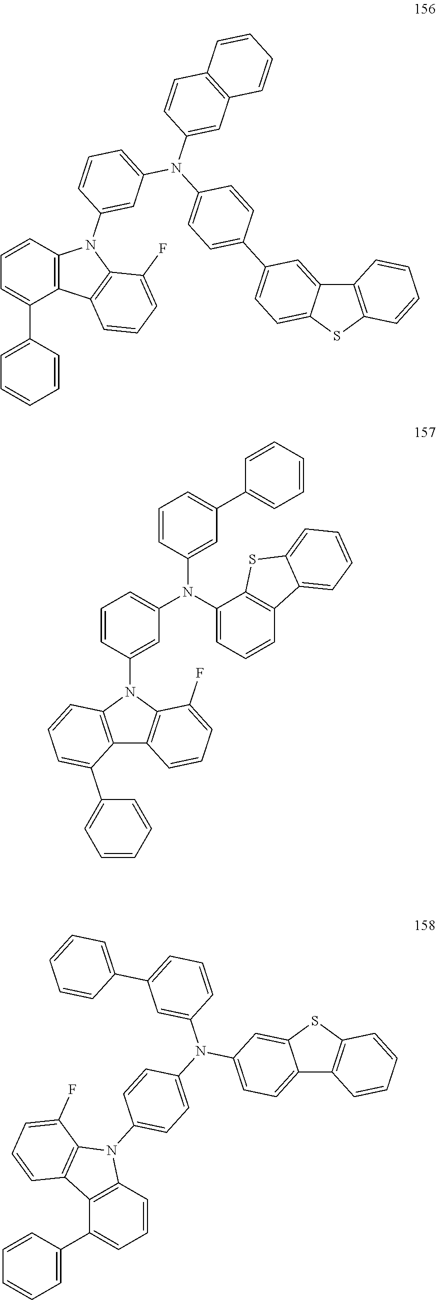 [SMILES: Fc1cccc2c3c(-c4ccccc4)cccc3n(-c3cccc(N(c4ccc(-c5ccc6sc7ccccc7c6c5)cc4)c4ccc5ccccc5c4)c3)c12 ; Fc1cccc2c3c(-c4ccccc4)cccc3n(-c3cccc(N(c4cccc(-c5ccccc5)c4)c4cccc5c4sc4ccccc45)c3)c12 ; Fc1cccc2c3c(-c4ccccc4)cccc3n(-c3ccc(N(c4cccc(-c5ccccc5)c4)c4ccc5c(c4)sc4ccccc45)cc3)c12]

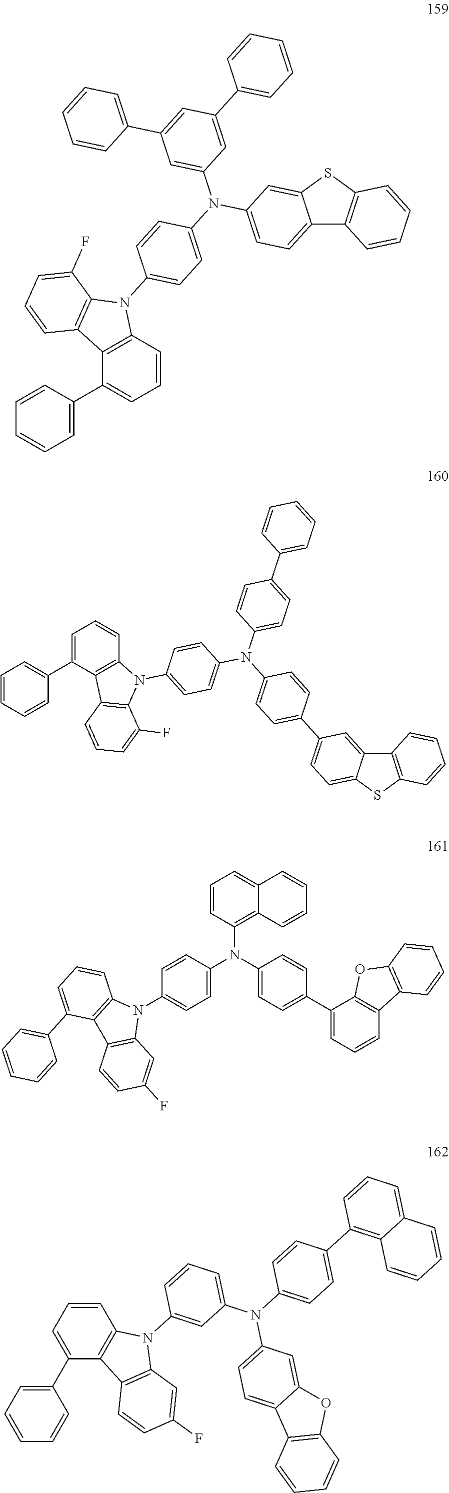 [SMILES: Fc1cccc2c3c(-c4ccccc4)cccc3n(-c3ccc(N(c4cc(-c5ccccc5)cc(-c5ccccc5)c4)c4ccc5c(c4)sc4ccccc45)cc3)c12 ; Fc1cccc2c3c(-c4ccccc4)cccc3n(-c3ccc(N(c4ccc(-c5ccccc5)cc4)c4ccc(-c5ccc6sc7ccccc7c6c5)cc4)cc3)c12 ; Fc1ccc2c3c(-c4ccccc4)cccc3n(-c3ccc(N(c4ccc(-c5cccc6c5oc5ccccc56)cc4)c4cccc5ccccc45)cc3)c2c1 ; Fc1ccc2c3c(-c4ccccc4)cccc3n(-c3cccc(N(c4ccc(-c5cccc6ccccc56)cc4)c4ccc5c(c4)oc4ccccc45)c3)c2c1]

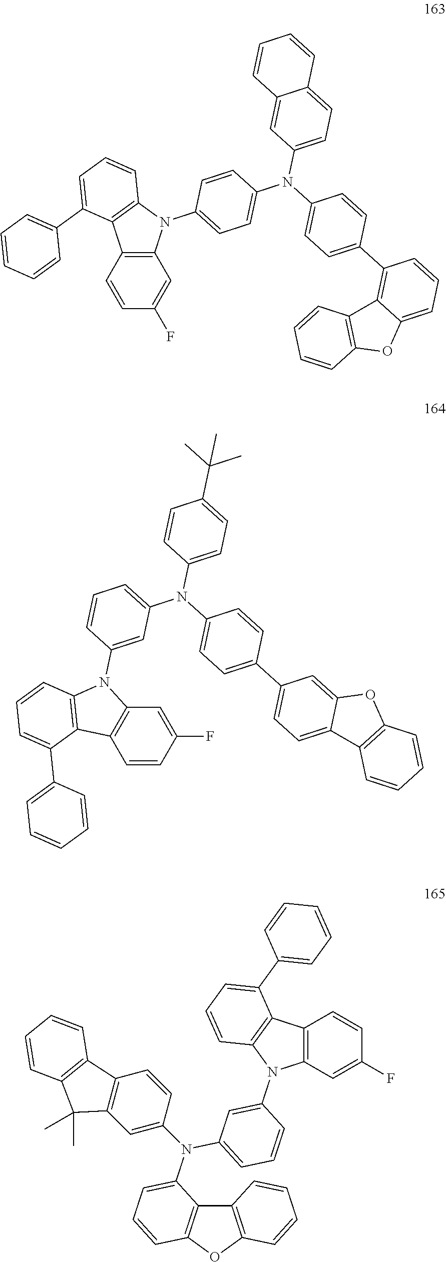 [SMILES: Fc1ccc2c3c(-c4ccccc4)cccc3n(-c3ccc(N(c4ccc(-c5cccc6oc7ccccc7c56)cc4)c4ccc5ccccc5c4)cc3)c2c1 ; CC(C)(C)c1ccc(N(c2ccc(-c3ccc4c(c3)oc3ccccc34)cc2)c2cccc(-n3c4cc(F)ccc4c4c(-c5ccccc5)cccc43)c2)cc1 ; CC1(C)c2ccccc2-c2ccc(N(c3cccc(-n4c5cc(F)ccc5c5c(-c6ccccc6)cccc54)c3)c3cccc4oc5ccccc5c34)cc21]

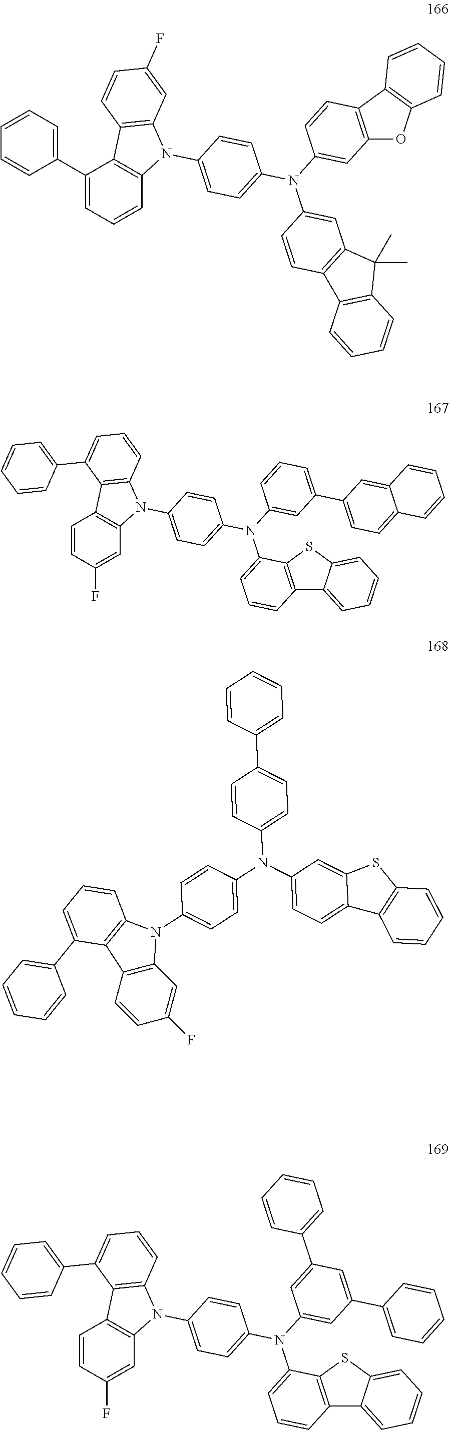 [SMILES: CC1(C)c2ccccc2-c2ccc(N(c3ccc(-n4c5cc(F)ccc5c5c(-c6ccccc6)cccc54)cc3)c3ccc4c(c3)oc3ccccc34)cc21 ; Fc1ccc2c3c(-c4ccccc4)cccc3n(-c3ccc(N(c4cccc(-c5ccc6ccccc6c5)c4)c4cccc5c4sc4ccccc45)cc3)c2c1 ; Fc1ccc2c3c(-c4ccccc4)cccc3n(-c3ccc(N(c4ccc(-c5ccccc5)cc4)c4ccc5c(c4)sc4ccccc45)cc3)c2c1 ; Fc1ccc2c3c(-c4ccccc4)cccc3n(-c3ccc(N(c4cc(-c5ccccc5)cc(-c5ccccc5)c4)c4cccc5c4sc4ccccc45)cc3)c2c1]

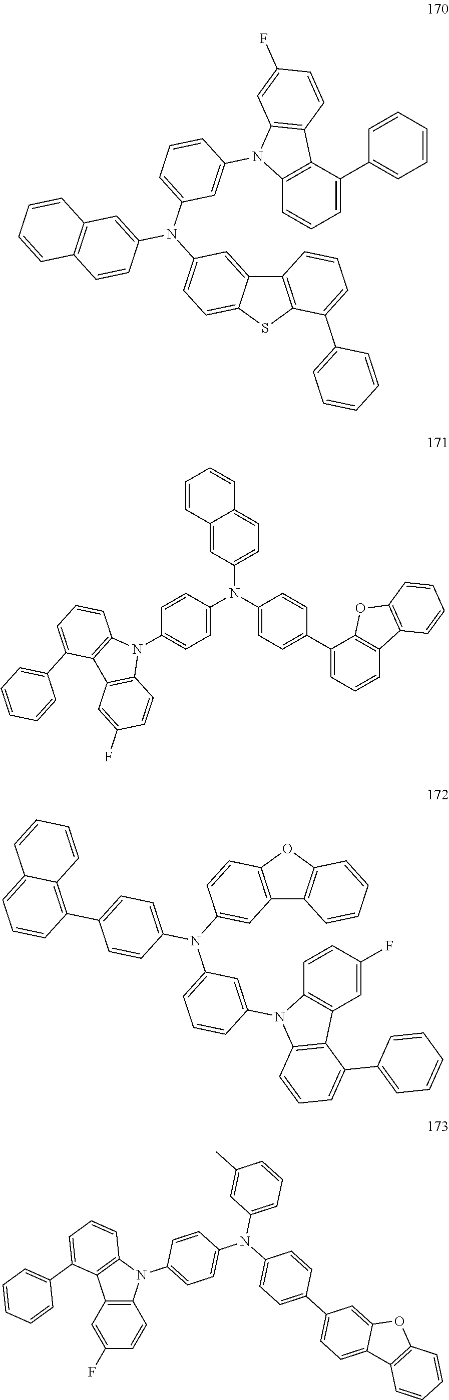 [SMILES: Fc1ccc2c3c(-c4ccccc4)cccc3n(-c3cccc(N(c4ccc5ccccc5c4)c4ccc5sc6c(-c7ccccc7)cccc6c5c4)c3)c2c1 ; Fc1ccc2c(c1)c1c(-c3ccccc3)cccc1n2-c1ccc(N(c2ccc(-c3cccc4c3oc3ccccc34)cc2)c2ccc3ccccc3c2)cc1 ; Fc1ccc2c(c1)c1c(-c3ccccc3)cccc1n2-c1cccc(N(c2ccc(-c3cccc4ccccc34)cc2)c2ccc3oc4ccccc4c3c2)c1 ; Cc1cccc(N(c2ccc(-c3ccc4c(c3)oc3ccccc34)cc2)c2ccc(-n3c4ccc(F)cc4c4c(-c5ccccc5)cccc43)cc2)c1]

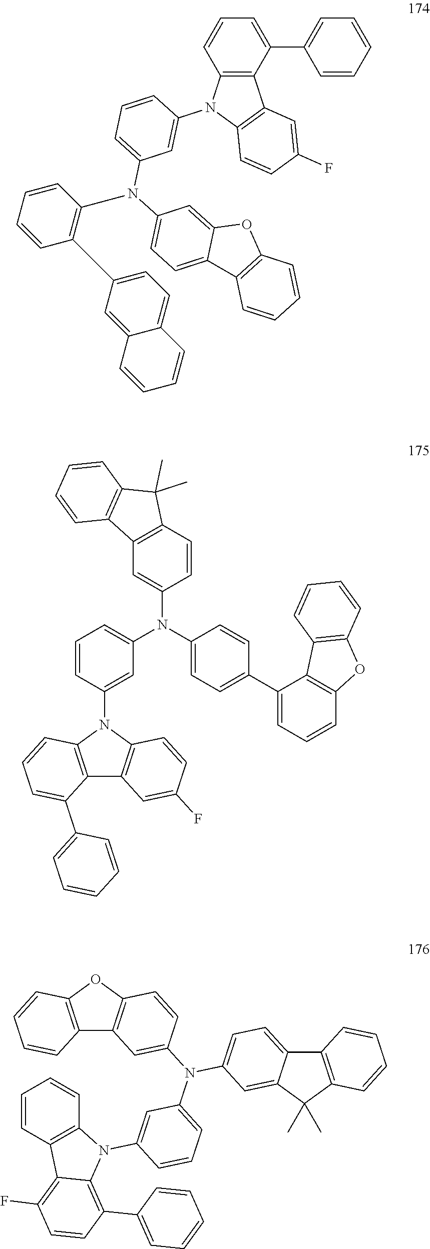 [SMILES: Fc1ccc2c(c1)c1c(-c3ccccc3)cccc1n2-c1cccc(N(c2ccc3c(c2)oc2ccccc23)c2ccccc2-c2ccc3ccccc3c2)c1 ; CC1(C)c2ccccc2-c2cc(N(c3ccc(-c4cccc5oc6ccccc6c45)cc3)c3cccc(-n4c5ccc(F)cc5c5c(-c6ccccc6)cccc54)c3)ccc21 ; CC1(C)c2ccccc2-c2ccc(N(c3cccc(-n4c5ccccc5c5c(F)ccc(-c6ccccc6)c54)c3)c3ccc4oc5ccccc5c4c3)cc21]

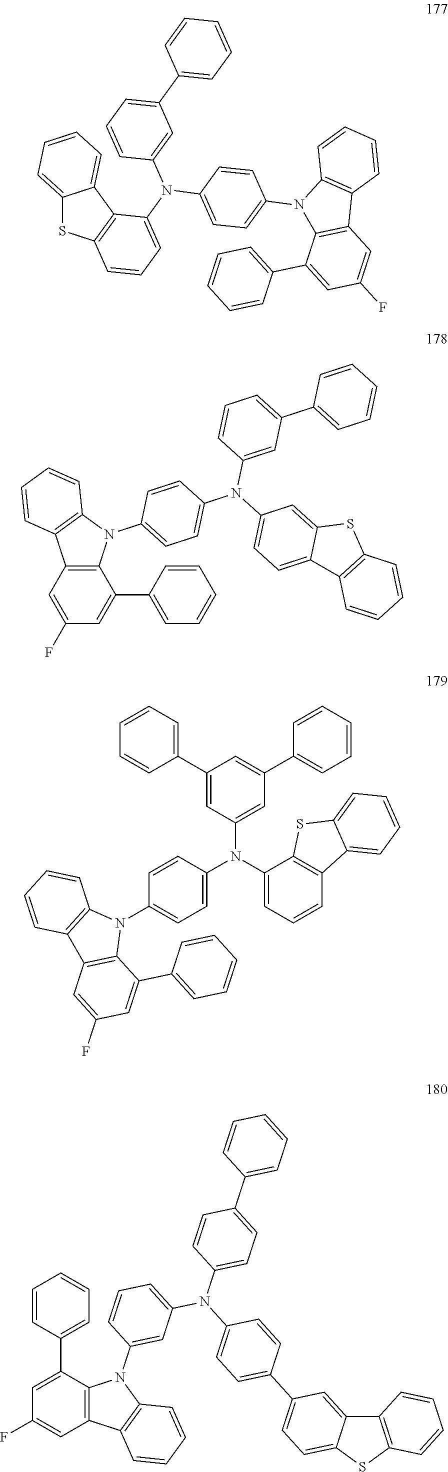 [SMILES: Fc1cc(-c2ccccc2)c2c(c1)c1ccccc1n2-c1ccc(N(c2cccc(-c3ccccc3)c2)c2cccc3sc4ccccc4c23)cc1 ; Fc1cc(-c2ccccc2)c2c(c1)c1ccccc1n2-c1ccc(N(c2cccc(-c3ccccc3)c2)c2ccc3c(c2)sc2ccccc23)cc1 ; Fc1cc(-c2ccccc2)c2c(c1)c1ccccc1n2-c1ccc(N(c2cc(-c3ccccc3)cc(-c3ccccc3)c2)c2cccc3c2sc2ccccc23)cc1 ; Fc1cc(-c2ccccc2)c2c(c1)c1ccccc1n2-c1cccc(N(c2ccc(-c3ccccc3)cc2)c2ccc(-c3ccc4sc5ccccc5c4c3)cc2)c1]

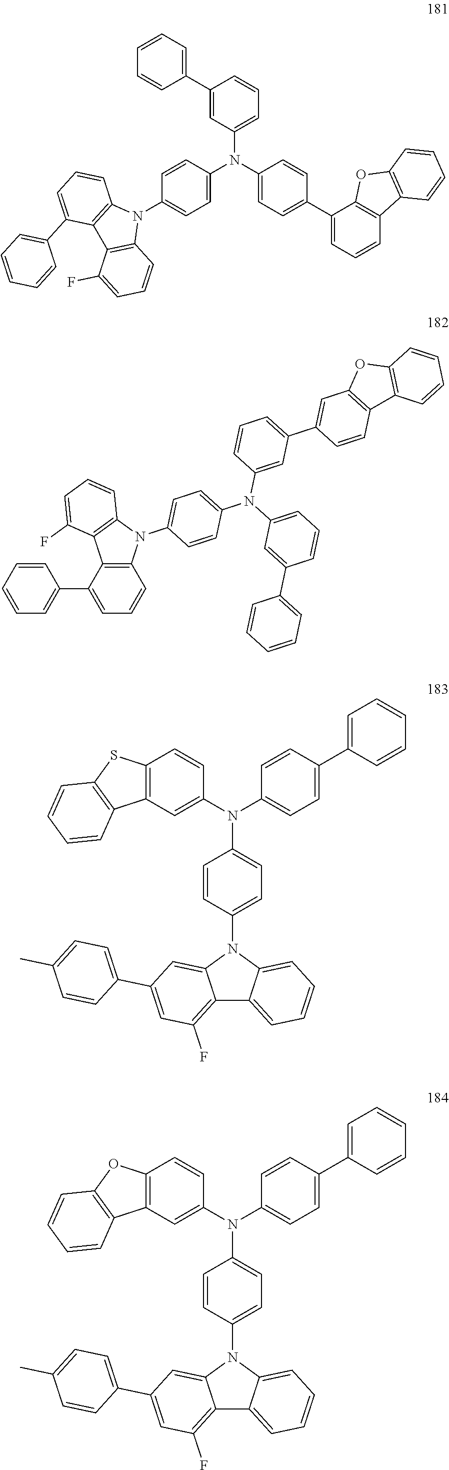 [SMILES: Fc1cccc2c1c1c(-c3ccccc3)cccc1n2-c1ccc(N(c2ccc(-c3cccc4c3oc3ccccc34)cc2)c2cccc(-c3ccccc3)c2)cc1 ; Fc1cccc2c1c1c(-c3ccccc3)cccc1n2-c1ccc(N(c2cccc(-c3ccccc3)c2)c2cccc(-c3ccc4c(c3)oc3ccccc34)c2)cc1 ; Cc1ccc(-c2cc(F)c3c4ccccc4n(-c4ccc(N(c5ccc(-c6ccccc6)cc5)c5ccc6sc7ccccc7c6c5)cc4)c3c2)cc1 ; Cc1ccc(-c2cc(F)c3c4ccccc4n(-c4ccc(N(c5ccc(-c6ccccc6)cc5)c5ccc6oc7ccccc7c6c5)cc4)c3c2)cc1]

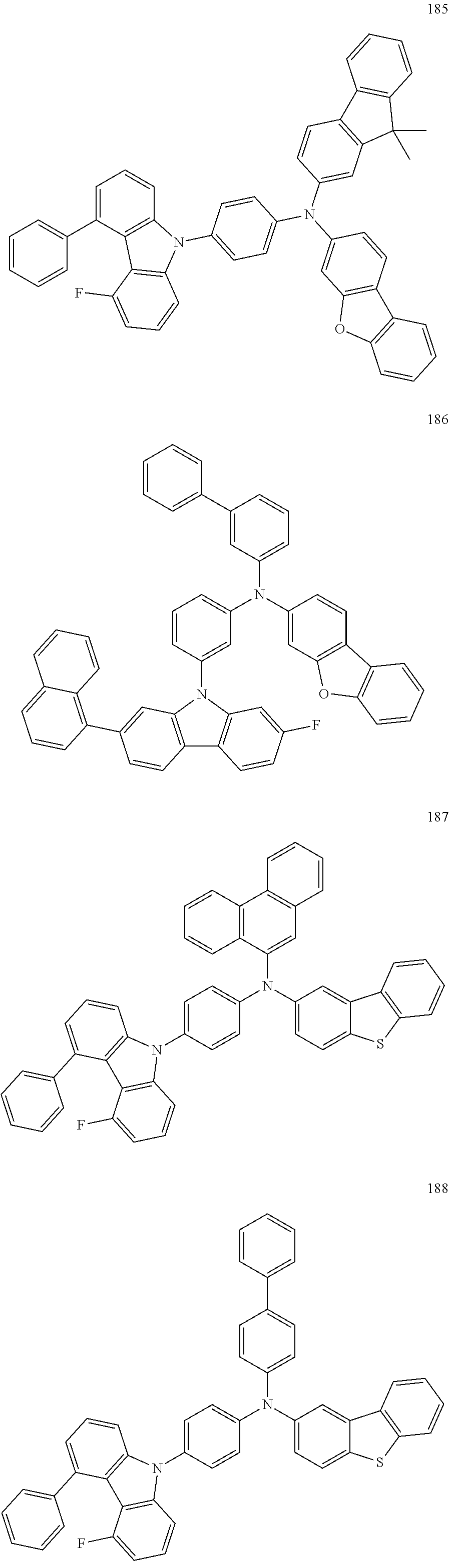 [SMILES: CC1(C)c2ccccc2-c2ccc(N(c3ccc(-n4c5cccc(F)c5c5c(-c6ccccc6)cccc54)cc3)c3ccc4c(c3)oc3ccccc34)cc21 ; Fc1ccc2c3ccc(-c4cccc5ccccc45)cc3n(-c3cccc(N(c4cccc(-c5ccccc5)c4)c4ccc5c(c4)oc4ccccc45)c3)c2c1 ; Fc1cccc2c1c1c(-c3ccccc3)cccc1n2-c1ccc(N(c2ccc3sc4ccccc4c3c2)c2cc3ccccc3c3ccccc23)cc1 ; Fc1cccc2c1c1c(-c3ccccc3)cccc1n2-c1ccc(N(c2ccc(-c3ccccc3)cc2)c2ccc3sc4ccccc4c3c2)cc1]

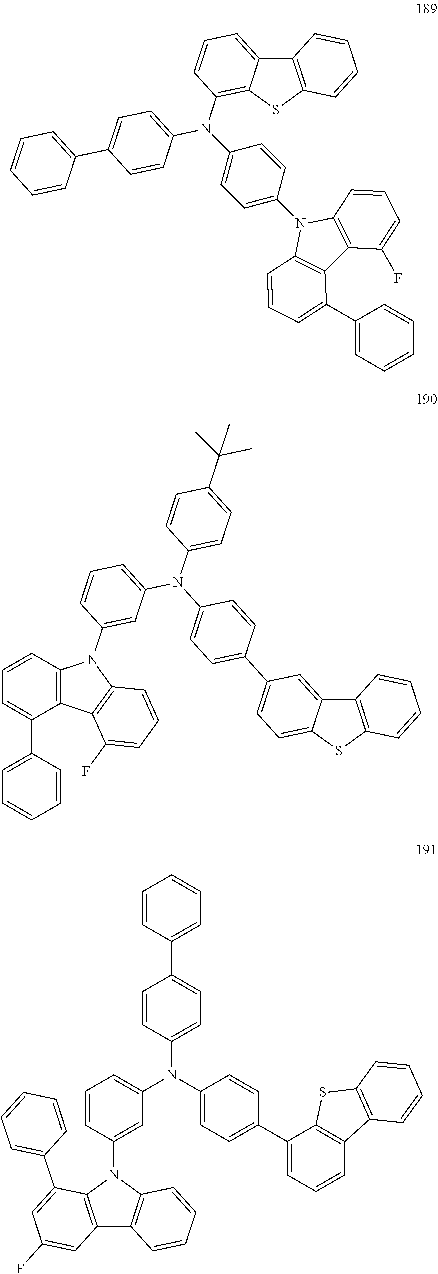 [SMILES: Fc1cccc2c1c1c(-c3ccccc3)cccc1n2-c1ccc(N(c2ccc(-c3ccccc3)cc2)c2cccc3c2sc2ccccc23)cc1 ; CC(C)(C)c1ccc(N(c2ccc(-c3ccc4sc5ccccc5c4c3)cc2)c2cccc(-n3c4cccc(F)c4c4c(-c5ccccc5)cccc43)c2)cc1 ; Fc1cc(-c2ccccc2)c2c(c1)c1ccccc1n2-c1cccc(N(c2ccc(-c3ccccc3)cc2)c2ccc(-c3cccc4c3sc3ccccc34)cc2)c1]

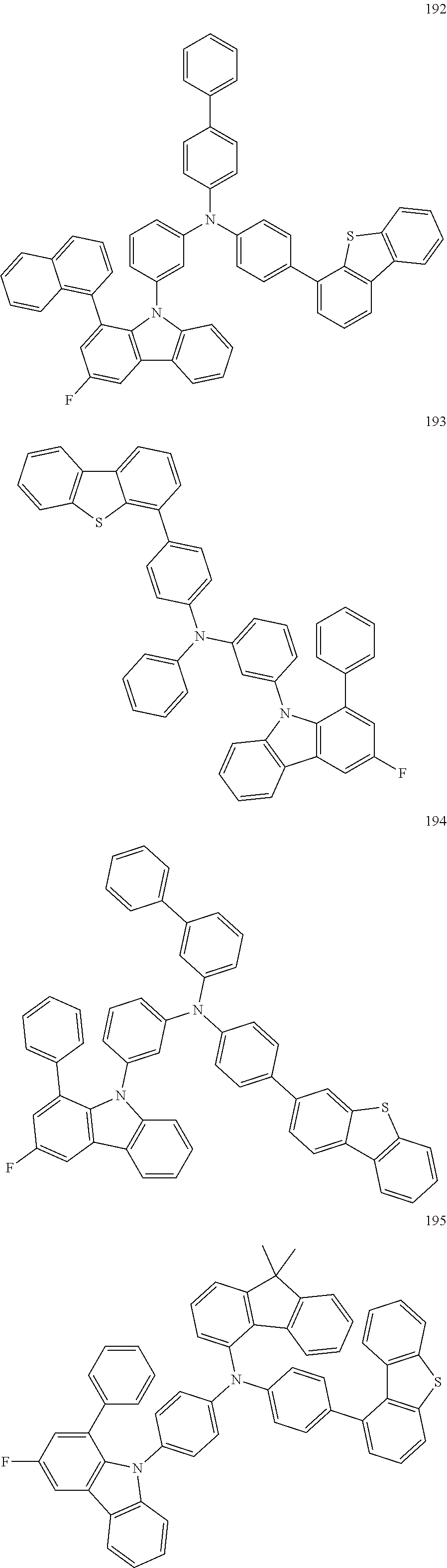 [SMILES: Fc1cc(-c2cccc3ccccc23)c2c(c1)c1ccccc1n2-c1cccc(N(c2ccc(-c3ccccc3)cc2)c2ccc(-c3cccc4c3sc3ccccc34)cc2)c1 ; Fc1cc(-c2ccccc2)c2c(c1)c1ccccc1n2-c1cccc(N(c2ccccc2)c2ccc(-c3cccc4c3sc3ccccc34)cc2)c1 ; Fc1cc(-c2ccccc2)c2c(c1)c1ccccc1n2-c1cccc(N(c2ccc(-c3ccc4c(c3)sc3ccccc34)cc2)c2cccc(-c3ccccc3)c2)c1 ; CC1(C)c2ccccc2-c2c(N(c3ccc(-c4cccc5sc6ccccc6c45)cc3)c3ccc(-n4c5ccccc5c5cc(F)cc(-c6ccccc6)c54)cc3)cccc21]

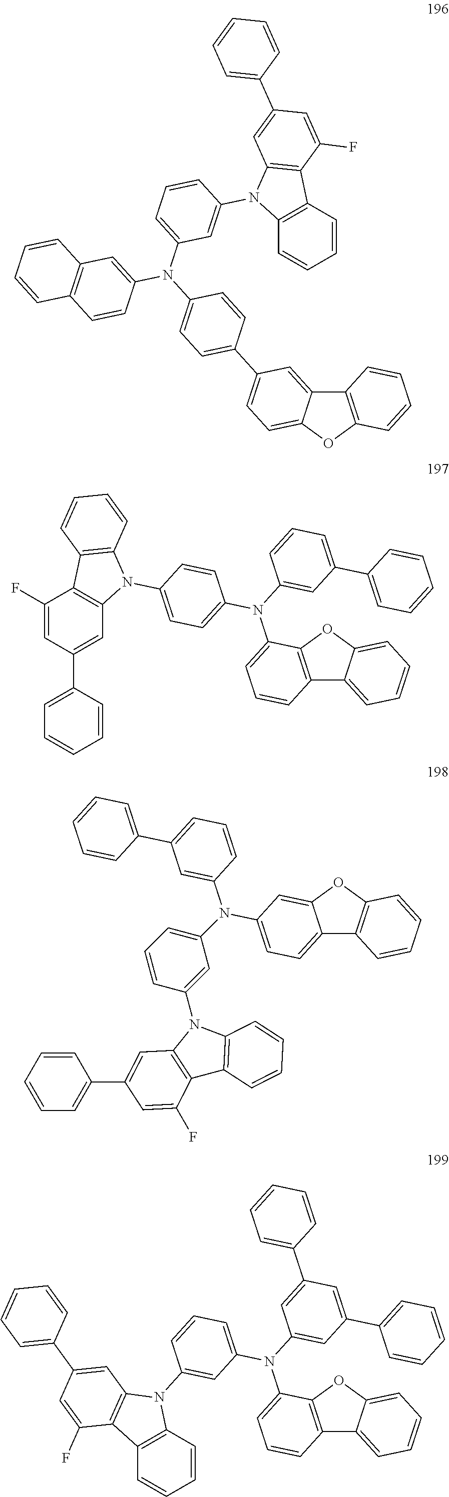 [SMILES: Fc1cc(-c2ccccc2)cc2c1c1ccccc1n2-c1cccc(N(c2ccc(-c3ccc4oc5ccccc5c4c3)cc2)c2ccc3ccccc3c2)c1 ; Fc1cc(-c2ccccc2)cc2c1c1ccccc1n2-c1ccc(N(c2cccc(-c3ccccc3)c2)c2cccc3c2oc2ccccc23)cc1 ; Fc1cc(-c2ccccc2)cc2c1c1ccccc1n2-c1cccc(N(c2cccc(-c3ccccc3)c2)c2ccc3c(c2)oc2ccccc23)c1 ; Fc1cc(-c2ccccc2)cc2c1c1ccccc1n2-c1cccc(N(c2cc(-c3ccccc3)cc(-c3ccccc3)c2)c2cccc3c2oc2ccccc23)c1]

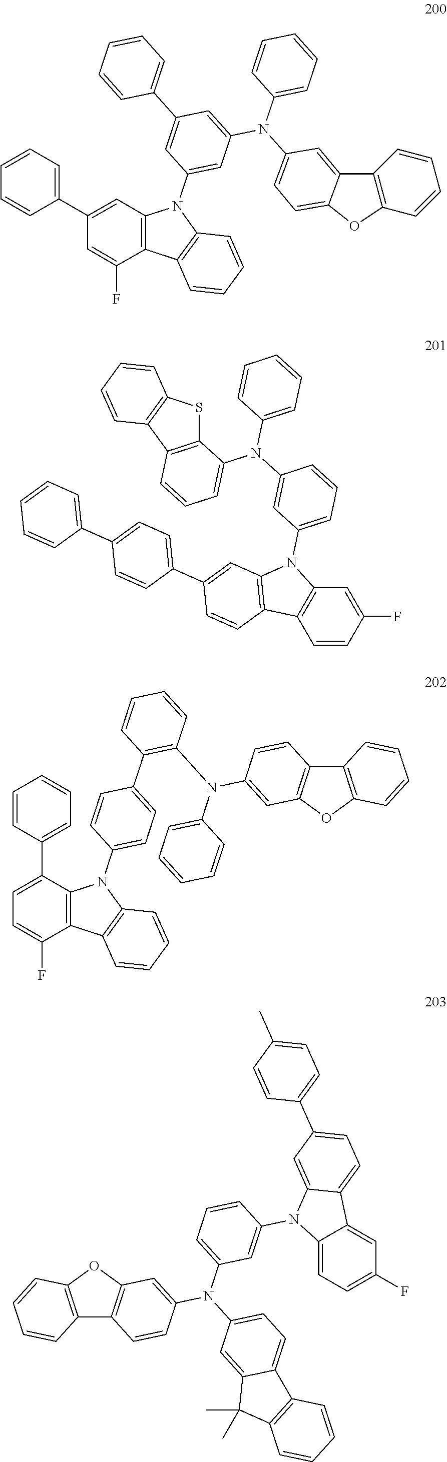 [SMILES: Fc1cc(-c2ccccc2)cc2c1c1ccccc1n2-c1cc(-c2ccccc2)cc(N(c2ccccc2)c2ccc3oc4ccccc4c3c2)c1 ; Fc1ccc2c3ccc(-c4ccc(-c5ccccc5)cc4)cc3n(-c3cccc(N(c4ccccc4)c4cccc5c4sc4ccccc45)c3)c2c1 ; Fc1ccc(-c2ccccc2)c2c1c1ccccc1n2-c1ccc(-c2ccccc2N(c2ccccc2)c2ccc3c(c2)oc2ccccc23)cc1 ; Cc1ccc(-c2ccc3c4cc(F)ccc4n(-c4cccc(N(c5ccc6c(c5)C(C)(C)c5ccccc5-6)c5ccc6c(c5)oc5ccccc56)c4)c3c2)cc1]

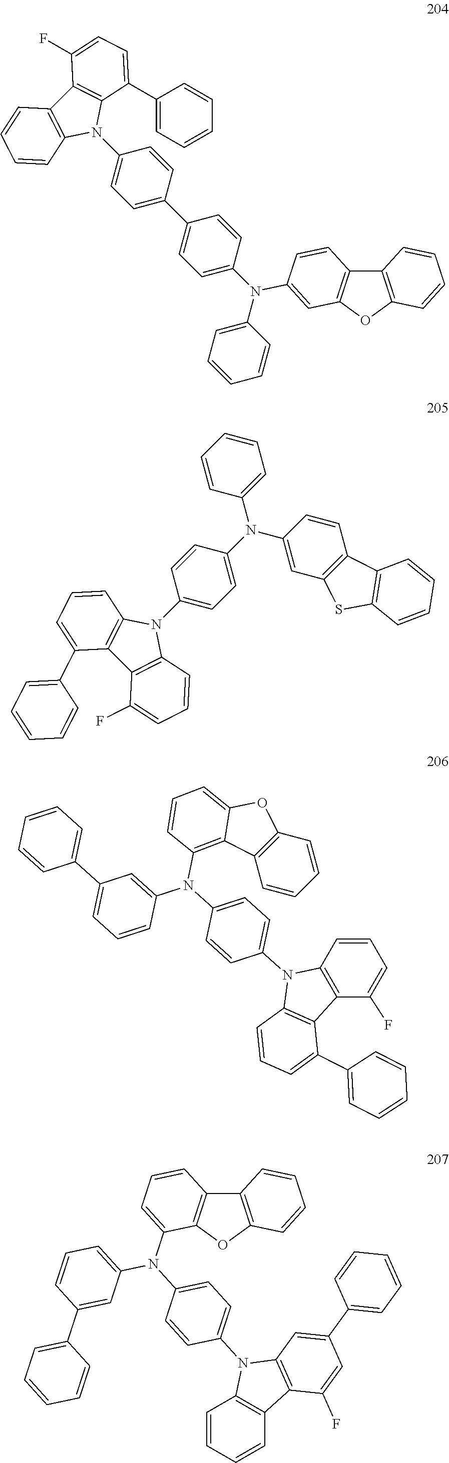 [SMILES: Fc1ccc(-c2ccccc2)c2c1c1ccccc1n2-c1ccc(-c2ccc(N(c3ccccc3)c3ccc4c(c3)oc3ccccc34)cc2)cc1 ; Fc1cccc2c1c1c(-c3ccccc3)cccc1n2-c1ccc(N(c2ccccc2)c2ccc3c(c2)sc2ccccc23)cc1 ; Fc1cccc2c1c1c(-c3ccccc3)cccc1n2-c1ccc(N(c2cccc(-c3ccccc3)c2)c2cccc3oc4ccccc4c23)cc1 ; Fc1cc(-c2ccccc2)cc2c1c1ccccc1n2-c1ccc(N(c2cccc(-c3ccccc3)c2)c2cccc3c2oc2ccccc23)cc1]

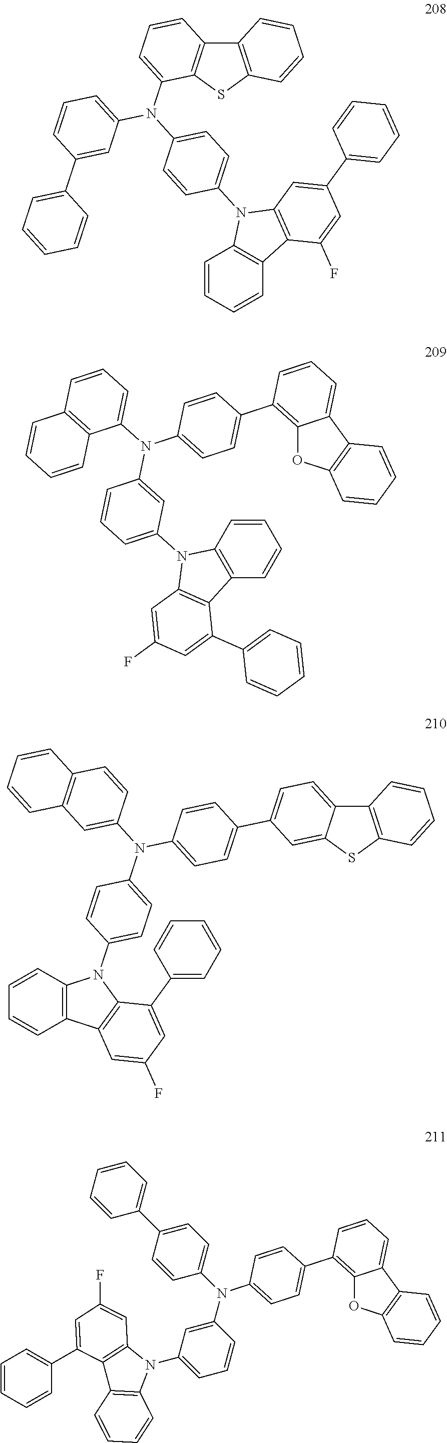 [SMILES: Fc1cc(-c2ccccc2)cc2c1c1ccccc1n2-c1ccc(N(c2cccc(-c3ccccc3)c2)c2cccc3c2sc2ccccc23)cc1 ; Fc1cc(-c2ccccc2)c2c3ccccc3n(-c3cccc(N(c4ccc(-c5cccc6c5oc5ccccc56)cc4)c4cccc5ccccc45)c3)c2c1 ; Fc1cc(-c2ccccc2)c2c(c1)c1ccccc1n2-c1ccc(N(c2ccc(-c3ccc4c(c3)sc3ccccc34)cc2)c2ccc3ccccc3c2)cc1 ; Fc1cc(-c2ccccc2)c2c3ccccc3n(-c3cccc(N(c4ccc(-c5ccccc5)cc4)c4ccc(-c5cccc6c5oc5ccccc56)cc4)c3)c2c1]

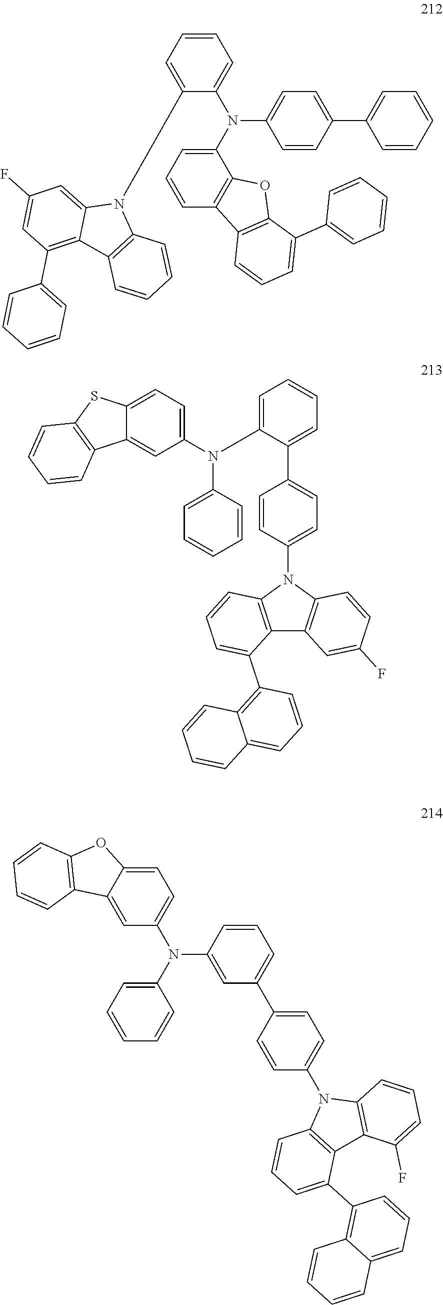 [SMILES: Fc1cc(-c2ccccc2)c2c3ccccc3n(-c3ccccc3N(c3ccc(-c4ccccc4)cc3)c3cccc4c3oc3c(-c5ccccc5)cccc34)c2c1 ; Fc1ccc2c(c1)c1c(-c3cccc4ccccc34)cccc1n2-c1ccc(-c2ccccc2N(c2ccccc2)c2ccc3sc4ccccc4c3c2)cc1 ; Fc1cccc2c1c1c(-c3cccc4ccccc34)cccc1n2-c1ccc(-c2cccc(N(c3ccccc3)c3ccc4oc5ccccc5c4c3)c2)cc1]

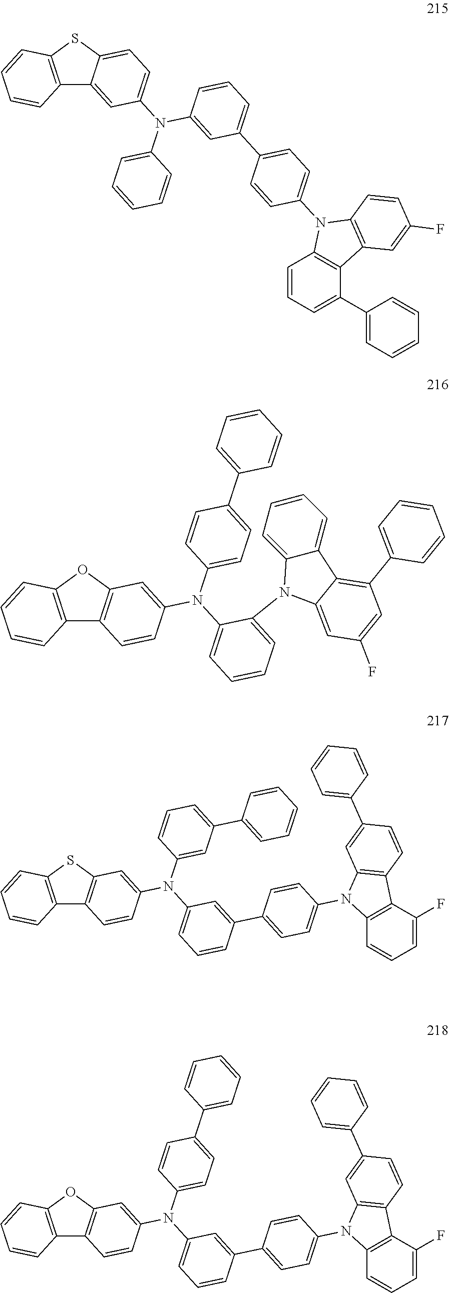 [SMILES: Fc1ccc2c(c1)c1c(-c3ccccc3)cccc1n2-c1ccc(-c2cccc(N(c3ccccc3)c3ccc4sc5ccccc5c4c3)c2)cc1 ; Fc1cc(-c2ccccc2)c2c3ccccc3n(-c3ccccc3N(c3ccc(-c4ccccc4)cc3)c3ccc4c(c3)oc3ccccc34)c2c1 ; Fc1cccc2c1c1ccc(-c3ccccc3)cc1n2-c1ccc(-c2cccc(N(c3cccc(-c4ccccc4)c3)c3ccc4c(c3)sc3ccccc34)c2)cc1 ; Fc1cccc2c1c1ccc(-c3ccccc3)cc1n2-c1ccc(-c2cccc(N(c3ccc(-c4ccccc4)cc3)c3ccc4c(c3)oc3ccccc34)c2)cc1]

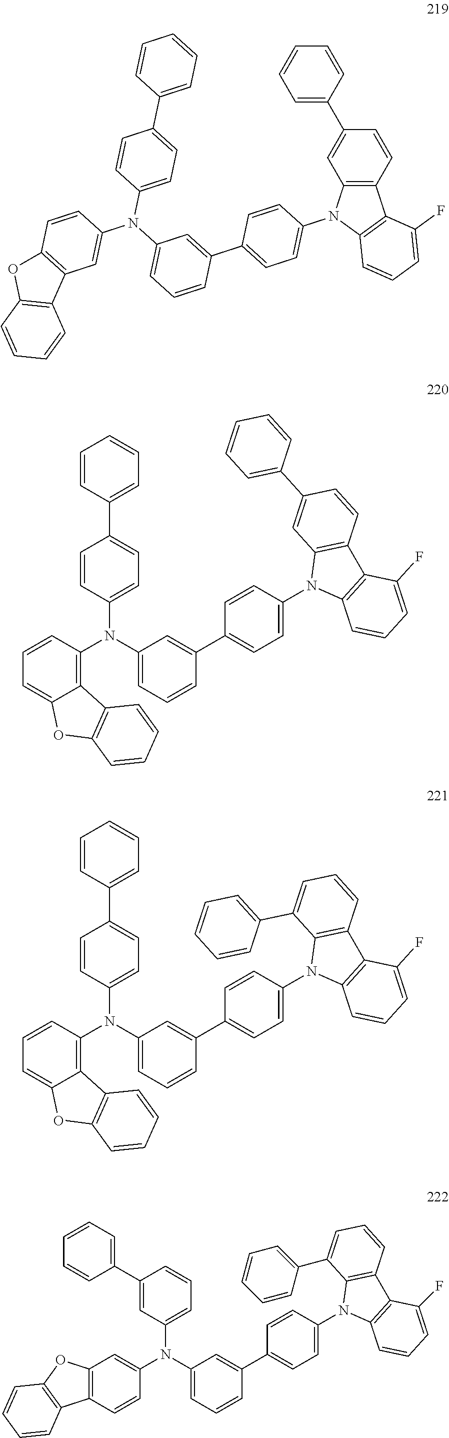 [SMILES: Fc1cccc2c1c1ccc(-c3ccccc3)cc1n2-c1ccc(-c2cccc(N(c3ccc(-c4ccccc4)cc3)c3ccc4oc5ccccc5c4c3)c2)cc1 ; Fc1cccc2c1c1ccc(-c3ccccc3)cc1n2-c1ccc(-c2cccc(N(c3ccc(-c4ccccc4)cc3)c3cccc4oc5ccccc5c34)c2)cc1 ; Fc1cccc2c1c1cccc(-c3ccccc3)c1n2-c1ccc(-c2cccc(N(c3ccc(-c4ccccc4)cc3)c3cccc4oc5ccccc5c34)c2)cc1 ; Fc1cccc2c1c1cccc(-c3ccccc3)c1n2-c1ccc(-c2cccc(N(c3cccc(-c4ccccc4)c3)c3ccc4c(c3)oc3ccccc34)c2)cc1]

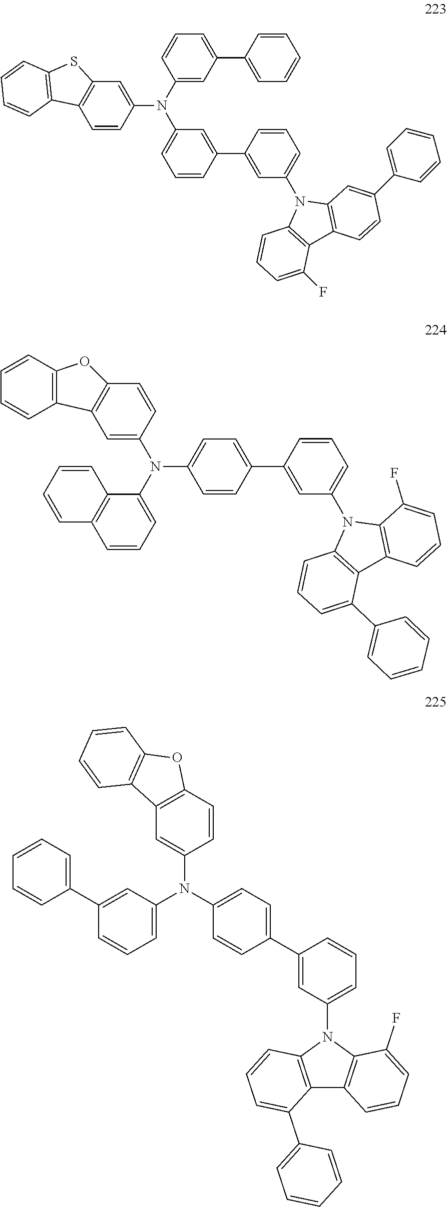 [SMILES: Fc1cccc2c1c1ccc(-c3ccccc3)cc1n2-c1cccc(-c2cccc(N(c3cccc(-c4ccccc4)c3)c3ccc4c(c3)sc3ccccc34)c2)c1 ; Fc1cccc2c3c(-c4ccccc4)cccc3n(-c3cccc(-c4ccc(N(c5ccc6oc7ccccc7c6c5)c5cccc6ccccc56)cc4)c3)c12 ; Fc1cccc2c3c(-c4ccccc4)cccc3n(-c3cccc(-c4ccc(N(c5cccc(-c6ccccc6)c5)c5ccc6oc7ccccc7c6c5)cc4)c3)c12]

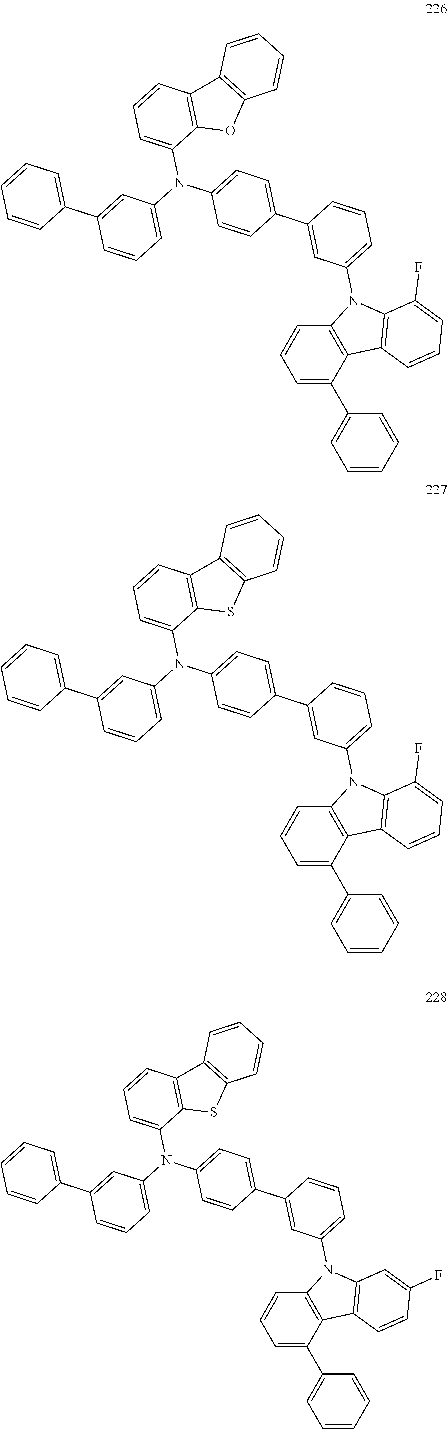 [SMILES: Fc1cccc2c3c(-c4ccccc4)cccc3n(-c3cccc(-c4ccc(N(c5cccc(-c6ccccc6)c5)c5cccc6c5oc5ccccc56)cc4)c3)c12 ; Fc1cccc2c3c(-c4ccccc4)cccc3n(-c3cccc(-c4ccc(N(c5cccc(-c6ccccc6)c5)c5cccc6c5sc5ccccc56)cc4)c3)c12 ; Fc1ccc2c3c(-c4ccccc4)cccc3n(-c3cccc(-c4ccc(N(c5cccc(-c6ccccc6)c5)c5cccc6c5sc5ccccc56)cc4)c3)c2c1]

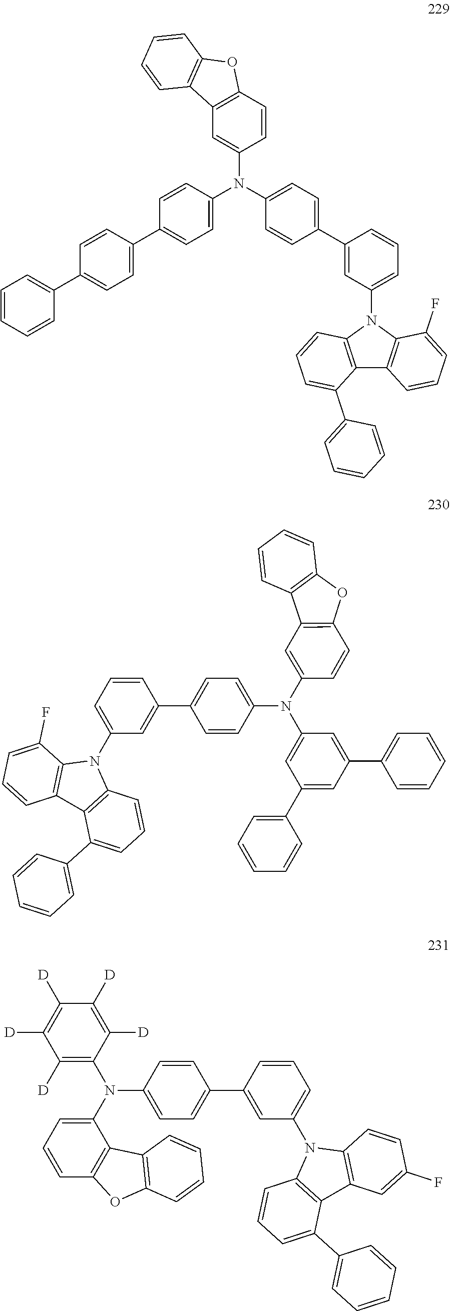 [SMILES: Fc1cccc2c3c(-c4ccccc4)cccc3n(-c3cccc(-c4ccc(N(c5ccc(-c6ccc(-c7ccccc7)cc6)cc5)c5ccc6oc7ccccc7c6c5)cc4)c3)c12 ; Fc1cccc2c3c(-c4ccccc4)cccc3n(-c3cccc(-c4ccc(N(c5cc(-c6ccccc6)cc(-c6ccccc6)c5)c5ccc6oc7ccccc7c6c5)cc4)c3)c12 ; [2H]c1c([2H])c([2H])c(N(c2ccc(-c3cccc(-n4c5ccc(F)cc5c5c(-c6ccccc6)cccc54)c3)cc2)c2cccc3oc4ccccc4c23)c([2H])c1[2H]]

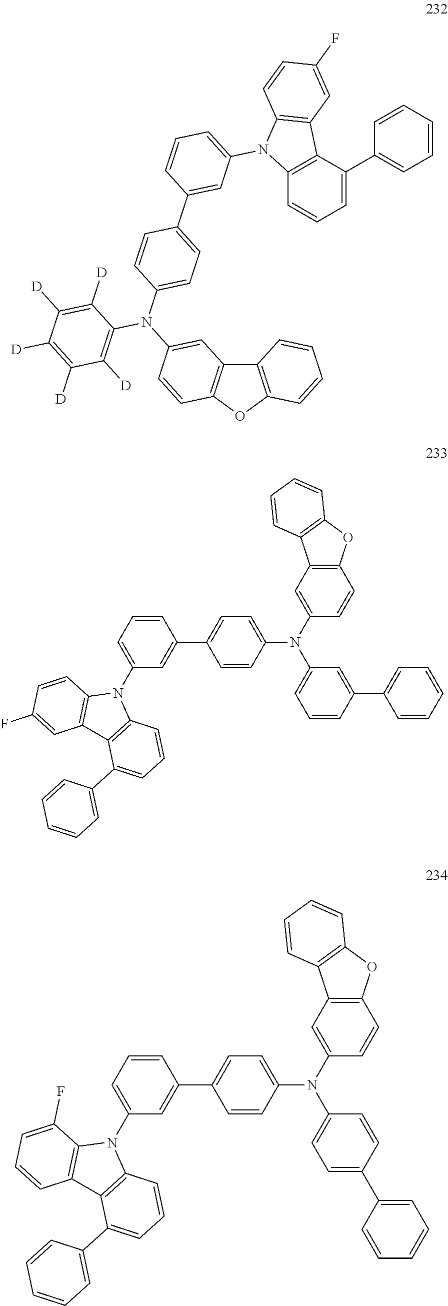 [SMILES: [2H]c1c([2H])c([2H])c(N(c2ccc(-c3cccc(-n4c5ccc(F)cc5c5c(-c6ccccc6)cccc54)c3)cc2)c2ccc3oc4ccccc4c3c2)c([2H])c1[2H] ; Fc1ccc2c(c1)c1c(-c3ccccc3)cccc1n2-c1cccc(-c2ccc(N(c3cccc(-c4ccccc4)c3)c3ccc4oc5ccccc5c4c3)cc2)c1 ; Fc1cccc2c3c(-c4ccccc4)cccc3n(-c3cccc(-c4ccc(N(c5ccc(-c6ccccc6)cc5)c5ccc6oc7ccccc7c6c5)cc4)c3)c12]

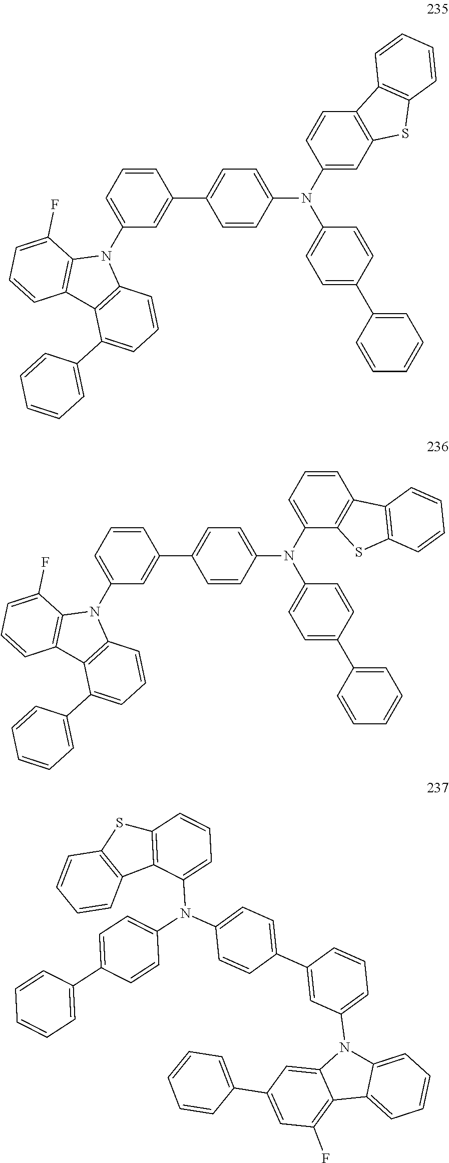 [SMILES: Fc1cccc2c3c(-c4ccccc4)cccc3n(-c3cccc(-c4ccc(N(c5ccc(-c6ccccc6)cc5)c5ccc6c(c5)sc5ccccc56)cc4)c3)c12 ; Fc1cccc2c3c(-c4ccccc4)cccc3n(-c3cccc(-c4ccc(N(c5ccc(-c6ccccc6)cc5)c5cccc6c5sc5ccccc56)cc4)c3)c12 ; Fc1cc(-c2ccccc2)cc2c1c1ccccc1n2-c1cccc(-c2ccc(N(c3ccc(-c4ccccc4)cc3)c3cccc4sc5ccccc5c34)cc2)c1]

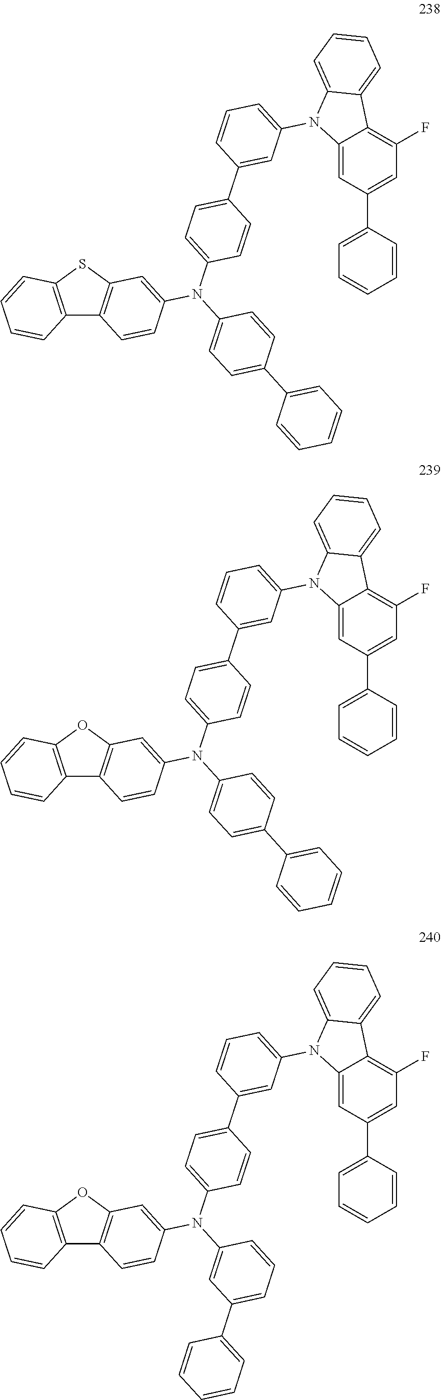 [SMILES: Fc1cc(-c2ccccc2)cc2c1c1ccccc1n2-c1cccc(-c2ccc(N(c3ccc(-c4ccccc4)cc3)c3ccc4c(c3)sc3ccccc34)cc2)c1 ; Fc1cc(-c2ccccc2)cc2c1c1ccccc1n2-c1cccc(-c2ccc(N(c3ccc(-c4ccccc4)cc3)c3ccc4c(c3)oc3ccccc34)cc2)c1 ; Fc1cc(-c2ccccc2)cc2c1c1ccccc1n2-c1cccc(-c2ccc(N(c3cccc(-c4ccccc4)c3)c3ccc4c(c3)oc3ccccc34)cc2)c1]

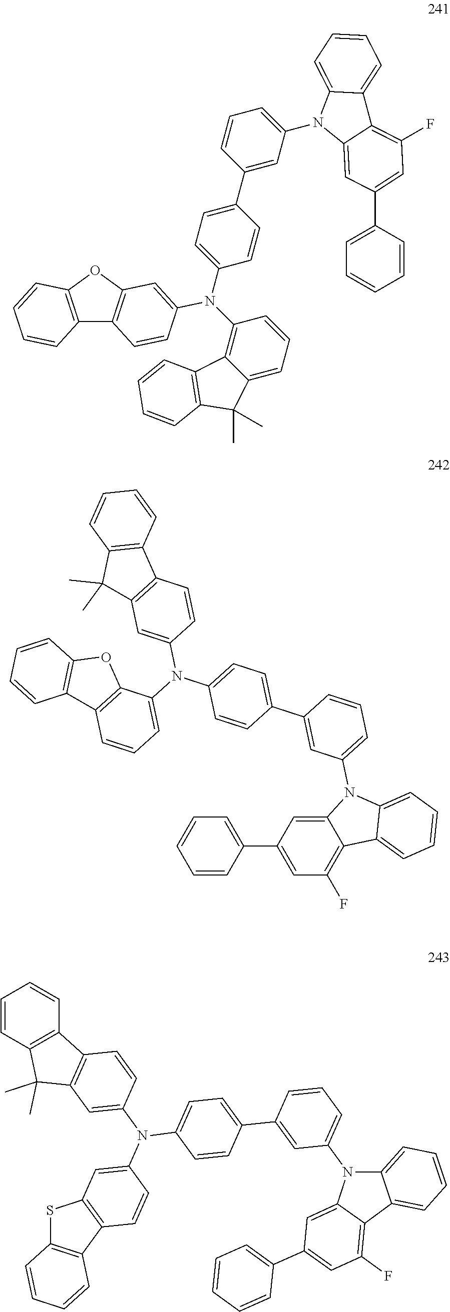 [SMILES: CC1(C)c2ccccc2-c2c(N(c3ccc(-c4cccc(-n5c6ccccc6c6c(F)cc(-c7ccccc7)cc65)c4)cc3)c3ccc4c(c3)oc3ccccc34)cccc21 ; CC1(C)c2ccccc2-c2ccc(N(c3ccc(-c4cccc(-n5c6ccccc6c6c(F)cc(-c7ccccc7)cc65)c4)cc3)c3cccc4c3oc3ccccc34)cc21 ; CC1(C)c2ccccc2-c2ccc(N(c3ccc(-c4cccc(-n5c6ccccc6c6c(F)cc(-c7ccccc7)cc65)c4)cc3)c3ccc4c(c3)sc3ccccc34)cc21]

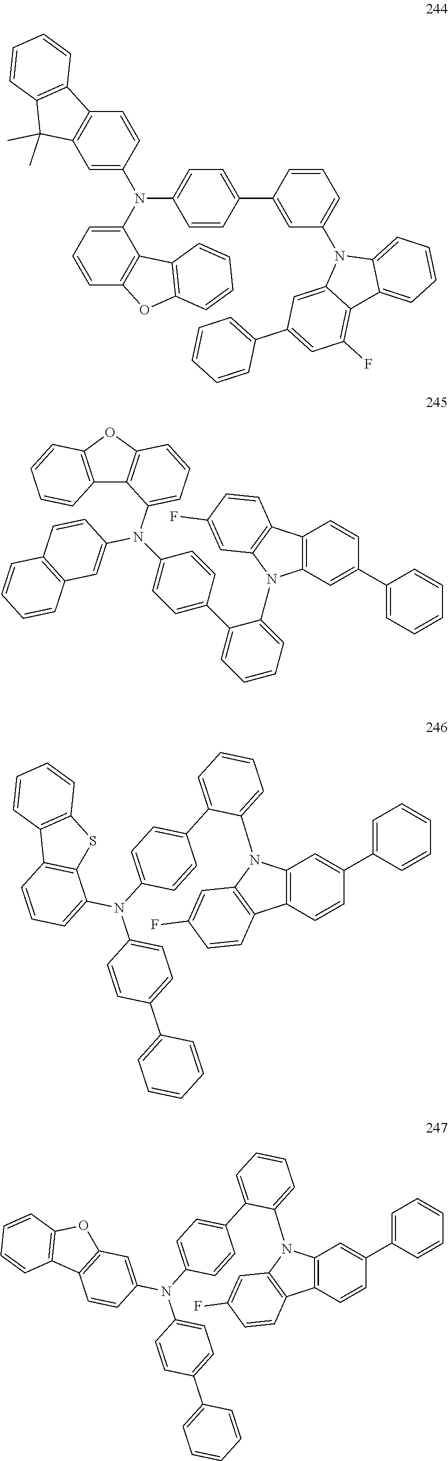 [SMILES: CC1(C)c2ccccc2-c2ccc(N(c3ccc(-c4cccc(-n5c6ccccc6c6c(F)cc(-c7ccccc7)cc65)c4)cc3)c3cccc4oc5ccccc5c34)cc21 ; Fc1ccc2c3ccc(-c4ccccc4)cc3n(-c3ccccc3-c3ccc(N(c4ccc5ccccc5c4)c4cccc5oc6ccccc6c45)cc3)c2c1 ; Fc1ccc2c3ccc(-c4ccccc4)cc3n(-c3ccccc3-c3ccc(N(c4ccc(-c5ccccc5)cc4)c4cccc5c4sc4ccccc45)cc3)c2c1 ; Fc1ccc2c3ccc(-c4ccccc4)cc3n(-c3ccccc3-c3ccc(N(c4ccc(-c5ccccc5)cc4)c4ccc5c(c4)oc4ccccc45)cc3)c2c1]

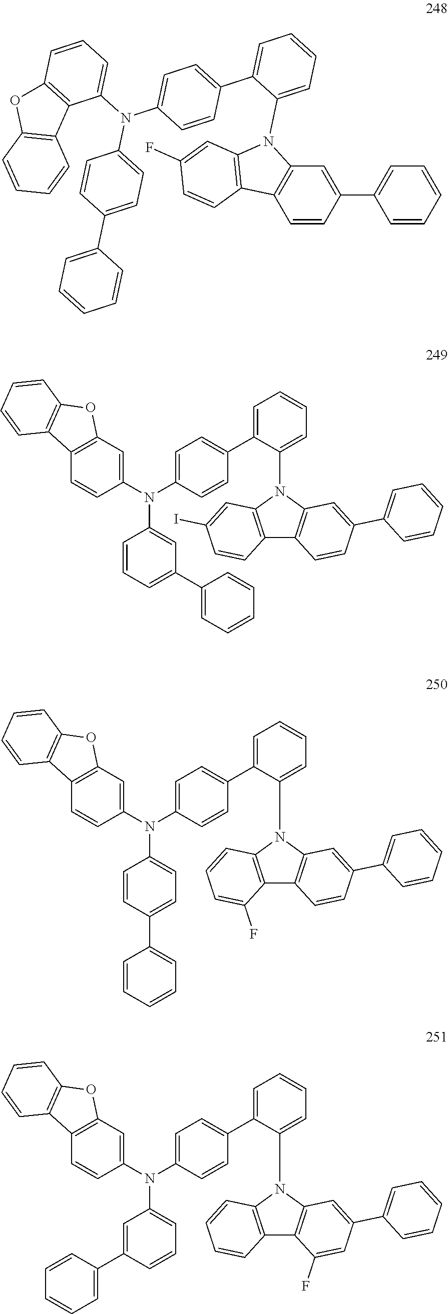 [SMILES: Fc1ccc2c3ccc(-c4ccccc4)cc3n(-c3ccccc3-c3ccc(N(c4ccc(-c5ccccc5)cc4)c4cccc5oc6ccccc6c45)cc3)c2c1 ; Ic1ccc2c3ccc(-c4ccccc4)cc3n(-c3ccccc3-c3ccc(N(c4cccc(-c5ccccc5)c4)c4ccc5c(c4)oc4ccccc45)cc3)c2c1 ; Fc1cccc2c1c1ccc(-c3ccccc3)cc1n2-c1ccccc1-c1ccc(N(c2ccc(-c3ccccc3)cc2)c2ccc3c(c2)oc2ccccc23)cc1 ; Fc1cc(-c2ccccc2)cc2c1c1ccccc1n2-c1ccccc1-c1ccc(N(c2cccc(-c3ccccc3)c2)c2ccc3c(c2)oc2ccccc23)cc1]

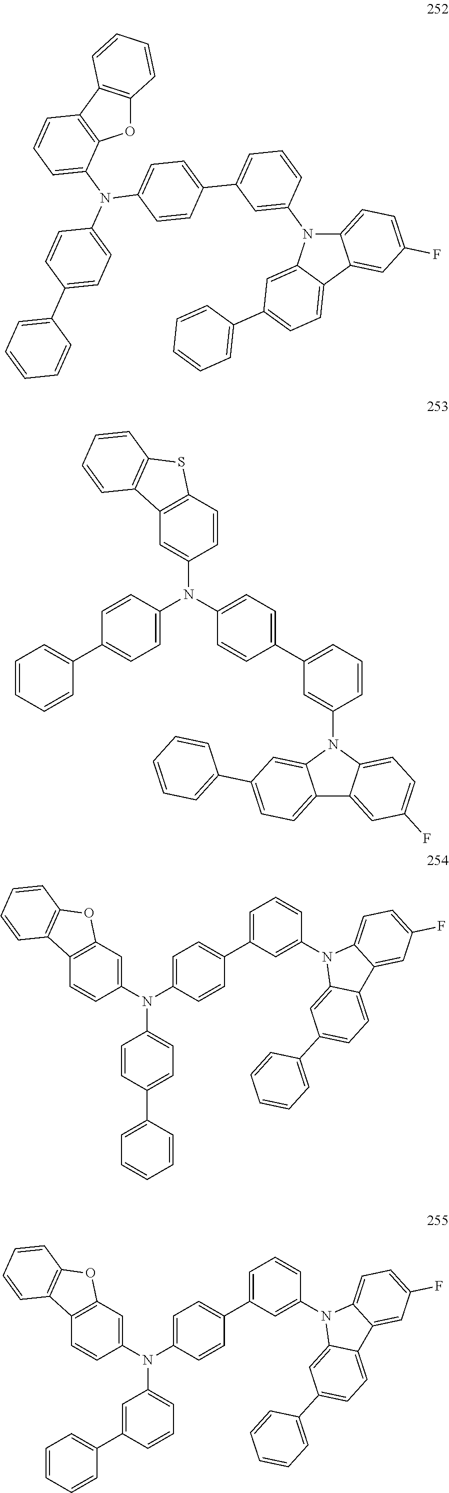 [SMILES: Fc1ccc2c(c1)c1ccc(-c3ccccc3)cc1n2-c1cccc(-c2ccc(N(c3ccc(-c4ccccc4)cc3)c3cccc4c3oc3ccccc34)cc2)c1 ; Fc1ccc2c(c1)c1ccc(-c3ccccc3)cc1n2-c1cccc(-c2ccc(N(c3ccc(-c4ccccc4)cc3)c3ccc4sc5ccccc5c4c3)cc2)c1 ; Fc1ccc2c(c1)c1ccc(-c3ccccc3)cc1n2-c1cccc(-c2ccc(N(c3ccc(-c4ccccc4)cc3)c3ccc4c(c3)oc3ccccc34)cc2)c1 ; Fc1ccc2c(c1)c1ccc(-c3ccccc3)cc1n2-c1cccc(-c2ccc(N(c3cccc(-c4ccccc4)c3)c3ccc4c(c3)oc3ccccc34)cc2)c1]

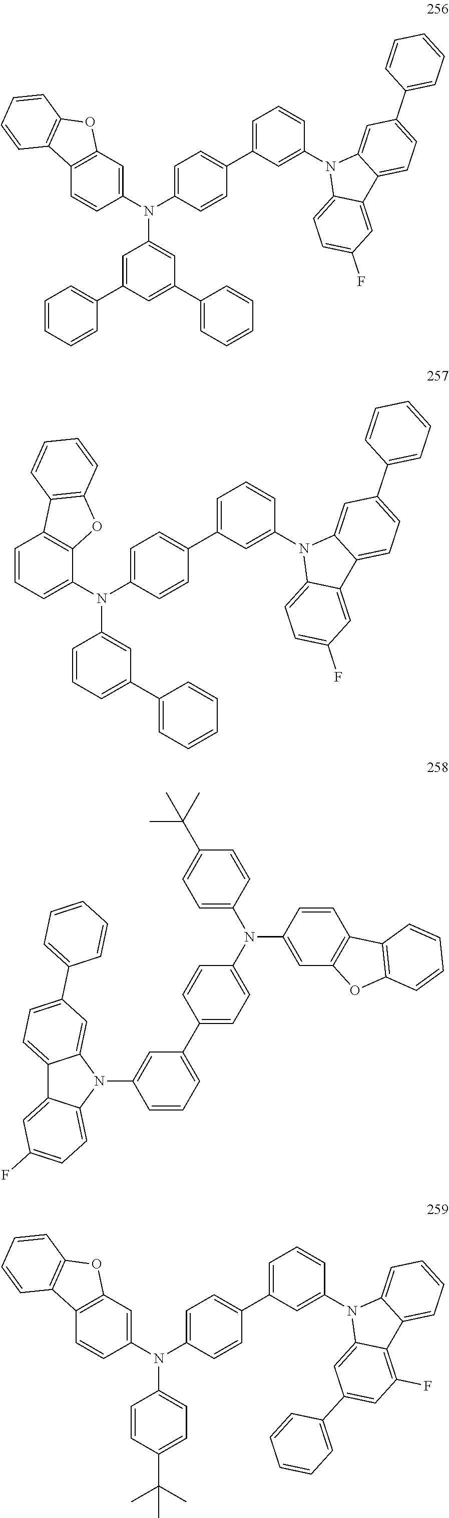 [SMILES: Fc1ccc2c(c1)c1ccc(-c3ccccc3)cc1n2-c1cccc(-c2ccc(N(c3cc(-c4ccccc4)cc(-c4ccccc4)c3)c3ccc4c(c3)oc3ccccc34)cc2)c1 ; Fc1ccc2c(c1)c1ccc(-c3ccccc3)cc1n2-c1cccc(-c2ccc(N(c3cccc(-c4ccccc4)c3)c3cccc4c3oc3ccccc34)cc2)c1 ; CC(C)(C)c1ccc(N(c2ccc(-c3cccc(-n4c5ccc(F)cc5c5ccc(-c6ccccc6)cc54)c3)cc2)c2ccc3c(c2)oc2ccccc23)cc1 ; CC(C)(C)c1ccc(N(c2ccc(-c3cccc(-n4c5ccccc5c5c(F)cc(-c6ccccc6)cc54)c3)cc2)c2ccc3c(c2)oc2ccccc23)cc1]

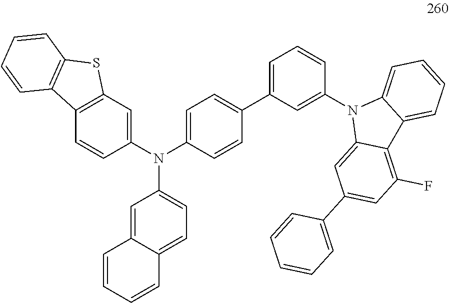 [SMILES: Fc1cc(-c2ccccc2)cc2c1c1ccccc1n2-c1cccc(-c2ccc(N(c3ccc4ccccc4c3)c3ccc4c(c3)sc3ccccc34)cc2)c1]

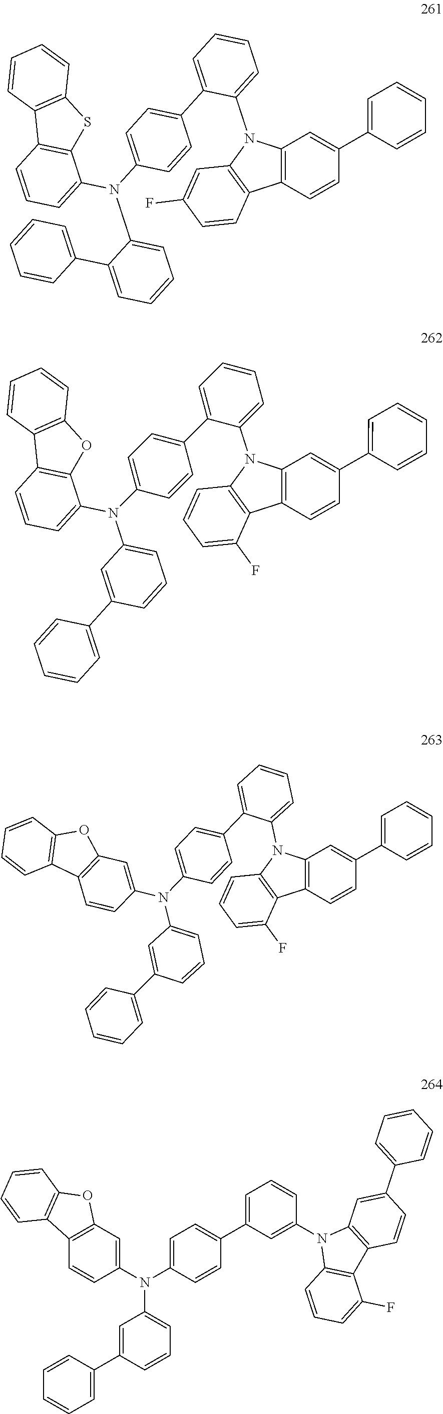 [SMILES: Fc1ccc2c3ccc(-c4ccccc4)cc3n(-c3ccccc3-c3ccc(N(c4ccccc4-c4ccccc4)c4cccc5c4sc4ccccc45)cc3)c2c1 ; Fc1cccc2c1c1ccc(-c3ccccc3)cc1n2-c1ccccc1-c1ccc(N(c2cccc(-c3ccccc3)c2)c2cccc3c2oc2ccccc23)cc1 ; Fc1cccc2c1c1ccc(-c3ccccc3)cc1n2-c1ccccc1-c1ccc(N(c2cccc(-c3ccccc3)c2)c2ccc3c(c2)oc2ccccc23)cc1 ; Fc1cccc2c1c1ccc(-c3ccccc3)cc1n2-c1cccc(-c2ccc(N(c3cccc(-c4ccccc4)c3)c3ccc4c(c3)oc3ccccc34)cc2)c1]

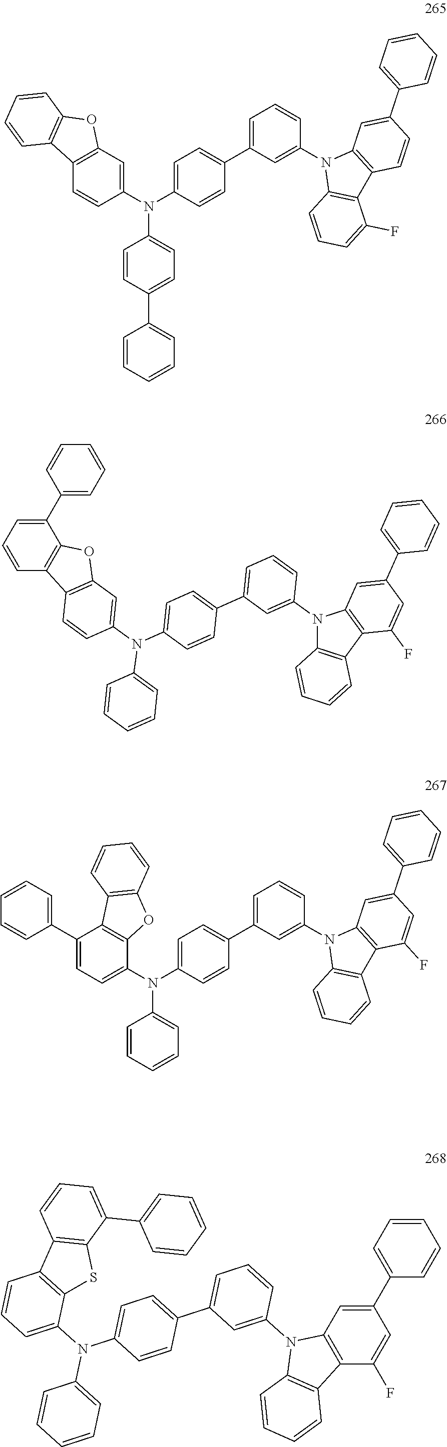 [SMILES: Fc1cccc2c1c1ccc(-c3ccccc3)cc1n2-c1cccc(-c2ccc(N(c3ccc(-c4ccccc4)cc3)c3ccc4c(c3)oc3ccccc34)cc2)c1 ; Fc1cc(-c2ccccc2)cc2c1c1ccccc1n2-c1cccc(-c2ccc(N(c3ccccc3)c3ccc4c(c3)oc3c(-c5ccccc5)cccc34)cc2)c1 ; Fc1cc(-c2ccccc2)cc2c1c1ccccc1n2-c1cccc(-c2ccc(N(c3ccccc3)c3ccc(-c4ccccc4)c4c3oc3ccccc34)cc2)c1 ; Fc1cc(-c2ccccc2)cc2c1c1ccccc1n2-c1cccc(-c2ccc(N(c3ccccc3)c3cccc4c3sc3c(-c5ccccc5)cccc34)cc2)c1]

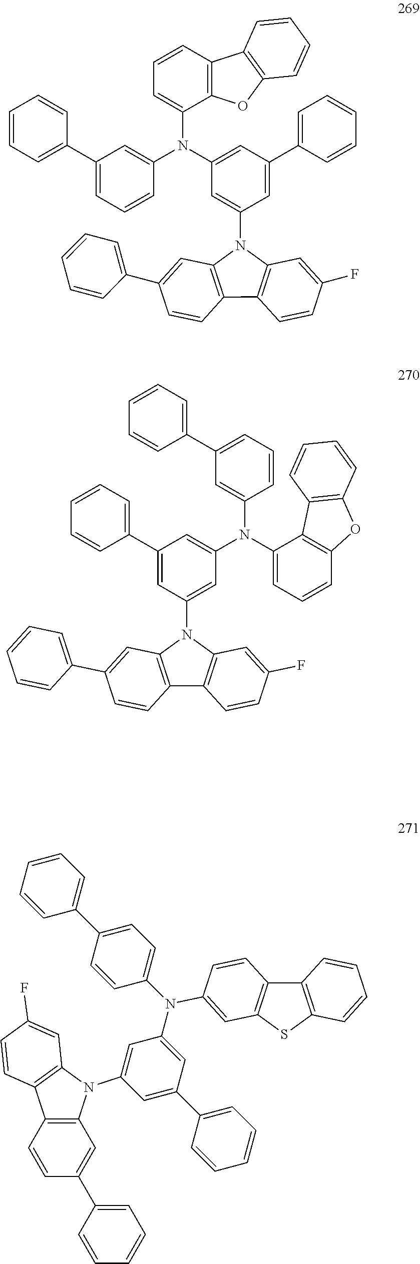 [SMILES: Fc1ccc2c3ccc(-c4ccccc4)cc3n(-c3cc(-c4ccccc4)cc(N(c4cccc(-c5ccccc5)c4)c4cccc5c4oc4ccccc45)c3)c2c1 ; Fc1ccc2c3ccc(-c4ccccc4)cc3n(-c3cc(-c4ccccc4)cc(N(c4cccc(-c5ccccc5)c4)c4cccc5oc6ccccc6c45)c3)c2c1 ; Fc1ccc2c3ccc(-c4ccccc4)cc3n(-c3cc(-c4ccccc4)cc(N(c4ccc(-c5ccccc5)cc4)c4ccc5c(c4)sc4ccccc45)c3)c2c1]

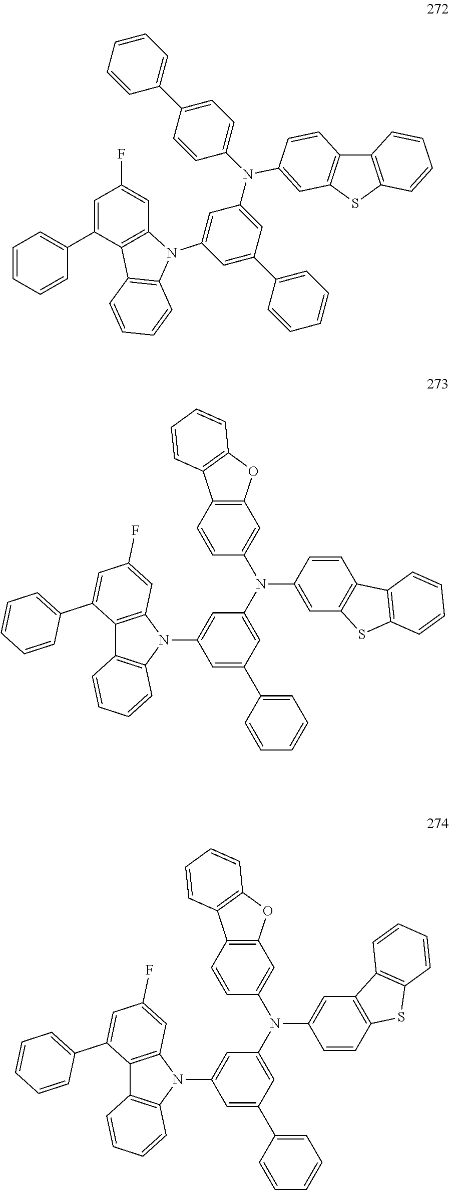 [SMILES: Fc1cc(-c2ccccc2)c2c3ccccc3n(-c3cc(-c4ccccc4)cc(N(c4ccc(-c5ccccc5)cc4)c4ccc5c(c4)sc4ccccc45)c3)c2c1 ; Fc1cc(-c2ccccc2)c2c3ccccc3n(-c3cc(-c4ccccc4)cc(N(c4ccc5c(c4)oc4ccccc45)c4ccc5c(c4)sc4ccccc45)c3)c2c1 ; Fc1cc(-c2ccccc2)c2c3ccccc3n(-c3cc(-c4ccccc4)cc(N(c4ccc5c(c4)oc4ccccc45)c4ccc5sc6ccccc6c5c4)c3)c2c1]

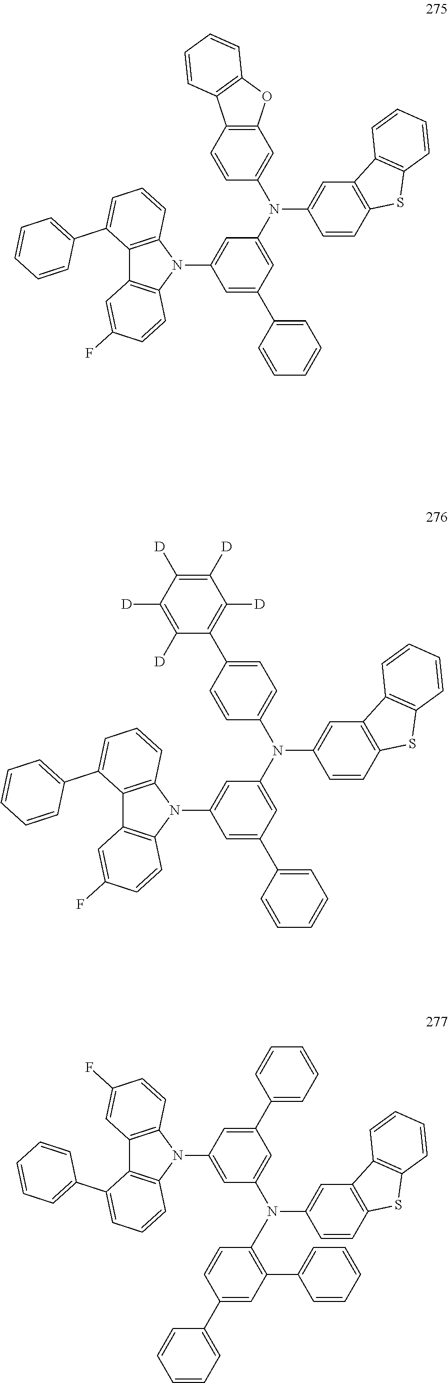 [SMILES: Fc1ccc2c(c1)c1c(-c3ccccc3)cccc1n2-c1cc(-c2ccccc2)cc(N(c2ccc3c(c2)oc2ccccc23)c2ccc3sc4ccccc4c3c2)c1 ; [2H]c1c([2H])c([2H])c(-c2ccc(N(c3cc(-c4ccccc4)cc(-n4c5ccc(F)cc5c5c(-c6ccccc6)cccc54)c3)c3ccc4sc5ccccc5c4c3)cc2)c([2H])c1[2H] ; Fc1ccc2c(c1)c1c(-c3ccccc3)cccc1n2-c1cc(-c2ccccc2)cc(N(c2ccc3sc4ccccc4c3c2)c2ccc(-c3ccccc3)cc2-c2ccccc2)c1]

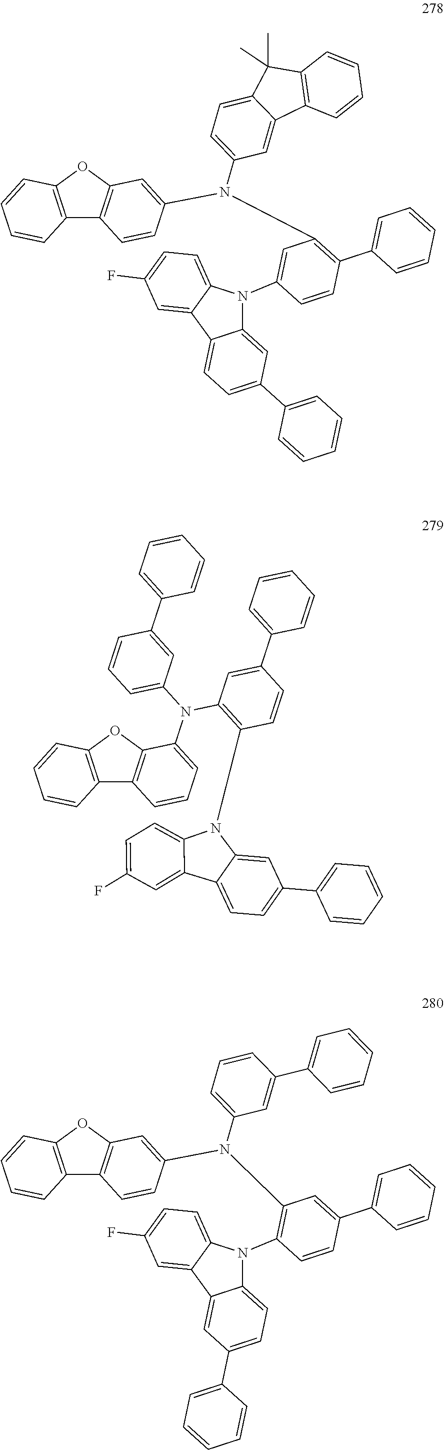 [SMILES: CC1(C)c2ccccc2-c2cc(N(c3ccc4c(c3)oc3ccccc34)c3cc(-n4c5ccc(F)cc5c5ccc(-c6ccccc6)cc54)ccc3-c3ccccc3)ccc21 ; Fc1ccc2c(c1)c1ccc(-c3ccccc3)cc1n2-c1ccc(-c2ccccc2)cc1N(c1cccc(-c2ccccc2)c1)c1cccc2c1oc1ccccc12 ; Fc1ccc2c(c1)c1cc(-c3ccccc3)ccc1n2-c1ccc(-c2ccccc2)cc1N(c1cccc(-c2ccccc2)c1)c1ccc2c(c1)oc1ccccc12]

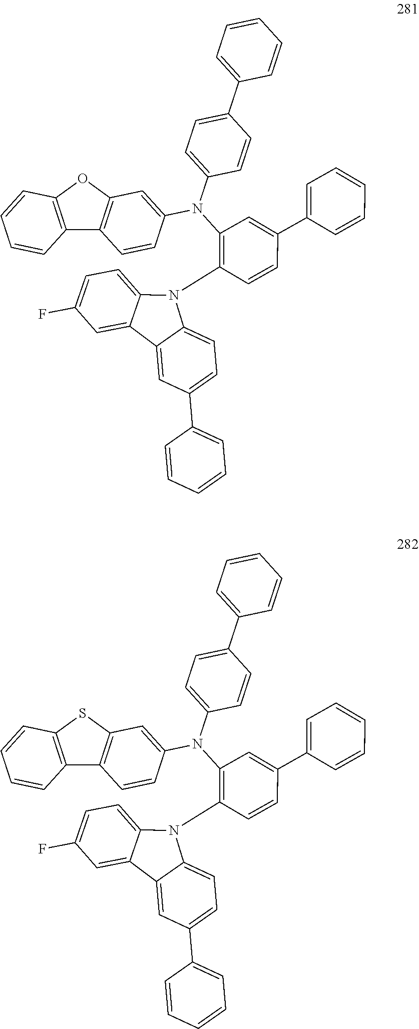 [SMILES: Fc1ccc2c(c1)c1cc(-c3ccccc3)ccc1n2-c1ccc(-c2ccccc2)cc1N(c1ccc(-c2ccccc2)cc1)c1ccc2c(c1)oc1ccccc12 ; Fc1ccc2c(c1)c1cc(-c3ccccc3)ccc1n2-c1ccc(-c2ccccc2)cc1N(c1ccc(-c2ccccc2)cc1)c1ccc2c(c1)sc1ccccc12]

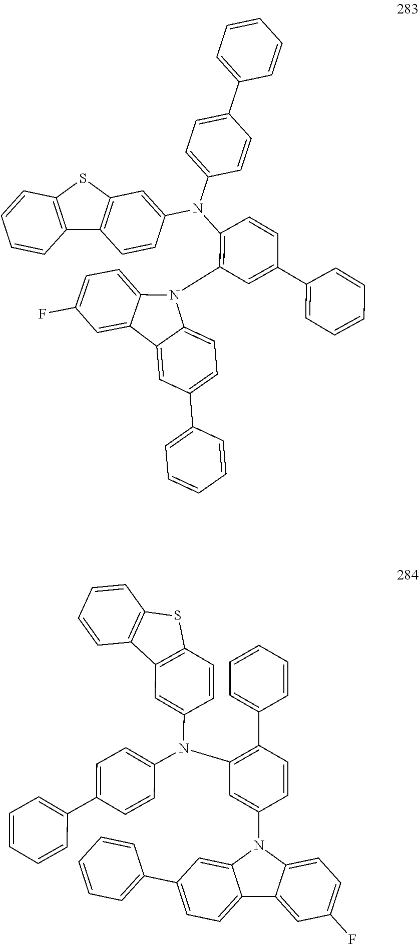 [SMILES: Fc1ccc2c(c1)c1cc(-c3ccccc3)ccc1n2-c1cc(-c2ccccc2)ccc1N(c1ccc(-c2ccccc2)cc1)c1ccc2c(c1)sc1ccccc12 ; Fc1ccc2c(c1)c1ccc(-c3ccccc3)cc1n2-c1ccc(-c2ccccc2)c(N(c2ccc(-c3ccccc3)cc2)c2ccc3sc4ccccc4c3c2)c1]

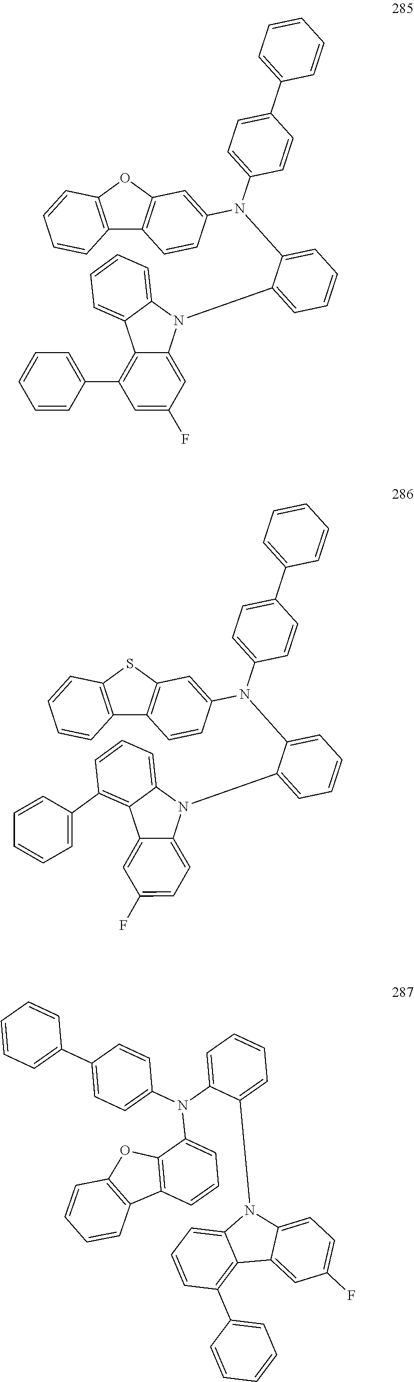 [SMILES: Fc1cc(-c2ccccc2)c2c3ccccc3n(-c3ccccc3N(c3ccc(-c4ccccc4)cc3)c3ccc4c(c3)oc3ccccc34)c2c1 ; Fc1ccc2c(c1)c1c(-c3ccccc3)cccc1n2-c1ccccc1N(c1ccc(-c2ccccc2)cc1)c1ccc2c(c1)sc1ccccc12 ; Fc1ccc2c(c1)c1c(-c3ccccc3)cccc1n2-c1ccccc1N(c1ccc(-c2ccccc2)cc1)c1cccc2c1oc1ccccc12]

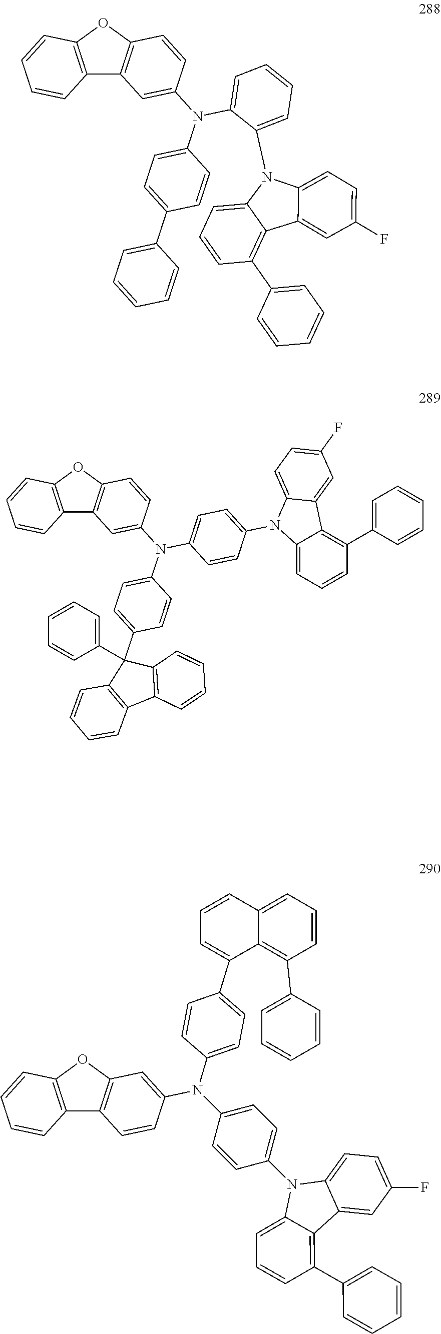 [SMILES: Fc1ccc2c(c1)c1c(-c3ccccc3)cccc1n2-c1ccccc1N(c1ccc(-c2ccccc2)cc1)c1ccc2oc3ccccc3c2c1 ; Fc1ccc2c(c1)c1c(-c3ccccc3)cccc1n2-c1ccc(N(c2ccc(C3(c4ccccc4)c4ccccc4-c4ccccc43)cc2)c2ccc3oc4ccccc4c3c2)cc1 ; Fc1ccc2c(c1)c1c(-c3ccccc3)cccc1n2-c1ccc(N(c2ccc(-c3cccc4cccc(-c5ccccc5)c34)cc2)c2ccc3c(c2)oc2ccccc23)cc1]

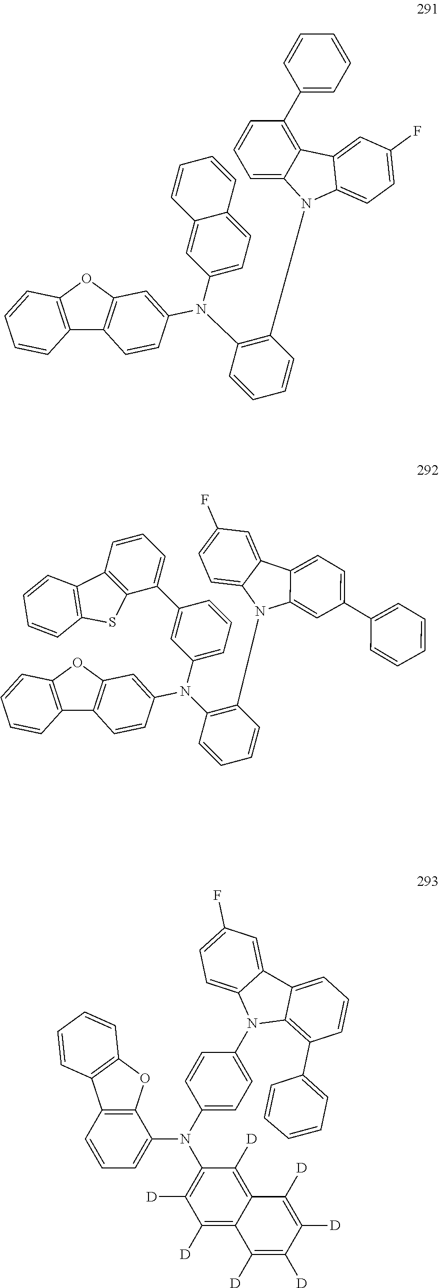 [SMILES: Fc1ccc2c(c1)c1c(-c3ccccc3)cccc1n2-c1ccccc1N(c1ccc2ccccc2c1)c1ccc2c(c1)oc1ccccc12 ; Fc1ccc2c(c1)c1ccc(-c3ccccc3)cc1n2-c1ccccc1N(c1cccc(-c2cccc3c2sc2ccccc23)c1)c1ccc2c(c1)oc1ccccc12 ; [2H]c1c([2H])c([2H])c2c([2H])c(N(c3ccc(-n4c5ccc(F)cc5c5cccc(-c6ccccc6)c54)cc3)c3cccc4c3oc3ccccc34)c([2H])c([2H])c2c1[2H]]

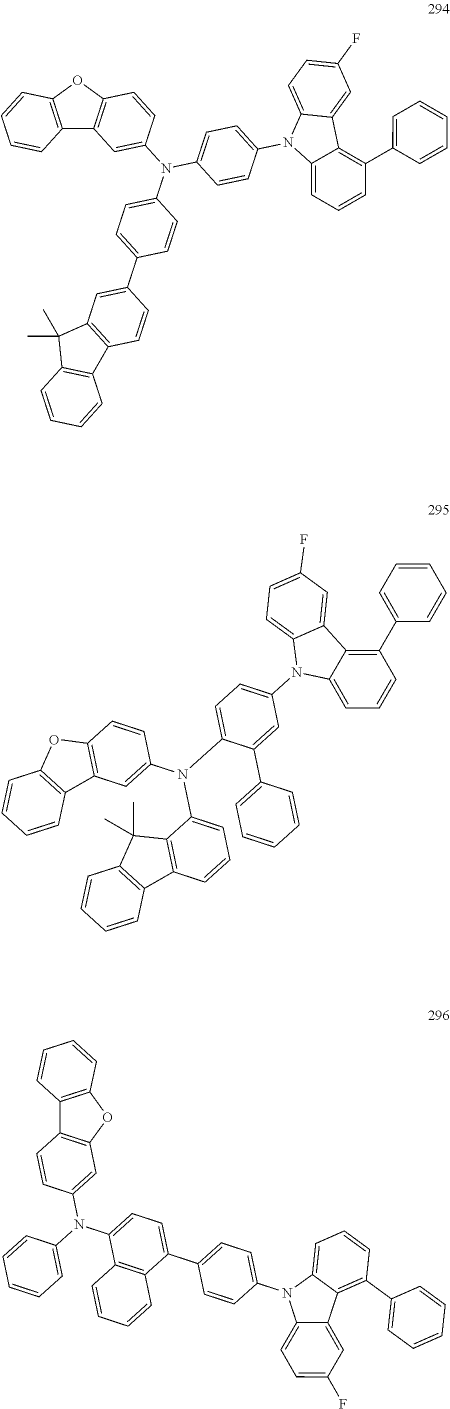 [SMILES: CC1(C)c2ccccc2-c2ccc(-c3ccc(N(c4ccc(-n5c6ccc(F)cc6c6c(-c7ccccc7)cccc65)cc4)c4ccc5oc6ccccc6c5c4)cc3)cc21 ; CC1(C)c2ccccc2-c2cccc(N(c3ccc4oc5ccccc5c4c3)c3ccc(-n4c5ccc(F)cc5c5c(-c6ccccc6)cccc54)cc3-c3ccccc3)c21 ; Fc1ccc2c(c1)c1c(-c3ccccc3)cccc1n2-c1ccc(-c2ccc(N(c3ccccc3)c3ccc4c(c3)oc3ccccc34)c3ccccc23)cc1]

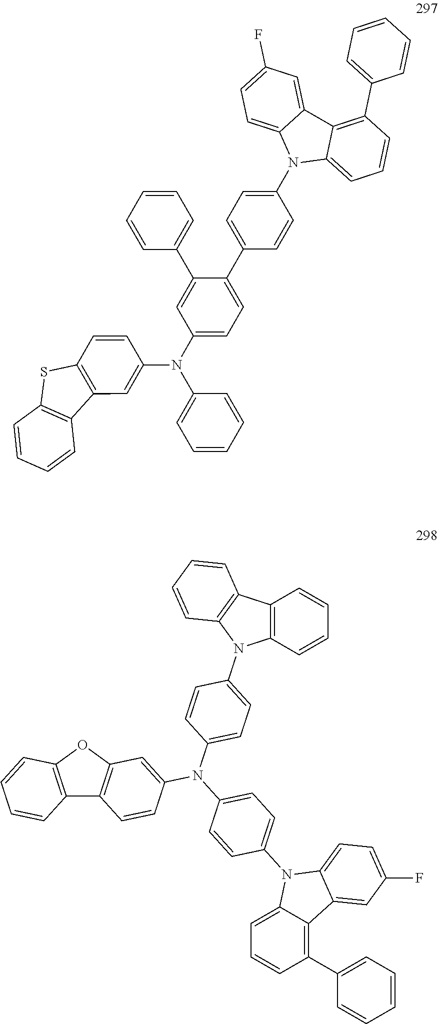 [SMILES: Fc1ccc2c(c1)c1c(-c3ccccc3)cccc1n2-c1ccc(-c2ccc(N(c3ccccc3)c3ccc4sc5ccccc5c4c3)cc2-c2ccccc2)cc1 ; Fc1ccc2c(c1)c1c(-c3ccccc3)cccc1n2-c1ccc(N(c2ccc(-n3c4ccccc4c4ccccc43)cc2)c2ccc3c(c2)oc2ccccc23)cc1]

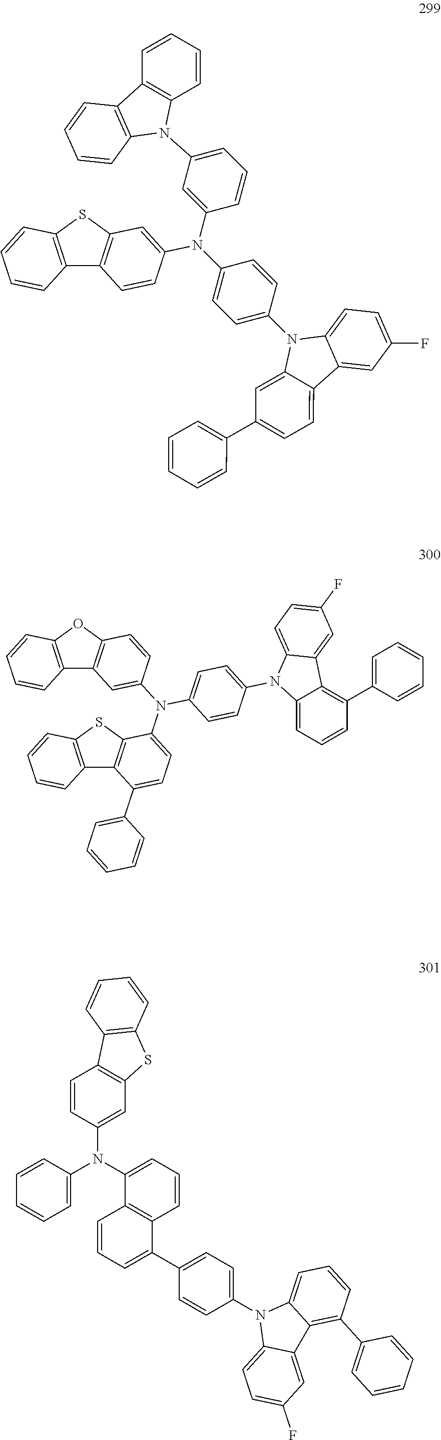 [SMILES: Fc1ccc2c(c1)c1ccc(-c3ccccc3)cc1n2-c1ccc(N(c2cccc(-n3c4ccccc4c4ccccc43)c2)c2ccc3c(c2)sc2ccccc23)cc1 ; Fc1ccc2c(c1)c1c(-c3ccccc3)cccc1n2-c1ccc(N(c2ccc3oc4ccccc4c3c2)c2ccc(-c3ccccc3)c3c2sc2ccccc23)cc1 ; Fc1ccc2c(c1)c1c(-c3ccccc3)cccc1n2-c1ccc(-c2cccc3c(N(c4ccccc4)c4ccc5c(c4)sc4ccccc45)cccc23)cc1]

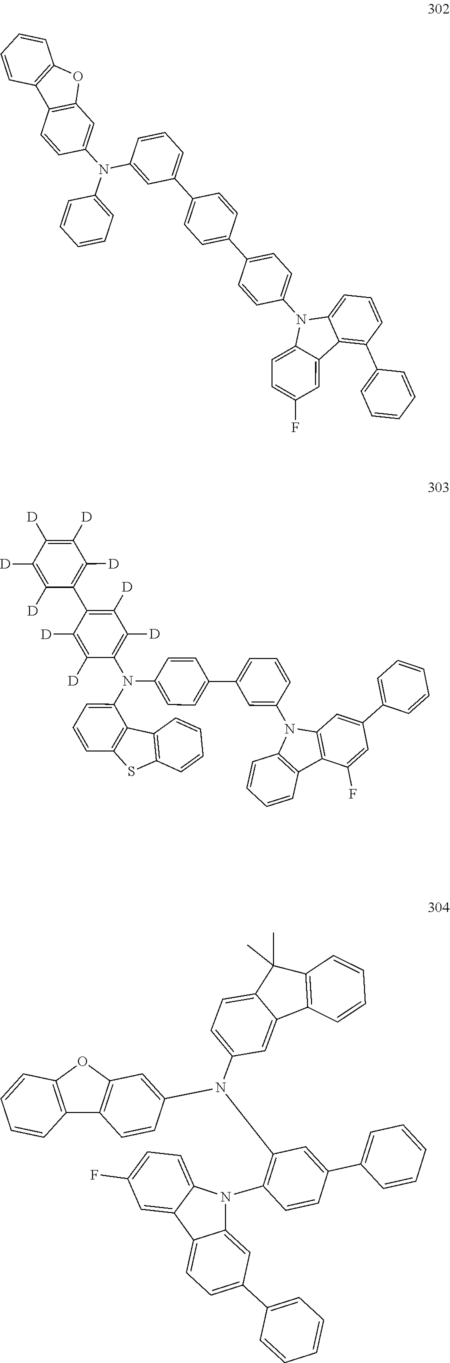 [SMILES: Fc1ccc2c(c1)c1c(-c3ccccc3)cccc1n2-c1ccc(-c2ccc(-c3cccc(N(c4ccccc4)c4ccc5c(c4)oc4ccccc45)c3)cc2)cc1 ; [2H]c1c([2H])c([2H])c(-c2c([2H])c([2H])c(N(c3ccc(-c4cccc(-n5c6ccccc6c6c(F)cc(-c7ccccc7)cc65)c4)cc3)c3cccc4sc5ccccc5c34)c([2H])c2[2H])c([2H])c1[2H] ; CC1(C)c2ccccc2-c2cc(N(c3ccc4c(c3)oc3ccccc34)c3cc(-c4ccccc4)ccc3-n3c4ccc(F)cc4c4ccc(-c5ccccc5)cc43)ccc21]

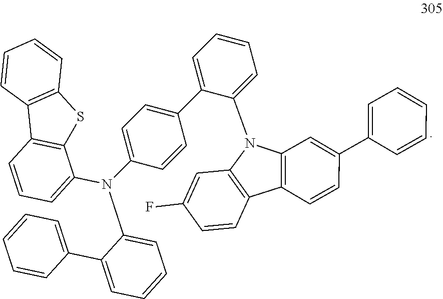 [SMILES: Fc1ccc2c3ccc(-c4ccccc4)cc3n(-c3ccccc3-c3ccc(N(c4ccccc4-c4ccccc4)c4cccc5c4sc4ccccc45)cc3)c2c1]

13. An organic electroluminescent device, comprising an anode, a cathode, and a functional layer disposed between the anode and the cathode, characterized in that the functional layer comprises the organic compound according to claim 1.

14. The organic electroluminescent device according to claim 13, wherein the functional layer comprises a hole transport layer and the hole transport comprises the organic compound;
optionally, the hole transport layer comprises a first hole transport layer and a second hole transport layer, and the first hole transport layer is closer to the anode than the second hole transport layer, wherein the second hole transport layer comprises the organic compound.

15. An electronic apparatus, comprising the organic electroluminescent device according to claim 13.
